(19) United States
(12) Patent Application Publication
(10) Pub. No.: US 2021/0184133 A1
(43) Pub. Date: Jun. 17, 2021

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Su-Hyun Lee, Gyeonggi-do (KR); So-Young JUNG, Gyeonggi-do (KR); Jin-Ri HONG, Gyeonggi-do (KR); Sang-Hee CHO, Gyeonggi-do (KR)

(21) Appl. No.: 17/114,005

(22) Filed: Dec. 7, 2020

(30) Foreign Application Priority Data
Dec. 16, 2019 (KR) 10-2019-0167626
Oct. 26, 2020 (KR) 10-2020-0139350

(51) Int. Cl. H01L 51/00

(52) U.S. Cl. CPC H01L 51/0069; H01L 51/006; H01L 51/0094; H01L 51/0067; H01L 51/5056; H01L 51/0074; H01L 51/0055; H01L 51/0061; H01L 51/0054; H01L 51/0073

(57) ABSTRACT

The present disclosure relates to a plurality of host materials comprising a first host material comprising a compound represented by formula 1 and a second host material comprising a compound represented by formula 2 and an organic electroluminescent device comprising the same. By comprising an organic electroluminescent compound according to the present disclosure as an organic electroluminescent material or the specific combination of the compound as host materials, an organic electroluminescent device having high luminous efficiency and/or long lifespan can be provided compared with a conventional organic electroluminescent device.

### TECHNICAL FIELD

The present disclosure relates to an organic electroluminescent compound, a plurality of host materials, and an organic electroluminescent device comprising the same.

### BACKGROUND ART

The TFD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an organic electroluminescent device have been rapidly commercialized. At present, an organic electroluminescent device mainly includes phosphorescent materials having excellent luminous efficiency in panel realization. For prolonged use and high resolution of the display, an OLED having high luminous efficiency and/or long lifespan is necessary.

KR 2017-0043439 A discloses a plurality of host materials using a compound such as a carbazole derivative; KR 2017-022865 A and KR 2018-0099487 A disclose a host compound having a phenanthrooxazole-based and/or phenanthrothiazole-based compound as a basic skeleton. However, said references do not specifically disclose a plurality of host materials as described in the present disclosure. In addition, compounds in which only substituents having hole characteristics are introduced into their basic skeletons, such as the host compounds disclosed in said references, have strong hole injection and transport characteristics, and thus, when used alone as a host material in a light-emitting layer, electron injection and transport characteristics in the light-emitting layer are insufficient. As a result, the charge is not balanced, which can cause a rapid decrease in efficiency and lifespan.

Therefore, there is still a need for development of a light-emitting material having improved performance, such as improved luminous efficiency and/or lifespan, compared to the conventional host material disclosed in said references.

### DISCLOSURE OF THE INVENTION

### Technical Problem

The object of the present disclosure is firstly, to provide a plurality of host materials which is able to produce an organic electroluminescent device having high luminous efficiency, and/or long lifespan, and secondly, to provide an organic electroluminescent device comprising the host materials. The other object of the present disclosure is to provide an organic electroluminescent compound having a new structure suitable for use as an organic electroluminescent material.

### Solution to Problem

As a result of intensive studies to solve the technical problem above, the present inventors found that the aforementioned objective can be achieved by a plurality of host materials comprising a first host material comprising a compound represented by the following formula 1 and a second host material comprising a compound represented by the following formula 2, so that the present invention was completed.

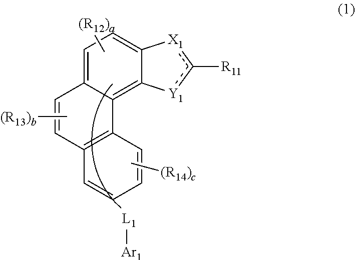

wherein,

X₁ and Y₁ each independently represent, —N═, —NR₅—, —O—, or —S—, provided that any one of X₁ and Y₁ is —N═, and other of X₁ and Y₁ is —NR₅—, —O— or —S—;

L₁ represents a single bond or a substituted or unsubstituted (C6-C30)arylene;

Ar₁ represents a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, or a substituted or unsubstituted (C6-C30)aryl(3- to 30-membered)heteroarylamino;

R₁₁ represents a substituted or unsubstituted (C6-C30)aryl or a substituted or unsubstituted (3- to 30-membered)heteroaryl;

R₁₂ to R₁₄ and R₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to the adjacent substituents to form a ring(s); and

a and b each independently represent, an integer of 1 or 2, c represents an integer of 1 to 4, and when a to c represent an integer of 2 or more, each of R₁ to R₁₄ may be the same or different;

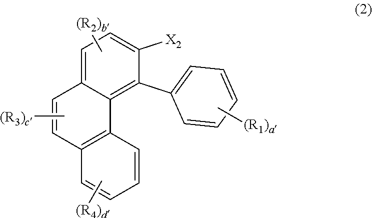

wherein,

X represents O, S, or CR₇R₈;

R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;

at least one of R₁ to R₄ is a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; provided that the case where any one of R₁ to R₄ is triphenylene is excluded;

R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s); and

a′ and d′ each independently represent, an integer of 1 to 4, b′ and c′ each independently represent, an integer of 1 or 2, and when a′ to d′ represent an integer of 2 or more, each of R₁ to R₄ may be the same or different.

### Advantageous Effects of Invention

By comprising an organic electroluminescent compound as an organic electroluminescent material or the specific combination of the compound as host materials according to the present disclosure, an organic electroluminescent device having a high luminous efficiency and/or long lifespan can be provided as compared with a conventional organic electroluminescent device, and a display device or a lighting device using the same can be manufactured.

### MODE FOR THE INVENTION

Hereinafter, the present disclosure will be described in detail. However, the following description is intended to explain the invention, and is not meant in any way to restrict the scope of the invention.

The present disclosure relates to a plurality of host materials comprising at least one first host material represented by the formula 1 and at least one second host material represented by the formula 2, and an organic electroluminescent device comprising the host materials.

The present disclosure relates to an organic electroluminescent compound represented by formula 2′ and/or formula 2″, an organic electroluminescent material comprising the organic electroluminescent compound, and an organic electroluminescent device comprising the organic electroluminescent compound.

The term “organic electroluminescent compound” in the present disclosure means a compound that may be used in an organic electroluminescent device, and may be comprised in any material layer constituting an organic electroluminescent device, as necessary.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers, and may be mixture-evaporated or co-evaporated, or may be individually evaporated.

Herein, “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-guaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 3 to 30, more preferably 5 to 20. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s) and may include a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrirnidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzothazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 5-indolyl, 6-indolyl, 7-indolyl, 1-isoindolyl, 2-isoindolyl, 3-isoindolyl, 4-isoindolyl, 5-isoindolyl, 6 isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 5-quinolyl, 6 quinolyl, 7-quinolyl, 8-quinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1 acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtha-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtha-[2,3-b]-benzofuranyl, 7-naphtho-[2,3-b]-benzofuranyl, 8-naphtha-[2,3-b]-benzofuranyl, 9-naphtha-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtha-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtha-[2,1-b]-benzofuranyl, 10-naphtha-[2,1-b]-benzofuranyl, 1-naphtho-[1,2-b]-benzothiophenyl, 2-naphtha-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtha-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtha-[2,3-b]-benzothiophenyl, 3-naphtho-[2,3-b]-benzothiophenyl, 4-naphtha-[2,3-b]-benzothiophenyl, 5-naphtho-[2,3-b]-benzothiophenyl, 1-naphtho-[2,1-b]-benzothiophenyl, 2-naphtha-[2,1-b]-benzothiophenyl, 3-naphtho-[2,1-b]-benzothiophenyl, 4-naphtha-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, “fused ring of (C3-C30)aliphatic ring and (C6-C30)aromatic ring” means a functional group of a ring in which at least one aliphatic ring having 3 to 30 ring backbone atoms, preferably 3 to 25, more preferably 3 to 18, and at least one aromatic ring having 6 to 30 ring backbone atoms, preferably 6 to 25, more preferably 6 to 18, are fused, e.g., a fused ring of at least one benzene and at least one cyclohexane, or a fused ring of at least one naphthalene and at least one cyclopentane. Wherein a carbon atom(s) of fused ring of (C3-C30)aliphatic ring and (C6-C30)aromatic ring may be replaced at least one heteroatom(s) selected from the group consisting of B, N, O, S, Si and P, preferably N, O and S. Herein, “Halogen” includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (5- to 25-membered) mono- or polycyclic, alicyclic, aromatic ring, or a combination thereof. Further, the formed ring may be included at least one heteroatom selected from the group consisting of B, N, O, S, Si and P, preferably, N, O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine, That is, pyridine-triazine may be heteroaryl or may be interpreted as one substituent in which two heteroaryls are connected. The substituents of the substituted (C1-C30)alkyl, the substituted (C6-C30)aryl(ene), the substituted (3- to 30-membered)heteroaryl(ene), the substituted (C1-C30)alkoxy, the fused ring of the substituted (C3-C30) aliphatic ring and the (C6-C30) aromatic ring, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C2-C30)alkenylamino, the substituted (C1-C30)alkyl(C2-C30)alkenylamino, the substituted mono- or di-(C6-C30)arylamino, the substituted (C1-C30)alkyl(C6-C30)arylamino, the substituted mono- or di-(3- to 30-membered)heteroarylamino, the substituted (C1-C30)alkyl(3- to 30-membered)heteroarylamino, the substituted (C2-C30)alkenyl(C6-C30)arylamino, the substituted (C2-C30)alkenyl(3- to 30-membered)heteroarylamino, the substituted (C6-C30)aryl(3- to 30-membered)heteroarylamino, and the substituted ring, in the formulas of the present disclosure, each independently represent, at least one selected from the group consisting of deuterium, halogen, cyano, carboxyl, nitro, hydroxy, phosphine oxide, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, a (5- to 30-membered)heteroaryl unsubstituted or substituted with (C6-C30)aryl, a (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, a mono- or di-(C6-C30)arylamino unsubstituted or substituted with (C1-C30)alkyl, (C1-C30)alkyl(C6-C30)arylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituents may be cyano, a substituted or unsubstituted methyl, a substituted or unsubstituted phenyl, a substituted or unsubstituted biphenyl, a substituted or unsubstituted naphthyl, a substituted or unsubstituted fluorenyl, a substituted or unsubstituted spirobifluorenyl, a substituted or unsubstituted phenanthrenyl, a substituted or unsubstituted dibenzofuranyl, a substituted or unsubstituted dibenzothiophenyl, or a substituted or unsubstituted carbazolyl, etc.

In the formulas of the present disclosure, heteroaryl(ene) each independently may contain at least one heteroatom selected from the group consisting of B, N, O, S, Si, and P. Further, the above heteroatom may be linked with at least one substituent selected from the group consisting of hydrogen, deuterium, halogen, cyano, a substituted or unsubstituted C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the host materials according to one embodiment will be described.

The host materials according to one embodiment comprise at least one first host compound represented by the above formula 1 and at least one second host compound represented by the above formula 2; and the host materials may be contained in the light-emitting layer of an organic electroluminescent device according to one embodiment.

The first host compound as the host material according to one embodiment may be represented by the following formula 1.

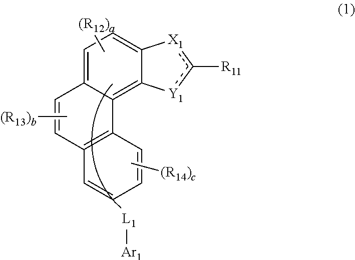

in formula 1,

X₁ and Y₁ each independently represent, —N═, —NR₅—, —O—, or —S—, provided that any one of X₁ and Y₁ is —N═, and other of X₁ and Y₁ is —NR₅—, —O— or —S—;

L₁ represents a single bond or a substituted or unsubstituted (C6-C30)arylene;

Ar₁ represents a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, or a substituted or unsubstituted (C6-C30)aryl(3- to 30-membered)heteroarylamino;

R₁₁ represents a substituted or unsubstituted (C6-C30)aryl or a substituted or unsubstituted (3- to 30-membered)heteroaryl;

R₁₂ to R₁₄ and R₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to the adjacent substituents to form a ring(s); and

a and b each independently represent, an integer of 1 or 2, c represents an integer of 1 to 4, and when a to c represent an integer of 2 or more, each of R₁₂ to R₁₄ may be the same or different.

According to one embodiment, the host material represented by the above formula 1 may be represented by any one of the following formulas 1-1 to 1-3.

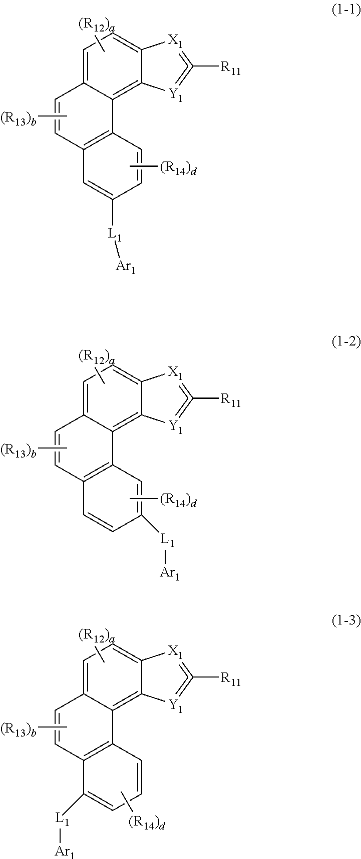

In formulas 1-1 to 1-3,

X₁, Y₁, Ar₁, L₁, R₁₁ to R₁₄, a, and b are as defined in the formula 1;

d represents an integer of 1 to 3, and when d represent an integer of 2 or more, each of R₁₄ may be the same or different.

In one embodiment, X₁ and Y₁ each independently represent, —N═, —NR₅—, —O—, or —S—, provided that any one of X₁ and Y₁ is —N═, and other of X₁ and Y₁ is —NR₅—, —O— or —S—. Preferably, when X₁ is —N═, Y₁ may be —NR₅—, —O—, or —S—, or when Y₁ is —N═, X₁ may be —NR₅—, —O—, or —S—. More preferably, when X₁ is —N═, Y₁ may be —O— or —S—, or when Y₁ is —N═, X₁ may be —NR₅—, —O—, or —S—. In this case, R₅ may be hydrogen, deuterium, halogen, cyano, a substituted or unsubstituted (C1-C30)alkyl, a substituted or unsubstituted (C6-C30)aryl, or a substituted or unsubstituted (3- to 30-membered)heteroaryl, preferably, hydrogen, deuterium, halogen, cyano, a substituted or unsubstituted (C1-C10)alkyl, or a substituted or unsubstituted (C6-C25)aryl, more preferably, hydrogen or a substituted or unsubstituted (C6-C18)aryl.

In one embodiment, L₁ represents a single bond or a substituted or unsubstituted (C6-C30)arylene, Preferably, L₁ may be a single bond or a substituted or unsubstituted (C6-C25)arylene, more preferably, a single bond or a substituted or unsubstituted (C6-C18)arylene. For example, L₁ may be a single bond, or a substituted or unsubstituted phenylene, a substituted or unsubstituted naphthylene, a substituted or unsubstituted o-biphenylene, a substituted or unsubstituted m-biphenylene, or a substituted or unsubstituted p-biphenylene.

In one embodiment, An represents a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, or a substituted or unsubstituted (C6-C30)aryl(3- to 30-membered)heteroarylamino. Preferably, An may be a substituted or unsubstituted (C6-C30)aryl, a substituted or unsubstituted (5- to 30-membered)heteroaryl, a substituted or unsubstituted di(C6-C25)arylamino, or a substituted or unsubstituted (C6-C25)aryl(5- to 25-membered)heteroarylamino, more preferably, a substituted or unsubstituted (C6-C25)aryl, a substituted or unsubstituted (5- to 25-membered)heteroaryl comprising at least one nitrogen, a substituted or unsubstituted di(C6-C18)arylamino, or a substituted or unsubstituted (C6-C18)aryl(5- to 18-membered)heteroarylamino. For example, An may be a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, a substituted or unsubstituted phenanthrenyl, a substituted or unsubstituted benzophenanthrenyl, a substituted or unsubstituted fluoranthenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted spiro[fluoren-benzofluoren]yl, a substituted or unsubstituted pyrimidyl, a substituted or unsubstituted triazinyl, a substituted or unsubstituted quinazolinyl, a substituted or unsubstituted quinoxalinyl, a substituted or unsubstituted benzofuropyrimidinyl, or an amino substituted with at least one selected from the group consisting of a substituted or unsubstituted phenyl, naphthyl, o-biphenyl, m-biphenyl, p-biphenyl, terphenyl, phenanthrenyl, substituted or unsubstituted fluorenyl, a substituted or unsubstituted carbazolyl, dibenzofuranyl, and dibenzothiophenyl.

In one embodiment, Rif represents a substituted or unsubstituted (C6-C30)aryl or a substituted or unsubstituted (3- to 30-membered)heteroaryl, preferably, R₁₁ may be a substituted or unsubstituted (C6-C25)aryl or a substituted or unsubstituted (5- to 25-membered)heteroaryl, more preferably, a substituted or unsubstituted (C6-C18)aryl or a substituted or unsubstituted (5- to 18-membered)heteroaryl. For example, R₁₁ may be a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted fluorenyl, a substituted or unsubstituted dibenzothiophenyl, a substituted or unsubstituted pyridyl, a substituted or unsubstituted quinolinyl, or a substituted or unsubstituted isoquinolinyl.

In one embodiment, R₁₂ to R₁₁ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, or may be linked to the adjacent substituents to form a ring(s). Preferably, R₁₂ to R₁₄ each independently may be hydrogen, deuterium, halogen, cyano, a substituted or unsubstituted (C1-C10)alkyl, a substituted or unsubstituted (C6-C25)aryl, or a substituted or unsubstituted (5- to 25-membered)heteroaryl, more preferably, hydrogen, deuterium, halogen, cyano, or a substituted or unsubstituted (C1-C10)alkyl.

In one embodiment, the first host material represented by the above formula 1 may be more specifically illustrated by the following compounds, but is not limited thereto.

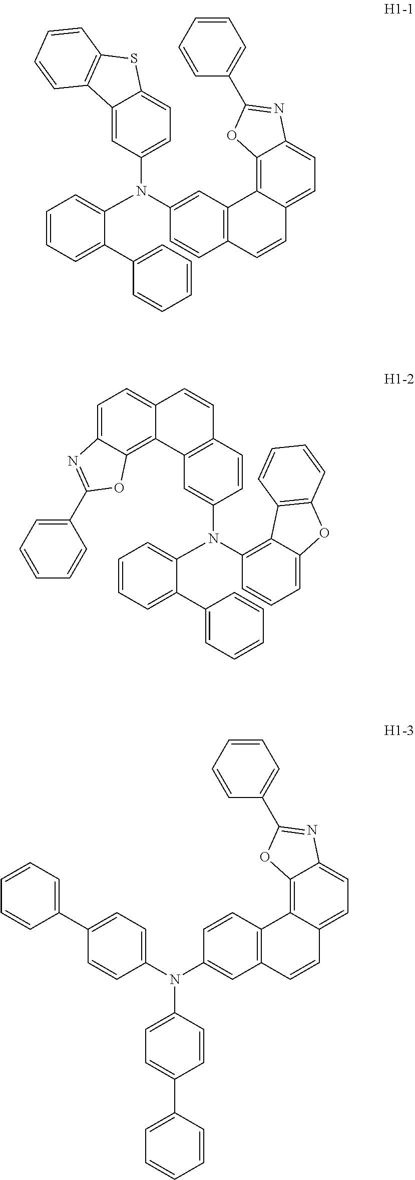 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

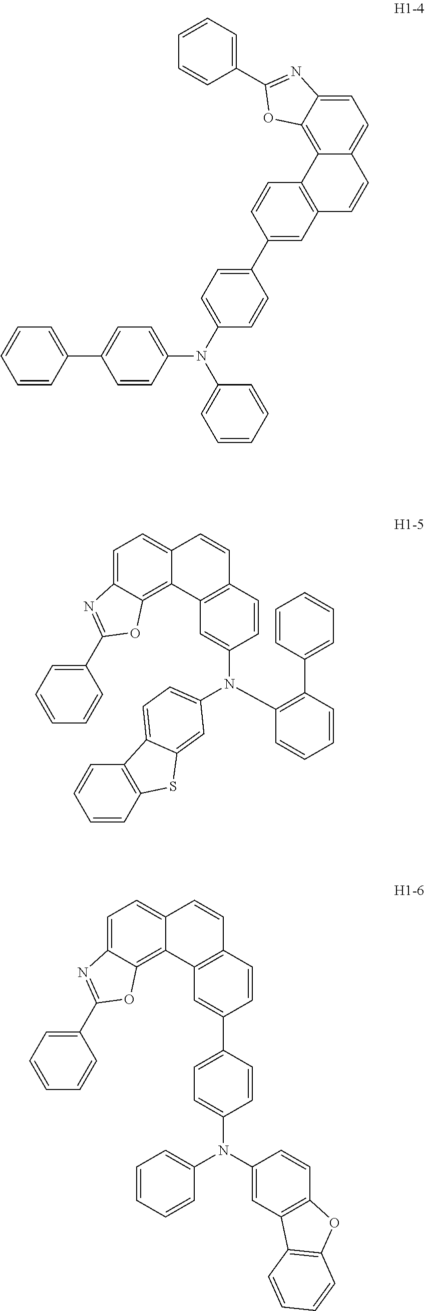 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

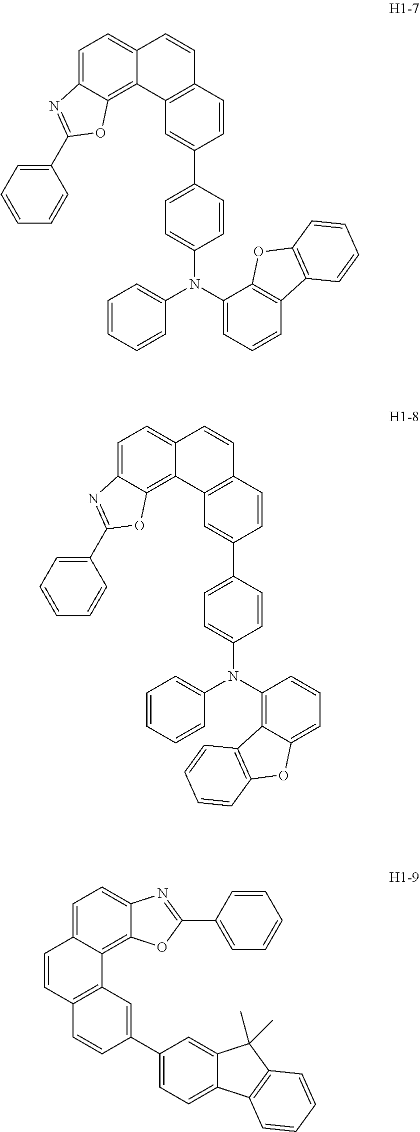 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; CC1(C)c2ccccc2-c2ccc(-c3ccc4ccc5ccc6nc(-c7ccccc7)oc6c5c4c3)cc21]

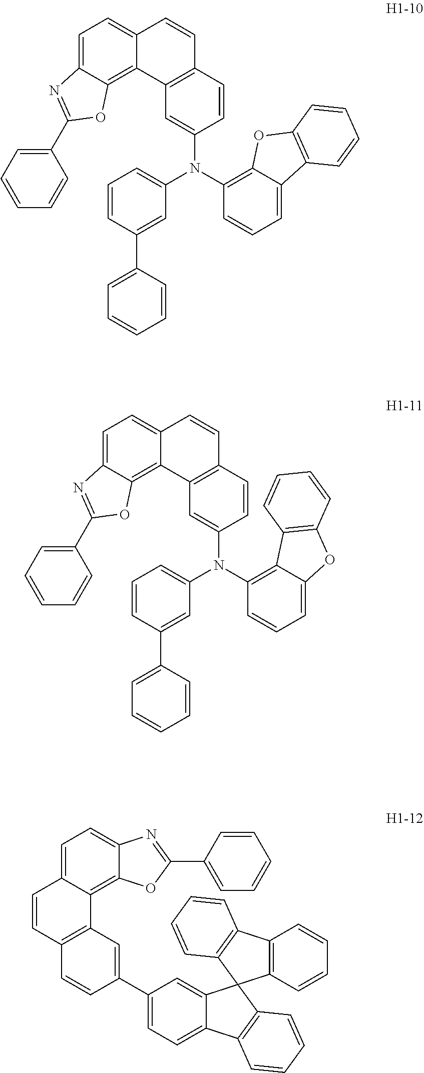 [SMILES: c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2nc3ccc4ccc5ccc(-c6ccc7c(c6)C6(c8ccccc8-c8ccccc86)c6ccccc6-7)cc5c4c3o2)cc1]

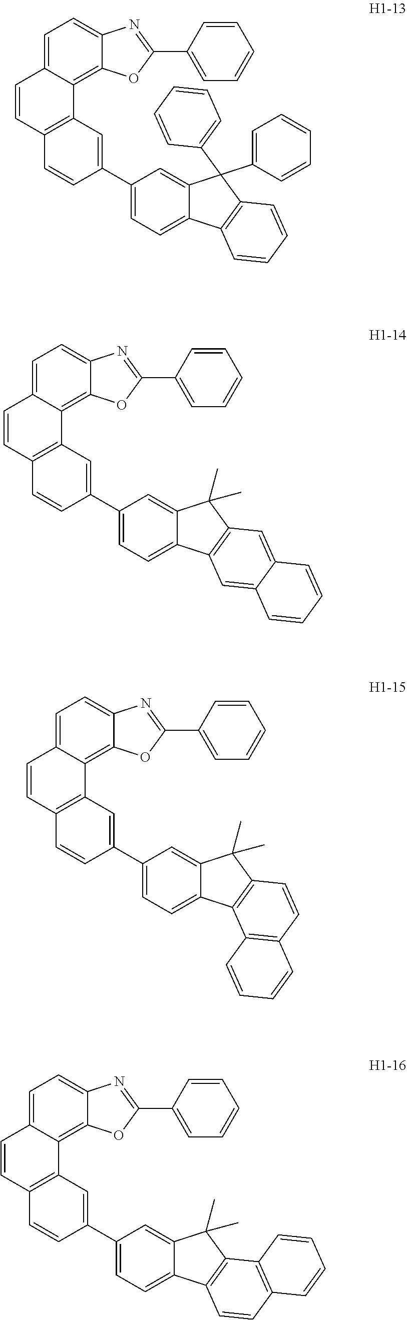 [SMILES: c1ccc(-c2nc3ccc4ccc5ccc(-c6ccc7c(c6)C(c6ccccc6)(c6ccccc6)c6ccccc6-7)cc5c4c3o2)cc1 ; CC1(C)c2cc(-c3ccc4ccc5ccc6nc(-c7ccccc7)oc6c5c4c3)ccc2-c2cc3ccccc3cc21 ; CC1(C)c2cc(-c3ccc4ccc5ccc6nc(-c7ccccc7)oc6c5c4c3)ccc2-c2c1ccc1ccccc21 ; CC1(C)c2cc(-c3ccc4ccc5ccc6nc(-c7ccccc7)oc6c5c4c3)ccc2-c2ccc3ccccc3c21]

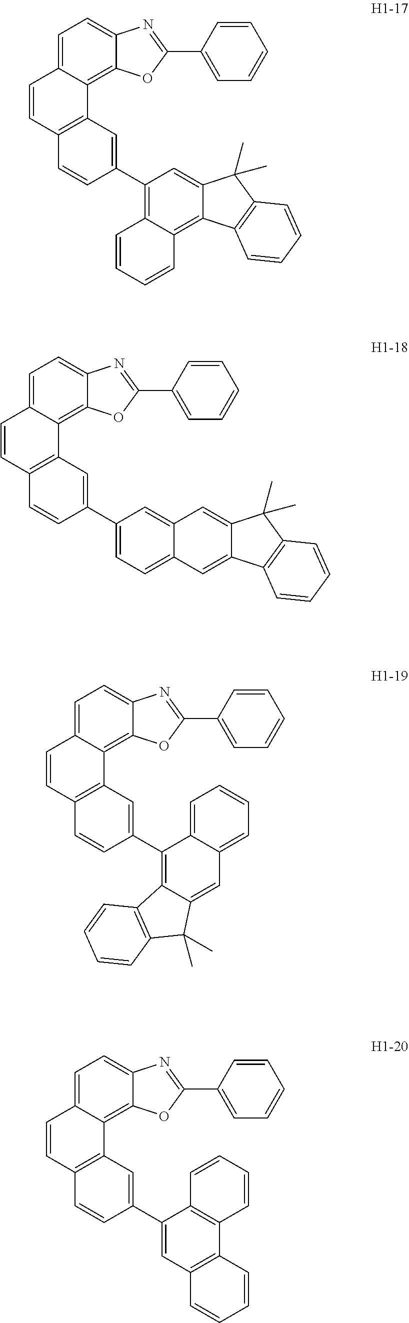 [SMILES: CC1(C)c2ccccc2-c2c1cc(-c1ccc3ccc4ccc5nc(-c6ccccc6)oc5c4c3c1)c1ccccc21 ; CC1(C)c2ccccc2-c2cc3ccc(-c4ccc5ccc6ccc7nc(-c8ccccc8)oc7c6c5c4)cc3cc21 ; CC1(C)c2ccccc2-c2c1cc1ccccc1c2-c1ccc2ccc3ccc4nc(-c5ccccc5)oc4c3c2c1 ; c1ccc(-c2nc3ccc4ccc5ccc(-c6cc7ccccc7c7ccccc67)cc5c4c3o2)cc1]

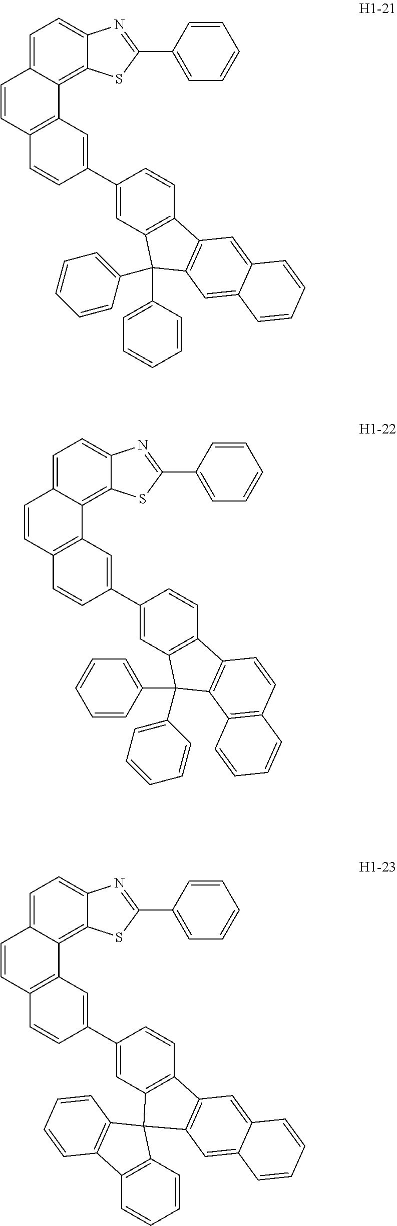 [SMILES: c1ccc(-c2nc3ccc4ccc5ccc(-c6ccc7c(c6)C(c6ccccc6)(c6ccccc6)c6cc8ccccc8cc6-7)cc5c4c3s2)cc1 ; c1ccc(-c2nc3ccc4ccc5ccc(-c6ccc7c(c6)C(c6ccccc6)(c6ccccc6)c6c-7ccc7ccccc67)cc5c4c3s2)cc1 ; c1ccc(-c2nc3ccc4ccc5ccc(-c6ccc7c(c6)C6(c8ccccc8-c8ccccc86)c6cc8ccccc8cc6-7)cc5c4c3s2)cc1]

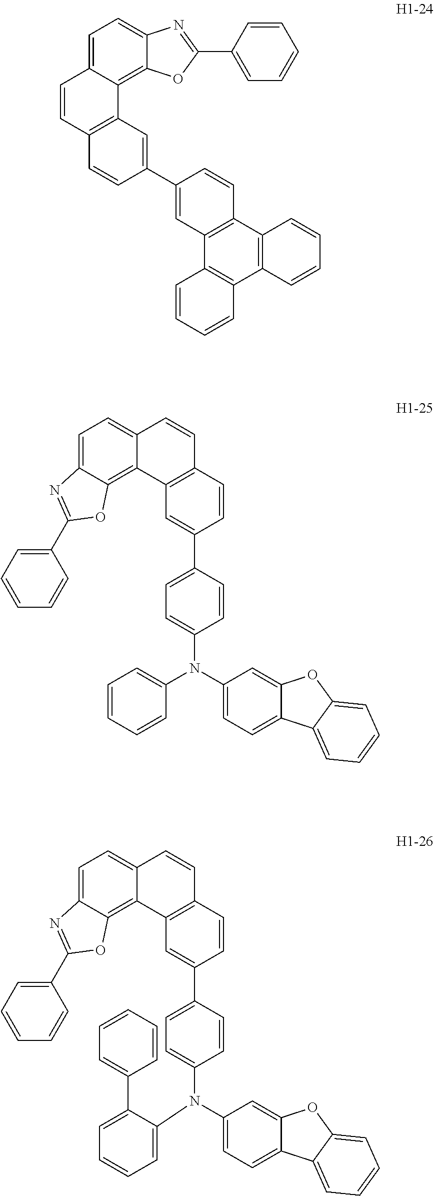 [SMILES: c1ccc(-c2nc3ccc4ccc5ccc(-c6ccc7c8ccccc8c8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1]

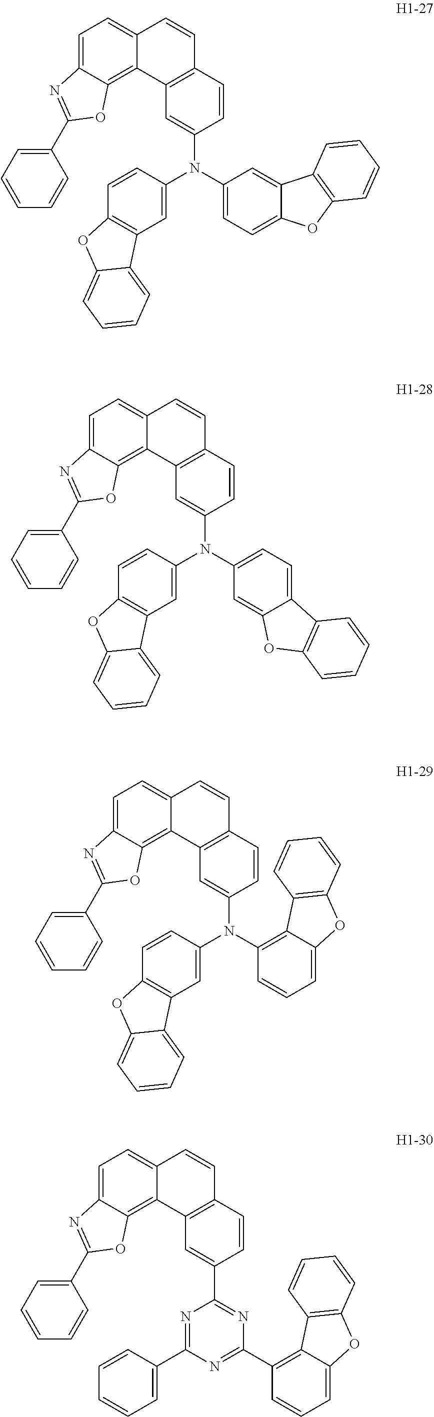 [SMILES: c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc(-c3ccc4ccc5ccc6nc(-c7ccccc7)oc6c5c4c3)nc(-c3cccc4oc5ccccc5c34)n2)cc1]

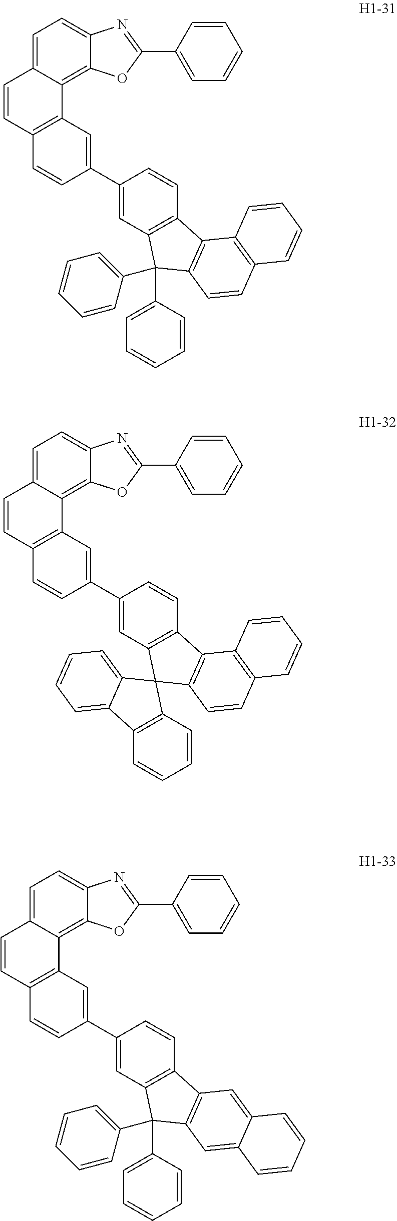 [SMILES: c1ccc(-c2nc3ccc4ccc5ccc(-c6ccc7c(c6)C(c6ccccc6)(c6ccccc6)c6ccc8ccccc8c6-7)cc5c4c3o2)cc1 ; c1ccc(-c2nc3ccc4ccc5ccc(-c6ccc7c(c6)C6(c8ccccc8-c8ccccc86)c6ccc8ccccc8c6-7)cc5c4c3o2)cc1 ; c1ccc(-c2nc3ccc4ccc5ccc(-c6ccc7c(c6)C(c6ccccc6)(c6ccccc6)c6cc8ccccc8cc6-7)cc5c4c3o2)cc1]

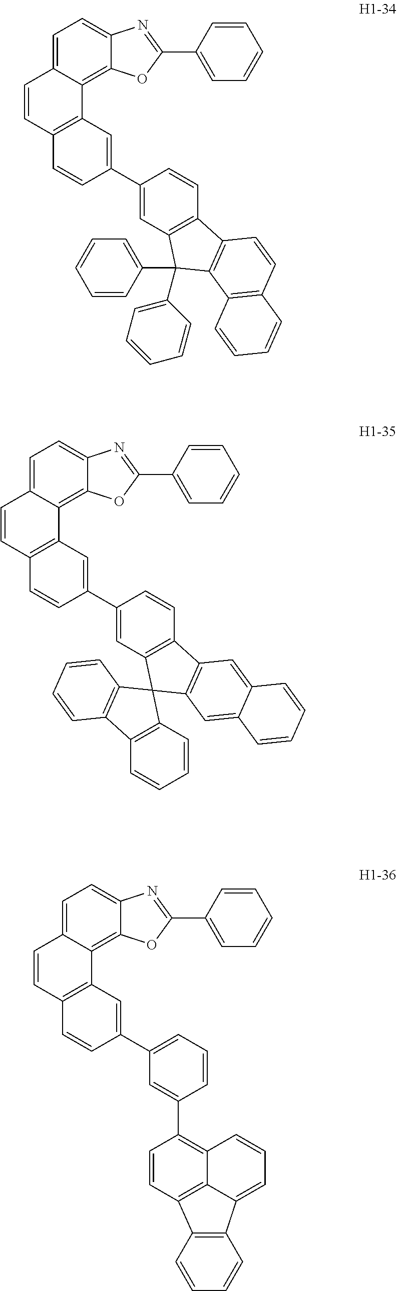 [SMILES: c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1 ; c1ccc(-c2nc3ccc4ccc5ccc(-c6cccc(-c7ccc8c9c(cccc79)-c7ccccc7-8)c6)cc5c4c3o2)cc1]

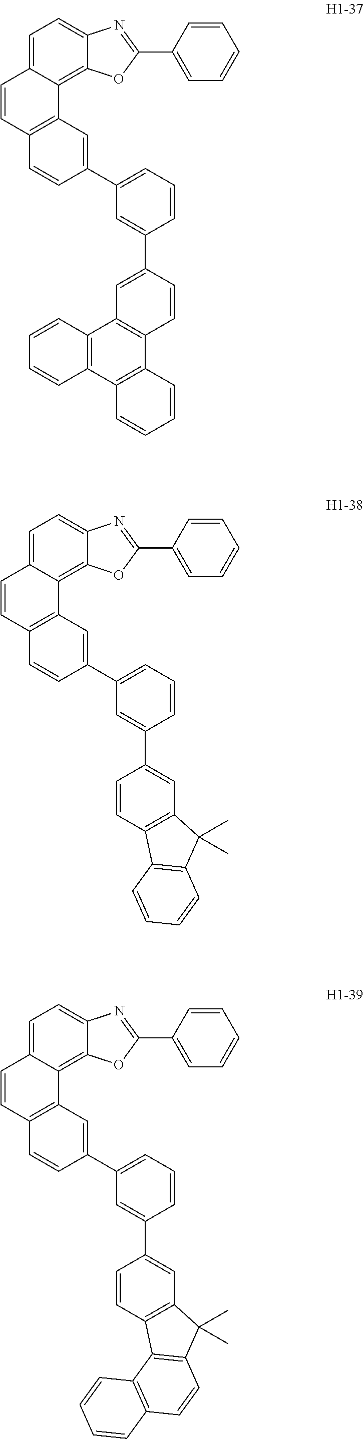 [SMILES: c1ccc(-c2nc3ccc4ccc5ccc(-c6cccc(-c7ccc8c9ccccc9c9ccccc9c8c7)c6)cc5c4c3o2)cc1 ; CC1(C)c2ccccc2-c2ccc(-c3cccc(-c4ccc5ccc6ccc7nc(-c8ccccc8)oc7c6c5c4)c3)cc21 ; CC1(C)c2cc(-c3cccc(-c4ccc5ccc6ccc7nc(-c8ccccc8)oc7c6c5c4)c3)ccc2-c2c1ccc1ccccc21]

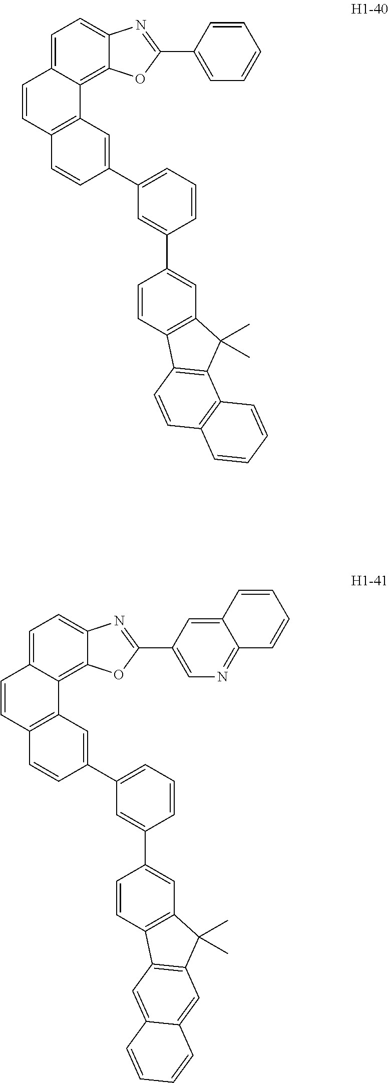 [SMILES: CC1(C)c2cc(-c3cccc(-c4ccc5ccc6ccc7nc(-c8ccccc8)oc7c6c5c4)c3)ccc2-c2ccc3ccccc3c21 ; CC1(C)c2cc(-c3cccc(-c4ccc5ccc6ccc7nc(-c8cnc9ccccc9c8)oc7c6c5c4)c3)ccc2-c2cc3ccccc3cc21]

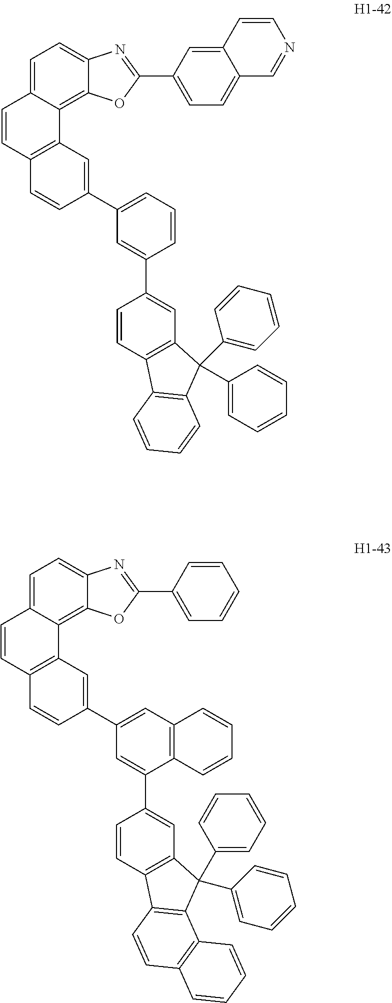 [SMILES: c1ccc(C2(c3ccccc3)c3ccccc3-c3ccc(-c4cccc(-c5ccc6ccc7ccc8nc(-c9ccc%10cnccc%10c9)oc8c7c6c5)c4)cc32)cc1 ; c1ccc(-c2nc3ccc4ccc5ccc(-c6cc(-c7ccc8c(c7)C(c7ccccc7)(c7ccccc7)c7c-8ccc8ccccc78)c7ccccc7c6)cc5c4c3o2)cc1]

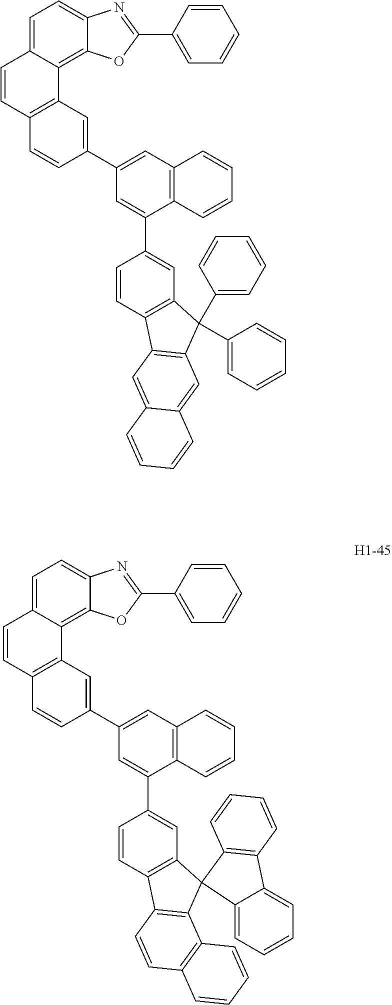 [SMILES: c1ccc(-c2nc3ccc4ccc5ccc(-c6cc(-c7ccc8c(c7)C(c7ccccc7)(c7ccccc7)c7cc9ccccc9cc7-8)c7ccccc7c6)cc5c4c3o2)cc1 ; c1ccc(-c2nc3ccc4ccc5ccc(-c6cc(-c7ccc8c(c7)C7(c9ccccc9-c9ccccc97)c7c-8ccc8ccccc78)c7ccccc7c6)cc5c4c3o2)cc1]

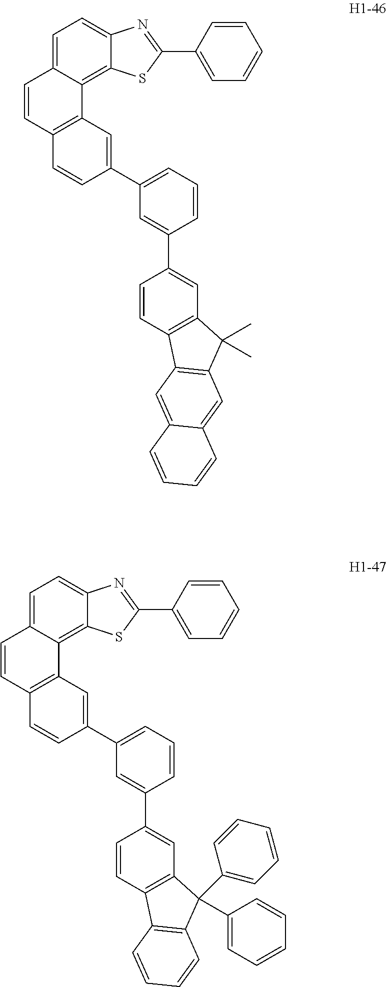 [SMILES: CC1(C)c2cc(-c3cccc(-c4ccc5ccc6ccc7nc(-c8ccccc8)sc7c6c5c4)c3)ccc2-c2cc3ccccc3cc21 ; c1ccc(-c2nc3ccc4ccc5ccc(-c6cccc(-c7ccc8c(c7)C(c7ccccc7)(c7ccccc7)c7ccccc7-8)c6)cc5c4c3s2)cc1]

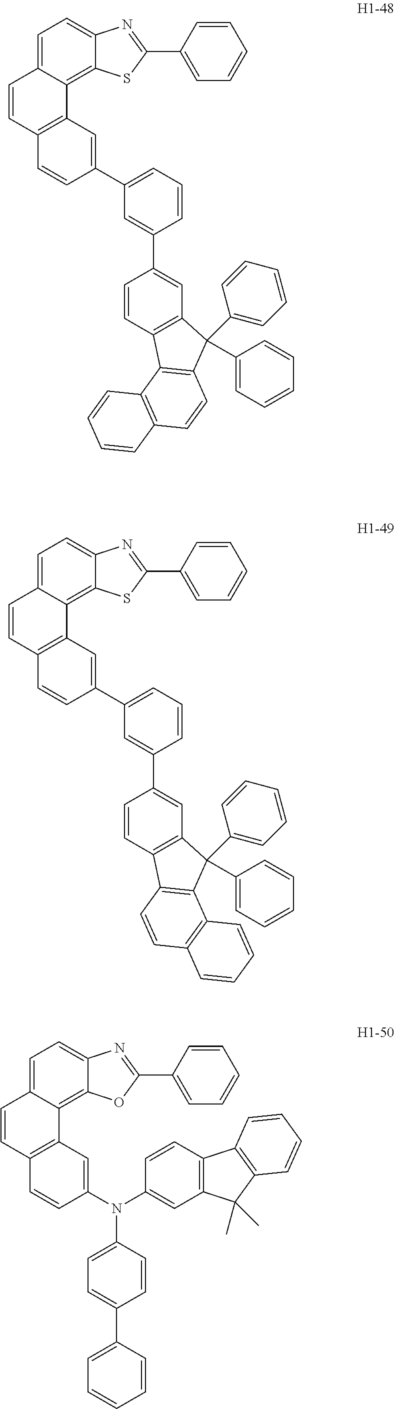 [SMILES: c1ccc(-c2nc3ccc4ccc5ccc(-c6cccc(-c7ccc8c(c7)C(c7ccccc7)(c7ccccc7)c7ccc9ccccc9c7-8)c6)cc5c4c3s2)cc1 ; c1ccc(-c2nc3ccc4ccc5ccc(-c6cccc(-c7ccc8c(c7)C(c7ccccc7)(c7ccccc7)c7c-8ccc8ccccc78)c6)cc5c4c3s2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

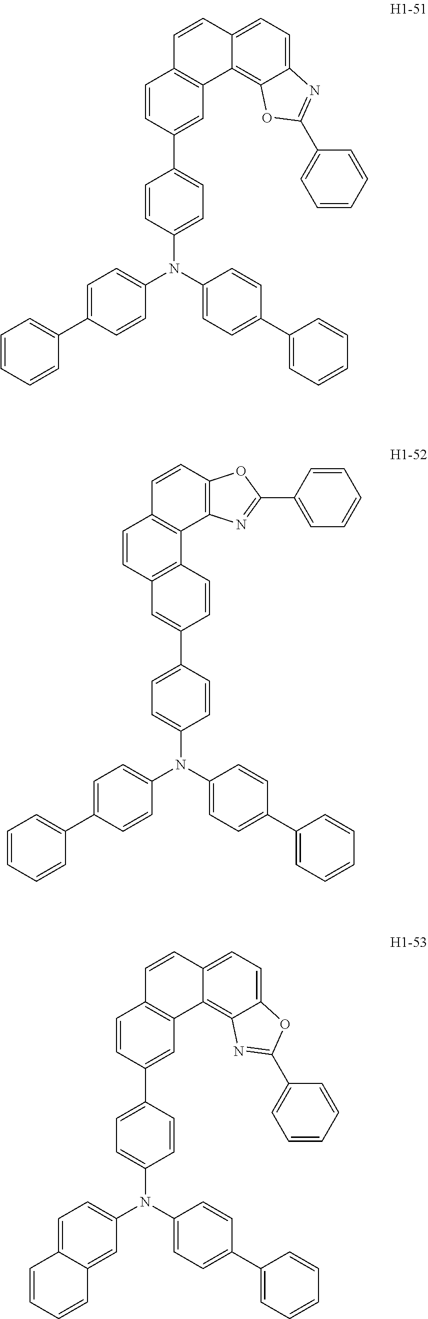 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

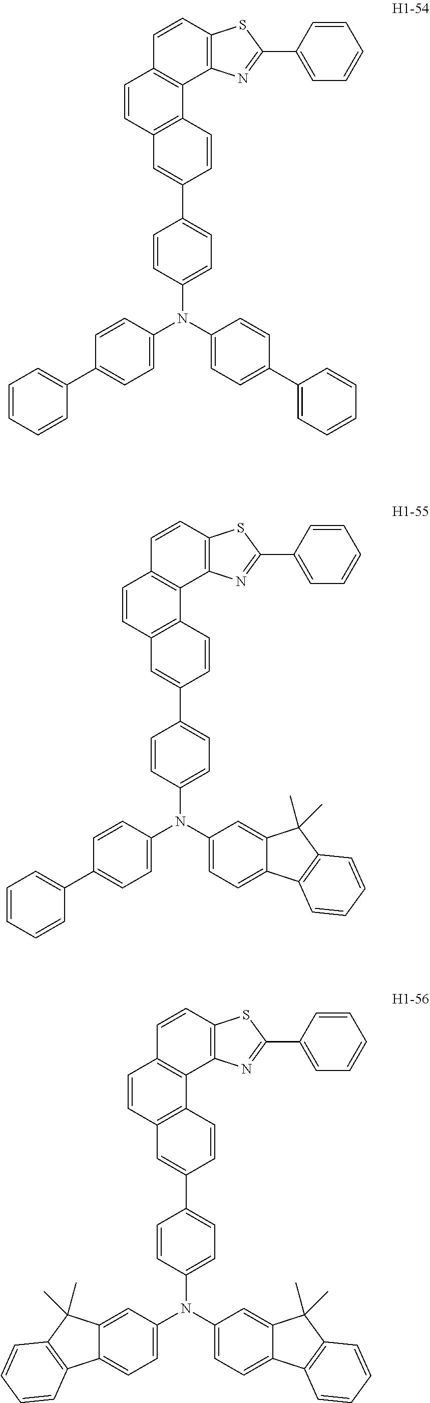 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

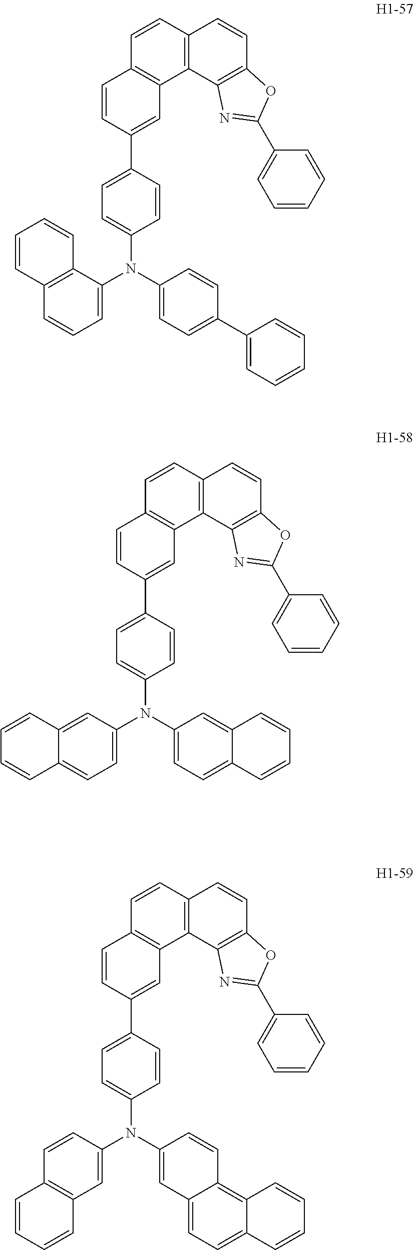 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

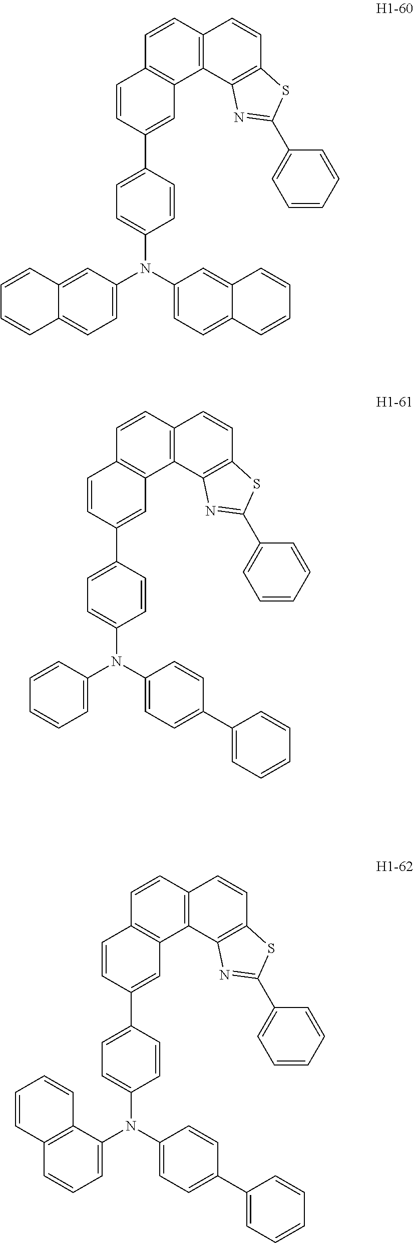 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

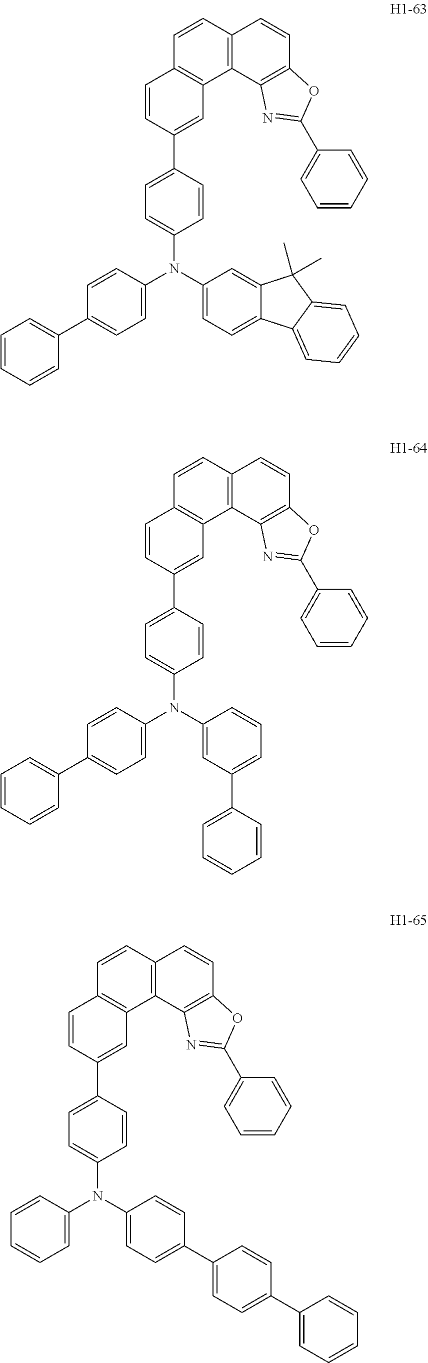 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

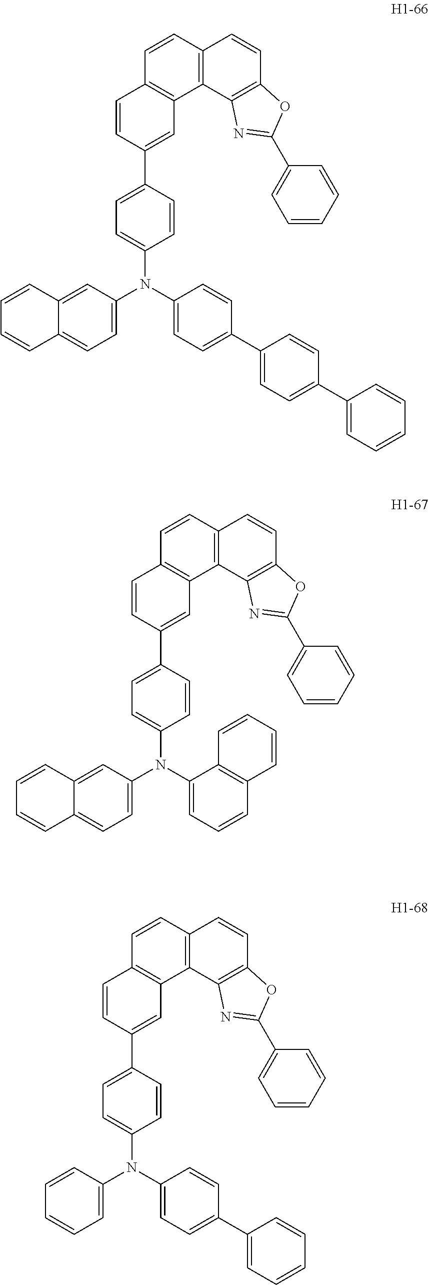 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

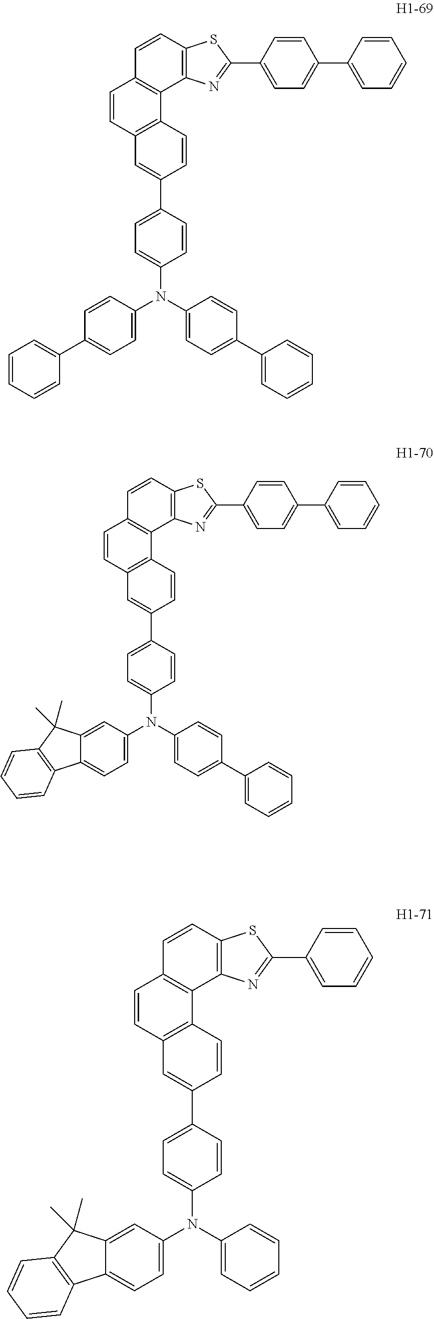 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

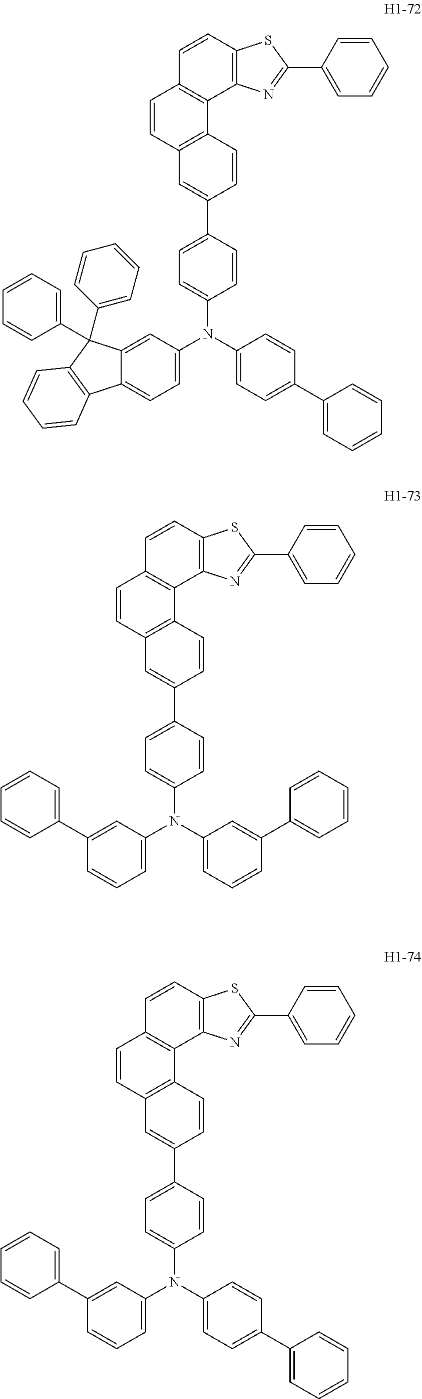 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

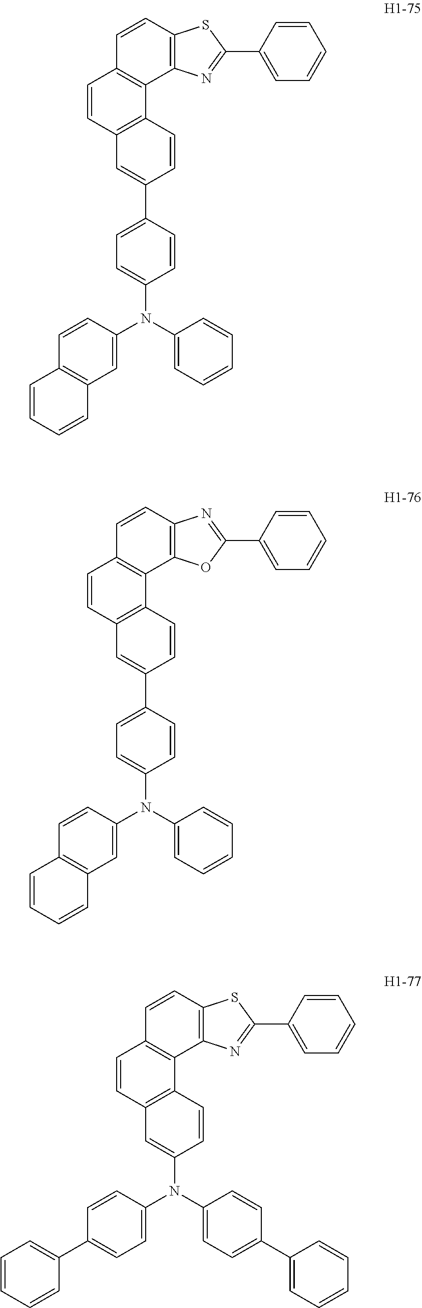 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

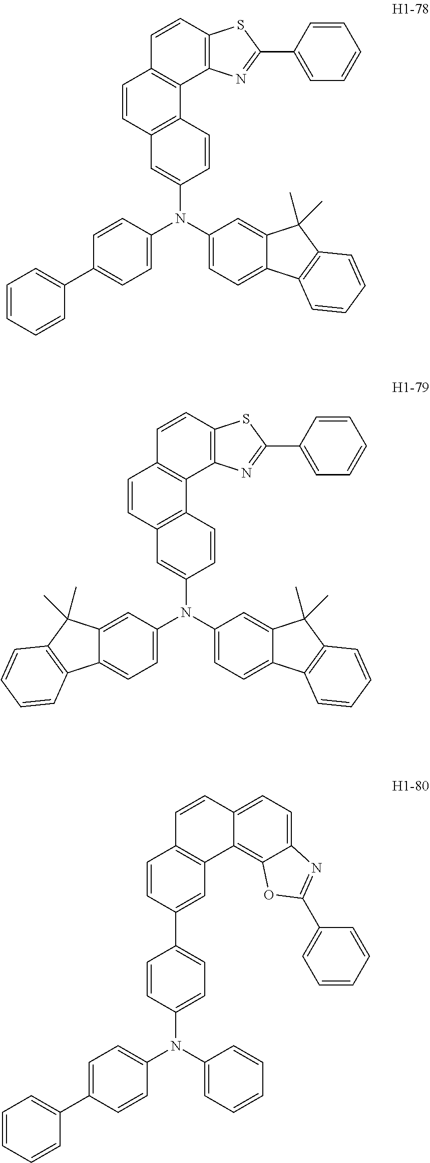 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

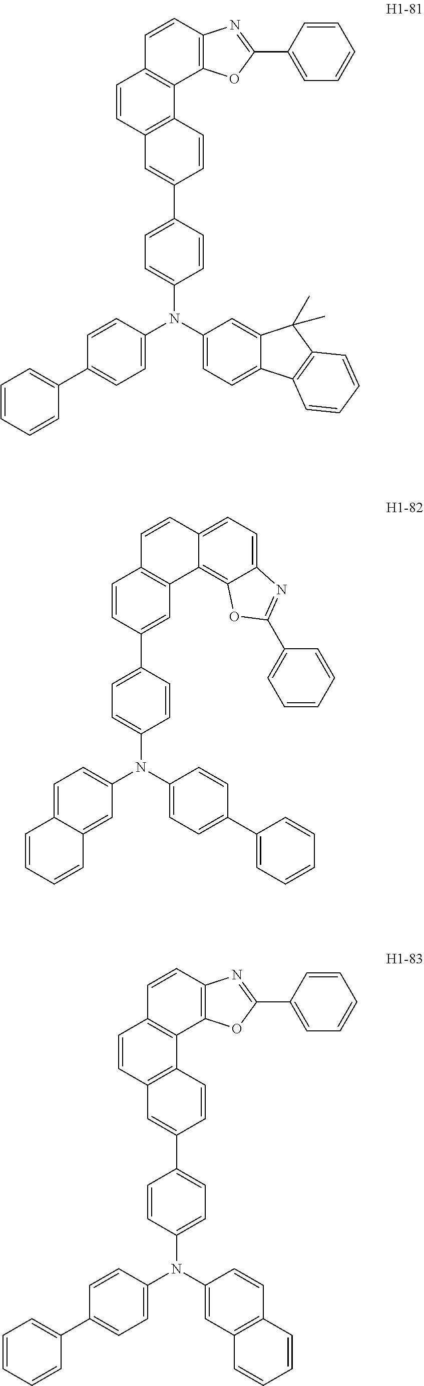 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7nc(-c8ccccc8)oc7c65)c4)cc3)c3ccc4ccccc4c3)cc2)cc1]

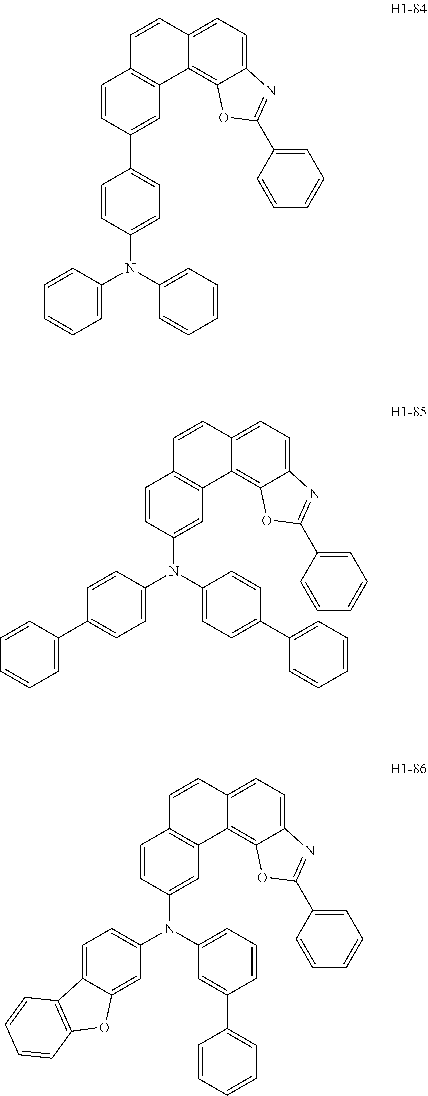 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1]

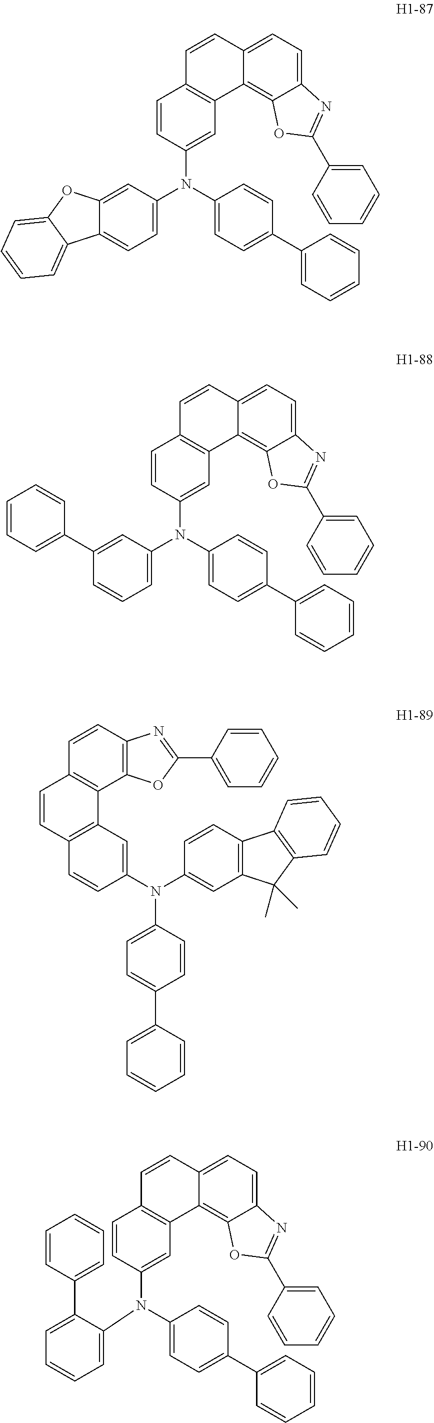 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

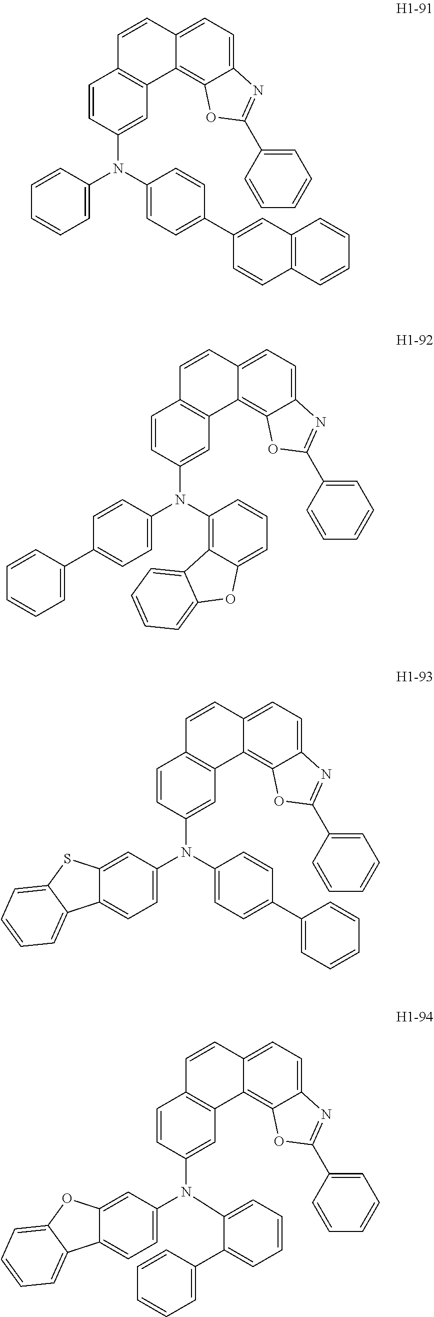 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1]

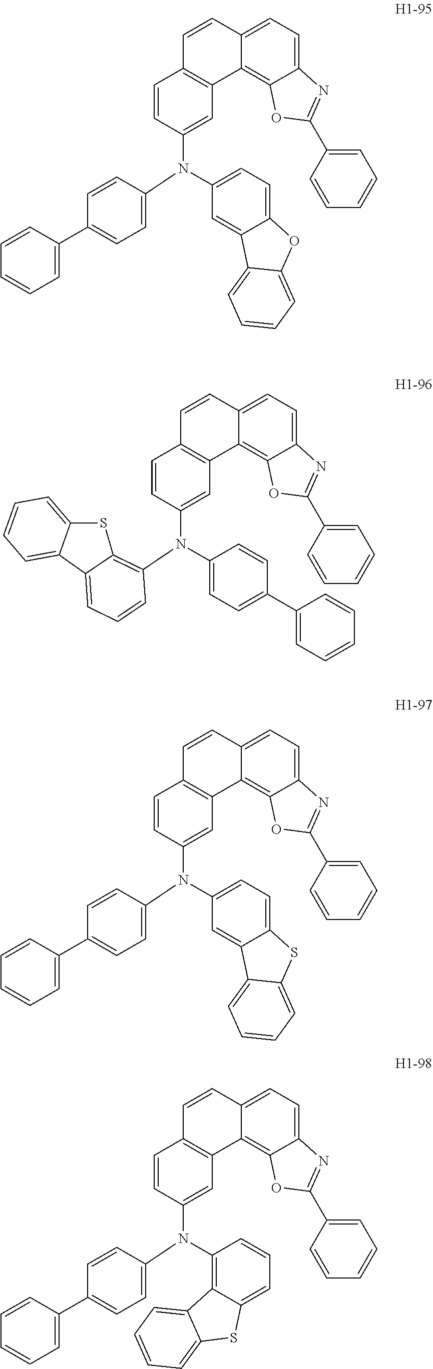 [SMILES: c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1]

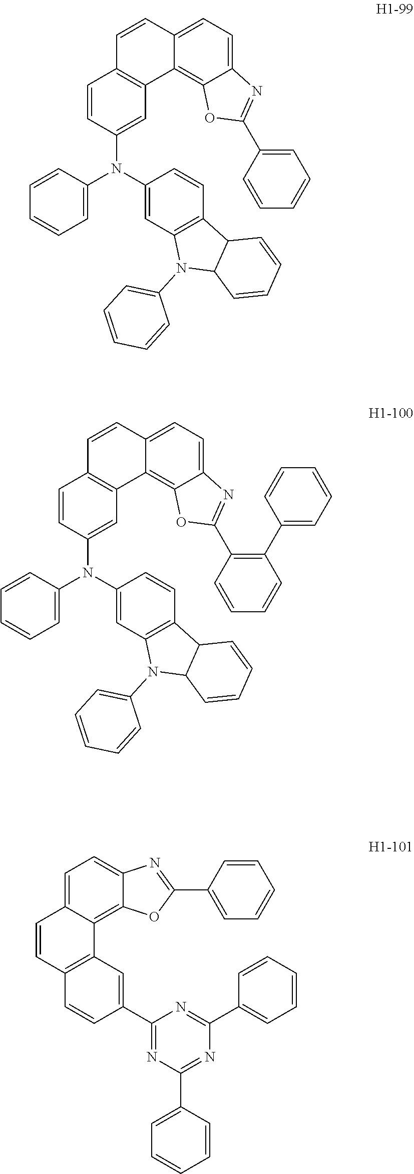 [SMILES: C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc(-c3ccccc3)nc(-c3ccc4ccc5ccc6nc(-c7ccccc7)oc6c5c4c3)n2)cc1]

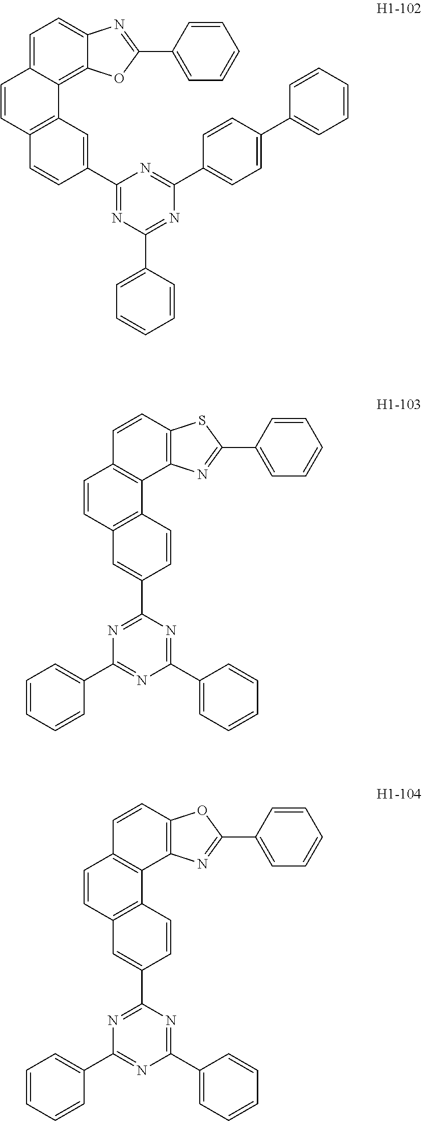 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6oc(-c7ccccc7)nc6c54)c3)n2)cc1]

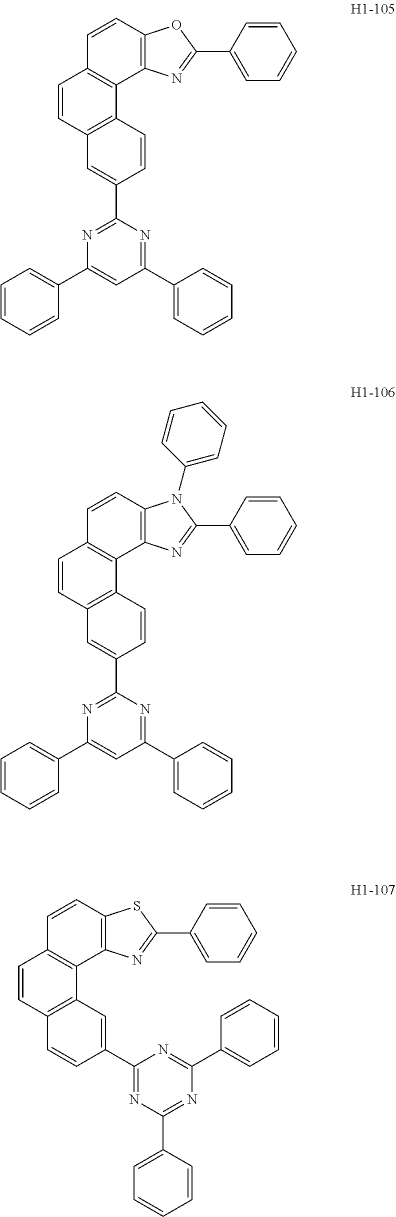 [SMILES: c1ccc(-c2cc(-c3ccccc3)nc(-c3ccc4c(ccc5ccc6oc(-c7ccccc7)nc6c54)c3)n2)cc1 ; c1ccc(-c2cc(-c3ccccc3)nc(-c3ccc4c(ccc5ccc6c(nc(-c7ccccc7)n6-c6ccccc6)c54)c3)n2)cc1 ; c1ccc(-c2nc(-c3ccccc3)nc(-c3ccc4ccc5ccc6sc(-c7ccccc7)nc6c5c4c3)n2)cc1]

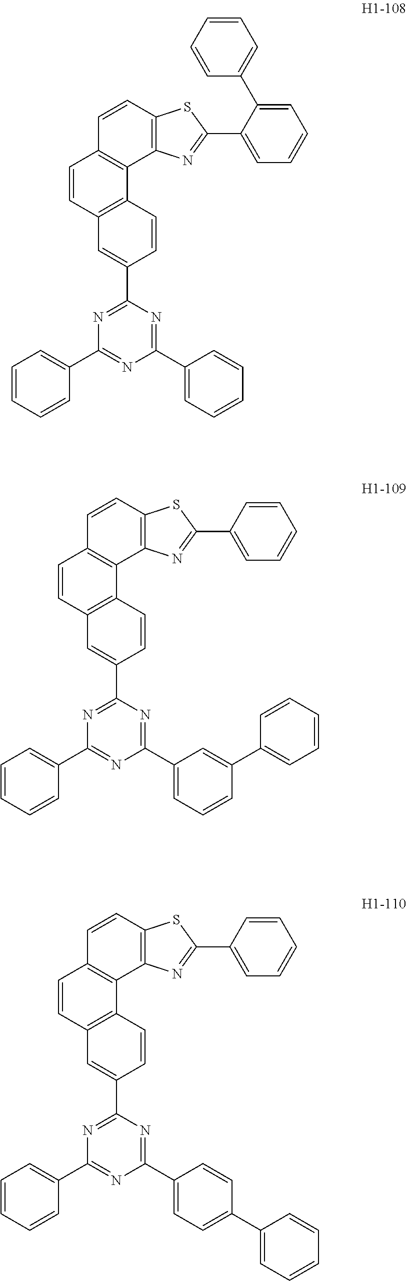 [SMILES: c1ccc(-c2nc(-c3ccccc3)nc(-c3ccc4c(ccc5ccc6sc(-c7ccccc7-c7ccccc7)nc6c54)c3)n2)cc1 ; c1ccc(-c2cccc(-c3nc(-c4ccccc4)nc(-c4ccc5c(ccc6ccc7sc(-c8ccccc8)nc7c65)c4)n3)c2)cc1 ; c1ccc(-c2ccc(-c3nc(-c4ccccc4)nc(-c4ccc5c(ccc6ccc7sc(-c8ccccc8)nc7c65)c4)n3)cc2)cc1]

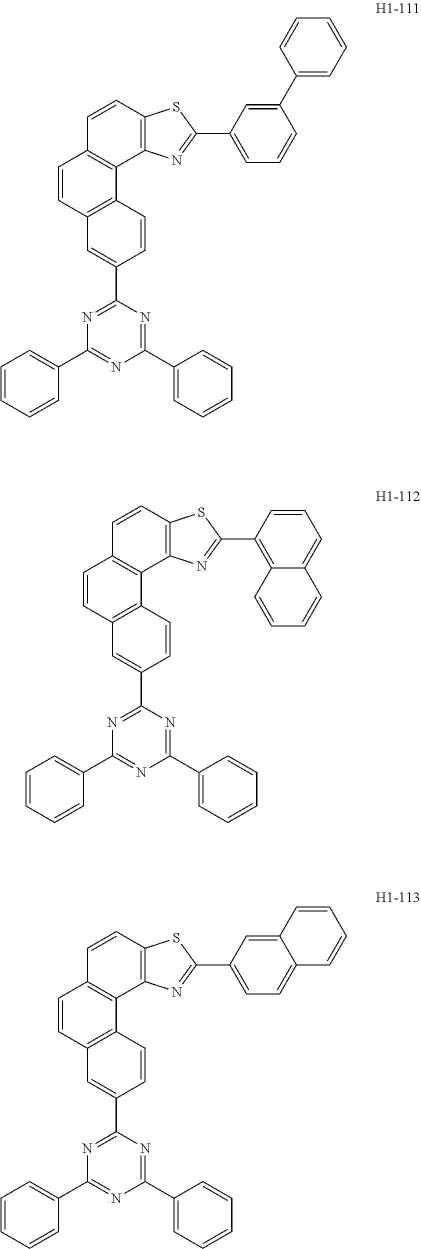 [SMILES: c1ccc(-c2cccc(-c3nc4c(ccc5ccc6cc(-c7nc(-c8ccccc8)nc(-c8ccccc8)n7)ccc6c54)s3)c2)cc1 ; c1ccc(-c2nc(-c3ccccc3)nc(-c3ccc4c(ccc5ccc6sc(-c7cccc8ccccc78)nc6c54)c3)n2)cc1 ; c1ccc(-c2nc(-c3ccccc3)nc(-c3ccc4c(ccc5ccc6sc(-c7ccc8ccccc8c7)nc6c54)c3)n2)cc1]

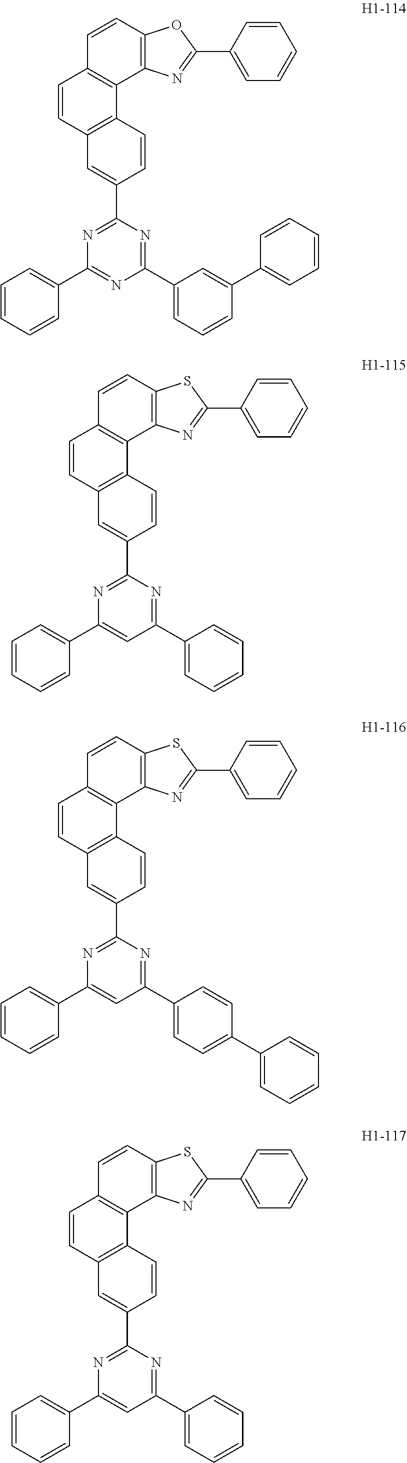

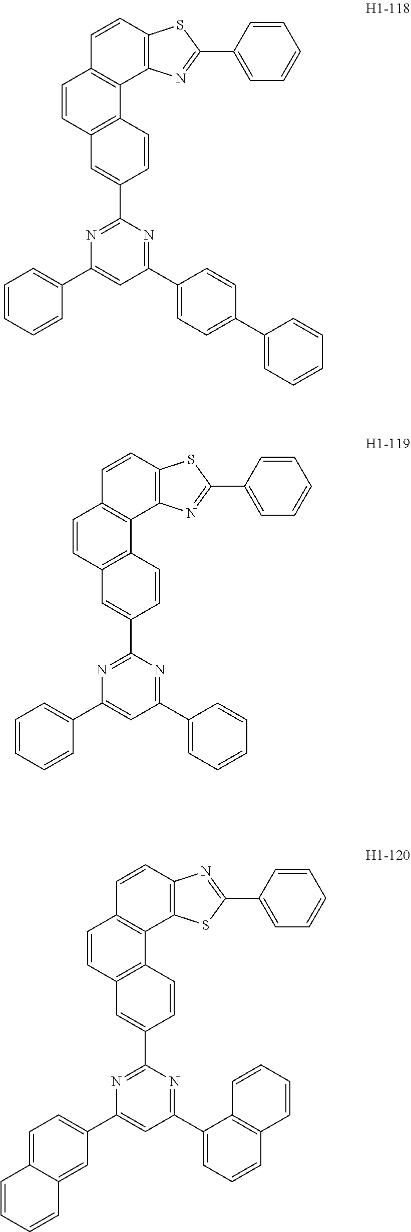 [SMILES: c1ccc(-c2ccc(-c3cc(-c4ccccc4)nc(-c4ccc5c(ccc6ccc7sc(-c8ccccc8)nc7c65)c4)n3)cc2)cc1 ; c1ccc(-c2cc(-c3ccccc3)nc(-c3ccc4c(ccc5ccc6sc(-c7ccccc7)nc6c54)c3)n2)cc1 ; c1ccc(-c2nc3ccc4ccc5cc(-c6nc(-c7ccc8ccccc8c7)cc(-c7cccc8ccccc78)n6)ccc5c4c3s2)cc1]

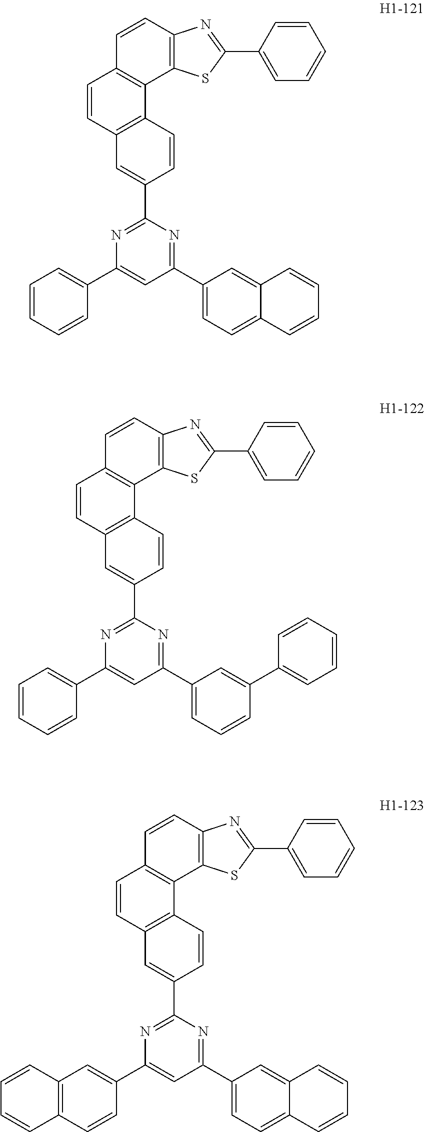 [SMILES: c1ccc(-c2cc(-c3ccc4ccccc4c3)nc(-c3ccc4c(ccc5ccc6nc(-c7ccccc7)sc6c54)c3)n2)cc1 ; c1ccc(-c2cccc(-c3cc(-c4ccccc4)nc(-c4ccc5c(ccc6ccc7nc(-c8ccccc8)sc7c65)c4)n3)c2)cc1 ; c1ccc(-c2nc3ccc4ccc5cc(-c6nc(-c7ccc8ccccc8c7)cc(-c7ccc8ccccc8c7)n6)ccc5c4c3s2)cc1]

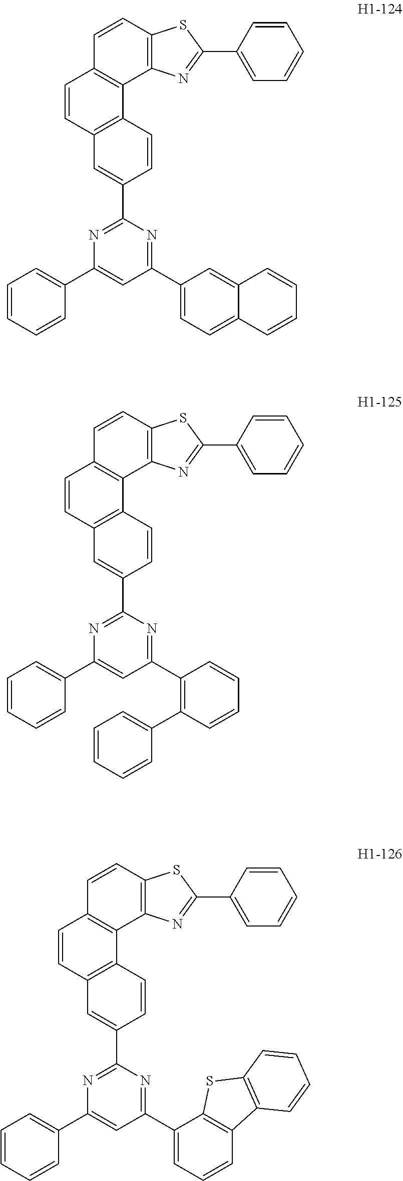 [SMILES: c1ccc(-c2cc(-c3ccc4ccccc4c3)nc(-c3ccc4c(ccc5ccc6sc(-c7ccccc7)nc6c54)c3)n2)cc1 ; c1ccc(-c2cc(-c3ccccc3-c3ccccc3)nc(-c3ccc4c(ccc5ccc6sc(-c7ccccc7)nc6c54)c3)n2)cc1 ; c1ccc(-c2cc(-c3cccc4c3sc3ccccc34)nc(-c3ccc4c(ccc5ccc6sc(-c7ccccc7)nc6c54)c3)n2)cc1]

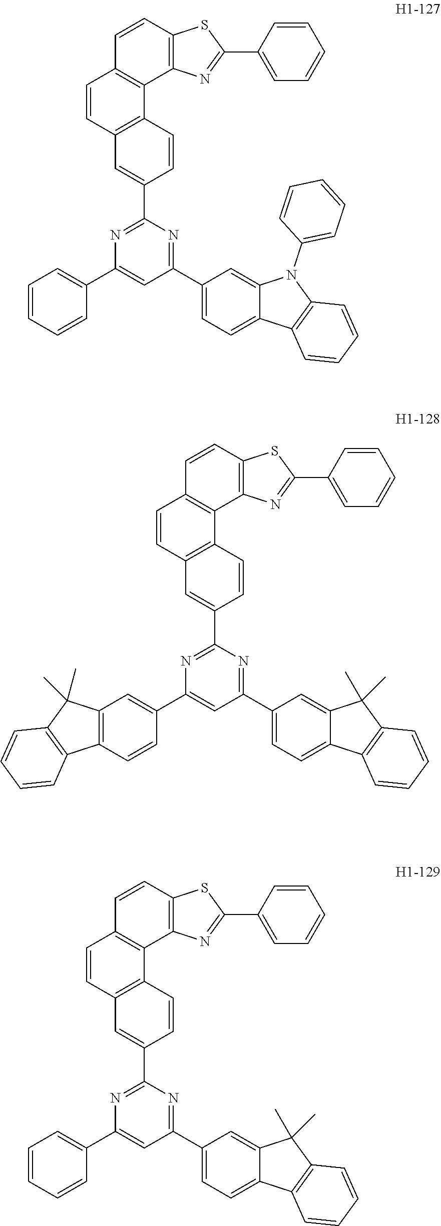 [SMILES: c1ccc(-c2cc(-c3ccc4c5ccccc5n(-c5ccccc5)c4c3)nc(-c3ccc4c(ccc5ccc6sc(-c7ccccc7)nc6c54)c3)n2)cc1 ; CC1(C)c2ccccc2-c2ccc(-c3cc(-c4ccc5c(c4)C(C)(C)c4ccccc4-5)nc(-c4ccc5c(ccc6ccc7sc(-c8ccccc8)nc7c65)c4)n3)cc21 ; CC1(C)c2ccccc2-c2ccc(-c3cc(-c4ccccc4)nc(-c4ccc5c(ccc6ccc7sc(-c8ccccc8)nc7c65)c4)n3)cc21]

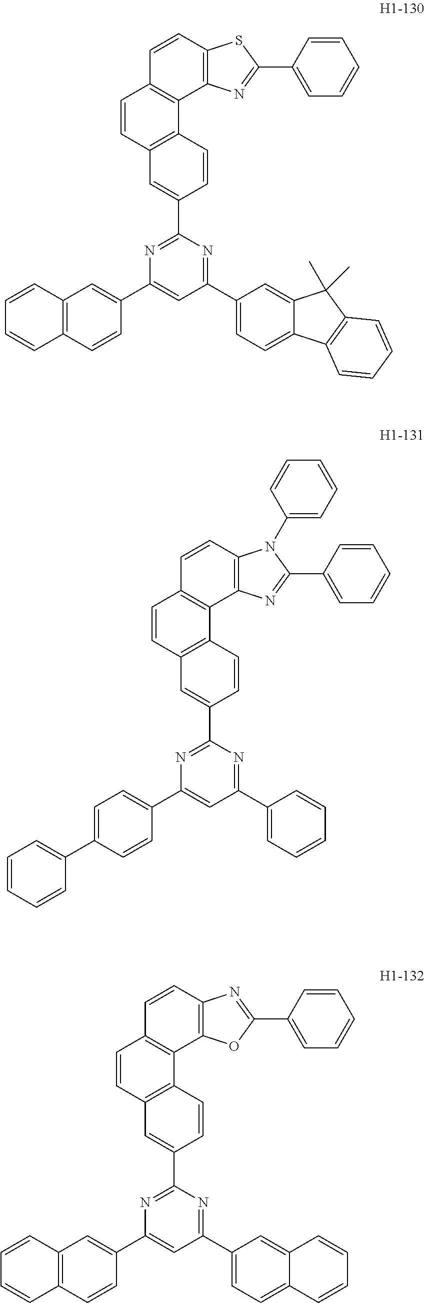 [SMILES: CC1(C)c2ccccc2-c2ccc(-c3cc(-c4ccc5ccccc5c4)nc(-c4ccc5c(ccc6ccc7sc(-c8ccccc8)nc7c65)c4)n3)cc21 ; c1ccc(-c2ccc(-c3cc(-c4ccccc4)nc(-c4ccc5c(ccc6ccc7c(nc(-c8ccccc8)n7-c7ccccc7)c65)c4)n3)cc2)cc1 ; c1ccc(-c2nc3ccc4ccc5cc(-c6nc(-c7ccc8ccccc8c7)cc(-c7ccc8ccccc8c7)n6)ccc5c4c3o2)cc1]

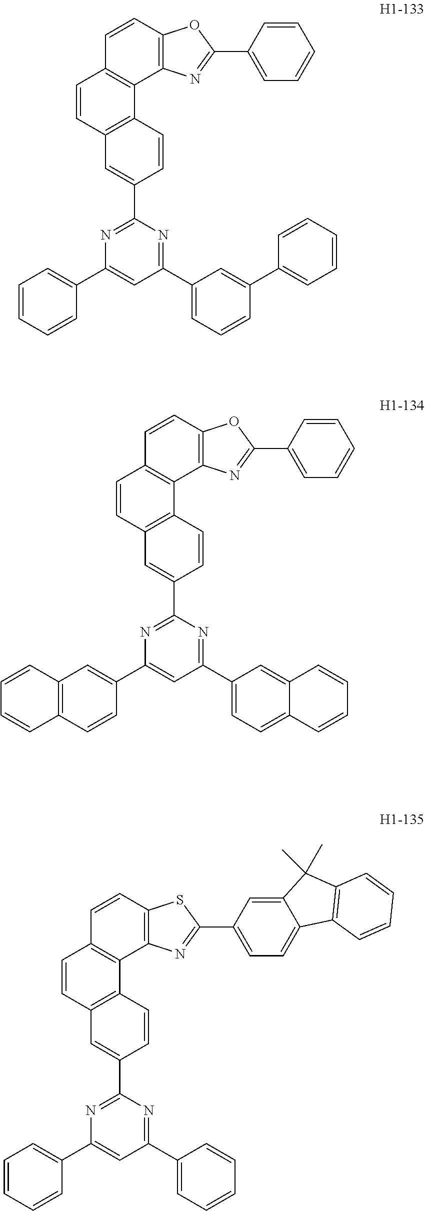 [SMILES: c1ccc(-c2cccc(-c3cc(-c4ccccc4)nc(-c4ccc5c(ccc6ccc7oc(-c8ccccc8)nc7c65)c4)n3)c2)cc1 ; c1ccc(-c2nc3c(ccc4ccc5cc(-c6nc(-c7ccc8ccccc8c7)cc(-c7ccc8ccccc8c7)n6)ccc5c43)o2)cc1 ; CC1(C)c2ccccc2-c2ccc(-c3nc4c(ccc5ccc6cc(-c7nc(-c8ccccc8)cc(-c8ccccc8)n7)ccc6c54)s3)cc21]

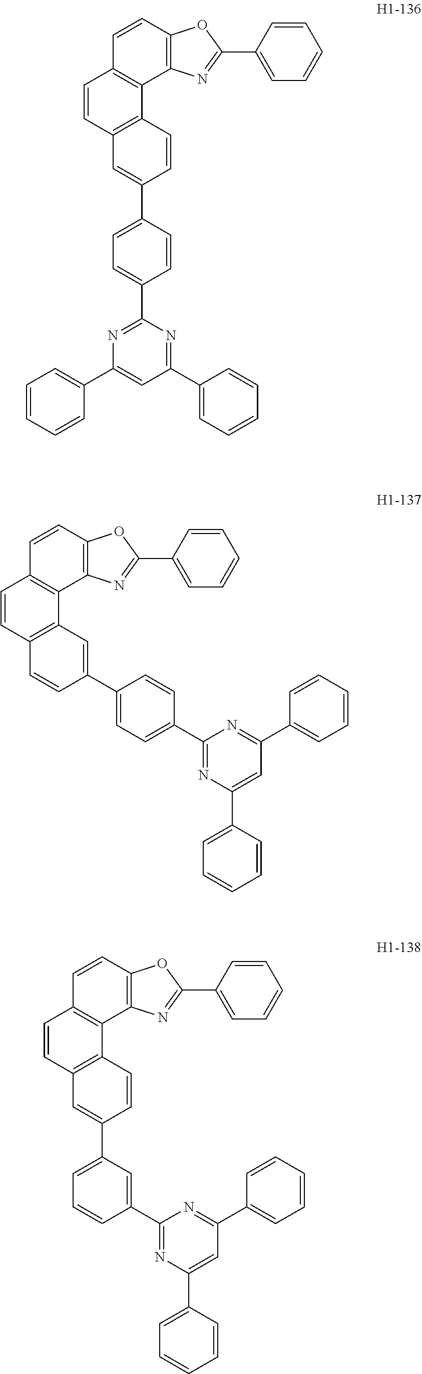 [SMILES: c1ccc(-c2cc(-c3ccccc3)nc(-c3ccc(-c4ccc5c(ccc6ccc7oc(-c8ccccc8)nc7c65)c4)cc3)n2)cc1 ; c1ccc(-c2cc(-c3ccccc3)nc(-c3ccc(-c4ccc5ccc6ccc7oc(-c8ccccc8)nc7c6c5c4)cc3)n2)cc1 ; c1ccc(-c2cc(-c3ccccc3)nc(-c3cccc(-c4ccc5c(ccc6ccc7oc(-c8ccccc8)nc7c65)c4)c3)n2)cc1]

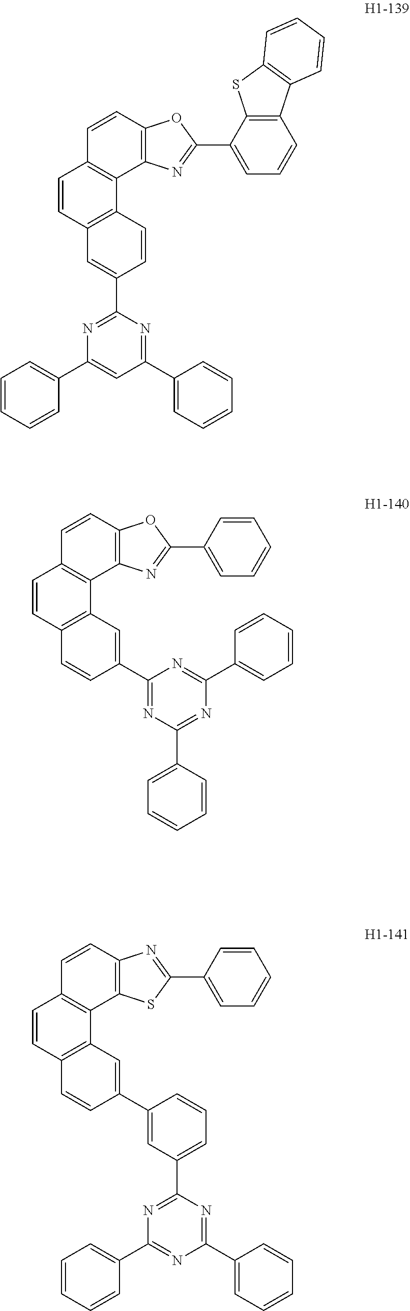 [SMILES: c1ccc(-c2cc(-c3ccccc3)nc(-c3ccc4c(ccc5ccc6oc(-c7cccc8c7sc7ccccc78)nc6c54)c3)n2)cc1 ; c1ccc(-c2nc(-c3ccccc3)nc(-c3ccc4ccc5ccc6oc(-c7ccccc7)nc6c5c4c3)n2)cc1 ; c1ccc(-c2nc(-c3ccccc3)nc(-c3cccc(-c4ccc5ccc6ccc7nc(-c8ccccc8)sc7c6c5c4)c3)n2)cc1]

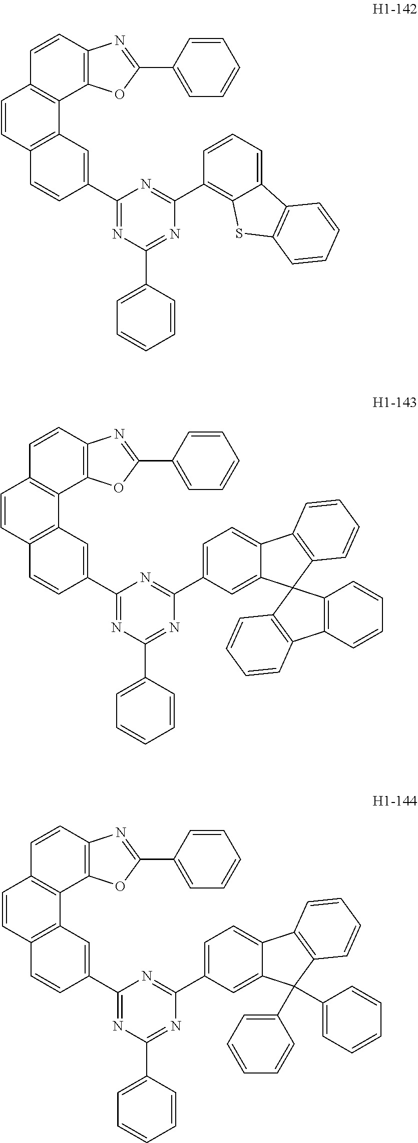 [SMILES: c1ccc(-c2nc(-c3ccc4ccc5ccc6nc(-c7ccccc7)oc6c5c4c3)nc(-c3cccc4c3sc3ccccc34)n2)cc1 ; c1ccc(-c2nc(-c3ccc4c(c3)C3(c5ccccc5-c5ccccc53)c3ccccc3-4)nc(-c3ccc4ccc5ccc6nc(-c7ccccc7)oc6c5c4c3)n2)cc1 ; c1ccc(-c2nc(-c3ccc4c(c3)C(c3ccccc3)(c3ccccc3)c3ccccc3-4)nc(-c3ccc4ccc5ccc6nc(-c7ccccc7)oc6c5c4c3)n2)cc1]

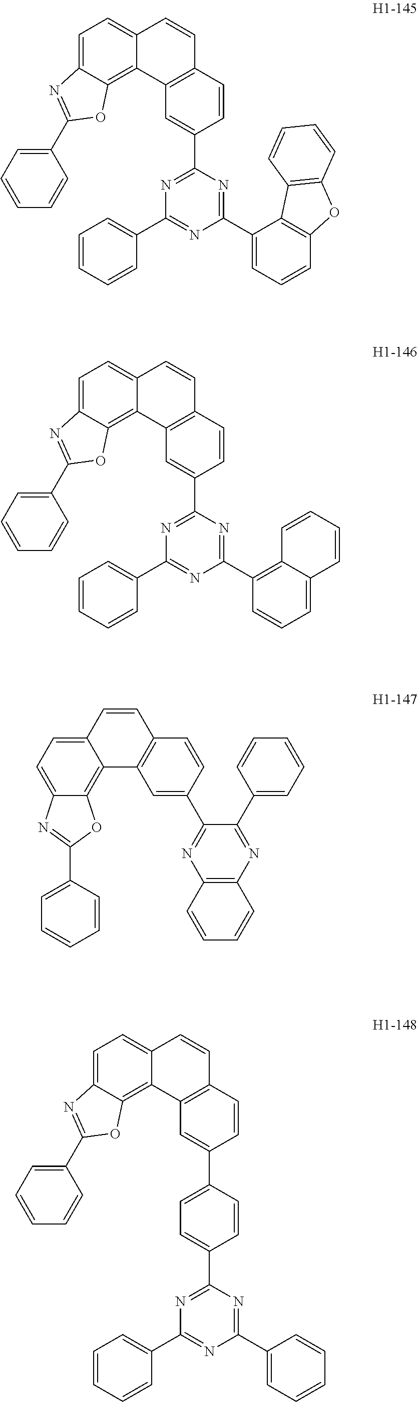 [SMILES: c1ccc(-c2nc(-c3ccc4ccc5ccc6nc(-c7ccccc7)oc6c5c4c3)nc(-c3cccc4oc5ccccc5c34)n2)cc1 ; c1ccc(-c2nc(-c3ccc4ccc5ccc6nc(-c7ccccc7)oc6c5c4c3)nc(-c3cccc4ccccc34)n2)cc1 ; c1ccc(-c2nc3ccc4ccc5ccc(-c6nc7ccccc7nc6-c6ccccc6)cc5c4c3o2)cc1 ; c1ccc(-c2nc(-c3ccccc3)nc(-c3ccc(-c4ccc5ccc6ccc7nc(-c8ccccc8)oc7c6c5c4)cc3)n2)cc1]

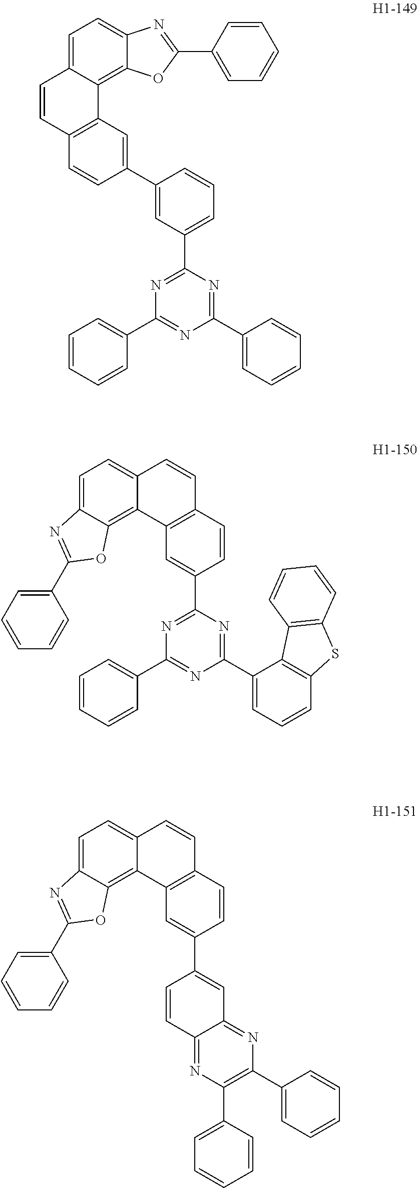 [SMILES: c1ccc(-c2nc(-c3ccccc3)nc(-c3cccc(-c4ccc5ccc6ccc7nc(-c8ccccc8)oc7c6c5c4)c3)n2)cc1 ; c1ccc(-c2nc(-c3ccc4ccc5ccc6nc(-c7ccccc7)oc6c5c4c3)nc(-c3cccc4sc5ccccc5c34)n2)cc1 ; c1ccc(-c2nc3ccc4ccc5ccc(-c6ccc7nc(-c8ccccc8)c(-c8ccccc8)nc7c6)cc5c4c3o2)cc1]

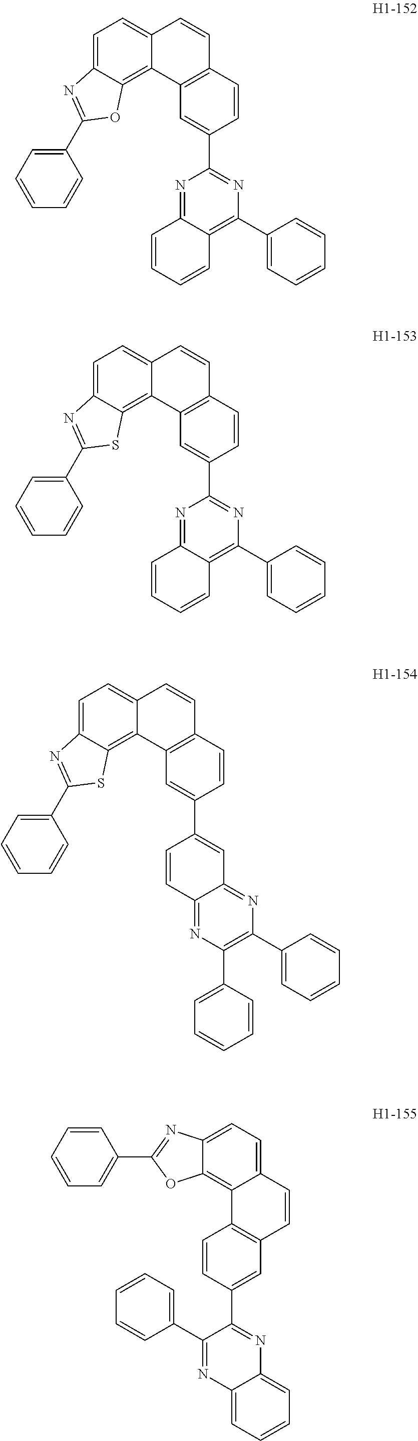 [SMILES: c1ccc(-c2nc3ccc4ccc5ccc(-c6nc(-c7ccccc7)c7ccccc7n6)cc5c4c3o2)cc1 ; c1ccc(-c2nc3ccc4ccc5ccc(-c6nc(-c7ccccc7)c7ccccc7n6)cc5c4c3s2)cc1 ; c1ccc(-c2nc3ccc4ccc5ccc(-c6ccc7nc(-c8ccccc8)c(-c8ccccc8)nc7c6)cc5c4c3s2)cc1 ; c1ccc(-c2nc3ccc4ccc5cc(-c6nc7ccccc7nc6-c6ccccc6)ccc5c4c3o2)cc1]

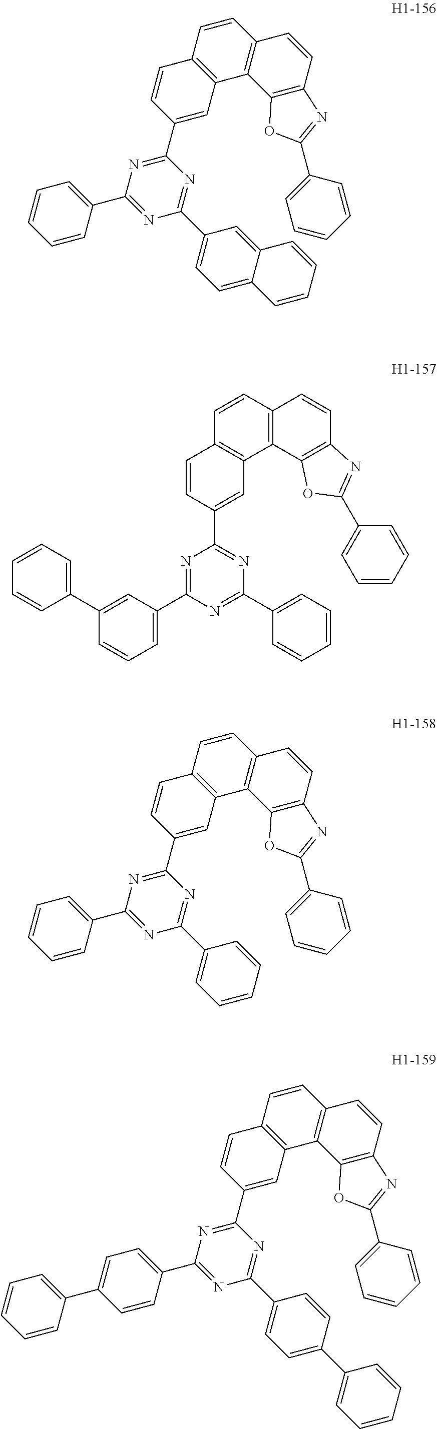 [SMILES: c1ccc(-c2nc(-c3ccc4ccccc4c3)nc(-c3ccc4ccc5ccc6nc(-c7ccccc7)oc6c5c4c3)n2)cc1 ; c1ccc(-c2cccc(-c3nc(-c4ccccc4)nc(-c4ccc5ccc6ccc7nc(-c8ccccc8)oc7c6c5c4)n3)c2)cc1 ; c1ccc(-c2nc(-c3ccccc3)nc(-c3ccc4ccc5ccc6nc(-c7ccccc7)oc6c5c4c3)n2)cc1 ; c1ccc(-c2ccc(-c3nc(-c4ccc(-c5ccccc5)cc4)nc(-c4ccc5ccc6ccc7nc(-c8ccccc8)oc7c6c5c4)n3)cc2)cc1]

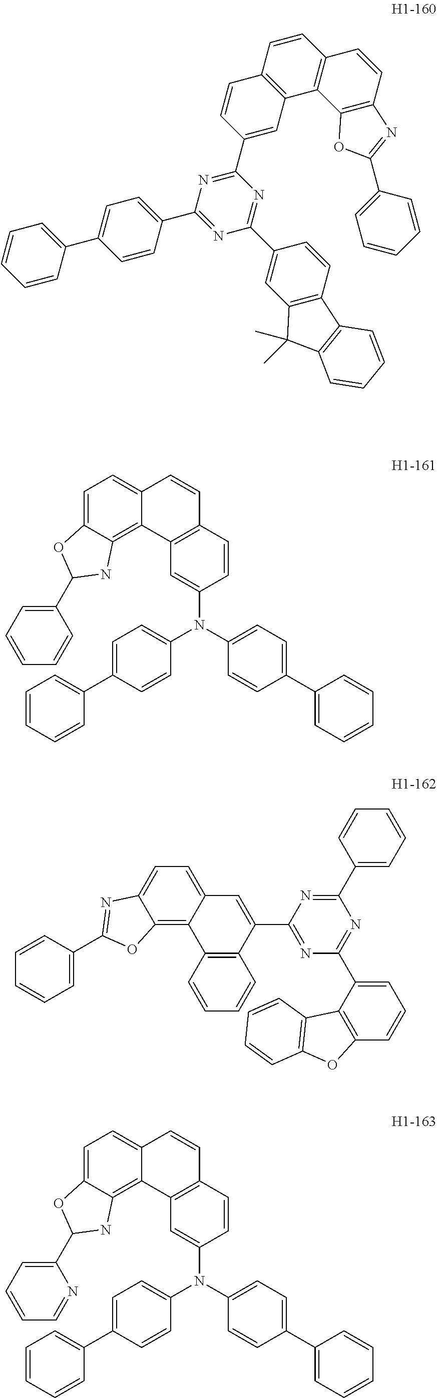 [SMILES: CC1(C)c2ccccc2-c2ccc(-c3nc(-c4ccc(-c5ccccc5)cc4)nc(-c4ccc5ccc6ccc7nc(-c8ccccc8)oc7c6c5c4)n3)cc21 ; c1ccc(-c2ccc(N(c3ccc(-c4ccccc4)cc3)c3ccc4ccc5ccc6c(c5c4c3)NC(c3ccccc3)O6)cc2)cc1 ; c1ccc(-c2nc(-c3cc4ccc5nc(-c6ccccc6)oc5c4c4ccccc34)nc(-c3cccc4oc5ccccc5c34)n2)cc1 ; c1ccc(-c2ccc(N(c3ccc(-c4ccccc4)cc3)c3ccc4ccc5ccc6c(c5c4c3)NC(c3ccccn3)O6)cc2)cc1]

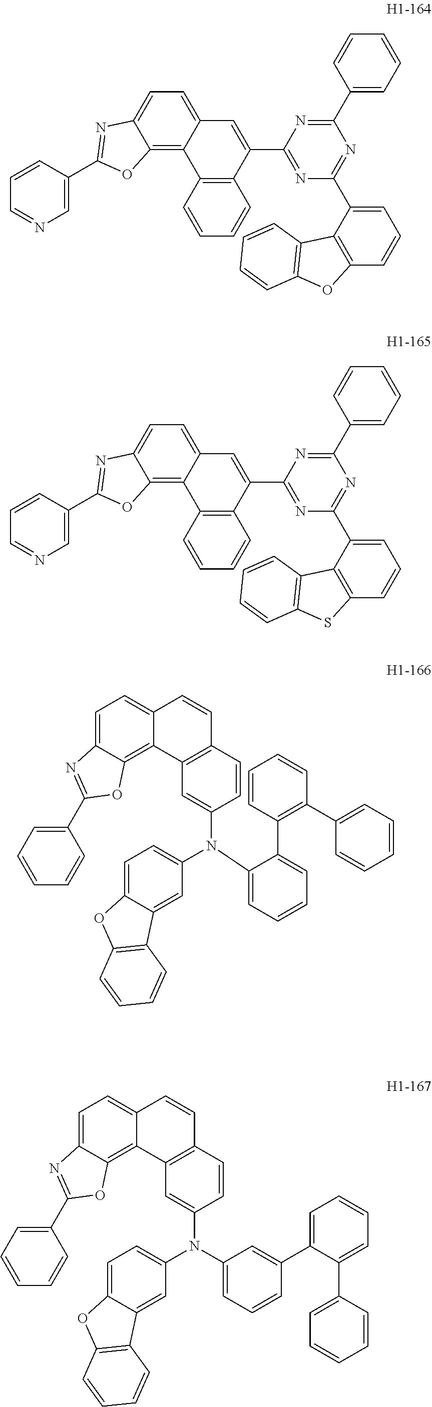 [SMILES: c1ccc(-c2nc(-c3cc4ccc5nc(-c6cccnc6)oc5c4c4ccccc34)nc(-c3cccc4oc5ccccc5c34)n2)cc1 ; c1ccc(-c2nc(-c3cc4ccc5nc(-c6cccnc6)oc5c4c4ccccc34)nc(-c3cccc4sc5ccccc5c34)n2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

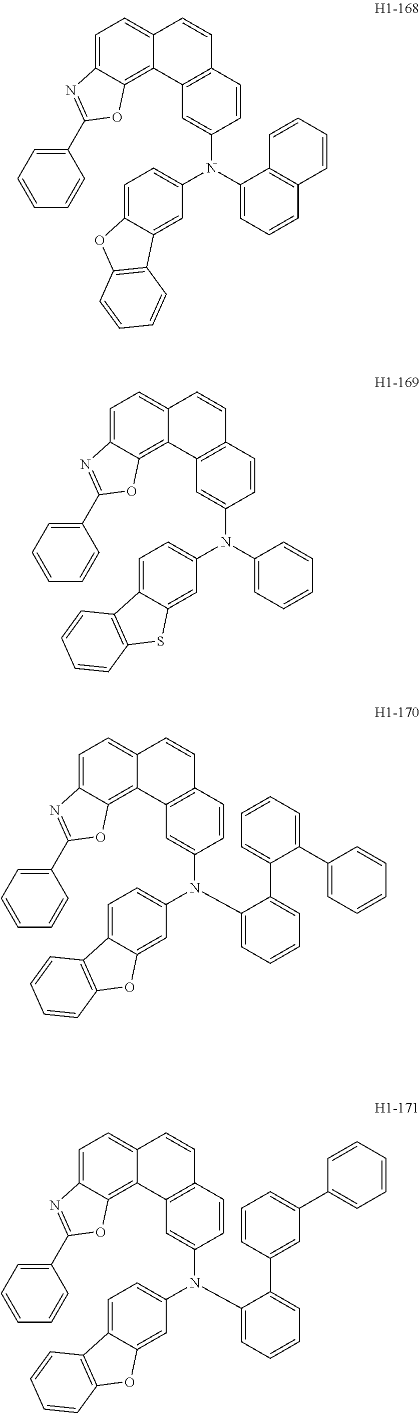 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccccc6)c6ccc7c(c6)sc6ccccc67)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

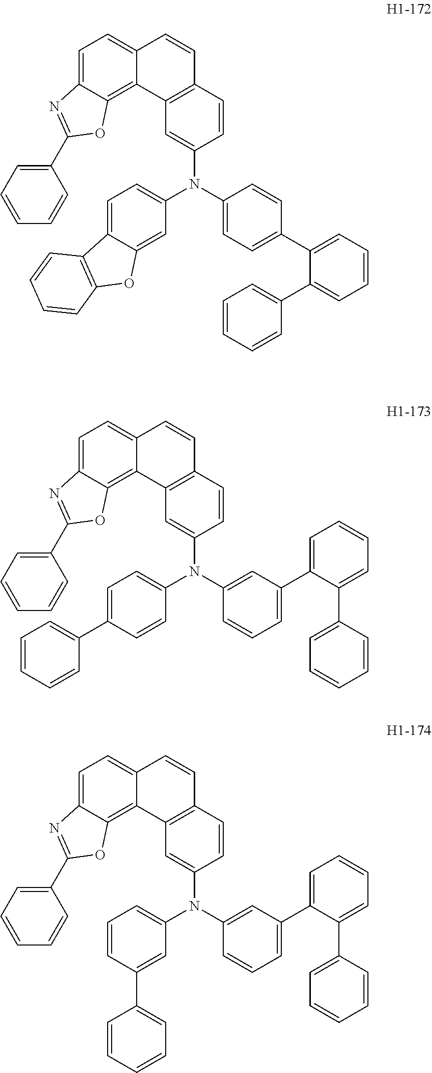 [SMILES: c1ccc(-c2nc3ccc4ccc5ccc(N(c6ccc(-c7ccccc7-c7ccccc7)cc6)c6ccc7c(c6)oc6ccccc67)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

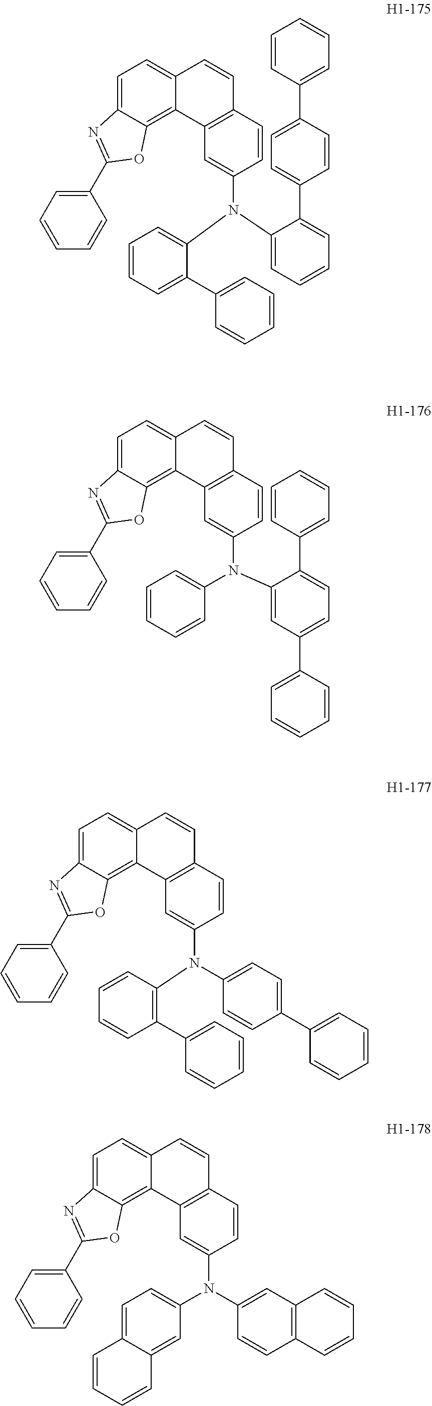 [SMILES: c1ccc(-c2ccc(-c3ccccc3N(c3ccc4ccc5ccc6nc(-c7ccccc7)oc6c5c4c3)c3ccccc3-c3ccccc3)cc2)cc1 ; c1ccc(-c2ccc(-c3ccccc3)c(N(c3ccccc3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccc7ccccc7c6)c6ccc7ccccc7c6)cc5c4c3o2)cc1]

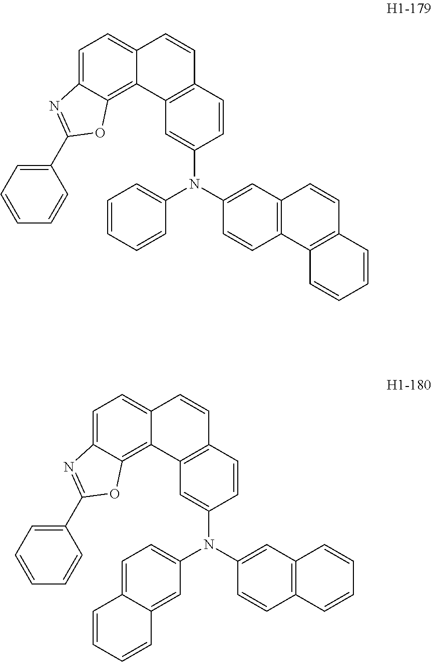 [SMILES: c1ccc(-c2nc3ccc4ccc5ccc(N(c6ccccc6)c6ccc7c(ccc8ccccc87)c6)cc5c4c3o2)cc1 ; c1ccc(-c2nc3ccc4ccc5ccc(N(c6ccc7ccccc7c6)c6ccc7ccccc7c6)cc5c4c3o2)cc1]

The compound represented by the formula 1 according to the present disclosure may be produced by a synthetic method known to a person skilled in the art, for example, the compound represented by the formula 1 may be prepared by referring to KR 2017-0022865 A (published on Mar. 2, 2017), but is not limited thereto:

The second host compound as another host material according to one embodiment may be represented by the following formula 2.

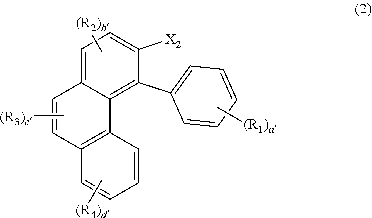

in formula 2,

X₂ represents O, S, or CR₇R₈;

R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered) heteroarylamino;

at least one of R₁ to R₄ is a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; provided that the case where any one of R₁ to R₄ is triphenylene is excluded;

R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s); and a′ and d′ each independently represent, an integer of 1 to 4, b′ and c′ each independently represent, an integer of 1 or 2, and when a′ to d′ represent an integer of 2 or more, each of R to R₄ may be the same or different.

In one embodiment, X₂ represents O, S, or CR₇R₈, in which R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s). Preferably, R₇ and R₈ each independently may be a substituted or unsubstituted (C1-C20)alkyl, more preferably, a substituted or unsubstituted (C1-C10)alkyl.

In one embodiment, R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; at least one of R₁ to R₄ may be a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino, preferably, at least one of R₁ to R₄ may be a substituted or unsubstituted (5- to 30-membered)heteroaryl, a substituted or unsubstituted di(C6-C30)arylamino, a substituted or unsubstituted di(5- to 30-membered)heteroarylamino, or a substituted or unsubstituted (C6-C30)aryl(5- to 30-membered)heteroarylamino, more preferably, R₄ may be a substituted or unsubstituted (5- to 25-membered)heteroaryl, a substituted or unsubstituted di(C6-025)arylamino, a substituted or unsubstituted di(5- to 25-membered)heteroarylamino, or a substituted or unsubstituted (C6-C25)aryl(5- to 25-membered)heteroarylamino. However, the case where any one of R₁ to R₄ is triphenylene is excluded.

In one embodiment, the second host material represented by the above formula 2 may be represented by the following formula 2-1 or 2-2.

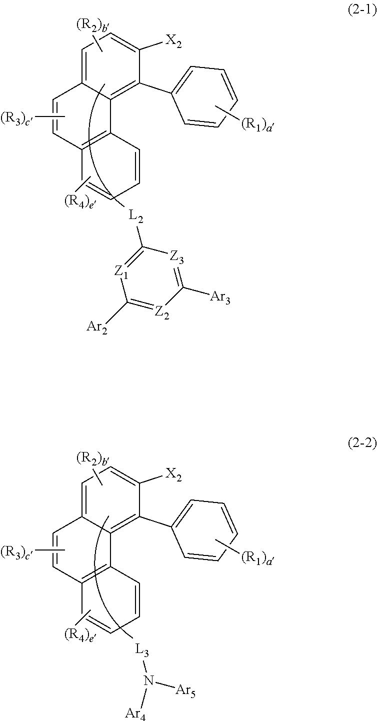

in formula 2-1 or 2-2,

X₂, R₁ to R₄, and a′ to c′ are as defined in the formula 2;

L₂ and L₃ each independently represent, a single bond, a substituted or unsubstituted (C6-C30)arylene, or a substituted or unsubstituted (3- to 30-membered)heteroarylene;

Z₁ to Z₃ each independently represent, N or CH, provided that at least one of Z₁ to Z₃ is N;

Ar₂ to Ar₅ each independently represent, a substituted or unsubstituted (C6-C30)aryl or a substituted or unsubstituted (3- to 30-membered)heteroaryl; and

e′ represents an integer of 1 to 4, and when e′ is an integer of 2 or more, each of R₄ may be the same or different.

In one embodiment, L₂ and L₃ each independently represent, a single bond, a substituted or unsubstituted (C6-C30)arylene, or a substituted or unsubstituted (3- to 30-membered)heteroarylene, preferably, L₂ and L₃ may be a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene. For example, L₂ and L₃ each independently may be a single bond, a substituted or unsubstituted phenylene, a substituted or unsubstituted naphthylene, a substituted or unsubstituted biphenylene, or a substituted or unsubstituted pyridylene.

In one embodiment, Z₁ to Z₃ each independently represent, N or CH, provided that at least one of to Z₃ may be N, preferably, at least two of Z₁ to Z₃ may be N, more preferably, all of Z₁ to Z₃ may be N.

In one embodiment, Ar₂ and Ar₃ in the formula 2-1, each independently represent, a substituted or unsubstituted (C6-C30)aryl or a substituted or unsubstituted (3- to 30-membered)heteroaryl, preferably, Ar₂ and Ar₃ may be a substituted or unsubstituted (C6-C25)aryl or a substituted or unsubstituted (5- to 25-membered)heteroaryl, more preferably, a substituted or unsubstituted (C6-C18)aryl or a substituted or unsubstituted (5- to 18-membered)heteroaryl. For example, Ar₂ and Ar₃ each independently may be a substituted or unsubstituted phenyl, a substituted or unsubstituted naphthyl, a substituted or unsubstituted p-biphenyl, a substituted or unsubstituted m-biphenyl, a substituted or unsubstituted p-terphenyl, a substituted or unsubstituted phenanthrenyl, a substituted or unsubstituted fluorenyl, a substituted or unsubstituted pyridyl, a substituted or unsubstituted dibenzofuranyl, or a substituted or unsubstituted dibenzothiophenyl.

In one embodiment, Ar₄ and Ar₅ in the formula 2-2, each independently represent, a substituted or unsubstituted (C6-C30)aryl or a substituted or unsubstituted (3- to 30-membered)heteroaryl, preferably, Ar₄ and Ar₅ may be a substituted or unsubstituted (C6-C25)aryl or a substituted or unsubstituted (5- to 25-membered)heteroaryl, more preferably, a substituted or unsubstituted (C6-C18)aryl or a substituted or unsubstituted (5- to 18-membered)heteroaryl. For example, Ar₄ and Ar₅ each independently may be a substituted or unsubstituted phenyl, a substituted or unsubstituted naphthyl, a substituted or unsubstituted phenanthrenyl, a substituted or unsubstituted p-biphenyl, a substituted or unsubstituted m-biphenyl, a substituted or unsubstituted o-biphenyl, a substituted or unsubstituted m-terphenyl, a substituted or unsubstituted fluorenyl, a substituted or unsubstituted spirobifluorenyl, a substituted or unsubstituted dibenzofuranyl, or a substituted or unsubstituted dibenzothiophenyl.

In one embodiment, the second host material represented by the above formula 2 may be more specifically illustrated by the following compounds, but is not limited thereto,

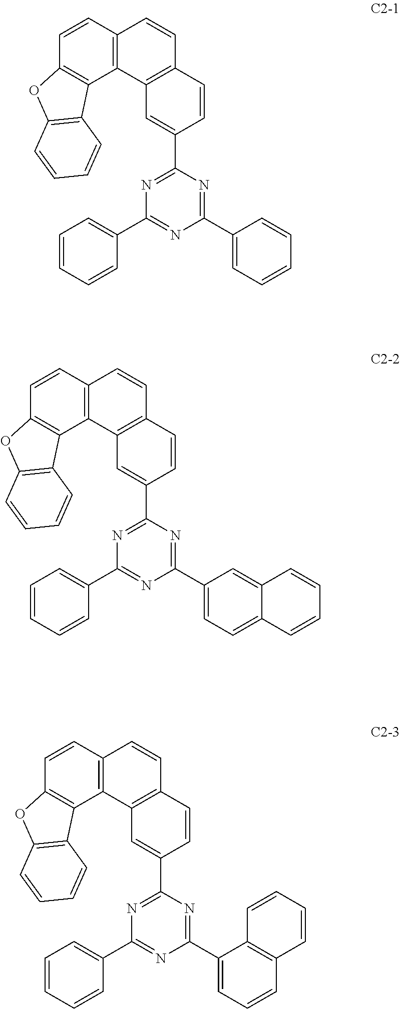 [SMILES: c1ccc(-c2nc(-c3ccccc3)nc(-c3ccc4ccc5ccc6oc7ccccc7c6c5c4c3)n2)cc1 ; c1ccc(-c2nc(-c3ccc4ccccc4c3)nc(-c3ccc4ccc5ccc6oc7ccccc7c6c5c4c3)n2)cc1 ; c1ccc(-c2nc(-c3ccc4ccc5ccc6oc7ccccc7c6c5c4c3)nc(-c3cccc4ccccc34)n2)cc1]

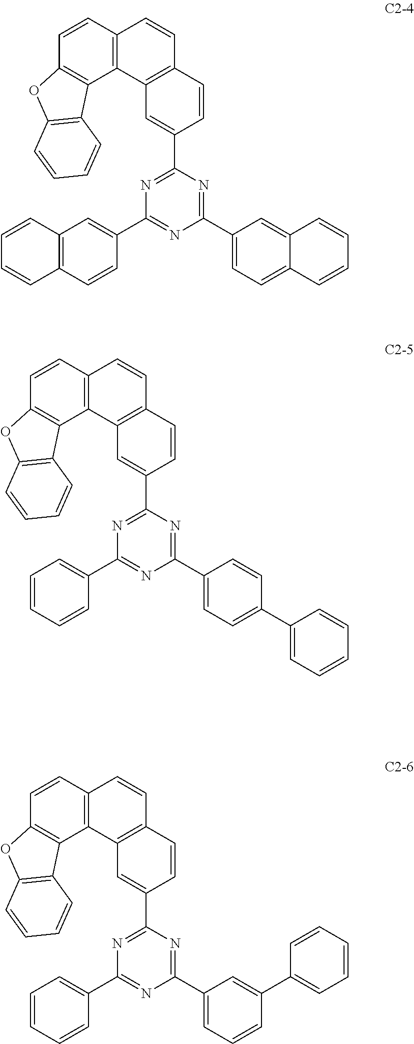 [SMILES: c1ccc2cc(-c3nc(-c4ccc5ccccc5c4)nc(-c4ccc5ccc6ccc7oc8ccccc8c7c6c5c4)n3)ccc2c1 ; c1ccc(-c2ccc(-c3nc(-c4ccccc4)nc(-c4ccc5ccc6ccc7oc8ccccc8c7c6c5c4)n3)cc2)cc1 ; c1ccc(-c2cccc(-c3nc(-c4ccccc4)nc(-c4ccc5ccc6ccc7oc8ccccc8c7c6c5c4)n3)c2)cc1]

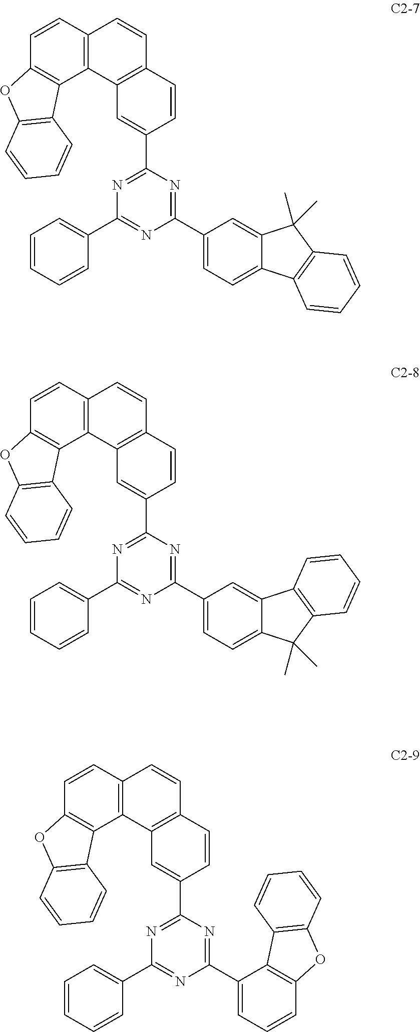 [SMILES: CC1(C)c2ccccc2-c2ccc(-c3nc(-c4ccccc4)nc(-c4ccc5ccc6ccc7oc8ccccc8c7c6c5c4)n3)cc21 ; CC1(C)c2ccccc2-c2cc(-c3nc(-c4ccccc4)nc(-c4ccc5ccc6ccc7oc8ccccc8c7c6c5c4)n3)ccc21 ; c1ccc(-c2nc(-c3ccc4ccc5ccc6oc7ccccc7c6c5c4c3)nc(-c3cccc4oc5ccccc5c34)n2)cc1]

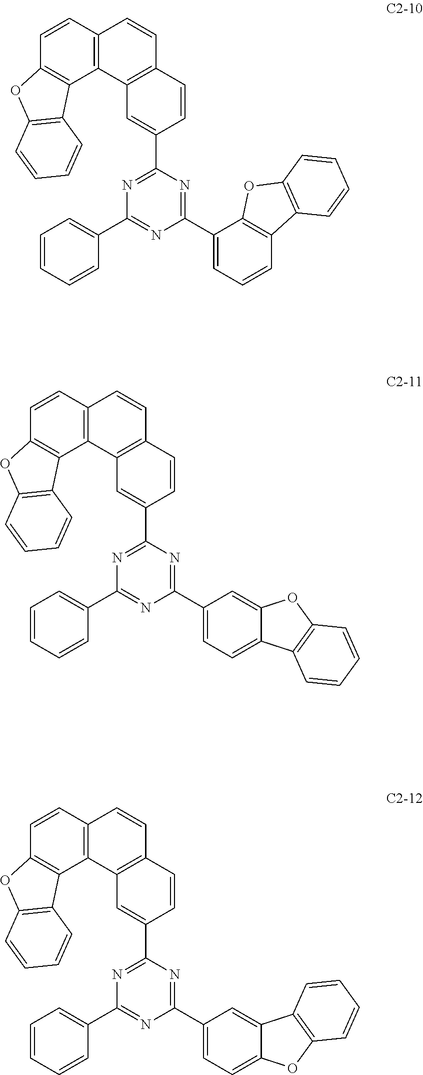 [SMILES: c1ccc(-c2nc(-c3ccc4ccc5ccc6oc7ccccc7c6c5c4c3)nc(-c3cccc4c3oc3ccccc34)n2)cc1 ; c1ccc(-c2nc(-c3ccc4c(c3)oc3ccccc34)nc(-c3ccc4ccc5ccc6oc7ccccc7c6c5c4c3)n2)cc1 ; c1ccc(-c2nc(-c3ccc4oc5ccccc5c4c3)nc(-c3ccc4ccc5ccc6oc7ccccc7c6c5c4c3)n2)cc1]

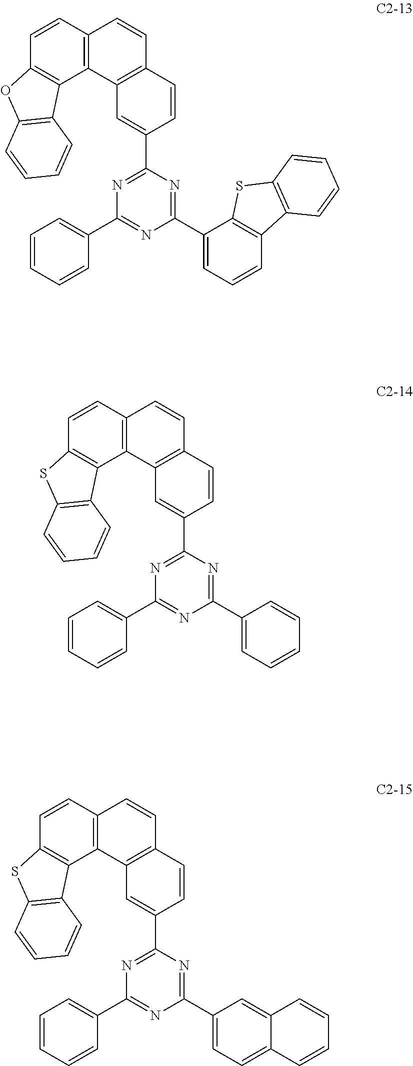 [SMILES: c1ccc(-c2nc(-c3ccc4ccc5ccc6oc7ccccc7c6c5c4c3)nc(-c3cccc4c3sc3ccccc34)n2)cc1 ; c1ccc(-c2nc(-c3ccccc3)nc(-c3ccc4ccc5ccc6sc7ccccc7c6c5c4c3)n2)cc1 ; c1ccc(-c2nc(-c3ccc4ccccc4c3)nc(-c3ccc4ccc5ccc6sc7ccccc7c6c5c4c3)n2)cc1]

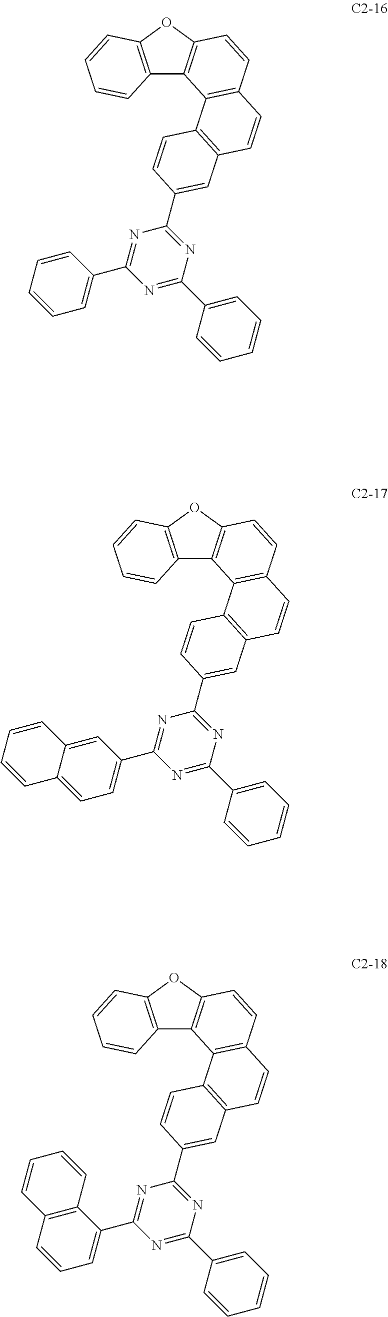 [SMILES: c1ccc(-c2nc(-c3ccccc3)nc(-c3ccc4c(ccc5ccc6oc7ccccc7c6c54)c3)n2)cc1 ; c1ccc(-c2nc(-c3ccc4ccccc4c3)nc(-c3ccc4c(ccc5ccc6oc7ccccc7c6c54)c3)n2)cc1 ; c1ccc(-c2nc(-c3ccc4c(ccc5ccc6oc7ccccc7c6c54)c3)nc(-c3cccc4ccccc34)n2)cc1]

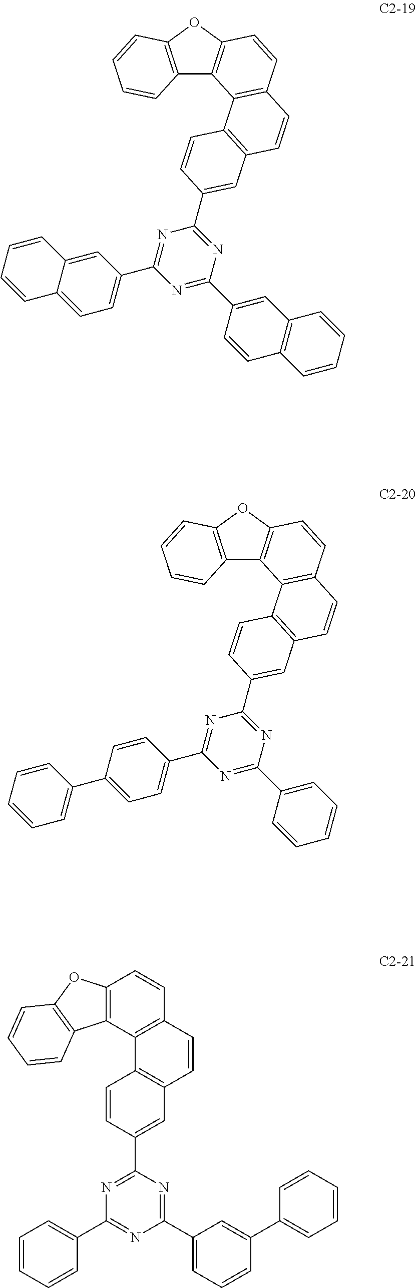 [SMILES: c1ccc2cc(-c3nc(-c4ccc5ccccc5c4)nc(-c4ccc5c(ccc6ccc7oc8ccccc8c7c65)c4)n3)ccc2c1 ; c1ccc(-c2ccc(-c3nc(-c4ccccc4)nc(-c4ccc5c(ccc6ccc7oc8ccccc8c7c65)c4)n3)cc2)cc1 ; c1ccc(-c2cccc(-c3nc(-c4ccccc4)nc(-c4ccc5c(ccc6ccc7oc8ccccc8c7c65)c4)n3)c2)cc1]

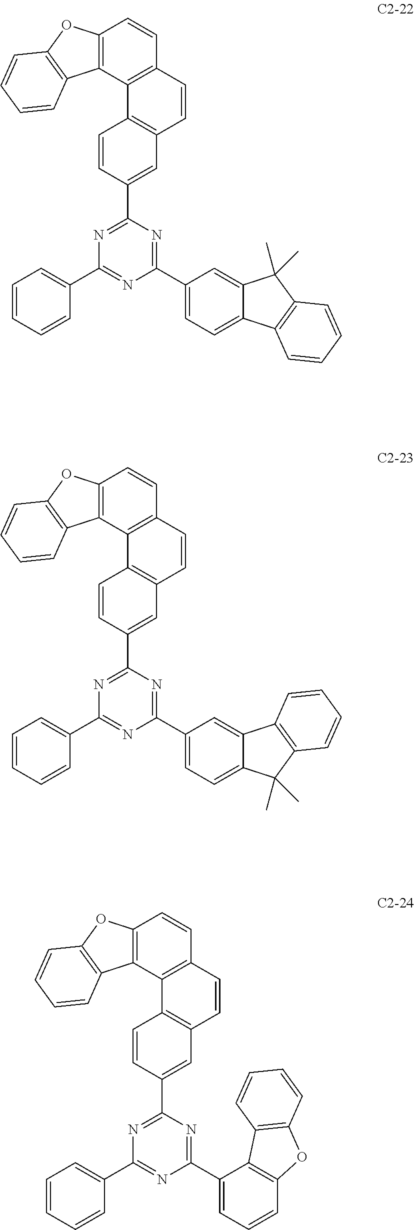 [SMILES: CC1(C)c2ccccc2-c2ccc(-c3nc(-c4ccccc4)nc(-c4ccc5c(ccc6ccc7oc8ccccc8c7c65)c4)n3)cc21 ; CC1(C)c2ccccc2-c2cc(-c3nc(-c4ccccc4)nc(-c4ccc5c(ccc6ccc7oc8ccccc8c7c65)c4)n3)ccc21 ; c1ccc(-c2nc(-c3ccc4c(ccc5ccc6oc7ccccc7c6c54)c3)nc(-c3cccc4oc5ccccc5c34)n2)cc1]

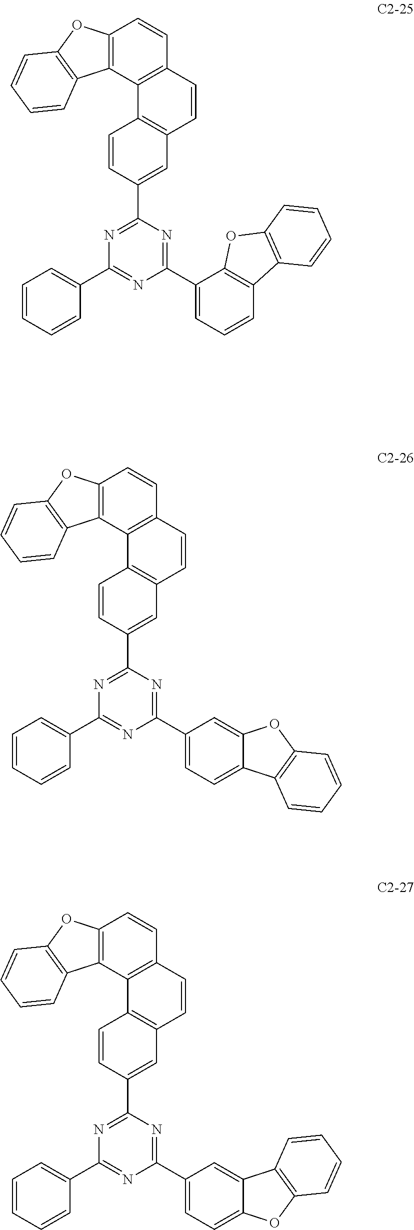 [SMILES: c1ccc(-c2nc(-c3ccc4c(ccc5ccc6oc7ccccc7c6c54)c3)nc(-c3cccc4c3oc3ccccc34)n2)cc1 ; c1ccc(-c2nc(-c3ccc4c(ccc5ccc6oc7ccccc7c6c54)c3)nc(-c3ccc4c(c3)oc3ccccc34)n2)cc1 ; c1ccc(-c2nc(-c3ccc4c(ccc5ccc6oc7ccccc7c6c54)c3)nc(-c3ccc4oc5ccccc5c4c3)n2)cc1]

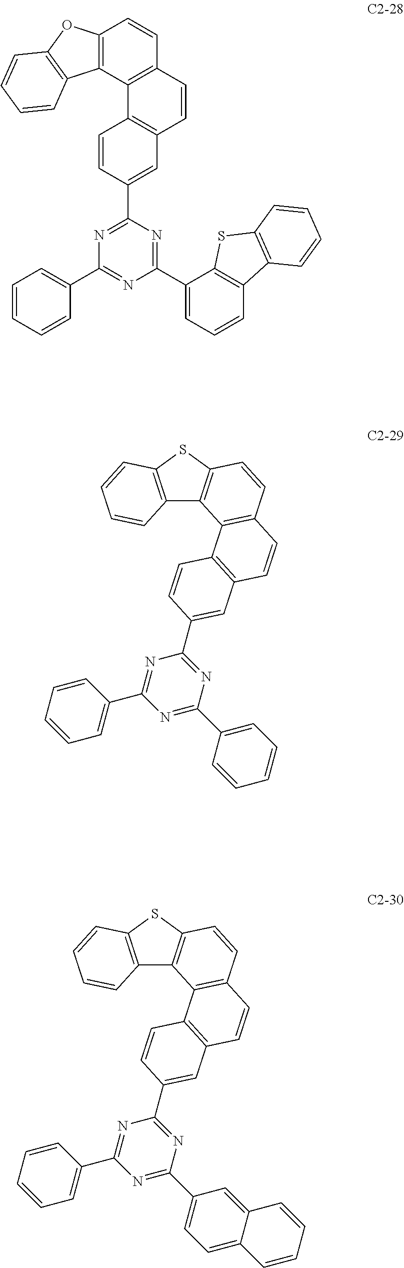 [SMILES: c1ccc(-c2nc(-c3ccc4c(ccc5ccc6oc7ccccc7c6c54)c3)nc(-c3cccc4c3sc3ccccc34)n2)cc1 ; c1ccc(-c2nc(-c3ccccc3)nc(-c3ccc4c(ccc5ccc6sc7ccccc7c6c54)c3)n2)cc1 ; c1ccc(-c2nc(-c3ccc4ccccc4c3)nc(-c3ccc4c(ccc5ccc6sc7ccccc7c6c54)c3)n2)cc1]

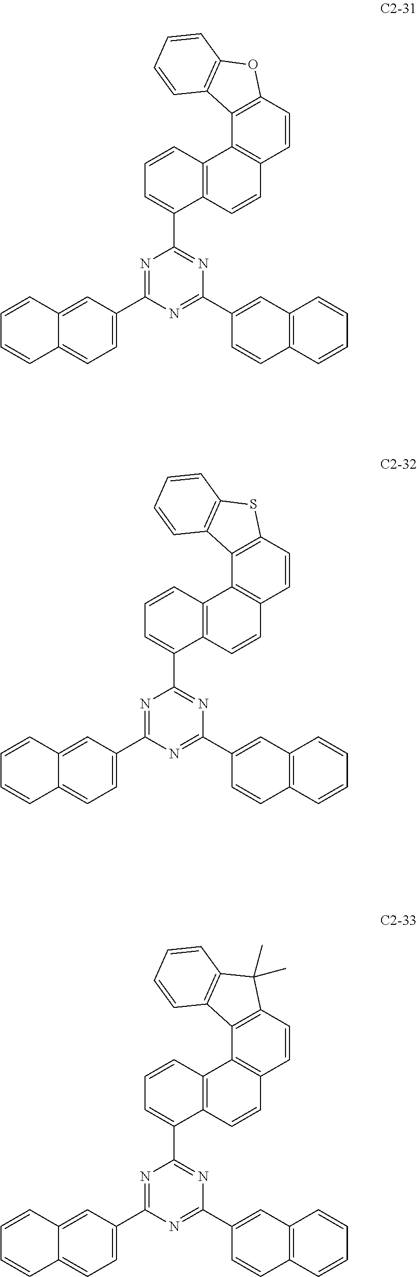 [SMILES: c1ccc2cc(-c3nc(-c4ccc5ccccc5c4)nc(-c4cccc5c4ccc4ccc6oc7ccccc7c6c45)n3)ccc2c1 ; c1ccc2cc(-c3nc(-c4ccc5ccccc5c4)nc(-c4cccc5c4ccc4ccc6sc7ccccc7c6c45)n3)ccc2c1 ; CC1(C)c2ccccc2-c2c1ccc1ccc3c(-c4nc(-c5ccc6ccccc6c5)nc(-c5ccc6ccccc6c5)n4)cccc3c21]

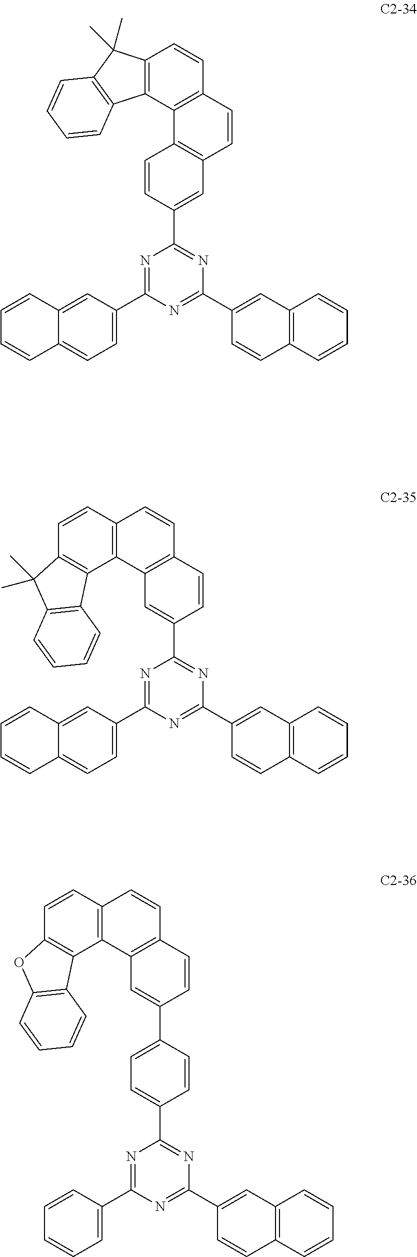 [SMILES: CC1(C)c2ccccc2-c2c1ccc1ccc3cc(-c4nc(-c5ccc6ccccc6c5)nc(-c5ccc6ccccc6c5)n4)ccc3c21 ; CC1(C)c2ccccc2-c2c1ccc1ccc3ccc(-c4nc(-c5ccc6ccccc6c5)nc(-c5ccc6ccccc6c5)n4)cc3c21 ; c1ccc(-c2nc(-c3ccc(-c4ccc5ccc6ccc7oc8ccccc8c7c6c5c4)cc3)nc(-c3ccc4ccccc4c3)n2)cc1]

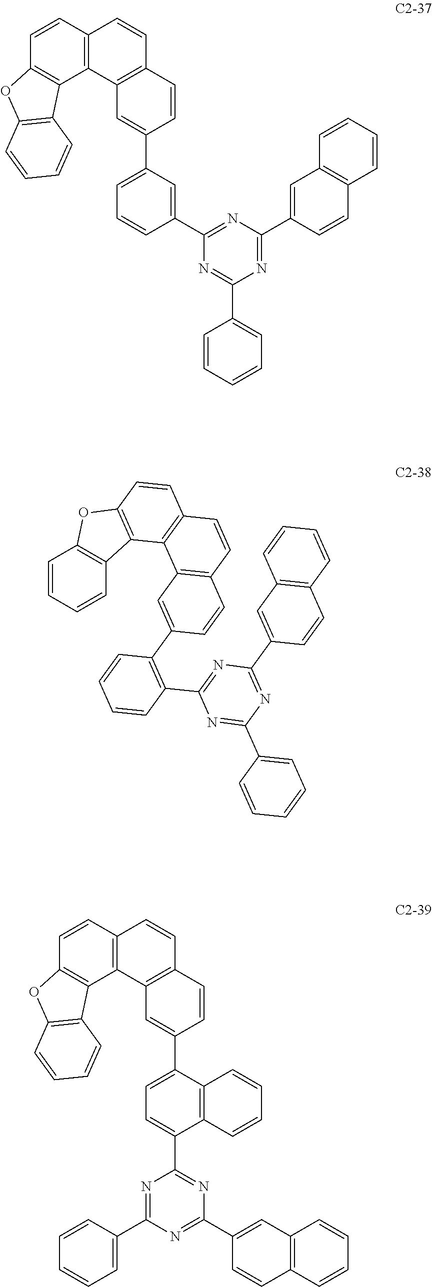 [SMILES: c1ccc(-c2nc(-c3cccc(-c4ccc5ccc6ccc7oc8ccccc8c7c6c5c4)c3)nc(-c3ccc4ccccc4c3)n2)cc1 ; c1ccc(-c2nc(-c3ccc4ccccc4c3)nc(-c3ccccc3-c3ccc4ccc5ccc6oc7ccccc7c6c5c4c3)n2)cc1 ; c1ccc(-c2nc(-c3ccc4ccccc4c3)nc(-c3ccc(-c4ccc5ccc6ccc7oc8ccccc8c7c6c5c4)c4ccccc34)n2)cc1]

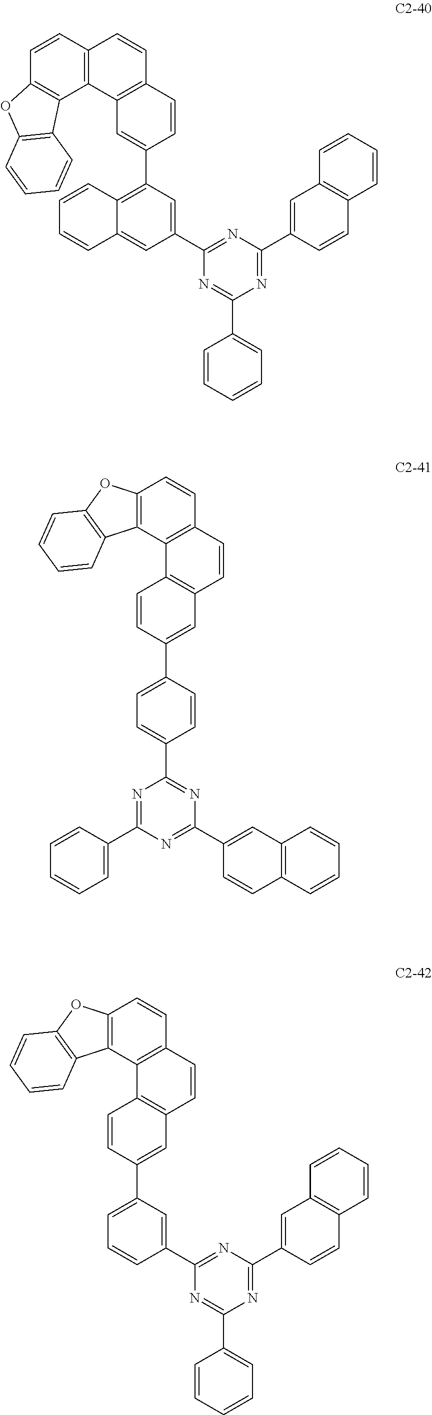 [SMILES: c1ccc(-c2nc(-c3ccc4ccccc4c3)nc(-c3cc(-c4ccc5ccc6ccc7oc8ccccc8c7c6c5c4)c4ccccc4c3)n2)cc1 ; c1ccc(-c2nc(-c3ccc(-c4ccc5c(ccc6ccc7oc8ccccc8c7c65)c4)cc3)nc(-c3ccc4ccccc4c3)n2)cc1 ; c1ccc(-c2nc(-c3cccc(-c4ccc5c(ccc6ccc7oc8ccccc8c7c65)c4)c3)nc(-c3ccc4ccccc4c3)n2)cc1]

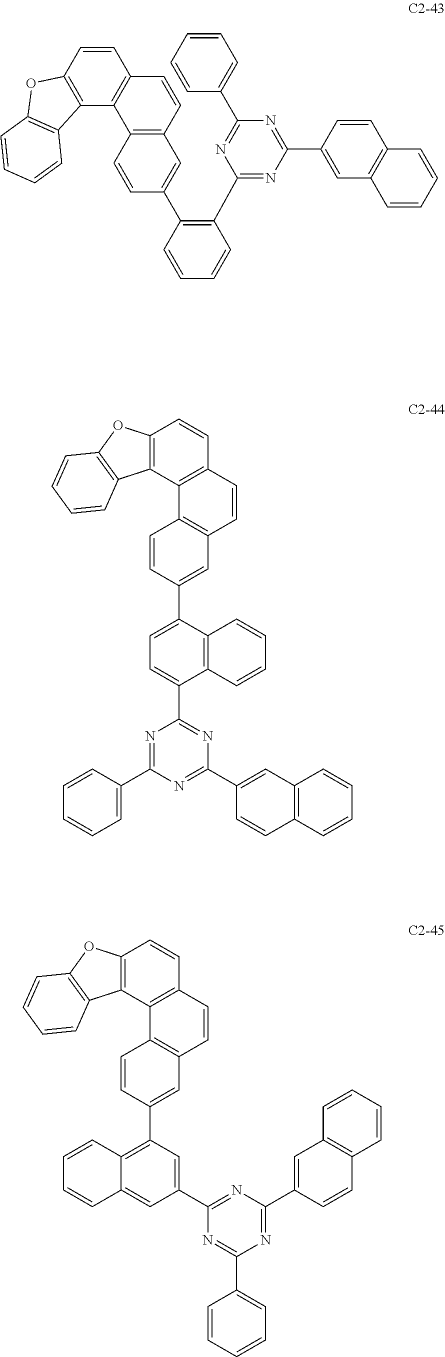 [SMILES: c1ccc(-c2nc(-c3ccc4ccccc4c3)nc(-c3ccccc3-c3ccc4c(ccc5ccc6oc7ccccc7c6c54)c3)n2)cc1 ; c1ccc(-c2nc(-c3ccc4ccccc4c3)nc(-c3ccc(-c4ccc5c(ccc6ccc7oc8ccccc8c7c65)c4)c4ccccc34)n2)cc1 ; c1ccc(-c2nc(-c3ccc4ccccc4c3)nc(-c3cc(-c4ccc5c(ccc6ccc7oc8ccccc8c7c65)c4)c4ccccc4c3)n2)cc1]

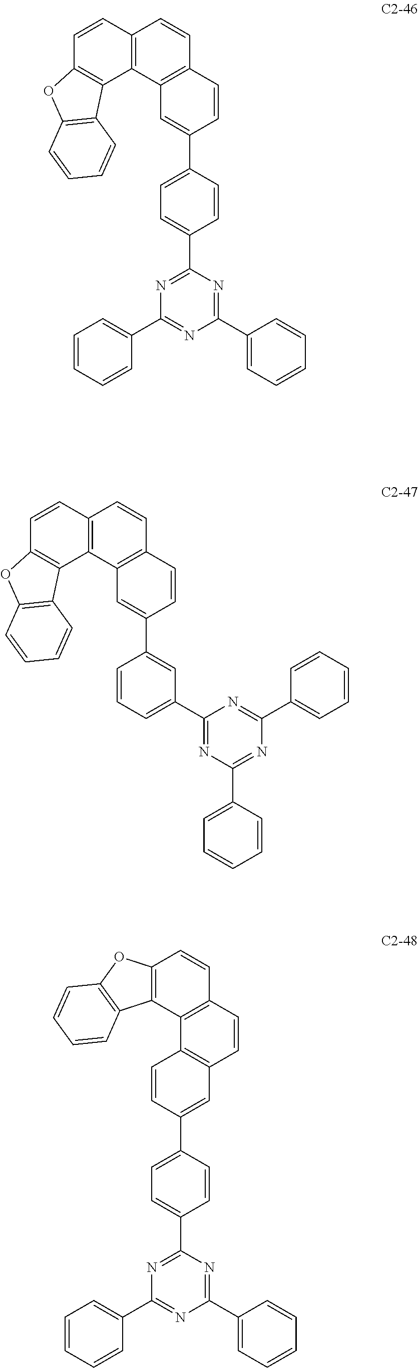 [SMILES: c1ccc(-c2nc(-c3ccccc3)nc(-c3ccc(-c4ccc5ccc6ccc7oc8ccccc8c7c6c5c4)cc3)n2)cc1 ; c1ccc(-c2nc(-c3ccccc3)nc(-c3cccc(-c4ccc5ccc6ccc7oc8ccccc8c7c6c5c4)c3)n2)cc1 ; c1ccc(-c2nc(-c3ccccc3)nc(-c3ccc(-c4ccc5c(ccc6ccc7oc8ccccc8c7c65)c4)cc3)n2)cc1]

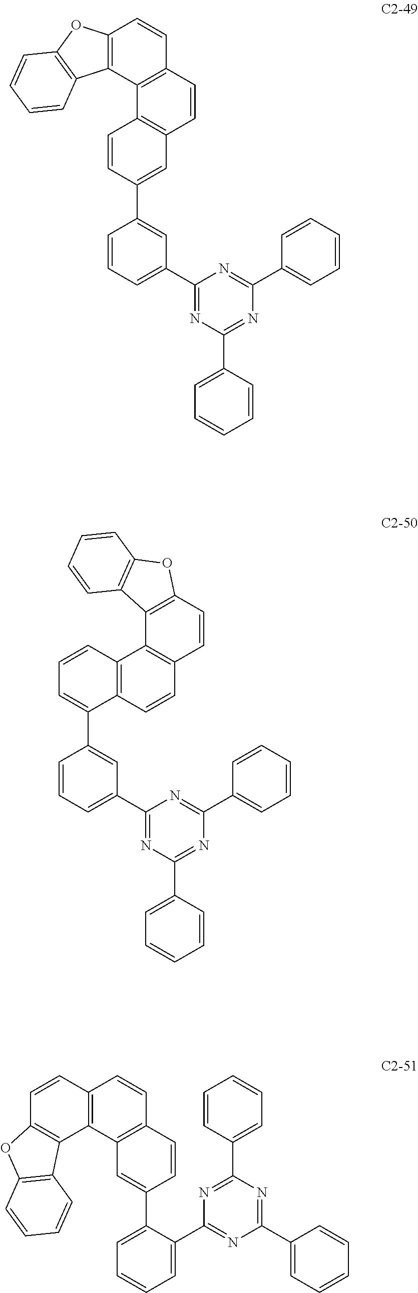 [SMILES: c1ccc(-c2nc(-c3ccccc3)nc(-c3cccc(-c4ccc5c(ccc6ccc7oc8ccccc8c7c65)c4)c3)n2)cc1 ; c1ccc(-c2nc(-c3ccccc3)nc(-c3cccc(-c4cccc5c4ccc4ccc6oc7ccccc7c6c45)c3)n2)cc1 ; c1ccc(-c2nc(-c3ccccc3)nc(-c3ccccc3-c3ccc4ccc5ccc6oc7ccccc7c6c5c4c3)n2)cc1]

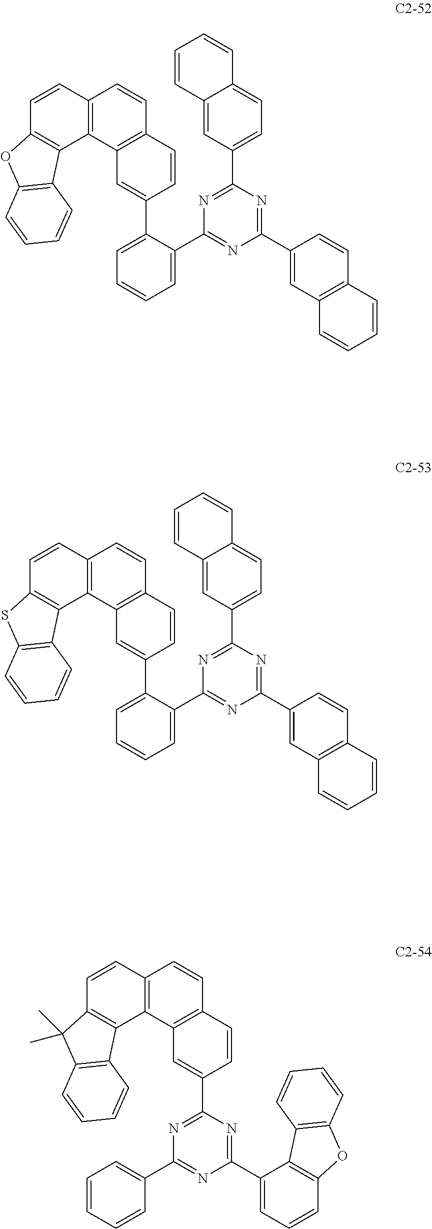 [SMILES: c1ccc(-c2nc(-c3ccc4ccccc4c3)nc(-c3ccc4ccccc4c3)n2)c(-c2ccc3ccc4ccc5oc6ccccc6c5c4c3c2)c1 ; c1ccc(-c2nc(-c3ccc4ccccc4c3)nc(-c3ccc4ccccc4c3)n2)c(-c2ccc3ccc4ccc5sc6ccccc6c5c4c3c2)c1 ; CC1(C)c2ccccc2-c2c1ccc1ccc3ccc(-c4nc(-c5ccccc5)nc(-c5cccc6oc7ccccc7c56)n4)cc3c21]

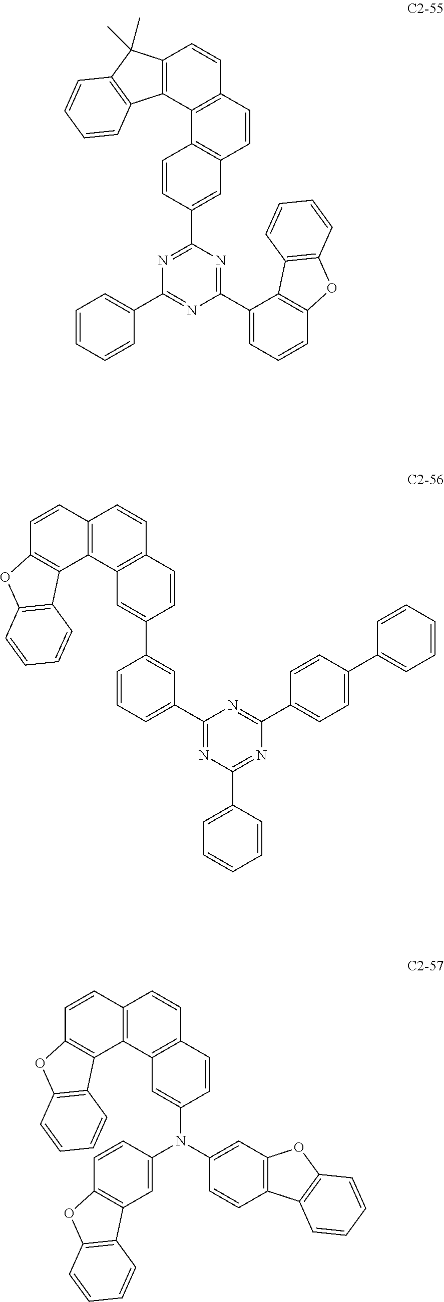 [SMILES: CC1(C)c2ccccc2-c2c1ccc1ccc3cc(-c4nc(-c5ccccc5)nc(-c5cccc6oc7ccccc7c56)n4)ccc3c21 ; c1ccc(-c2ccc(-c3nc(-c4ccccc4)nc(-c4cccc(-c5ccc6ccc7ccc8oc9ccccc9c8c7c6c5)c4)n3)cc2)cc1 ; c1ccc2c(c1)oc1cc(N(c3ccc4oc5ccccc5c4c3)c3ccc4ccc5ccc6oc7ccccc7c6c5c4c3)ccc12]

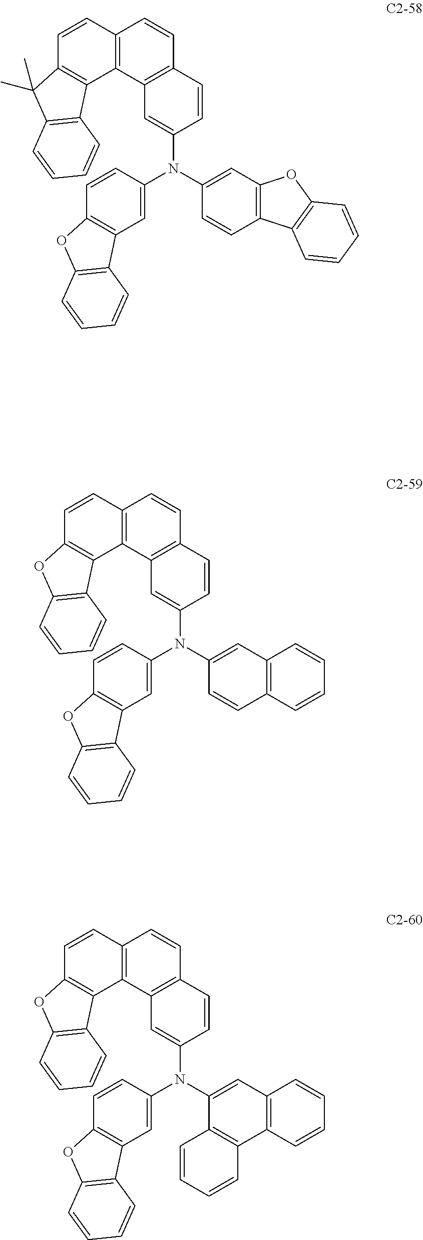 [SMILES: CC1(C)c2ccccc2-c2c1ccc1ccc3ccc(N(c4ccc5c(c4)oc4ccccc45)c4ccc5oc6ccccc6c5c4)cc3c21 ; c1ccc2cc(N(c3ccc4oc5ccccc5c4c3)c3ccc4ccc5ccc6oc7ccccc7c6c5c4c3)ccc2c1 ; c1ccc2c(c1)cc(N(c1ccc3oc4ccccc4c3c1)c1ccc3ccc4ccc5oc6ccccc6c5c4c3c1)c1ccccc12]

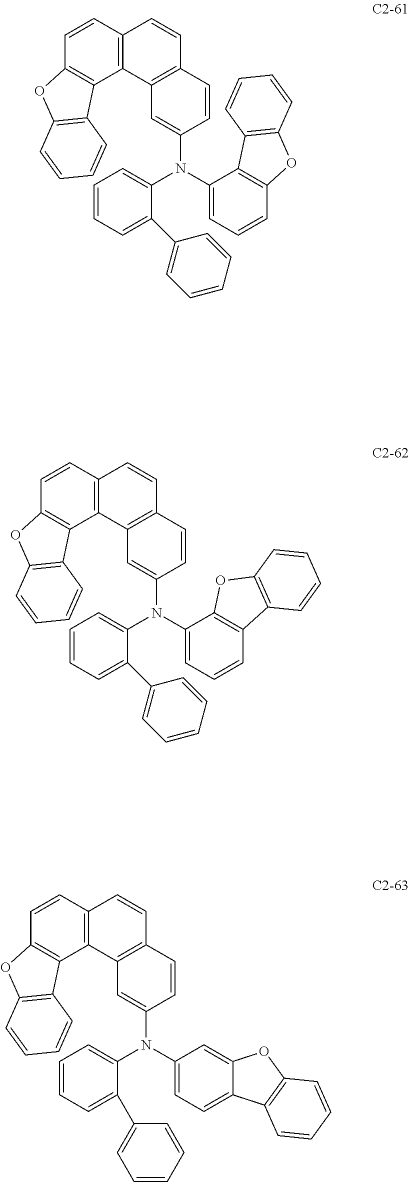 [SMILES: c1ccc(-c2ccccc2N(c2ccc3ccc4ccc5oc6ccccc6c5c4c3c2)c2cccc3oc4ccccc4c23)cc1 ; c1ccc(-c2ccccc2N(c2ccc3ccc4ccc5oc6ccccc6c5c4c3c2)c2cccc3c2oc2ccccc23)cc1 ; c1ccc(-c2ccccc2N(c2ccc3c(c2)oc2ccccc23)c2ccc3ccc4ccc5oc6ccccc6c5c4c3c2)cc1]

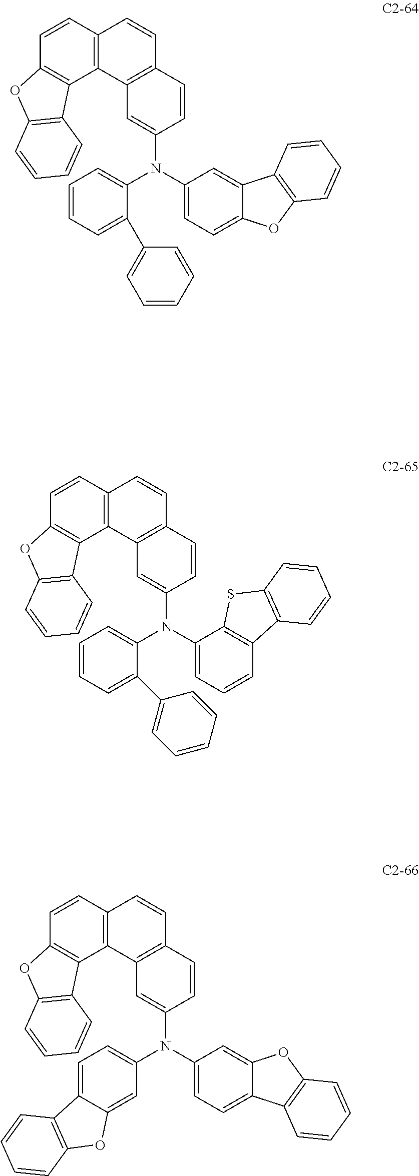 [SMILES: c1ccc(-c2ccccc2N(c2ccc3oc4ccccc4c3c2)c2ccc3ccc4ccc5oc6ccccc6c5c4c3c2)cc1 ; c1ccc(-c2ccccc2N(c2ccc3ccc4ccc5oc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6oc7ccccc7c6c5c4c3)ccc12]

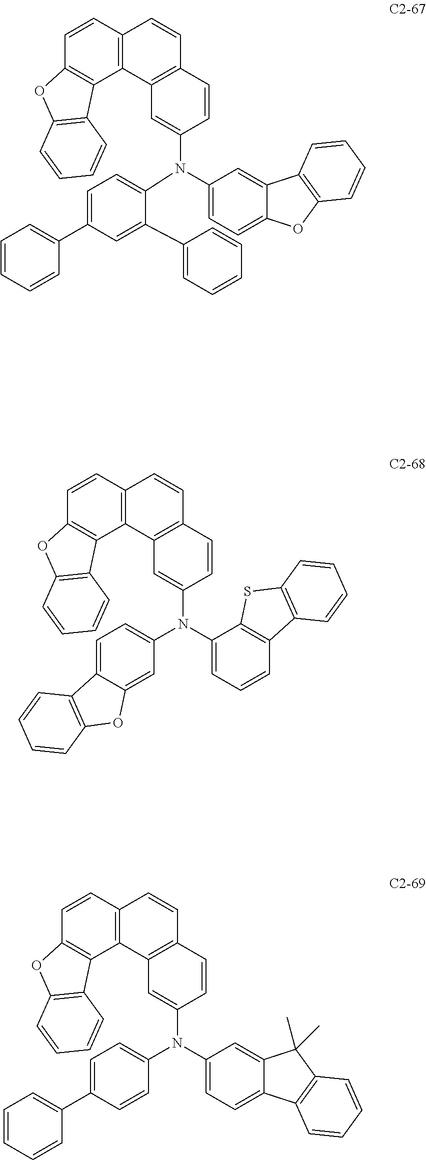 [SMILES: c1ccc(-c2ccc(N(c3ccc4oc5ccccc5c4c3)c3ccc4ccc5ccc6oc7ccccc7c6c5c4c3)c(-c3ccccc3)c2)cc1 ; c1ccc2c(c1)oc1cc(N(c3ccc4ccc5ccc6oc7ccccc7c6c5c4c3)c3cccc4c3sc3ccccc34)ccc12 ; CC1(C)c2ccccc2-c2ccc(N(c3ccc(-c4ccccc4)cc3)c3ccc4ccc5ccc6oc7ccccc7c6c5c4c3)cc21]

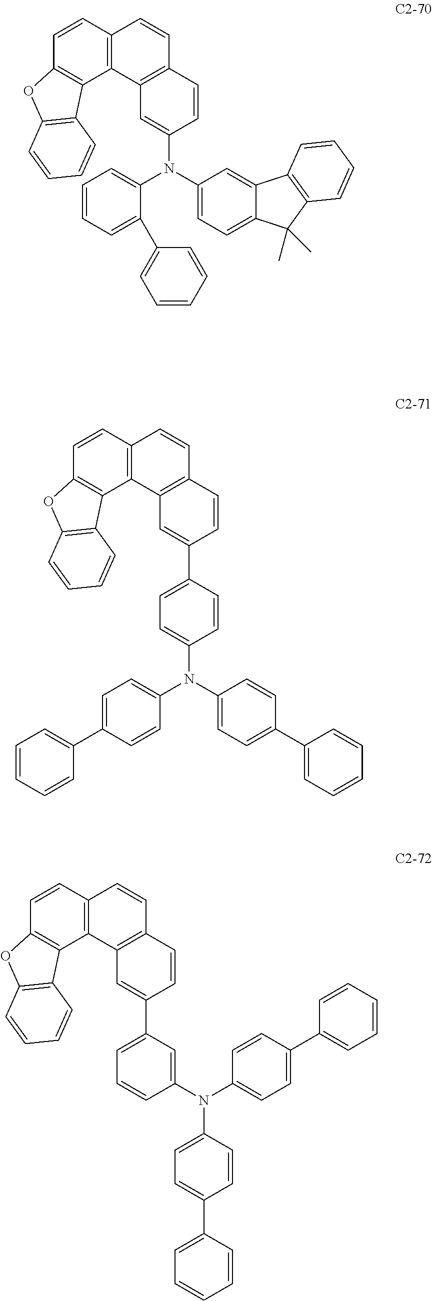 [SMILES: CC1(C)c2ccccc2-c2cc(N(c3ccc4ccc5ccc6oc7ccccc7c6c5c4c3)c3ccccc3-c3ccccc3)ccc21 ; c1ccc(-c2ccc(N(c3ccc(-c4ccccc4)cc3)c3ccc(-c4ccc5ccc6ccc7oc8ccccc8c7c6c5c4)cc3)cc2)cc1 ; c1ccc(-c2ccc(N(c3ccc(-c4ccccc4)cc3)c3cccc(-c4ccc5ccc6ccc7oc8ccccc8c7c6c5c4)c3)cc2)cc1]

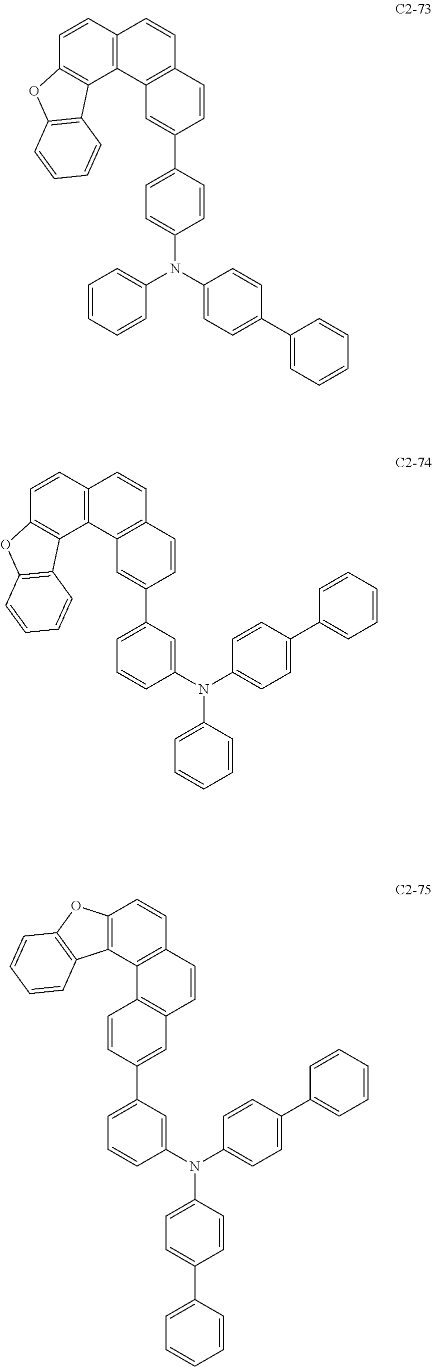 [SMILES: c1ccc(-c2ccc(N(c3ccccc3)c3ccc(-c4ccc5ccc6ccc7oc8ccccc8c7c6c5c4)cc3)cc2)cc1 ; c1ccc(-c2ccc(N(c3ccccc3)c3cccc(-c4ccc5ccc6ccc7oc8ccccc8c7c6c5c4)c3)cc2)cc1 ; c1ccc(-c2ccc(N(c3ccc(-c4ccccc4)cc3)c3cccc(-c4ccc5c(ccc6ccc7oc8ccccc8c7c65)c4)c3)cc2)cc1]

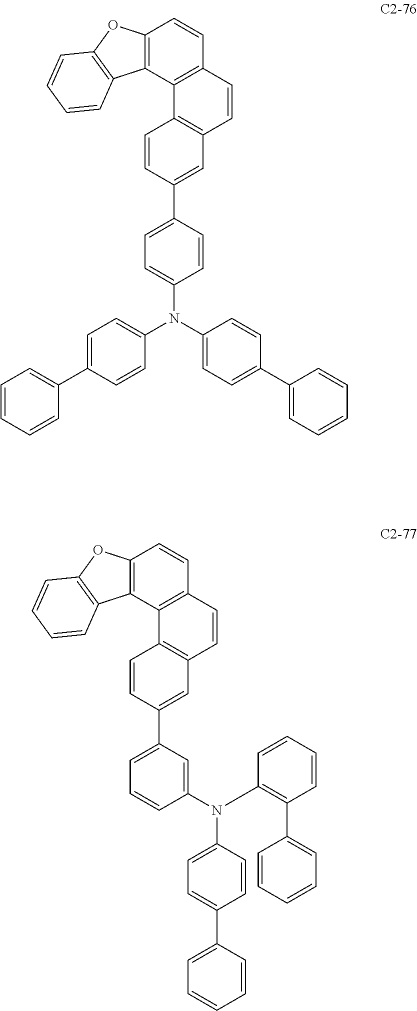 [SMILES: c1ccc(-c2ccc(N(c3ccc(-c4ccccc4)cc3)c3ccc(-c4ccc5c(ccc6ccc7oc8ccccc8c7c65)c4)cc3)cc2)cc1 ; c1ccc(-c2ccc(N(c3cccc(-c4ccc5c(ccc6ccc7oc8ccccc8c7c65)c4)c3)c3ccccc3-c3ccccc3)cc2)cc1]

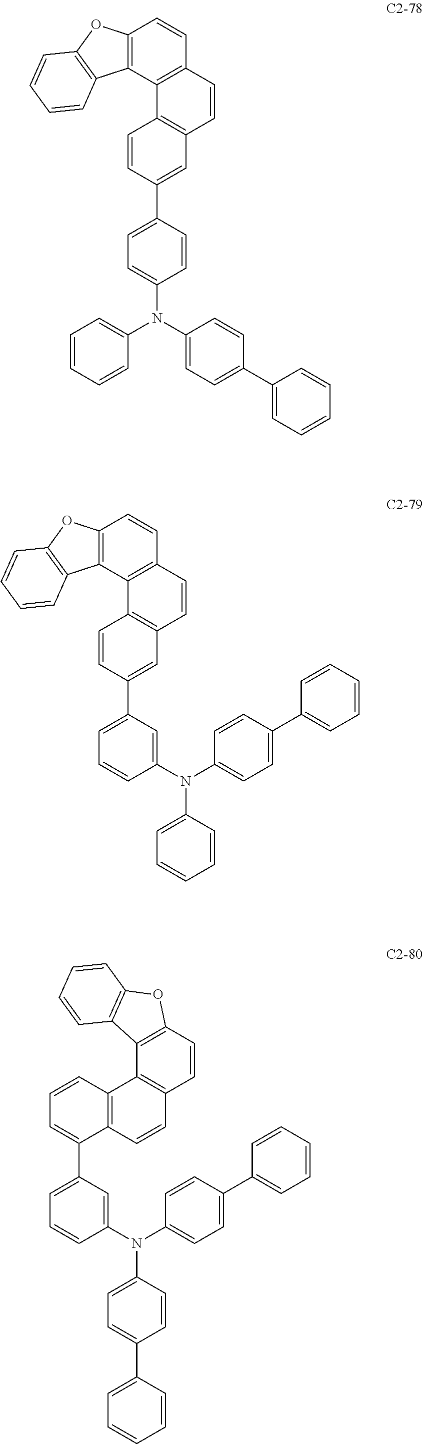 [SMILES: c1ccc(-c2ccc(N(c3ccccc3)c3ccc(-c4ccc5c(ccc6ccc7oc8ccccc8c7c65)c4)cc3)cc2)cc1 ; c1ccc(-c2ccc(N(c3ccccc3)c3cccc(-c4ccc5c(ccc6ccc7oc8ccccc8c7c65)c4)c3)cc2)cc1 ; c1ccc(-c2ccc(N(c3ccc(-c4ccccc4)cc3)c3cccc(-c4cccc5c4ccc4ccc6oc7ccccc7c6c45)c3)cc2)cc1]

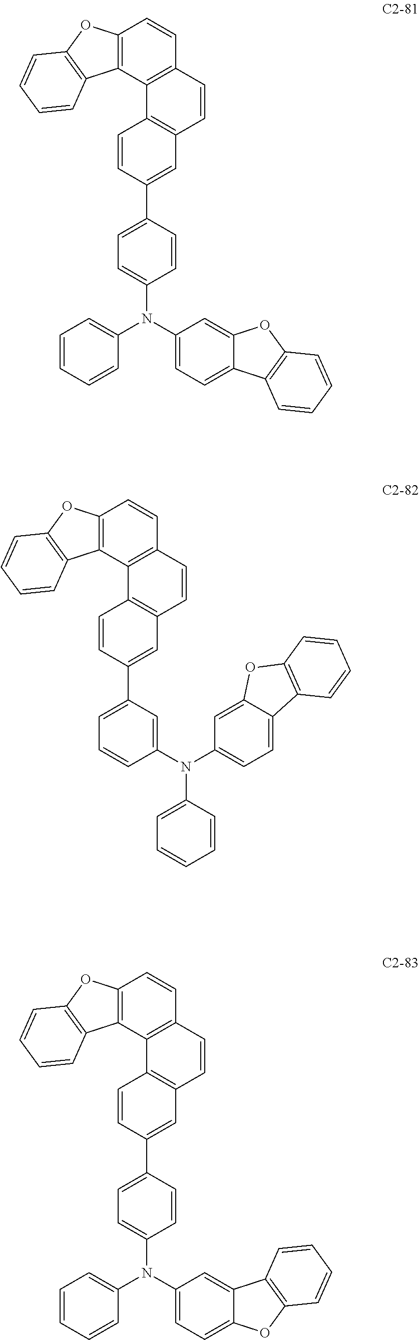 [SMILES: c1ccc(N(c2ccc(-c3ccc4c(ccc5ccc6oc7ccccc7c6c54)c3)cc2)c2ccc3c(c2)oc2ccccc23)cc1 ; c1ccc(N(c2cccc(-c3ccc4c(ccc5ccc6oc7ccccc7c6c54)c3)c2)c2ccc3c(c2)oc2ccccc23)cc1 ; c1ccc(N(c2ccc(-c3ccc4c(ccc5ccc6oc7ccccc7c6c54)c3)cc2)c2ccc3oc4ccccc4c3c2)cc1]

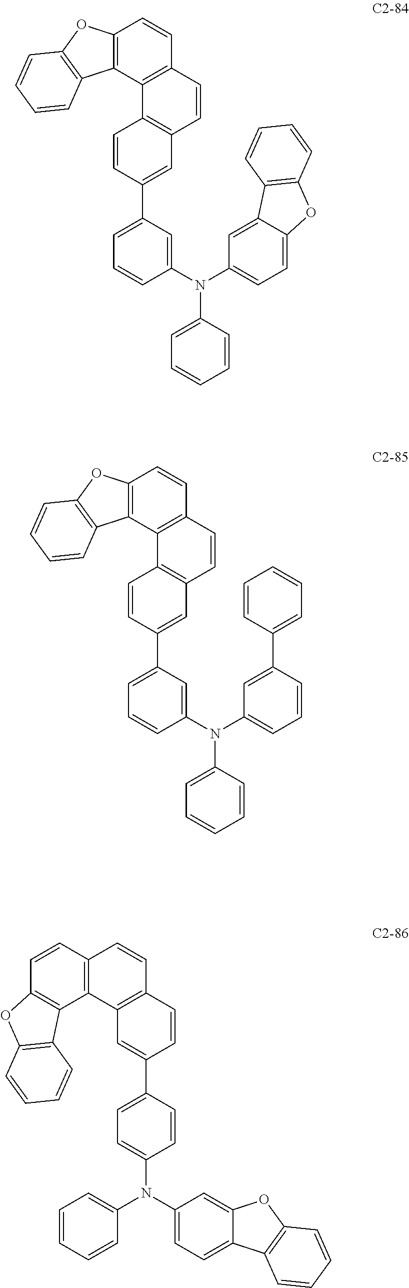 [SMILES: c1ccc(N(c2cccc(-c3ccc4c(ccc5ccc6oc7ccccc7c6c54)c3)c2)c2ccc3oc4ccccc4c3c2)cc1 ; c1ccc(-c2cccc(N(c3ccccc3)c3cccc(-c4ccc5c(ccc6ccc7oc8ccccc8c7c65)c4)c3)c2)cc1 ; c1ccc(N(c2ccc(-c3ccc4ccc5ccc6oc7ccccc7c6c5c4c3)cc2)c2ccc3c(c2)oc2ccccc23)cc1]

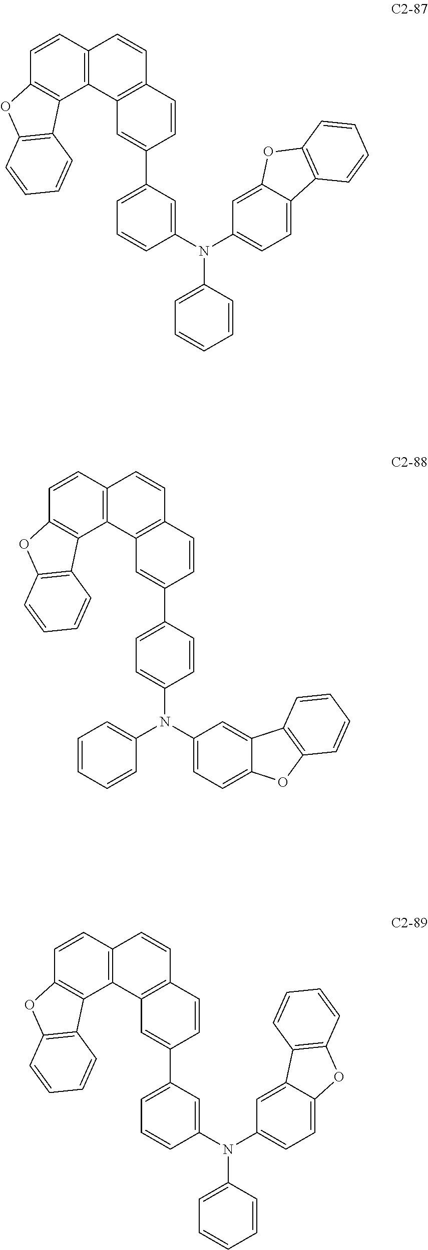 [SMILES: c1ccc(N(c2cccc(-c3ccc4ccc5ccc6oc7ccccc7c6c5c4c3)c2)c2ccc3c(c2)oc2ccccc23)cc1 ; c1ccc(N(c2ccc(-c3ccc4ccc5ccc6oc7ccccc7c6c5c4c3)cc2)c2ccc3oc4ccccc4c3c2)cc1 ; c1ccc(N(c2cccc(-c3ccc4ccc5ccc6oc7ccccc7c6c5c4c3)c2)c2ccc3oc4ccccc4c3c2)cc1]

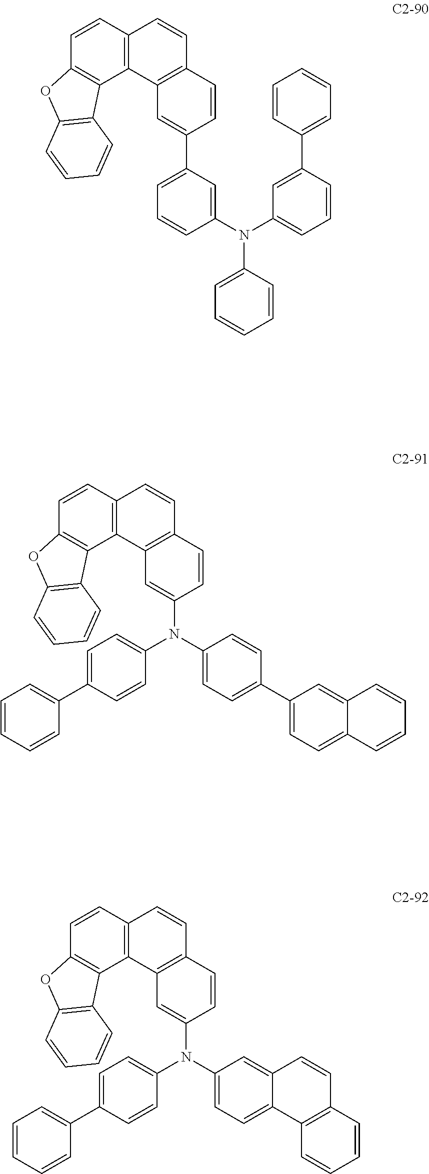 [SMILES: c1ccc(-c2cccc(N(c3ccccc3)c3cccc(-c4ccc5ccc6ccc7oc8ccccc8c7c6c5c4)c3)c2)cc1 ; c1ccc(-c2ccc(N(c3ccc(-c4ccc5ccccc5c4)cc3)c3ccc4ccc5ccc6oc7ccccc7c6c5c4c3)cc2)cc1 ; c1ccc(-c2ccc(N(c3ccc4c(ccc5ccccc54)c3)c3ccc4ccc5ccc6oc7ccccc7c6c5c4c3)cc2)cc1]

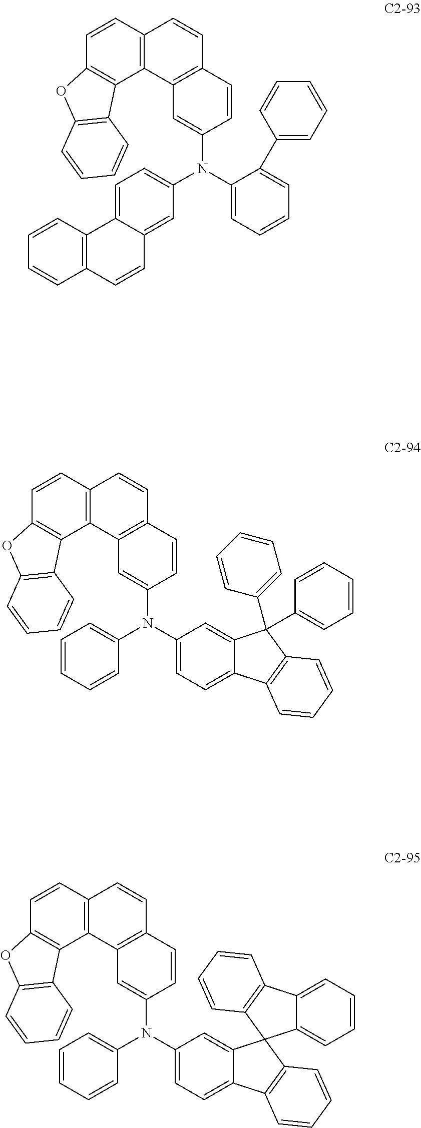 [SMILES: c1ccc(-c2ccccc2N(c2ccc3c(ccc4ccccc43)c2)c2ccc3ccc4ccc5oc6ccccc6c5c4c3c2)cc1 ; c1ccc(N(c2ccc3c(c2)C(c2ccccc2)(c2ccccc2)c2ccccc2-3)c2ccc3ccc4ccc5oc6ccccc6c5c4c3c2)cc1 ; c1ccc(N(c2ccc3c(c2)C2(c4ccccc4-c4ccccc42)c2ccccc2-3)c2ccc3ccc4ccc5oc6ccccc6c5c4c3c2)cc1]

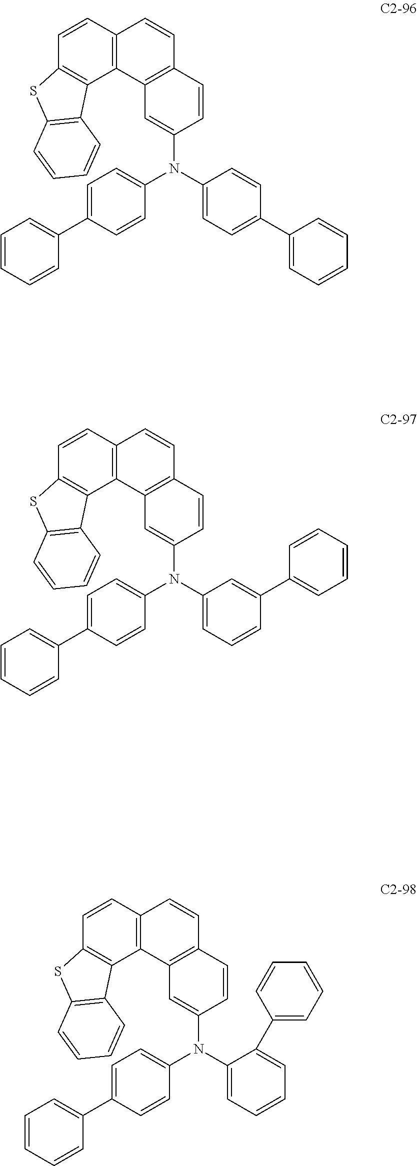 [SMILES: c1ccc(-c2ccc(N(c3ccc(-c4ccccc4)cc3)c3ccc4ccc5ccc6sc7ccccc7c6c5c4c3)cc2)cc1 ; c1ccc(-c2ccc(N(c3cccc(-c4ccccc4)c3)c3ccc4ccc5ccc6sc7ccccc7c6c5c4c3)cc2)cc1 ; c1ccc(-c2ccc(N(c3ccc4ccc5ccc6sc7ccccc7c6c5c4c3)c3ccccc3-c3ccccc3)cc2)cc1]

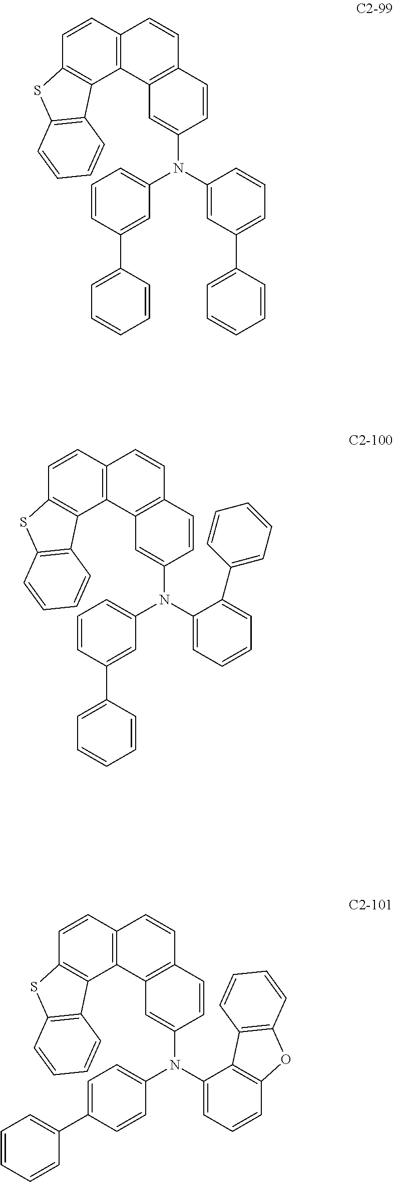 [SMILES: c1ccc(-c2cccc(N(c3cccc(-c4ccccc4)c3)c3ccc4ccc5ccc6sc7ccccc7c6c5c4c3)c2)cc1 ; c1ccc(-c2cccc(N(c3ccc4ccc5ccc6sc7ccccc7c6c5c4c3)c3ccccc3-c3ccccc3)c2)cc1 ; c1ccc(-c2ccc(N(c3ccc4ccc5ccc6sc7ccccc7c6c5c4c3)c3cccc4oc5ccccc5c34)cc2)cc1]

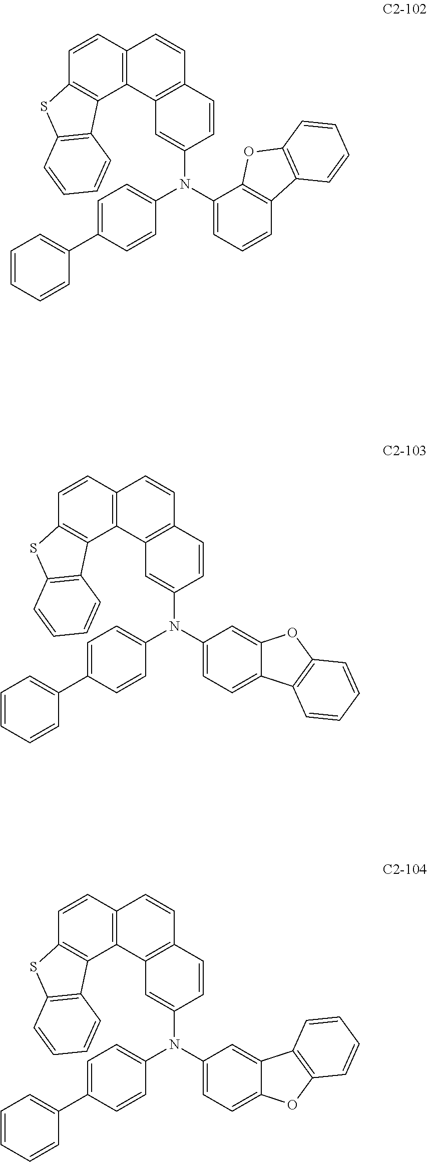 [SMILES: c1ccc(-c2ccc(N(c3ccc4ccc5ccc6sc7ccccc7c6c5c4c3)c3cccc4c3oc3ccccc34)cc2)cc1 ; c1ccc(-c2ccc(N(c3ccc4c(c3)oc3ccccc34)c3ccc4ccc5ccc6sc7ccccc7c6c5c4c3)cc2)cc1 ; c1ccc(-c2ccc(N(c3ccc4oc5ccccc5c4c3)c3ccc4ccc5ccc6sc7ccccc7c6c5c4c3)cc2)cc1]

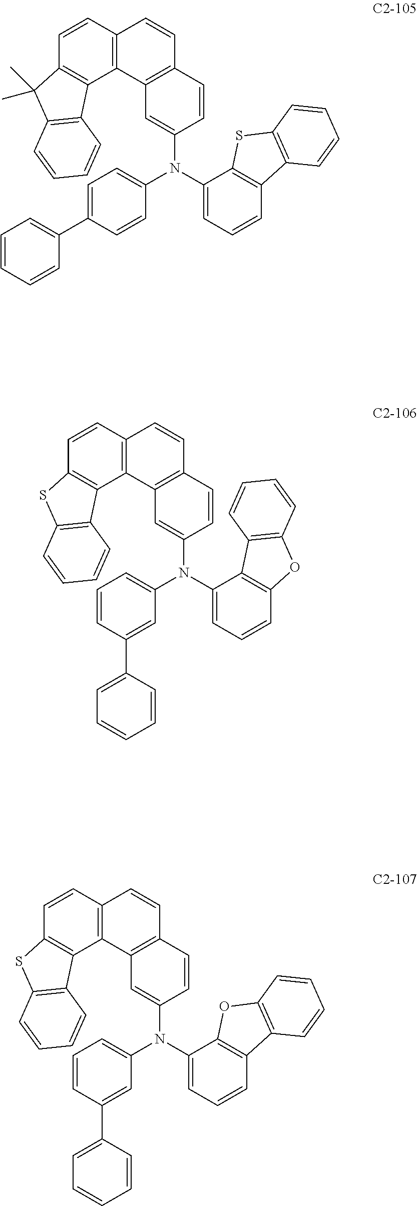 [SMILES: CC1(C)c2ccccc2-c2c1ccc1ccc3ccc(N(c4ccc(-c5ccccc5)cc4)c4cccc5c4sc4ccccc45)cc3c21 ; c1ccc(-c2cccc(N(c3ccc4ccc5ccc6sc7ccccc7c6c5c4c3)c3cccc4oc5ccccc5c34)c2)cc1 ; c1ccc(-c2cccc(N(c3ccc4ccc5ccc6sc7ccccc7c6c5c4c3)c3cccc4c3oc3ccccc34)c2)cc1]

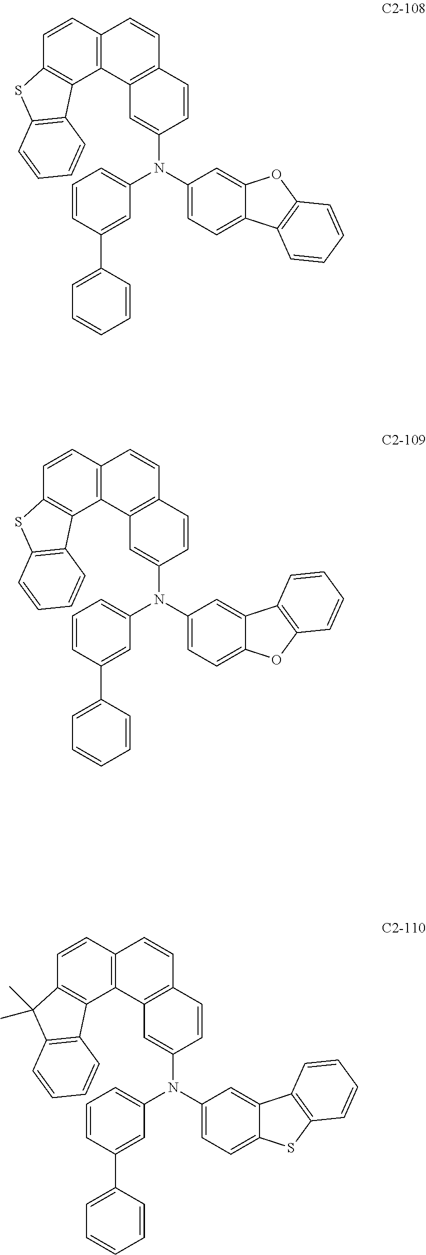 [SMILES: c1ccc(-c2cccc(N(c3ccc4c(c3)oc3ccccc34)c3ccc4ccc5ccc6sc7ccccc7c6c5c4c3)c2)cc1 ; c1ccc(-c2cccc(N(c3ccc4oc5ccccc5c4c3)c3ccc4ccc5ccc6sc7ccccc7c6c5c4c3)c2)cc1 ; CC1(C)c2ccccc2-c2c1ccc1ccc3ccc(N(c4cccc(-c5ccccc5)c4)c4ccc5sc6ccccc6c5c4)cc3c21]

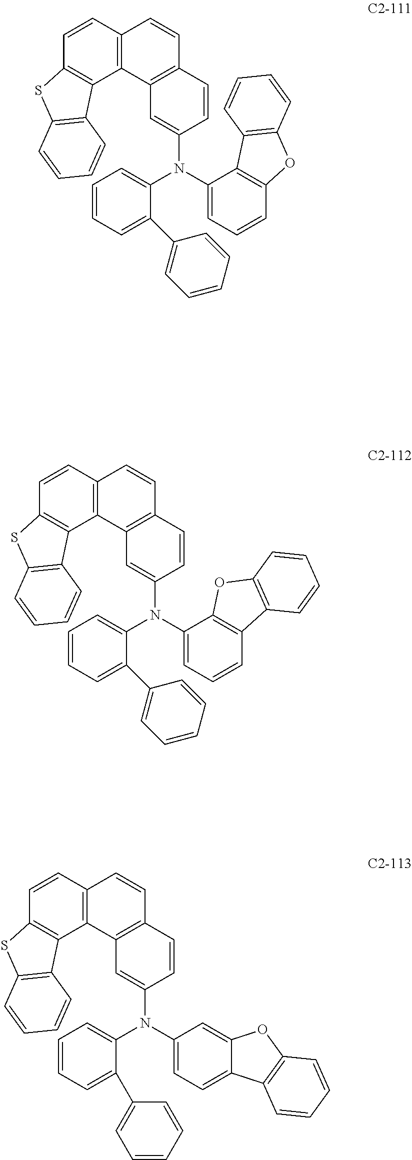 [SMILES: c1ccc(-c2ccccc2N(c2ccc3ccc4ccc5sc6ccccc6c5c4c3c2)c2cccc3oc4ccccc4c23)cc1 ; c1ccc(-c2ccccc2N(c2ccc3ccc4ccc5sc6ccccc6c5c4c3c2)c2cccc3c2oc2ccccc23)cc1 ; c1ccc(-c2ccccc2N(c2ccc3c(c2)oc2ccccc23)c2ccc3ccc4ccc5sc6ccccc6c5c4c3c2)cc1]

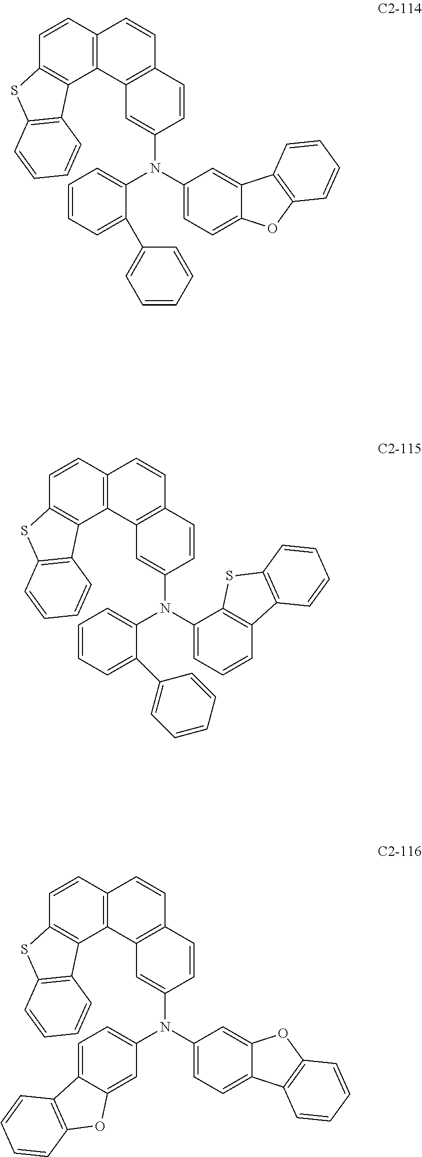 [SMILES: c1ccc(-c2ccccc2N(c2ccc3oc4ccccc4c3c2)c2ccc3ccc4ccc5sc6ccccc6c5c4c3c2)cc1 ; c1ccc(-c2ccccc2N(c2ccc3ccc4ccc5sc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6sc7ccccc7c6c5c4c3)ccc12]

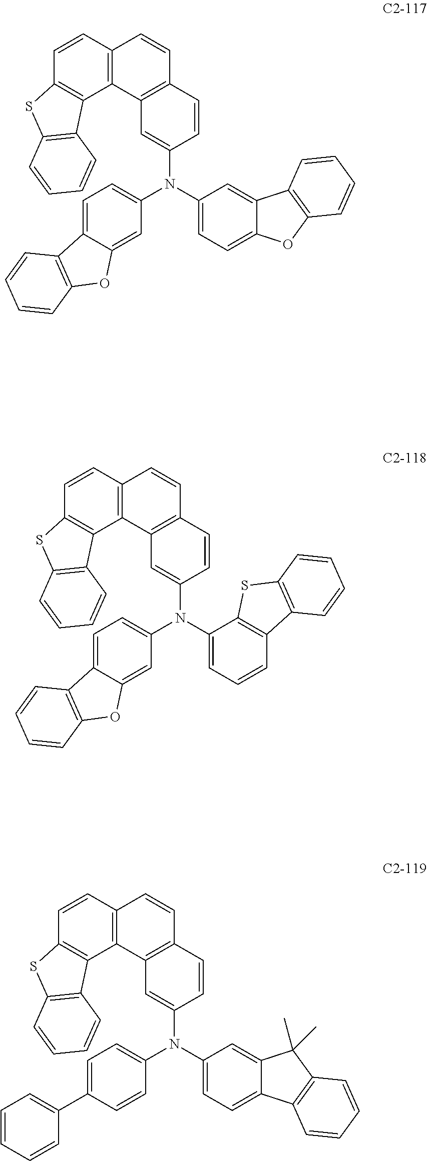 [SMILES: c1ccc2c(c1)oc1cc(N(c3ccc4oc5ccccc5c4c3)c3ccc4ccc5ccc6sc7ccccc7c6c5c4c3)ccc12 ; c1ccc2c(c1)oc1cc(N(c3ccc4ccc5ccc6sc7ccccc7c6c5c4c3)c3cccc4c3sc3ccccc34)ccc12 ; CC1(C)c2ccccc2-c2ccc(N(c3ccc(-c4ccccc4)cc3)c3ccc4ccc5ccc6sc7ccccc7c6c5c4c3)cc21]

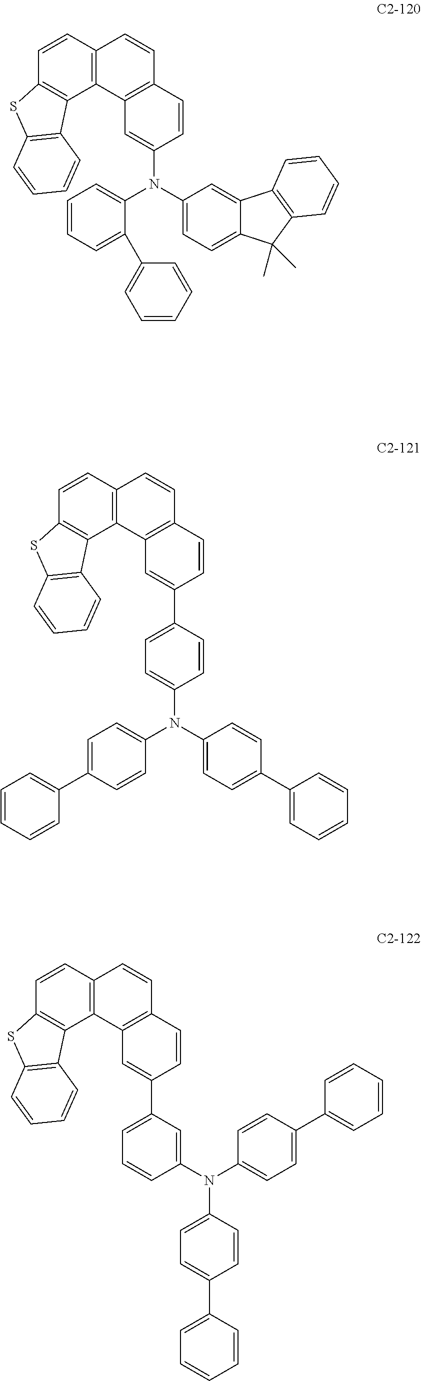 [SMILES: CC1(C)c2ccccc2-c2cc(N(c3ccc4ccc5ccc6sc7ccccc7c6c5c4c3)c3ccccc3-c3ccccc3)ccc21 ; c1ccc(-c2ccc(N(c3ccc(-c4ccccc4)cc3)c3ccc(-c4ccc5ccc6ccc7sc8ccccc8c7c6c5c4)cc3)cc2)cc1 ; c1ccc(-c2ccc(N(c3ccc(-c4ccccc4)cc3)c3cccc(-c4ccc5ccc6ccc7sc8ccccc8c7c6c5c4)c3)cc2)cc1]

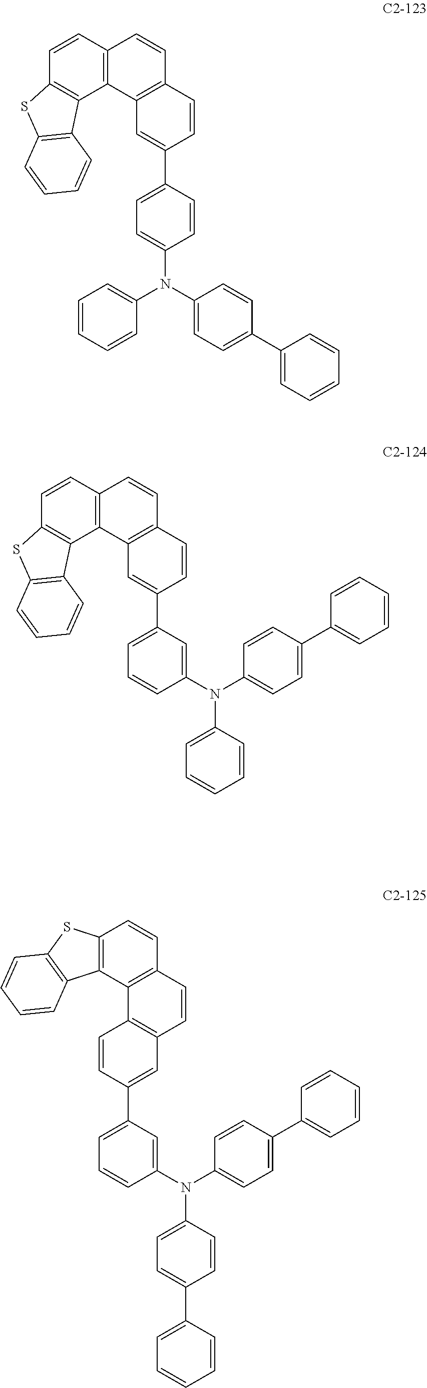 [SMILES: c1ccc(-c2ccc(N(c3ccccc3)c3ccc(-c4ccc5ccc6ccc7sc8ccccc8c7c6c5c4)cc3)cc2)cc1 ; c1ccc(-c2ccc(N(c3ccccc3)c3cccc(-c4ccc5ccc6ccc7sc8ccccc8c7c6c5c4)c3)cc2)cc1 ; c1ccc(-c2ccc(N(c3ccc(-c4ccccc4)cc3)c3cccc(-c4ccc5c(ccc6ccc7sc8ccccc8c7c65)c4)c3)cc2)cc1]

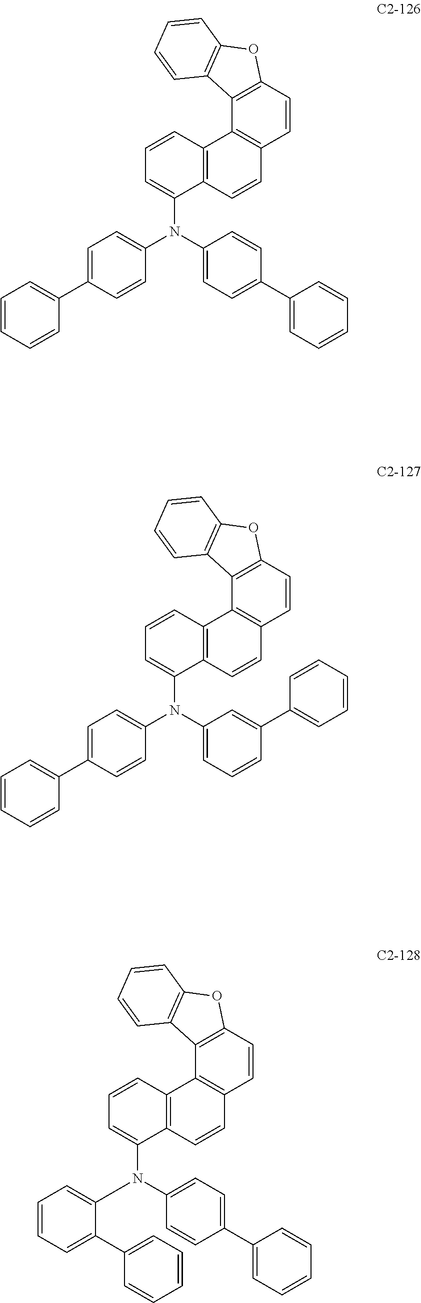 [SMILES: c1ccc(-c2ccc(N(c3ccc(-c4ccccc4)cc3)c3cccc4c3ccc3ccc5oc6ccccc6c5c34)cc2)cc1 ; c1ccc(-c2ccc(N(c3cccc(-c4ccccc4)c3)c3cccc4c3ccc3ccc5oc6ccccc6c5c34)cc2)cc1 ; c1ccc(-c2ccc(N(c3ccccc3-c3ccccc3)c3cccc4c3ccc3ccc5oc6ccccc6c5c34)cc2)cc1]

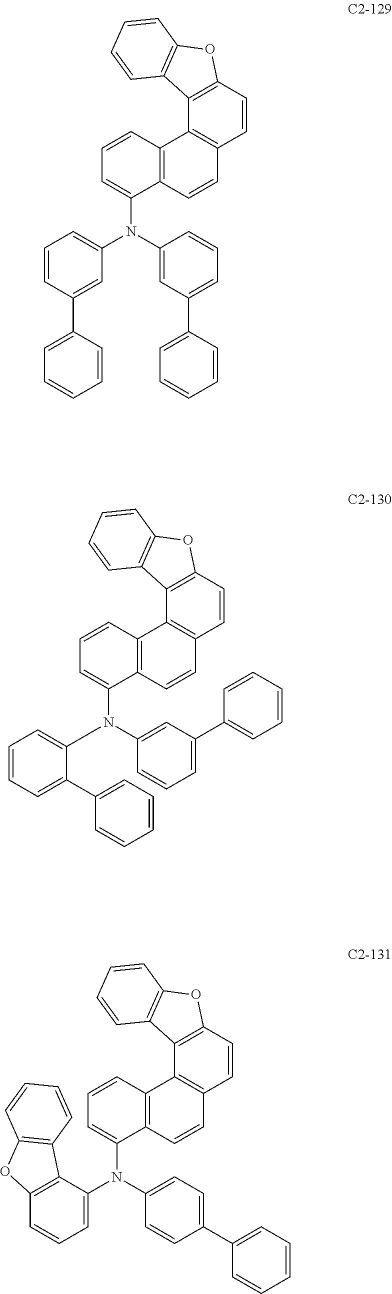 [SMILES: c1ccc(-c2cccc(N(c3cccc(-c4ccccc4)c3)c3cccc4c3ccc3ccc5oc6ccccc6c5c34)c2)cc1 ; c1ccc(-c2cccc(N(c3ccccc3-c3ccccc3)c3cccc4c3ccc3ccc5oc6ccccc6c5c34)c2)cc1 ; c1ccc(-c2ccc(N(c3cccc4c3ccc3ccc5oc6ccccc6c5c34)c3cccc4oc5ccccc5c34)cc2)cc1]

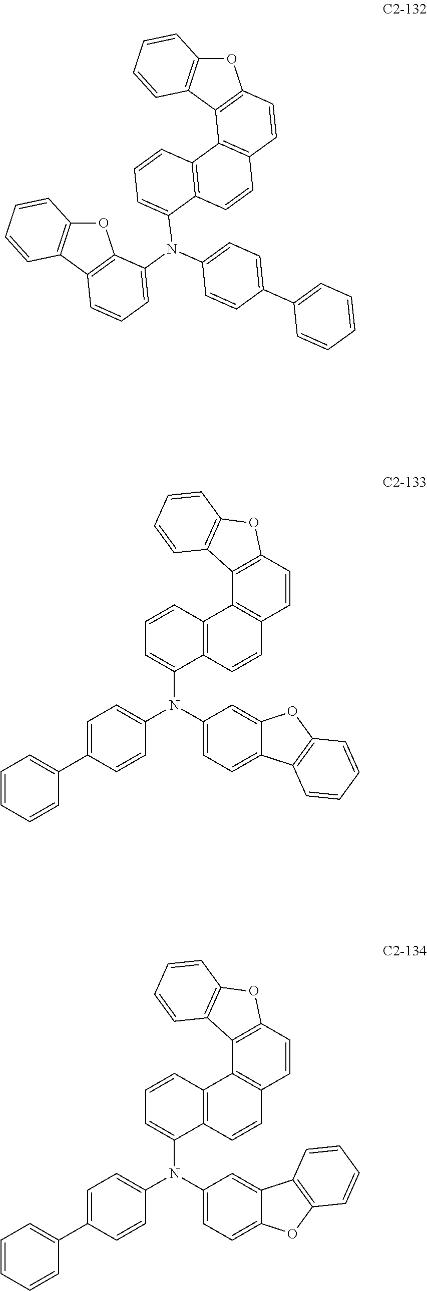 [SMILES: c1ccc(-c2ccc(N(c3cccc4c3ccc3ccc5oc6ccccc6c5c34)c3cccc4c3oc3ccccc34)cc2)cc1 ; c1ccc(-c2ccc(N(c3ccc4c(c3)oc3ccccc34)c3cccc4c3ccc3ccc5oc6ccccc6c5c34)cc2)cc1 ; c1ccc(-c2ccc(N(c3ccc4oc5ccccc5c4c3)c3cccc4c3ccc3ccc5oc6ccccc6c5c34)cc2)cc1]

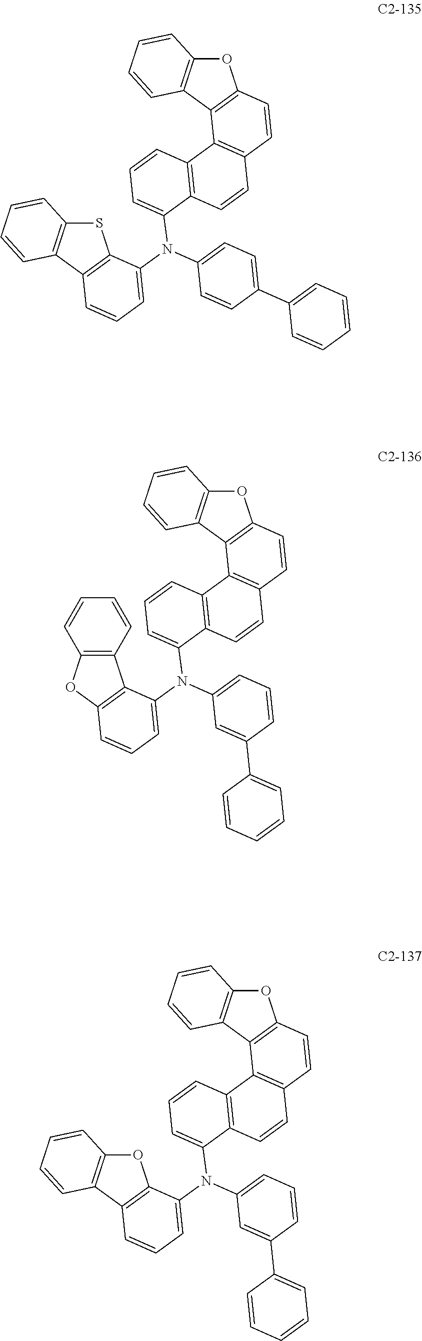 [SMILES: c1ccc(-c2ccc(N(c3cccc4c3ccc3ccc5oc6ccccc6c5c34)c3cccc4c3sc3ccccc34)cc2)cc1 ; c1ccc(-c2cccc(N(c3cccc4c3ccc3ccc5oc6ccccc6c5c34)c3cccc4oc5ccccc5c34)c2)cc1 ; c1ccc(-c2cccc(N(c3cccc4c3ccc3ccc5oc6ccccc6c5c34)c3cccc4c3oc3ccccc34)c2)cc1]

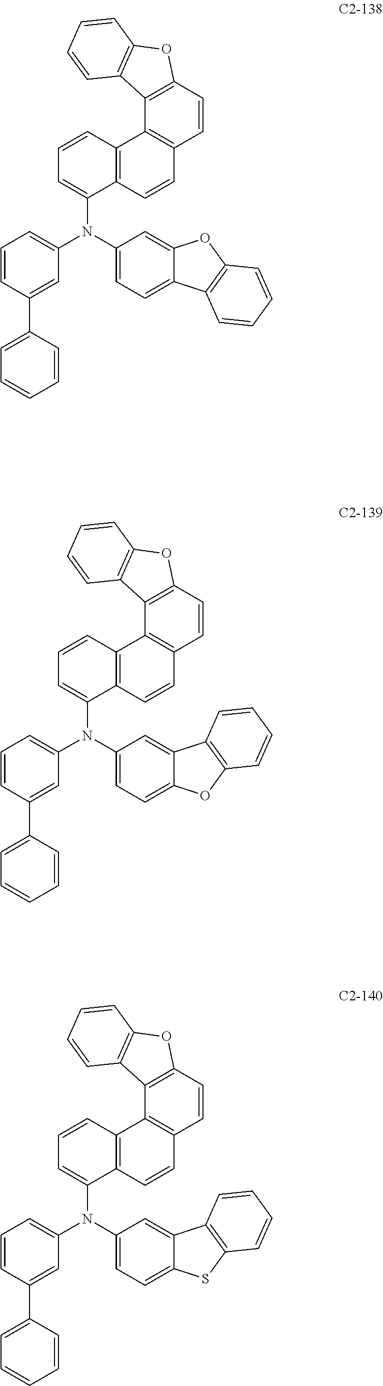 [SMILES: c1ccc(-c2cccc(N(c3ccc4c(c3)oc3ccccc34)c3cccc4c3ccc3ccc5oc6ccccc6c5c34)c2)cc1 ; c1ccc(-c2cccc(N(c3ccc4oc5ccccc5c4c3)c3cccc4c3ccc3ccc5oc6ccccc6c5c34)c2)cc1 ; c1ccc(-c2cccc(N(c3ccc4sc5ccccc5c4c3)c3cccc4c3ccc3ccc5oc6ccccc6c5c34)c2)cc1]

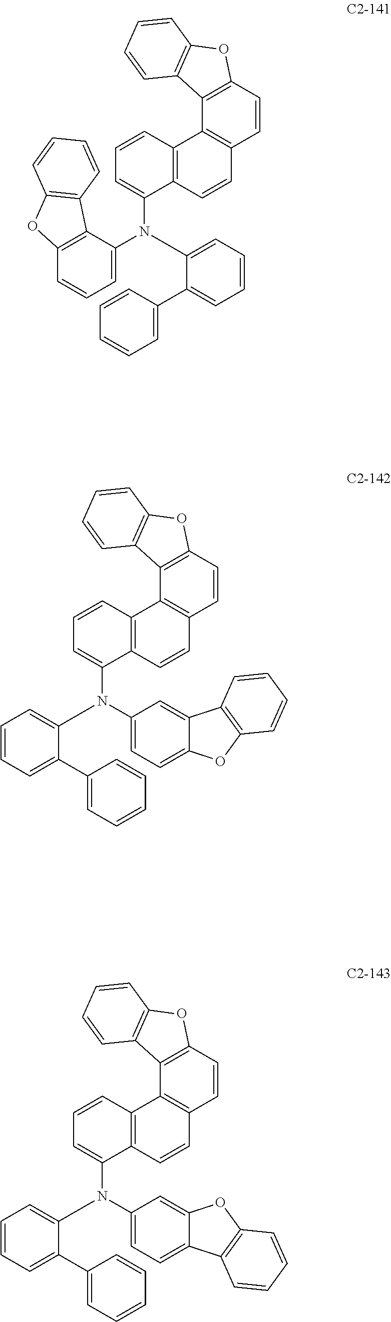 [SMILES: c1ccc(-c2ccccc2N(c2cccc3c2ccc2ccc4oc5ccccc5c4c23)c2cccc3oc4ccccc4c23)cc1 ; c1ccc(-c2ccccc2N(c2ccc3oc4ccccc4c3c2)c2cccc3c2ccc2ccc4oc5ccccc5c4c23)cc1 ; c1ccc(-c2ccccc2N(c2ccc3c(c2)oc2ccccc23)c2cccc3c2ccc2ccc4oc5ccccc5c4c23)cc1]

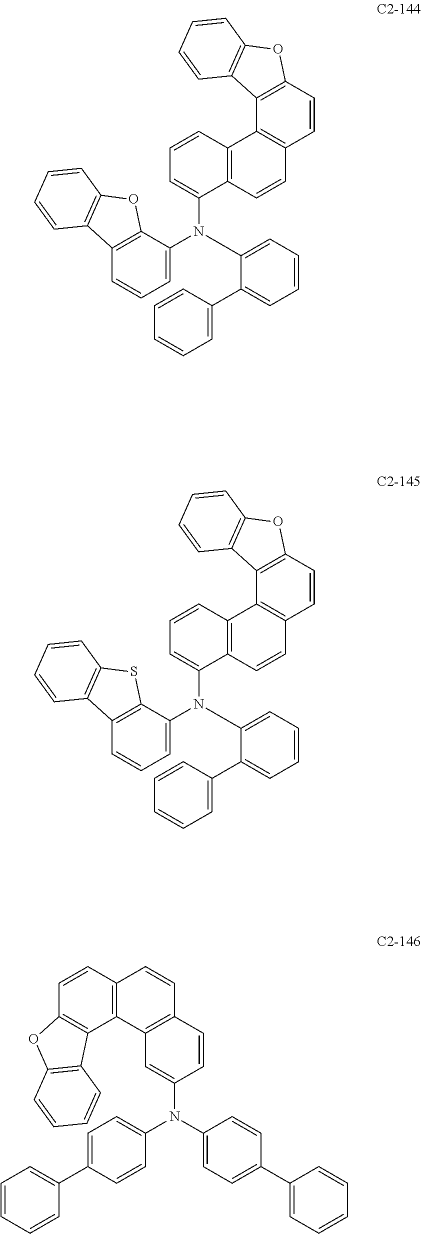 [SMILES: c1ccc(-c2ccccc2N(c2cccc3c2ccc2ccc4oc5ccccc5c4c23)c2cccc3c2oc2ccccc23)cc1 ; c1ccc(-c2ccccc2N(c2cccc3c2ccc2ccc4oc5ccccc5c4c23)c2cccc3c2sc2ccccc23)cc1 ; c1ccc(-c2ccc(N(c3ccc(-c4ccccc4)cc3)c3ccc4ccc5ccc6oc7ccccc7c6c5c4c3)cc2)cc1]

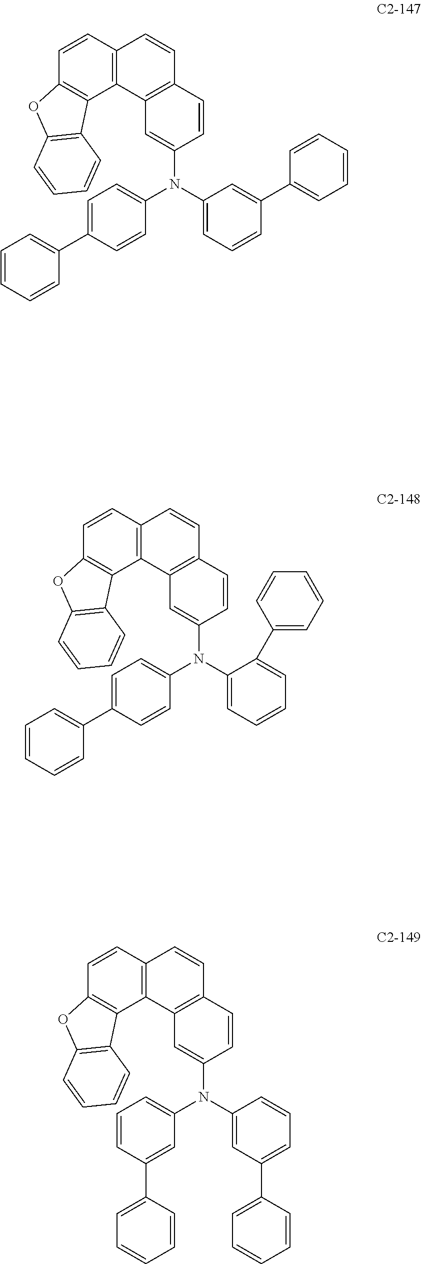 [SMILES: c1ccc(-c2ccc(N(c3cccc(-c4ccccc4)c3)c3ccc4ccc5ccc6oc7ccccc7c6c5c4c3)cc2)cc1 ; c1ccc(-c2ccc(N(c3ccc4ccc5ccc6oc7ccccc7c6c5c4c3)c3ccccc3-c3ccccc3)cc2)cc1 ; c1ccc(-c2cccc(N(c3cccc(-c4ccccc4)c3)c3ccc4ccc5ccc6oc7ccccc7c6c5c4c3)c2)cc1]

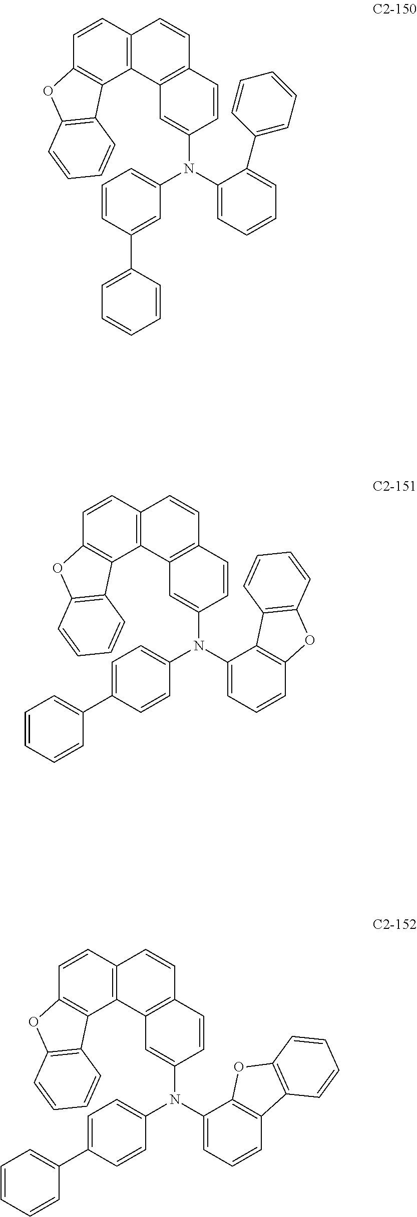 [SMILES: c1ccc(-c2cccc(N(c3ccc4ccc5ccc6oc7ccccc7c6c5c4c3)c3ccccc3-c3ccccc3)c2)cc1 ; c1ccc(-c2ccc(N(c3ccc4ccc5ccc6oc7ccccc7c6c5c4c3)c3cccc4oc5ccccc5c34)cc2)cc1 ; c1ccc(-c2ccc(N(c3ccc4ccc5ccc6oc7ccccc7c6c5c4c3)c3cccc4c3oc3ccccc34)cc2)cc1]

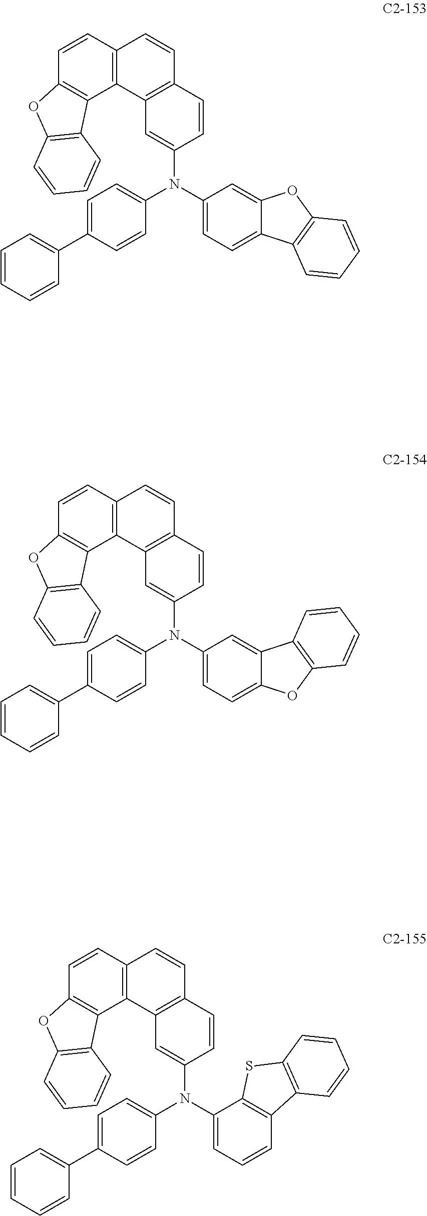 [SMILES: c1ccc(-c2ccc(N(c3ccc4c(c3)oc3ccccc34)c3ccc4ccc5ccc6oc7ccccc7c6c5c4c3)cc2)cc1 ; c1ccc(-c2ccc(N(c3ccc4oc5ccccc5c4c3)c3ccc4ccc5ccc6oc7ccccc7c6c5c4c3)cc2)cc1 ; c1ccc(-c2ccc(N(c3ccc4ccc5ccc6oc7ccccc7c6c5c4c3)c3cccc4c3sc3ccccc34)cc2)cc1]

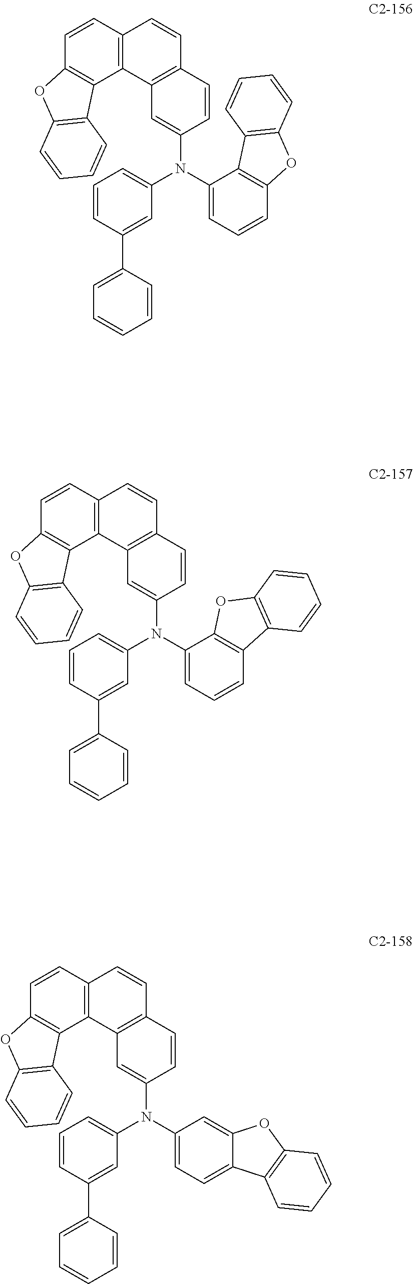 [SMILES: c1ccc(-c2cccc(N(c3ccc4ccc5ccc6oc7ccccc7c6c5c4c3)c3cccc4oc5ccccc5c34)c2)cc1 ; c1ccc(-c2cccc(N(c3ccc4ccc5ccc6oc7ccccc7c6c5c4c3)c3cccc4c3oc3ccccc34)c2)cc1 ; c1ccc(-c2cccc(N(c3ccc4c(c3)oc3ccccc34)c3ccc4ccc5ccc6oc7ccccc7c6c5c4c3)c2)cc1]

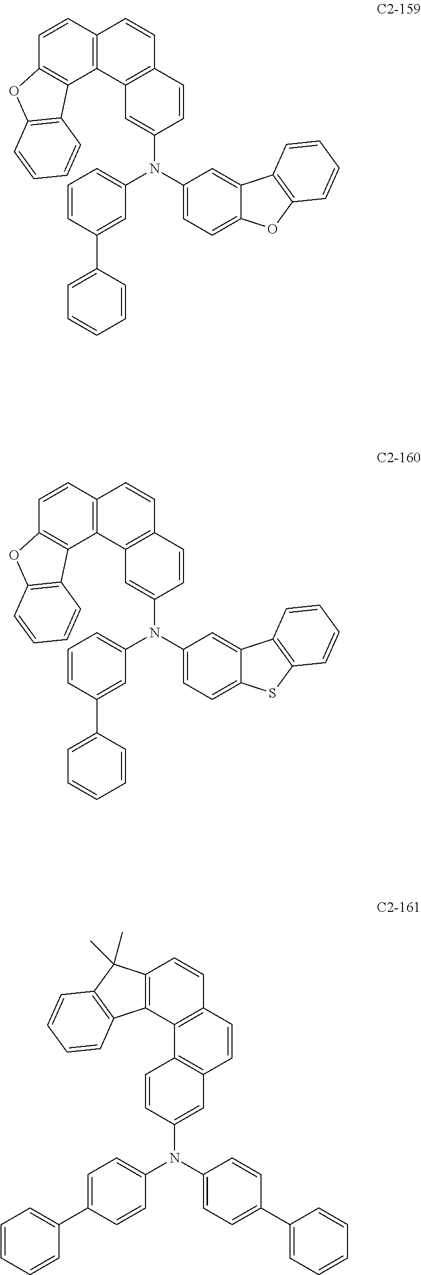 [SMILES: c1ccc(-c2cccc(N(c3ccc4oc5ccccc5c4c3)c3ccc4ccc5ccc6oc7ccccc7c6c5c4c3)c2)cc1 ; c1ccc(-c2cccc(N(c3ccc4sc5ccccc5c4c3)c3ccc4ccc5ccc6oc7ccccc7c6c5c4c3)c2)cc1 ; CC1(C)c2ccccc2-c2c1ccc1ccc3cc(N(c4ccc(-c5ccccc5)cc4)c4ccc(-c5ccccc5)cc4)ccc3c21]

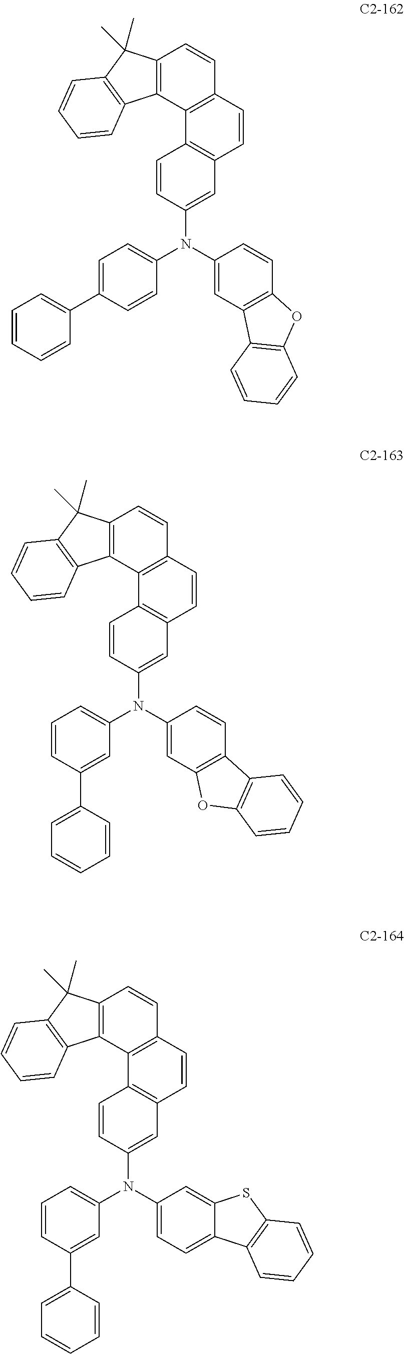 [SMILES: CC1(C)c2ccccc2-c2c1ccc1ccc3cc(N(c4ccc(-c5ccccc5)cc4)c4ccc5oc6ccccc6c5c4)ccc3c21 ; CC1(C)c2ccccc2-c2c1ccc1ccc3cc(N(c4cccc(-c5ccccc5)c4)c4ccc5c(c4)oc4ccccc45)ccc3c21 ; CC1(C)c2ccccc2-c2c1ccc1ccc3cc(N(c4cccc(-c5ccccc5)c4)c4ccc5c(c4)sc4ccccc45)ccc3c21]

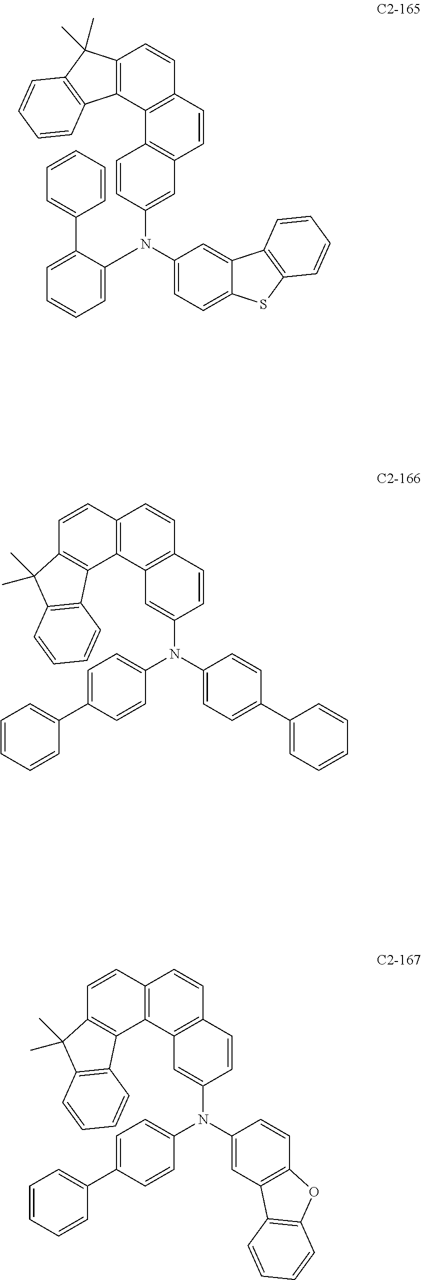 [SMILES: CC1(C)c2ccccc2-c2c1ccc1ccc3cc(N(c4ccc5sc6ccccc6c5c4)c4ccccc4-c4ccccc4)ccc3c21 ; CC1(C)c2ccccc2-c2c1ccc1ccc3ccc(N(c4ccc(-c5ccccc5)cc4)c4ccc(-c5ccccc5)cc4)cc3c21 ; CC1(C)c2ccccc2-c2c1ccc1ccc3ccc(N(c4ccc(-c5ccccc5)cc4)c4ccc5oc6ccccc6c5c4)cc3c21]

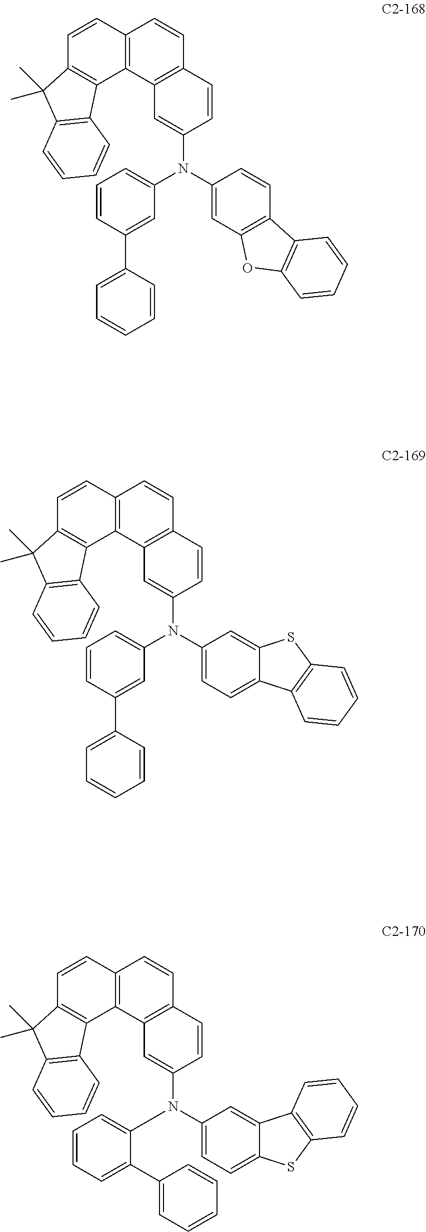 [SMILES: CC1(C)c2ccccc2-c2c1ccc1ccc3ccc(N(c4cccc(-c5ccccc5)c4)c4ccc5c(c4)oc4ccccc45)cc3c21 ; CC1(C)c2ccccc2-c2c1ccc1ccc3ccc(N(c4cccc(-c5ccccc5)c4)c4ccc5c(c4)sc4ccccc45)cc3c21 ; CC1(C)c2ccccc2-c2c1ccc1ccc3ccc(N(c4ccc5sc6ccccc6c5c4)c4ccccc4-c4ccccc4)cc3c21]

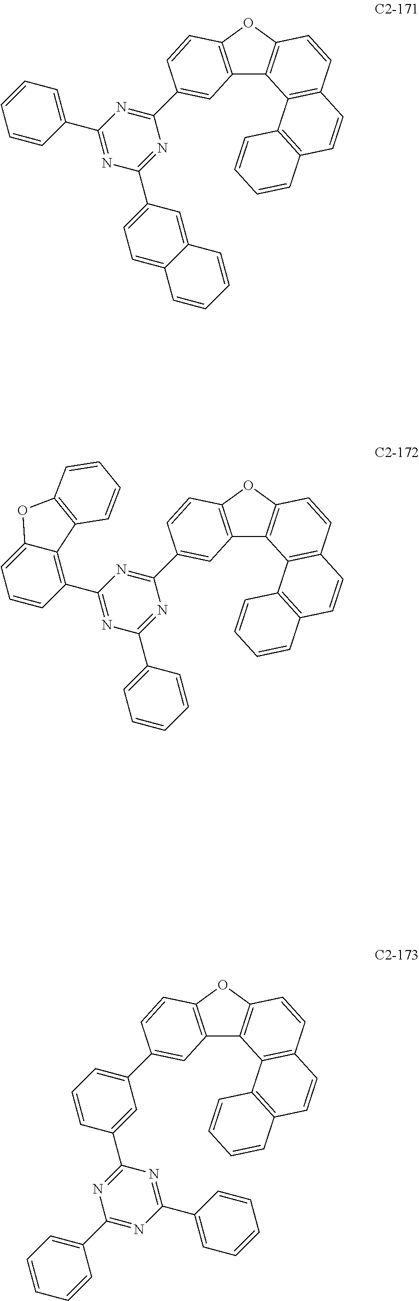 [SMILES: c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1 ; c1ccc(-c2nc(-c3ccccc3)nc(-c3cccc(-c4ccc5oc6ccc7ccc8ccccc8c7c6c5c4)c3)n2)cc1]

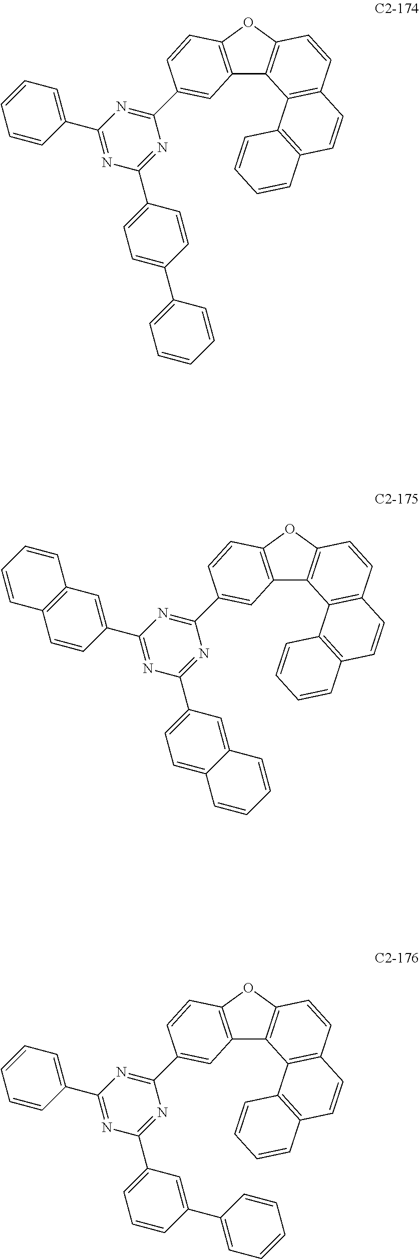 [SMILES: c1ccc(-c2ccc(-c3nc(-c4ccccc4)nc(-c4ccc5oc6ccc7ccc8ccccc8c7c6c5c4)n3)cc2)cc1 ; c1ccc2cc(-c3nc(-c4ccc5ccccc5c4)nc(-c4ccc5oc6ccc7ccc8ccccc8c7c6c5c4)n3)ccc2c1 ; c1ccc(-c2cccc(-c3nc(-c4ccccc4)nc(-c4ccc5oc6ccc7ccc8ccccc8c7c6c5c4)n3)c2)cc1]

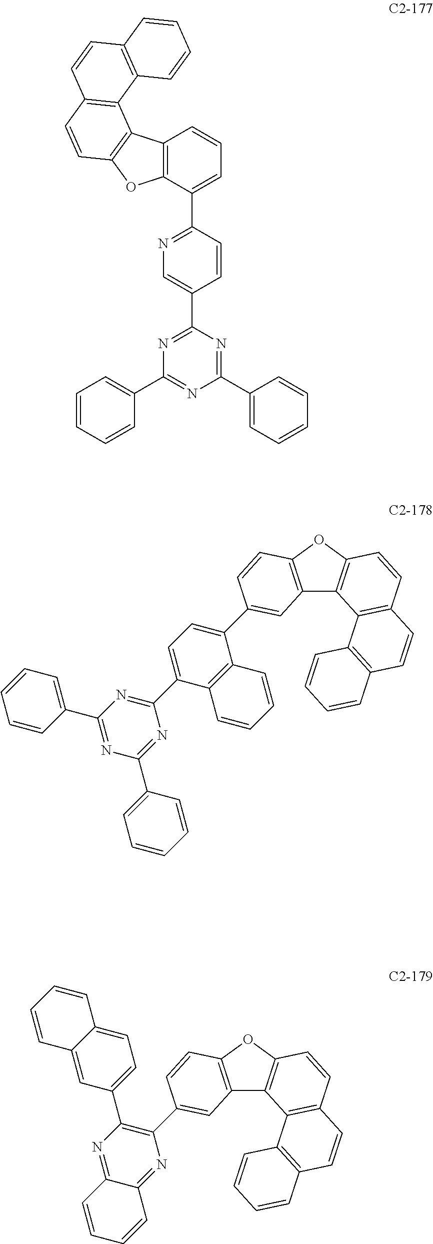 [SMILES: c1ccc(-c2nc(-c3ccccc3)nc(-c3ccc(-c4cccc5c4oc4ccc6ccc7ccccc7c6c45)nc3)n2)cc1 ; c1ccc(-c2nc(-c3ccccc3)nc(-c3ccc(-c4ccc5oc6ccc7ccc8ccccc8c7c6c5c4)c4ccccc34)n2)cc1 ; c1ccc2cc(-c3nc4ccccc4nc3-c3ccc4oc5ccc6ccc7ccccc7c6c5c4c3)ccc2c1]

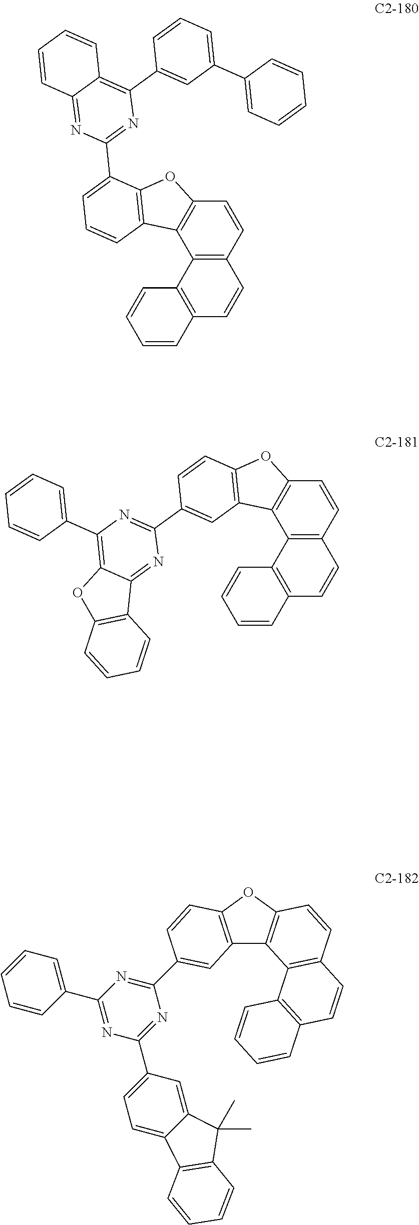 [SMILES: c1ccc(-c2cccc(-c3nc(-c4cccc5c4oc4ccc6ccc7ccccc7c6c45)nc4ccccc34)c2)cc1 ; c1ccc(-c2nc(-c3ccc4oc5ccc6ccc7ccccc7c6c5c4c3)nc3c2oc2ccccc23)cc1 ; CC1(C)c2ccccc2-c2ccc(-c3nc(-c4ccccc4)nc(-c4ccc5oc6ccc7ccc8ccccc8c7c6c5c4)n3)cc21]

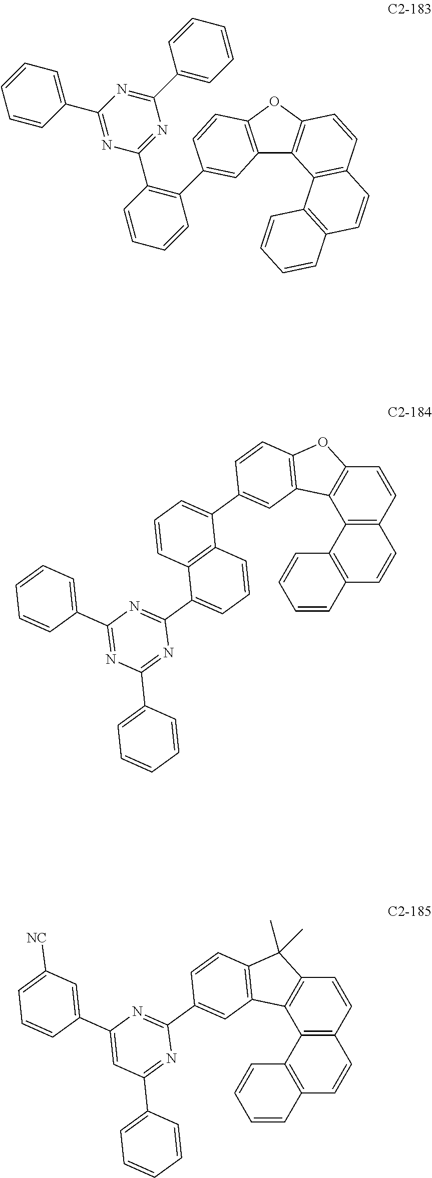 [SMILES: c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1 ; CC1(C)c2ccc(-c3nc(-c4ccccc4)cc(-c4cccc(C#N)c4)n3)cc2-c2c1ccc1ccc3ccccc3c21]

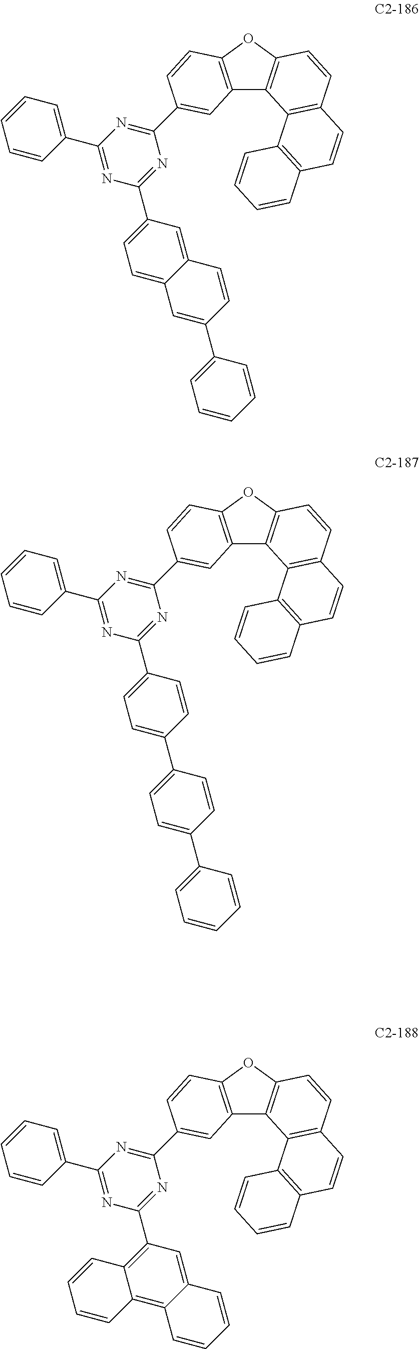 [SMILES: c1ccc(-c2ccc3cc(-c4nc(-c5ccccc5)nc(-c5ccc6oc7ccc8ccc9ccccc9c8c7c6c5)n4)ccc3c2)cc1 ; c1ccc(-c2ccc(-c3ccc(-c4nc(-c5ccccc5)nc(-c5ccc6oc7ccc8ccc9ccccc9c8c7c6c5)n4)cc3)cc2)cc1 ; c1ccc(-c2nc(-c3ccc4oc5ccc6ccc7ccccc7c6c5c4c3)nc(-c3cc4ccccc4c4ccccc34)n2)cc1]

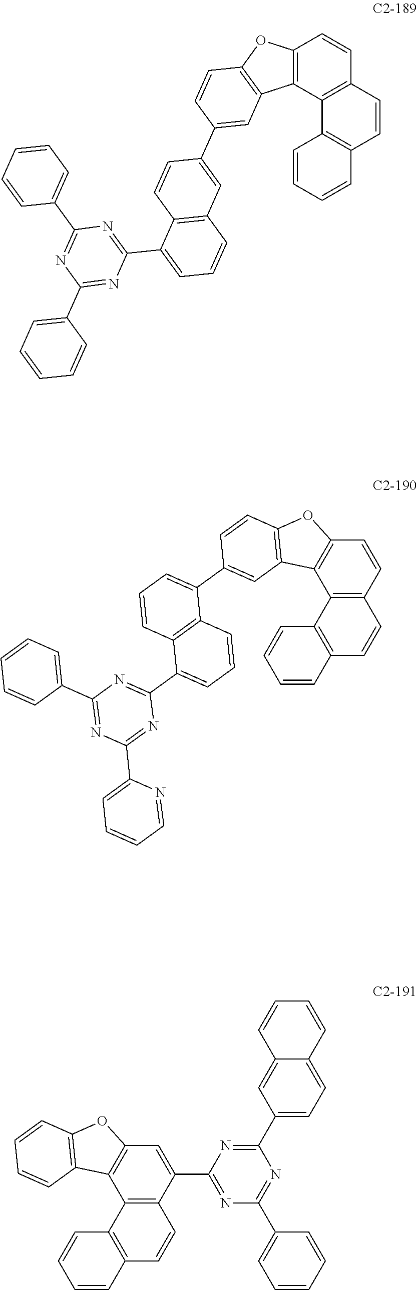 [SMILES: c1ccc(-c2nc(-c3ccccc3)nc(-c3cccc4cc(-c5ccc6oc7ccc8ccc9ccccc9c8c7c6c5)ccc34)n2)cc1 ; c1ccc(-c2nc(-c3ccccn3)nc(-c3cccc4c(-c5ccc6oc7ccc8ccc9ccccc9c8c7c6c5)cccc34)n2)cc1 ; c1ccc(-c2nc(-c3ccc4ccccc4c3)nc(-c3cc4oc5ccccc5c4c4c3ccc3ccccc34)n2)cc1]

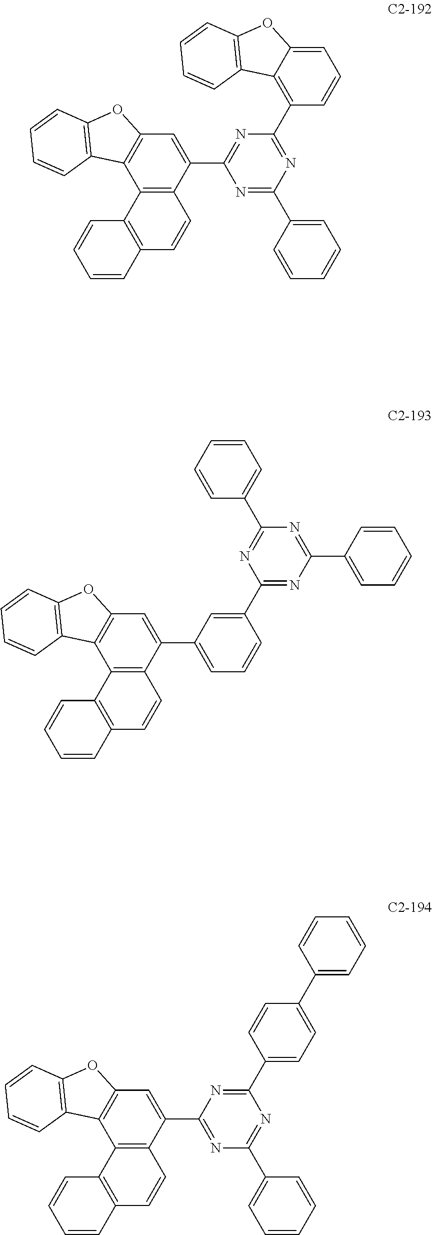 [SMILES: c1ccc(-c2nc(-c3cc4oc5ccccc5c4c4c3ccc3ccccc34)nc(-c3cccc4oc5ccccc5c34)n2)cc1 ; c1ccc(-c2nc(-c3ccccc3)nc(-c3cccc(-c4cc5oc6ccccc6c5c5c4ccc4ccccc45)c3)n2)cc1 ; c1ccc(-c2ccc(-c3nc(-c4ccccc4)nc(-c4cc5oc6ccccc6c5c5c4ccc4ccccc45)n3)cc2)cc1]

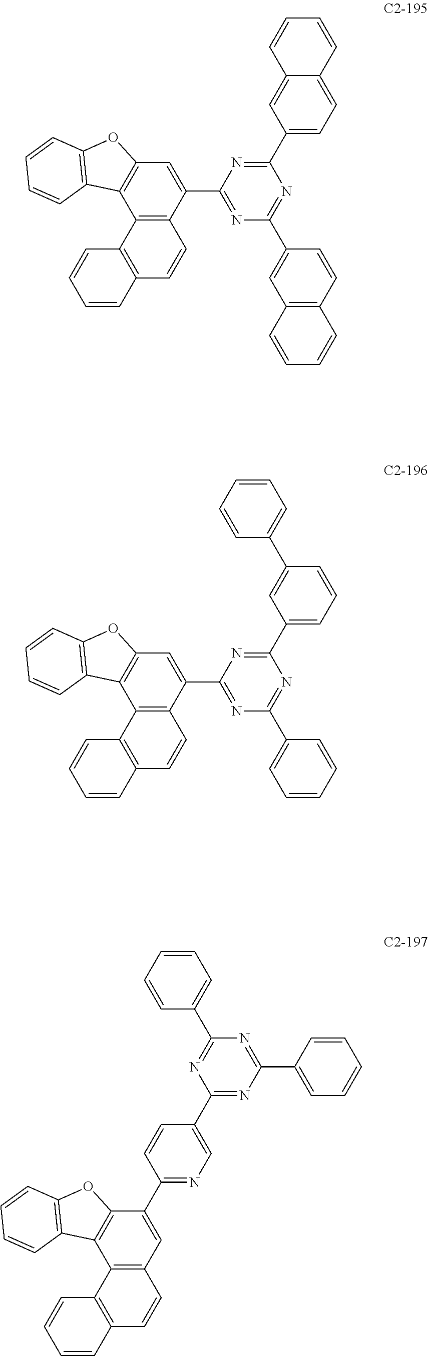 [SMILES: c1ccc2cc(-c3nc(-c4ccc5ccccc5c4)nc(-c4cc5oc6ccccc6c5c5c4ccc4ccccc45)n3)ccc2c1 ; c1ccc(-c2cccc(-c3nc(-c4ccccc4)nc(-c4cc5oc6ccccc6c5c5c4ccc4ccccc45)n3)c2)cc1 ; c1ccc(-c2nc(-c3ccccc3)nc(-c3ccc(-c4cc5ccc6ccccc6c5c5c4oc4ccccc45)nc3)n2)cc1]

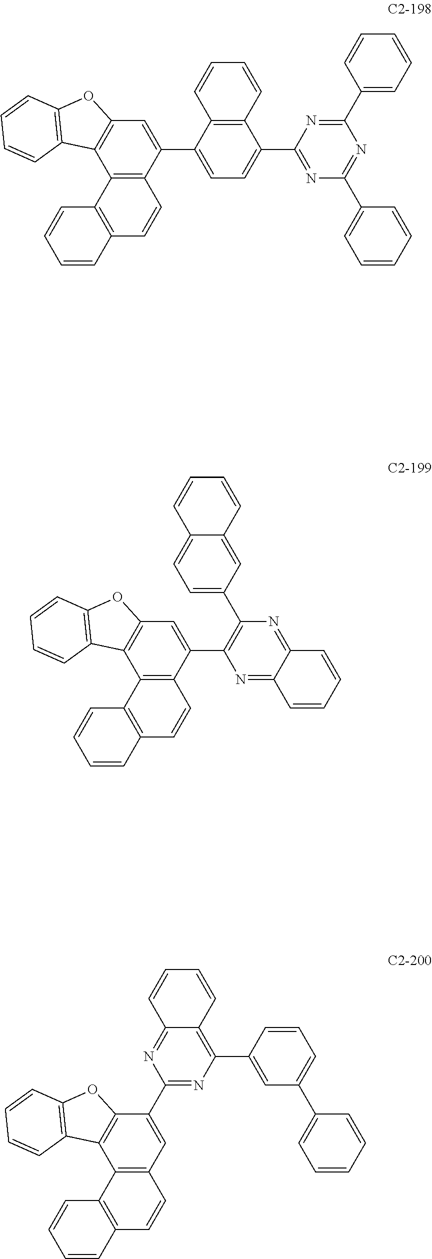 [SMILES: c1ccc(-c2nc(-c3ccccc3)nc(-c3ccc(-c4cc5oc6ccccc6c5c5c4ccc4ccccc45)c4ccccc34)n2)cc1 ; c1ccc2cc(-c3nc4ccccc4nc3-c3cc4oc5ccccc5c4c4c3ccc3ccccc34)ccc2c1 ; c1ccc(-c2cccc(-c3nc(-c4cc5ccc6ccccc6c5c5c4oc4ccccc45)nc4ccccc34)c2)cc1]

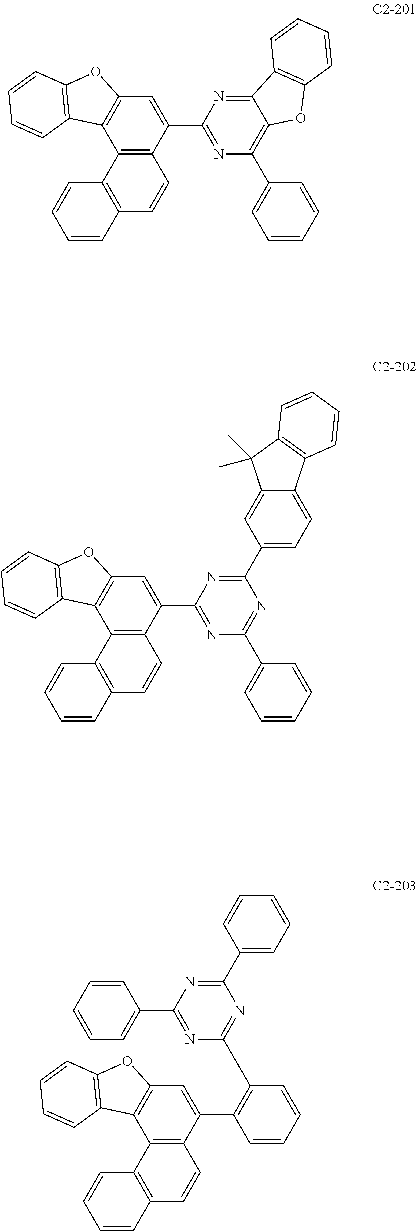 [SMILES: c1ccc(-c2nc(-c3cc4oc5ccccc5c4c4c3ccc3ccccc34)nc3c2oc2ccccc23)cc1 ; CC1(C)c2ccccc2-c2ccc(-c3nc(-c4ccccc4)nc(-c4cc5oc6ccccc6c5c5c4ccc4ccccc45)n3)cc21 ; c1ccc(-c2nc(-c3ccccc3)nc(-c3ccccc3-c3cc4oc5ccccc5c4c4c3ccc3ccccc34)n2)cc1]

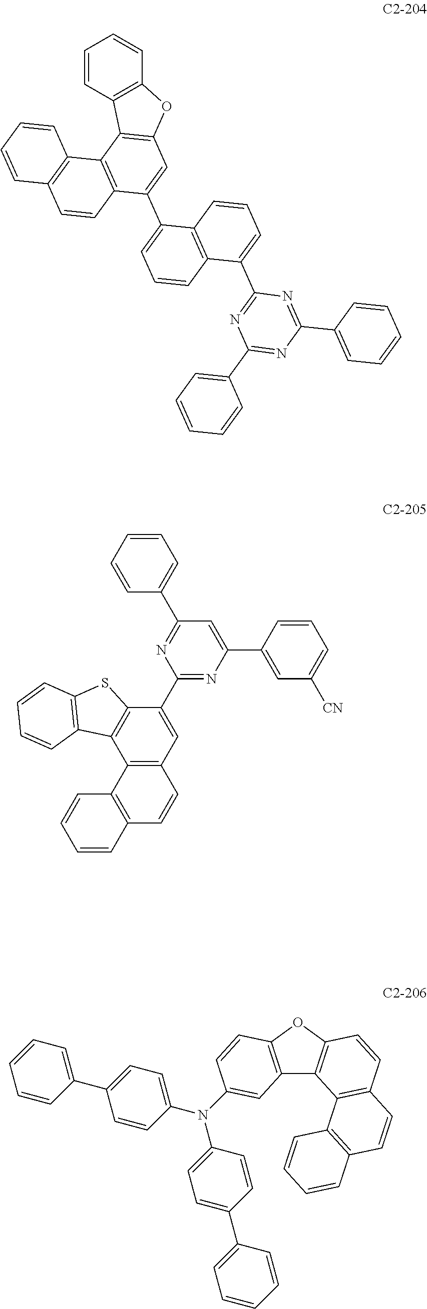 [SMILES: c1ccc(-c2nc(-c3ccccc3)nc(-c3cccc4c(-c5cc6oc7ccccc7c6c6c5ccc5ccccc56)cccc34)n2)cc1 ; N#Cc1cccc(-c2cc(-c3ccccc3)nc(-c3cc4ccc5ccccc5c4c4c3sc3ccccc34)n2)c1 ; c1ccc(-c2ccc(N(c3ccc(-c4ccccc4)cc3)c3ccc4oc5ccc6ccc7ccccc7c6c5c4c3)cc2)cc1]

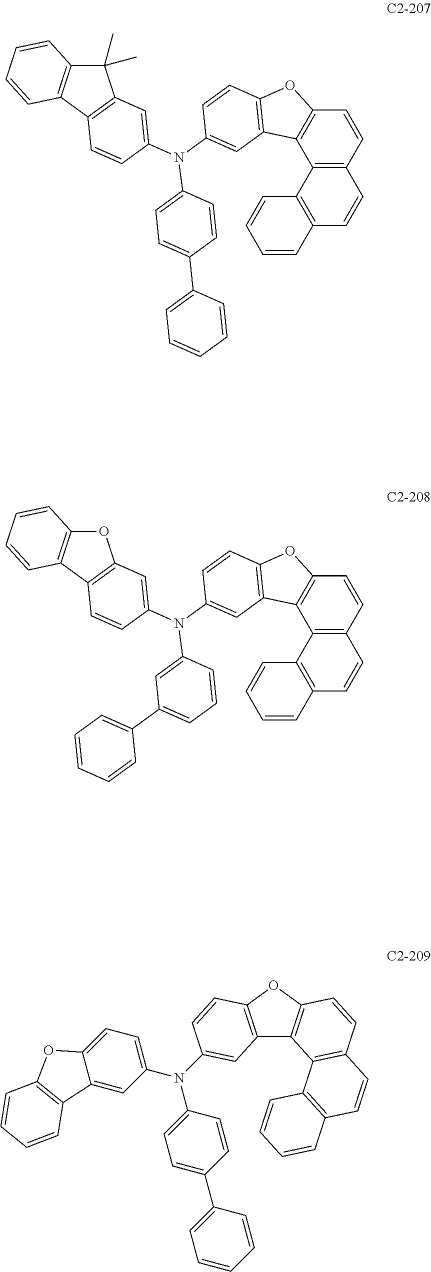 [SMILES: CC1(C)c2ccccc2-c2ccc(N(c3ccc(-c4ccccc4)cc3)c3ccc4oc5ccc6ccc7ccccc7c6c5c4c3)cc21 ; c1ccc(-c2cccc(N(c3ccc4c(c3)oc3ccccc34)c3ccc4oc5ccc6ccc7ccccc7c6c5c4c3)c2)cc1 ; c1ccc(-c2ccc(N(c3ccc4oc5ccccc5c4c3)c3ccc4oc5ccc6ccc7ccccc7c6c5c4c3)cc2)cc1]

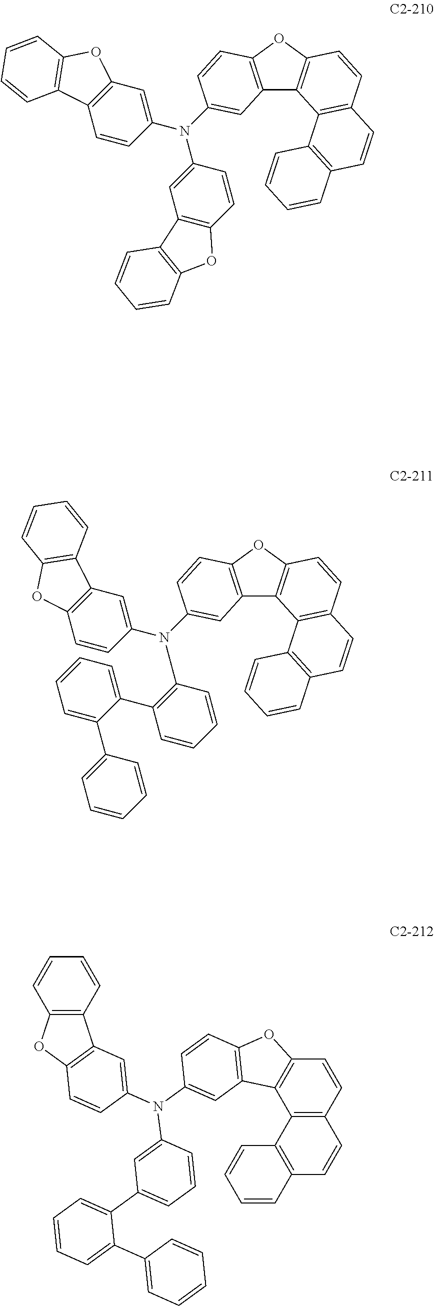 [SMILES: c1ccc2c(c1)ccc1ccc3oc4ccc(N(c5ccc6c(c5)oc5ccccc56)c5ccc6oc7ccccc7c6c5)cc4c3c12 ; c1ccc(-c2ccccc2-c2ccccc2N(c2ccc3oc4ccccc4c3c2)c2ccc3oc4ccc5ccc6ccccc6c5c4c3c2)cc1 ; c1ccc(-c2ccccc2-c2cccc(N(c3ccc4oc5ccccc5c4c3)c3ccc4oc5ccc6ccc7ccccc7c6c5c4c3)c2)cc1]

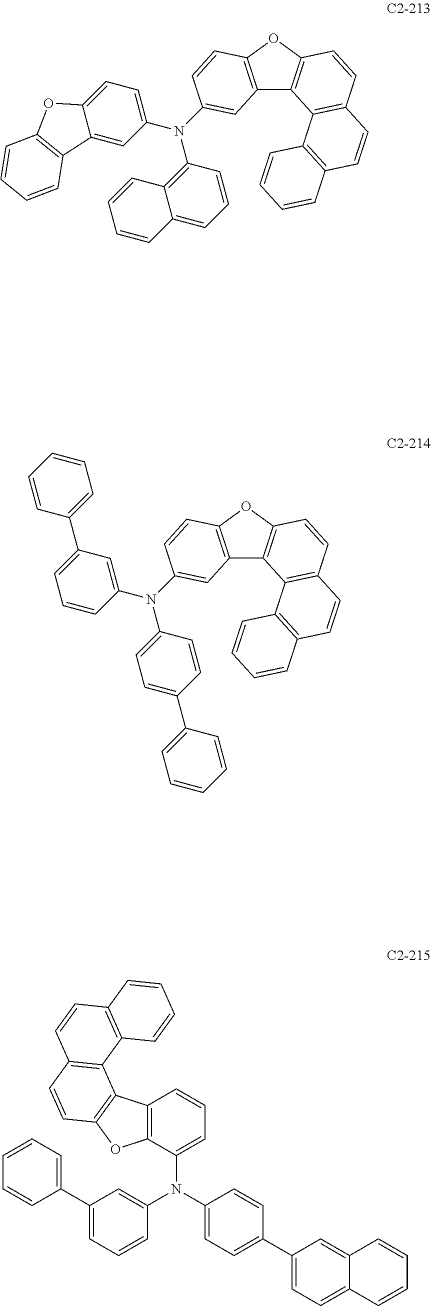 [SMILES: c1ccc2c(N(c3ccc4oc5ccccc5c4c3)c3ccc4oc5ccc6ccc7ccccc7c6c5c4c3)cccc2c1 ; c1ccc(-c2ccc(N(c3cccc(-c4ccccc4)c3)c3ccc4oc5ccc6ccc7ccccc7c6c5c4c3)cc2)cc1 ; c1ccc(-c2cccc(N(c3ccc(-c4ccc5ccccc5c4)cc3)c3cccc4c3oc3ccc5ccc6ccccc6c5c34)c2)cc1]

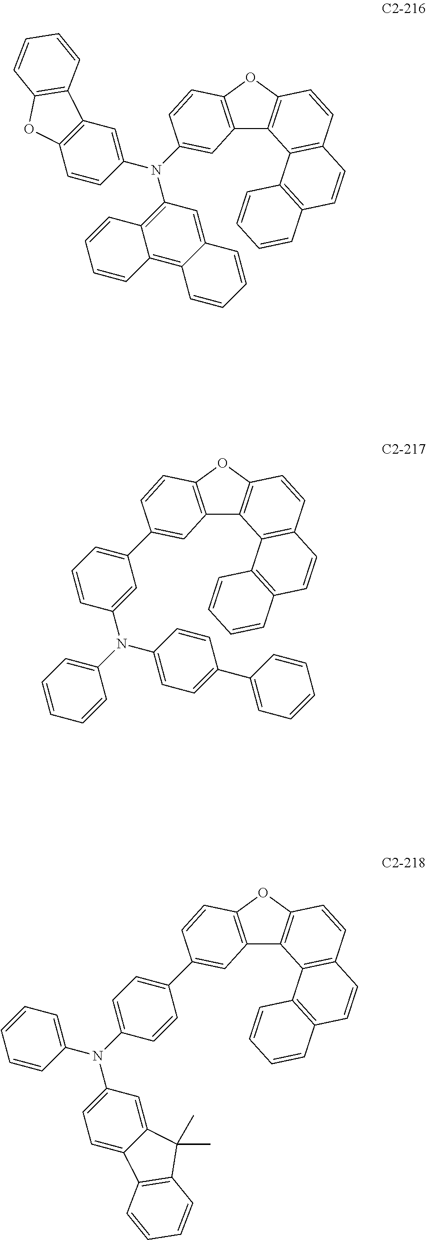 [SMILES: c1ccc2c(c1)cc(N(c1ccc3oc4ccccc4c3c1)c1ccc3oc4ccc5ccc6ccccc6c5c4c3c1)c1ccccc12 ; c1ccc(-c2ccc(N(c3ccccc3)c3cccc(-c4ccc5oc6ccc7ccc8ccccc8c7c6c5c4)c3)cc2)cc1 ; CC1(C)c2ccccc2-c2ccc(N(c3ccccc3)c3ccc(-c4ccc5oc6ccc7ccc8ccccc8c7c6c5c4)cc3)cc21]

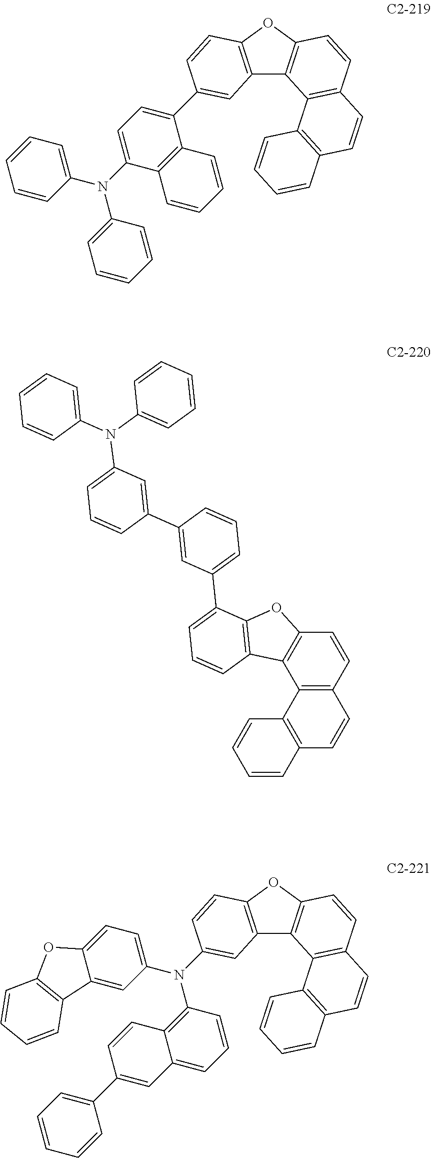 [SMILES: c1ccc(N(c2ccccc2)c2ccc(-c3ccc4oc5ccc6ccc7ccccc7c6c5c4c3)c3ccccc23)cc1 ; c1ccc(N(c2ccccc2)c2cccc(-c3cccc(-c4cccc5c4oc4ccc6ccc7ccccc7c6c45)c3)c2)cc1 ; c1ccc(-c2ccc3c(N(c4ccc5oc6ccccc6c5c4)c4ccc5oc6ccc7ccc8ccccc8c7c6c5c4)cccc3c2)cc1]

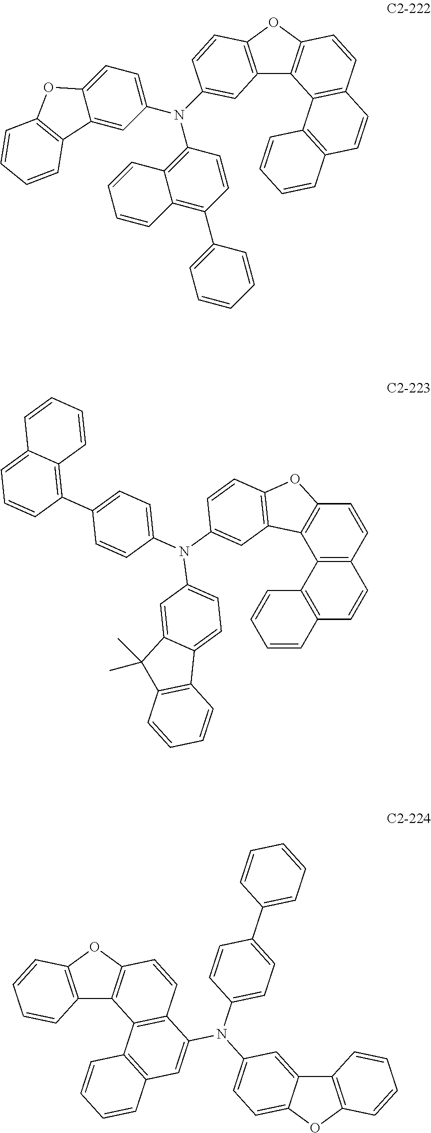 [SMILES: c1ccc(-c2ccc(N(c3ccc4oc5ccccc5c4c3)c3ccc4oc5ccc6ccc7ccccc7c6c5c4c3)c3ccccc23)cc1 ; CC1(C)c2ccccc2-c2ccc(N(c3ccc(-c4cccc5ccccc45)cc3)c3ccc4oc5ccc6ccc7ccccc7c6c5c4c3)cc21 ; c1ccc(-c2ccc(N(c3ccc4oc5ccccc5c4c3)c3cc4ccccc4c4c3ccc3oc5ccccc5c34)cc2)cc1]

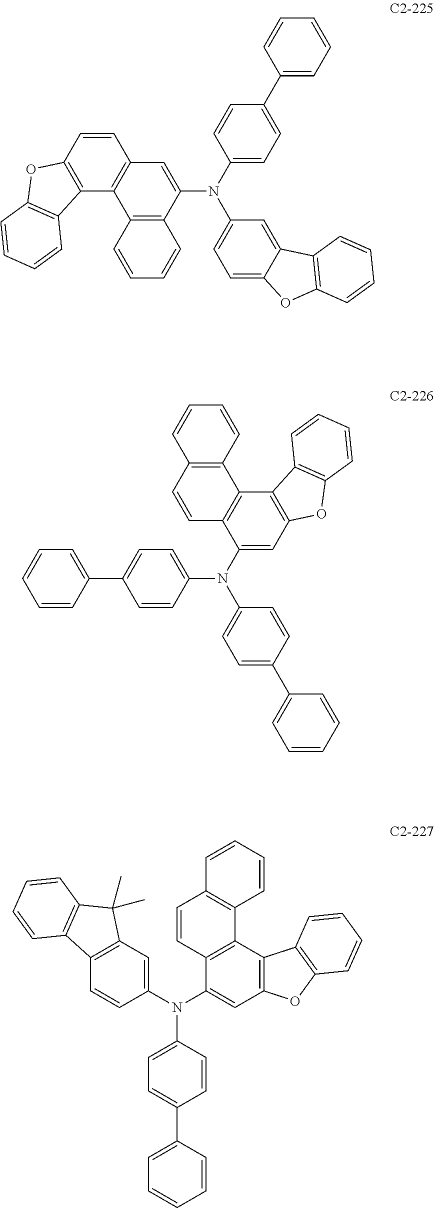 [SMILES: c1ccc(-c2ccc(N(c3ccc4oc5ccccc5c4c3)c3cc4ccc5oc6ccccc6c5c4c4ccccc34)cc2)cc1 ; c1ccc(-c2ccc(N(c3ccc(-c4ccccc4)cc3)c3cc4oc5ccccc5c4c4c3ccc3ccccc34)cc2)cc1 ; CC1(C)c2ccccc2-c2ccc(N(c3ccc(-c4ccccc4)cc3)c3cc4oc5ccccc5c4c4c3ccc3ccccc34)cc21]

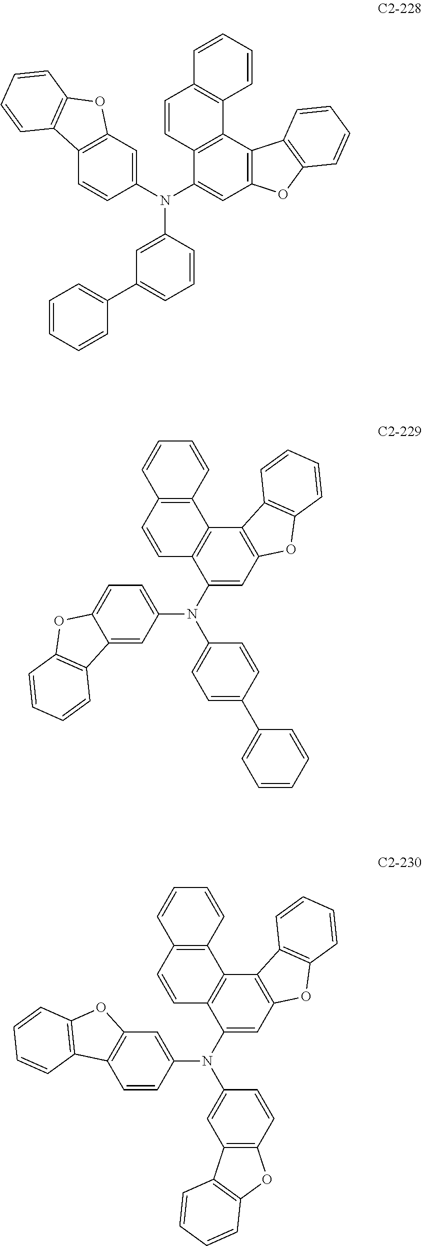 [SMILES: c1ccc(-c2cccc(N(c3ccc4c(c3)oc3ccccc34)c3cc4oc5ccccc5c4c4c3ccc3ccccc34)c2)cc1 ; c1ccc(-c2ccc(N(c3ccc4oc5ccccc5c4c3)c3cc4oc5ccccc5c4c4c3ccc3ccccc34)cc2)cc1 ; c1ccc2c(c1)ccc1c(N(c3ccc4c(c3)oc3ccccc34)c3ccc4oc5ccccc5c4c3)cc3oc4ccccc4c3c12]

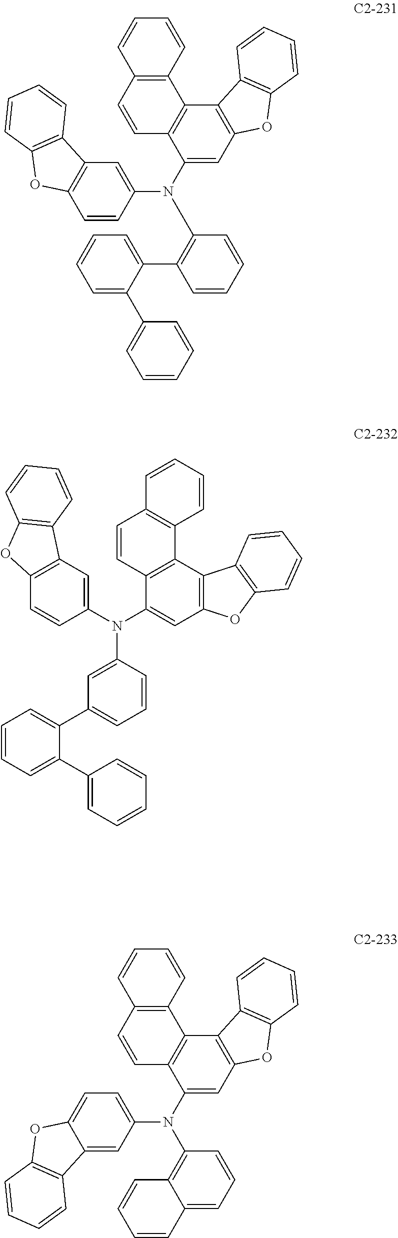 [SMILES: c1ccc(-c2ccccc2-c2ccccc2N(c2ccc3oc4ccccc4c3c2)c2cc3oc4ccccc4c3c3c2ccc2ccccc23)cc1 ; c1ccc(-c2ccccc2-c2cccc(N(c3ccc4oc5ccccc5c4c3)c3cc4oc5ccccc5c4c4c3ccc3ccccc34)c2)cc1 ; c1ccc2c(N(c3ccc4oc5ccccc5c4c3)c3cc4oc5ccccc5c4c4c3ccc3ccccc34)cccc2c1]

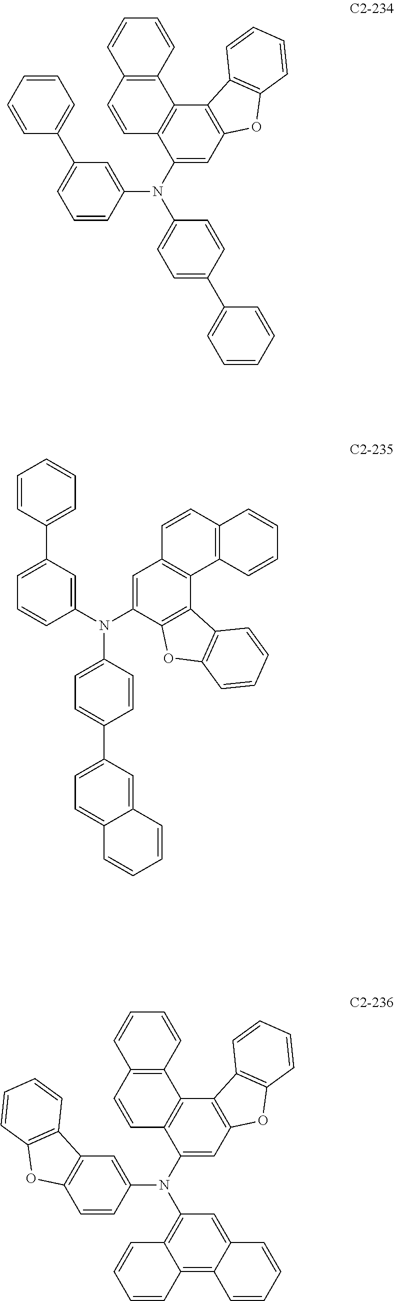 [SMILES: c1ccc(-c2ccc(N(c3cccc(-c4ccccc4)c3)c3cc4oc5ccccc5c4c4c3ccc3ccccc34)cc2)cc1 ; c1ccc(-c2cccc(N(c3ccc(-c4ccc5ccccc5c4)cc3)c3cc4ccc5ccccc5c4c4c3oc3ccccc34)c2)cc1 ; c1ccc2c(c1)cc(N(c1ccc3oc4ccccc4c3c1)c1cc3oc4ccccc4c3c3c1ccc1ccccc13)c1ccccc12]

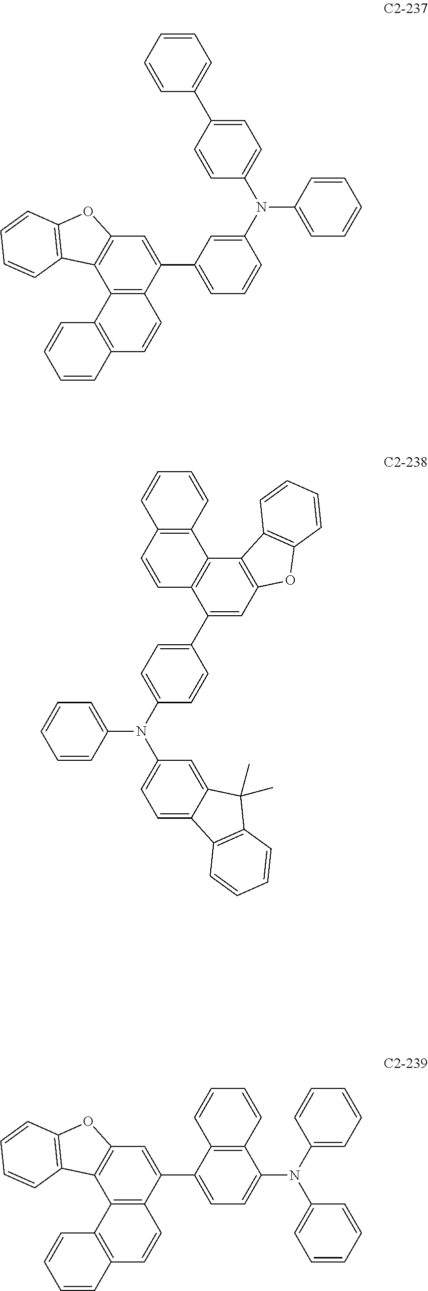 [SMILES: c1ccc(-c2ccc(N(c3ccccc3)c3cccc(-c4cc5oc6ccccc6c5c5c4ccc4ccccc45)c3)cc2)cc1 ; CC1(C)c2ccccc2-c2ccc(N(c3ccccc3)c3ccc(-c4cc5oc6ccccc6c5c5c4ccc4ccccc45)cc3)cc21 ; c1ccc(N(c2ccccc2)c2ccc(-c3cc4oc5ccccc5c4c4c3ccc3ccccc34)c3ccccc23)cc1]

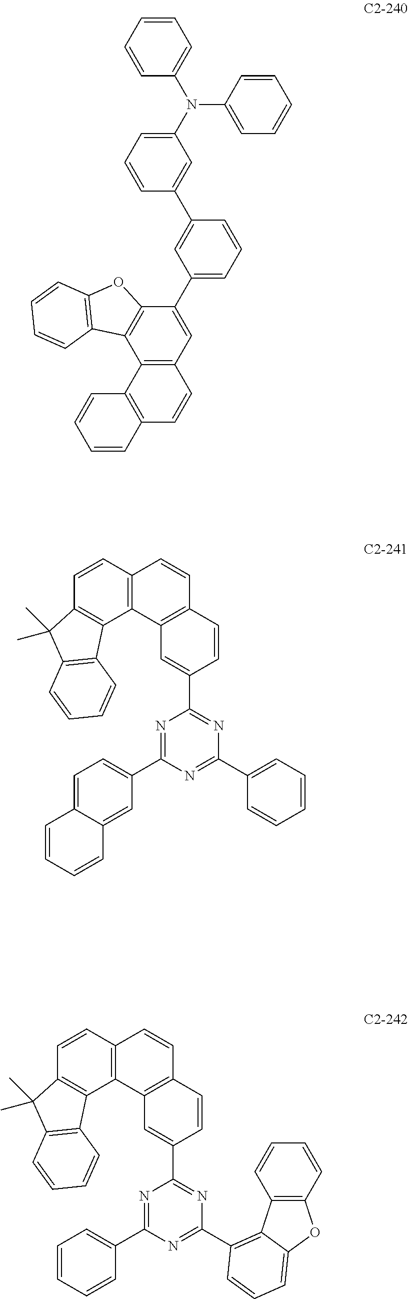 [SMILES: c1ccc(N(c2ccccc2)c2cccc(-c3cccc(-c4cc5ccc6ccccc6c5c5c4oc4ccccc45)c3)c2)cc1 ; CC1(C)c2ccccc2-c2c1ccc1ccc3ccc(-c4nc(-c5ccccc5)nc(-c5ccc6ccccc6c5)n4)cc3c21 ; CC1(C)c2ccccc2-c2c1ccc1ccc3ccc(-c4nc(-c5ccccc5)nc(-c5cccc6oc7ccccc7c56)n4)cc3c21]

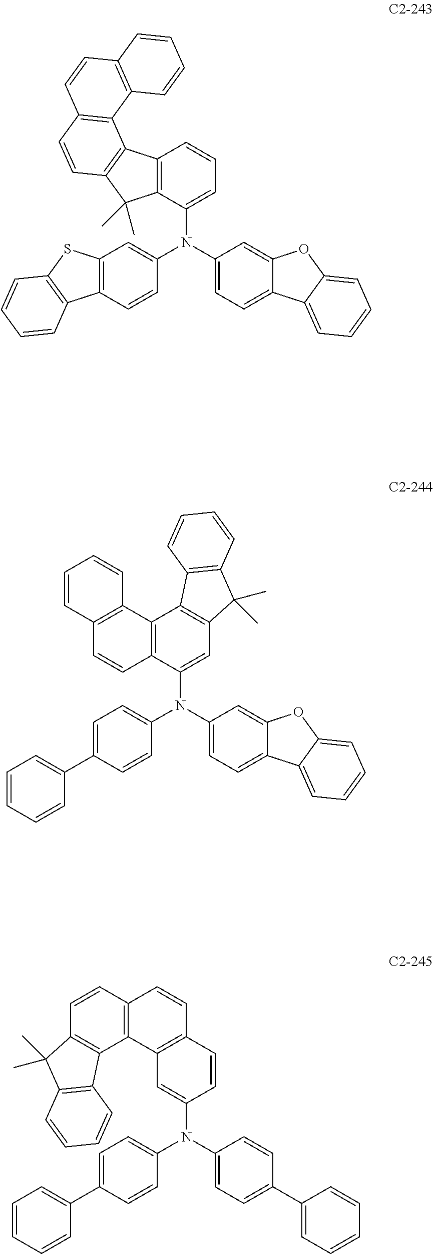 [SMILES: CC1(C)c2ccc3ccc4ccccc4c3c2-c2cccc(N(c3ccc4c(c3)oc3ccccc34)c3ccc4c(c3)sc3ccccc34)c21 ; CC1(C)c2ccccc2-c2c1cc(N(c1ccc(-c3ccccc3)cc1)c1ccc3c(c1)oc1ccccc13)c1ccc3ccccc3c21 ; CC1(C)c2ccccc2-c2c1ccc1ccc3ccc(N(c4ccc(-c5ccccc5)cc4)c4ccc(-c5ccccc5)cc4)cc3c21]

The compound represented by the formula 2 according to the present disclosure may be produced by a synthetic method known to a person skilled in the art, for example, the compound represented by the formula 2 may be prepared by referring to KR 2017-0043439 A (published on Apr. 21, 2017), but is not limited thereto.

According to another embodiment of the present disclosure, an organic electroluminescent compound represented by the following formula 2′ is provided.

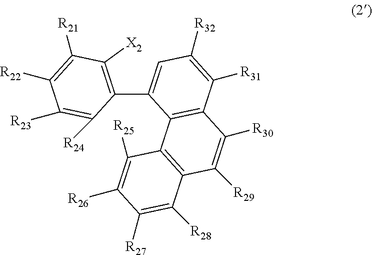

in formula 2′,

X₂ represents O, S, or CR₇R₈;

R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);

R₂₁ to R₃₂ each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a fused ring of a substituted or unsubstituted (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-NAr₁Ar₂; provided that at least one of R₂₁ to R₃₂ is *-L₁-NAr₁Ar₂;

L₁ represents a single bond, a substituted or unsubstituted (C10-C30)arylene, or a substituted or unsubstituted (3- to 30-membered)heteroarylene; and

Ar₁ and Ar₂ each independently represent, a substituted or unsubstituted (3- to 30-membered)heteroaryl, provided that the case where all of Ar₁ and Ar₂ are carbazole, is excluded.

In one embodiment, R₂₁ to R₃₂ in the formula 2′, each independently may be hydrogen, deuterium, halogen, cyano, or *-L₁-NAr₁Ar₂, provided that at least one of R₂₁ to R₃₂ is *-L₁-NAr₁Ar₂. For example, at least one of R₂₁ to R₂₄, or at least one of R₂₅ to R₂₈, or at least one of R₃₁ and R₃₂ may be *-L₁-NAr₁Ar₂.

In one embodiment, X₂ in the formula 2′, represents O, S, or CR₇R₈, in which, for example, R₇ and R₈ each independently may be a substituted or unsubstituted (C1-C30)alkyl, preferably, a substituted or unsubstituted (C1-C10)alkyl, more preferably a substituted or unsubstituted (C1-C4)alkyl.

In one embodiment, L₅ in the formula 2′, may be a single bond.

In one embodiment, Ar₁ and Ar₂ in the formula 2′, each independently may be a substituted or unsubstituted (5- to 30-membered)heteroaryl, preferably a substituted or unsubstituted (5- to 25-membered)heteroaryl, more preferably a substituted or unsubstituted (5- to 18-membered)heteroaryl. However, in the organic electroluminescent compound represented by the above formula 2′, the case where all of Ar₁ and Ar₂ are carbazole, is excluded. For example, Ar₁ and Ar₂ each independently may be a substituted or unsubstituted dibenzofuranyl or a substituted or unsubstituted dibenzothiophenyl.

In one embodiment, the organic electroluminescent compound represented by the above formula 2′ may be more specifically illustrated by the following compounds, but is not limited thereto.

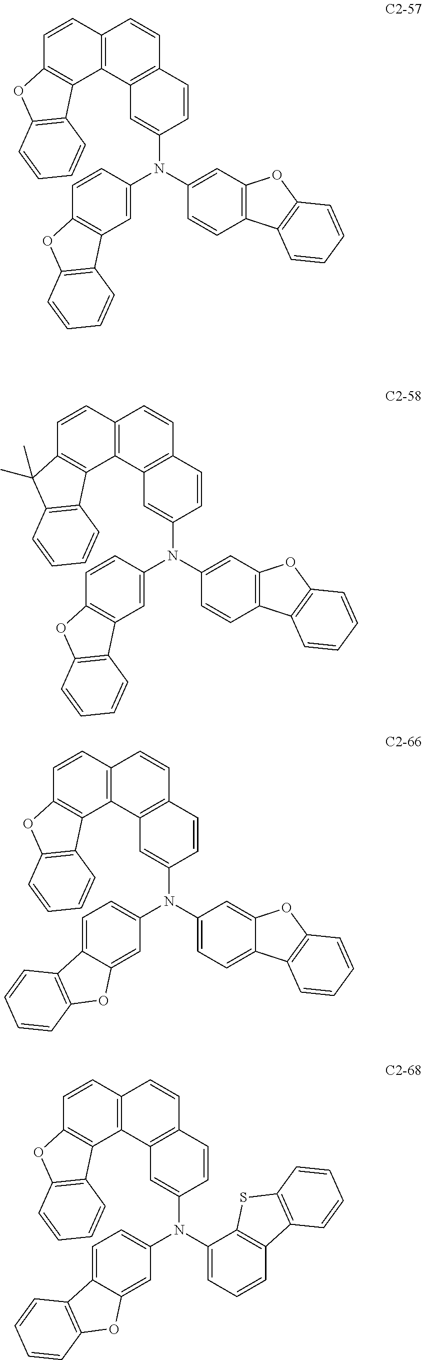

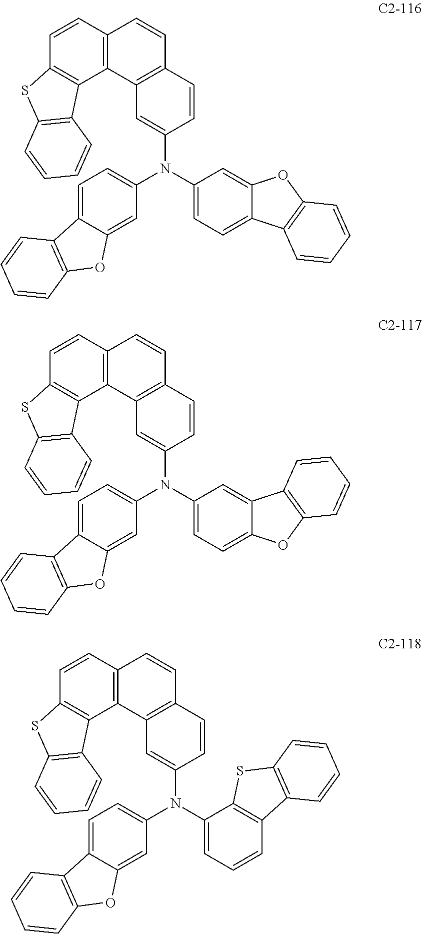 [SMILES: c1ccc2c(c1)oc1cc(N(c3ccc4c(c3)oc3ccccc34)c3ccc4ccc5ccc6sc7ccccc7c6c5c4c3)ccc12 ; c1ccc2c(c1)oc1cc(N(c3ccc4oc5ccccc5c4c3)c3ccc4ccc5ccc6sc7ccccc7c6c5c4c3)ccc12 ; c1ccc2c(c1)oc1cc(N(c3ccc4ccc5ccc6sc7ccccc7c6c5c4c3)c3cccc4c3sc3ccccc34)ccc12]

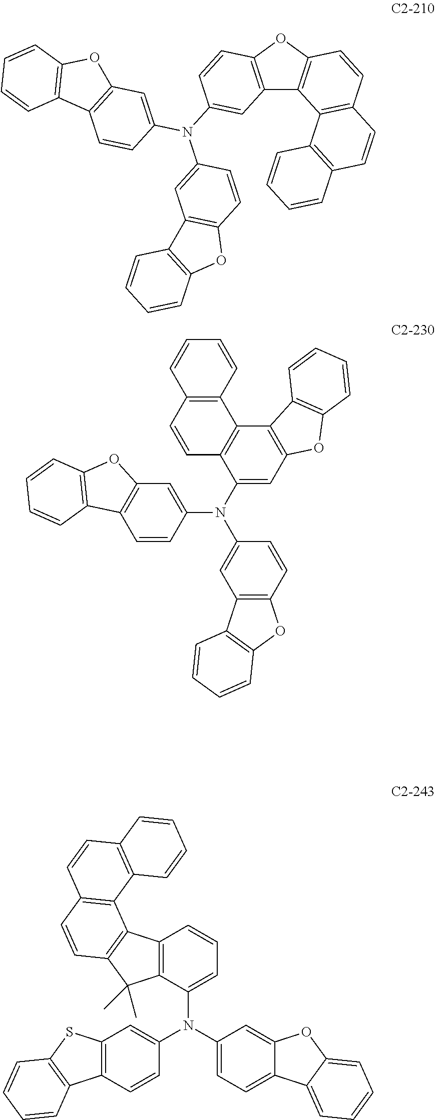

According to another embodiment of the present disclosure, an organic electroluminescent compound represented by the following formula 2″ is provided.

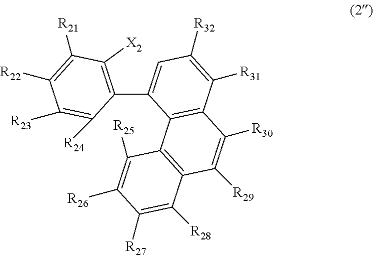

in formula 2″,

X₂ represents O or S;

R₂₁ to R₃₂ each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a fused ring of a substituted or unsubstituted (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-030)alkyldi(C6-C30)arylsilyl, a substituted or unsubstituted tri(C6-C30)arylsilyl, or *-L₂-HAr; provided that at least one of R₂₁ to R₂₄ and R₂₉ to R₃₂ is*-L₂-HAr;

L₂ represents a single bond, a substituted or unsubstituted (C6-C30)arylene, or a substituted or unsubstituted (3- to 30-membered)heteroarylene;

HAr represents a substituted or unsubstituted triazinyl or a substituted or unsubstituted quinoxalinyl:

with the proviso that the following compounds are excluded from the organic electroluminescent compound represented by the above formula 2″,

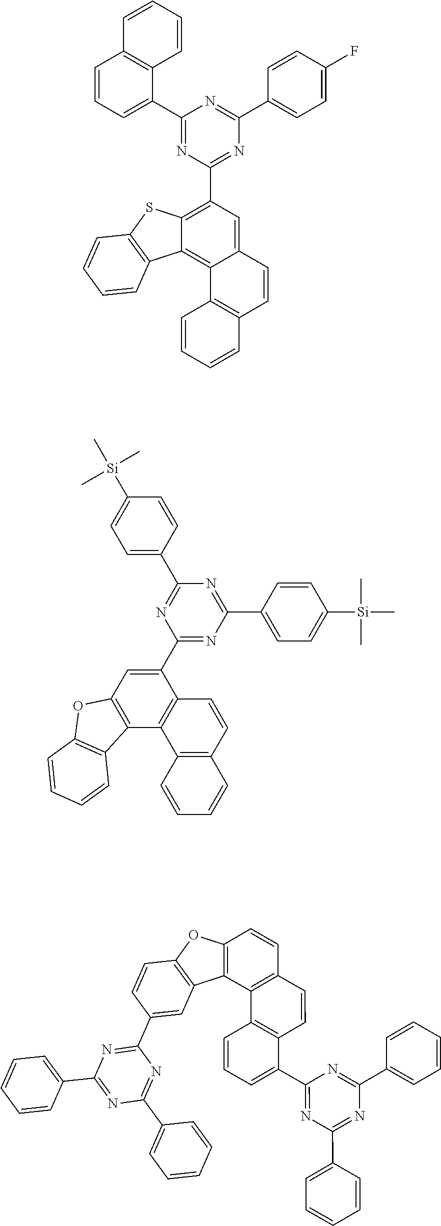 [SMILES: Fc1ccc(-c2nc(-c3cccc4ccccc34)nc(-c3cc4ccc5ccccc5c4c4c3sc3ccccc34)n2)cc1 ; C[Si](C)(C)c1ccc(-c2nc(-c3ccc([Si](C)(C)C)cc3)nc(-c3cc4oc5ccccc5c4c4c3ccc3ccccc34)n2)cc1 ; c1ccc(-c2nc(-c3ccccc3)nc(-c3ccc4oc5ccc6ccc7c(-c8nc(-c9ccccc9)nc(-c9ccccc9)n8)cccc7c6c5c4c3)n2)cc1]

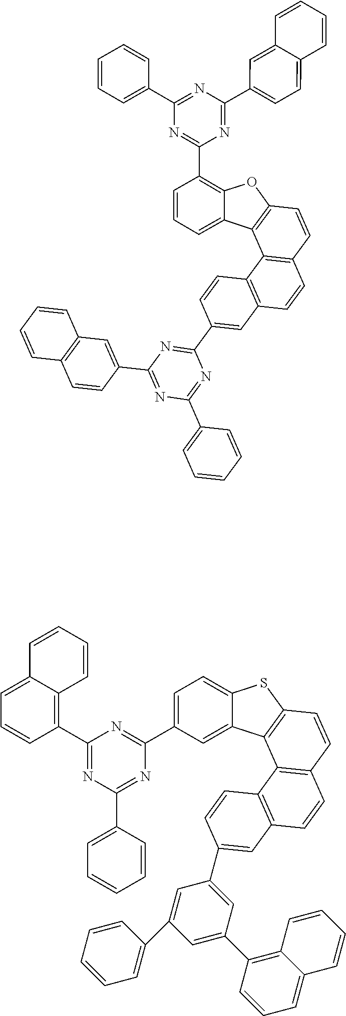 [SMILES: c1ccc(-c2nc(-c3ccc4ccccc4c3)nc(-c3ccc4c(ccc5ccc6oc7c(-c8nc(-c9ccccc9)nc(-c9ccc%10ccccc%10c9)n8)cccc7c6c54)c3)n2)cc1 ; c1ccc(-c2cc(-c3ccc4c(ccc5ccc6sc7ccc(-c8nc(-c9ccccc9)nc(-c9cccc%10ccccc9%10)n8)cc7c6c54)c3)cc(-c3cccc4ccccc34)c2)cc1]

In one embodiment, R₂₁ to R₃₂ in the formula 2″, each independently may be hydrogen, deuterium, halogen, cyano, or *-L₂-HAr; provided that at least one of R₂₁ to R₂₄ and R₂₉ to R₃₂ is *-L₂-HAr. For example, at least one of R₂₁ to R₂₄ or at least one of R₃₁ and R₃₂ may be *-L₂-HAr.

In one embodiment, L₂ in the formula 2″, may be a single bond, a substituted or unsubstituted phenylene, a substituted or unsubstituted pyridylene, or a substituted or unsubstituted naphthylene.

In one embodiment, HAr in the formula 2″, represents a substituted or unsubstituted triazinyl or a substituted or unsubstituted quinoxalinyl, in which the substituents in the substituted triazinyl or the substituted quinoxalinyl each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably, a substituted or unsubstituted (C6-C18)aryl or a substituted or unsubstituted (5- to 18-membered)heteroaryl, for example, the substituents may be phenyl, naphthyl, p-biphenyl, m-biphenyl, dimethylfluorenyl, phenylnaphthyl, p-terphenyl, phenanthrenyl, pyridyl, or dibenzofuranyl.

According to one embodiment, the organic electroluminescent compound represented by the above formula 2″ may be more specifically illustrated by the following compounds, but is not limited thereto.

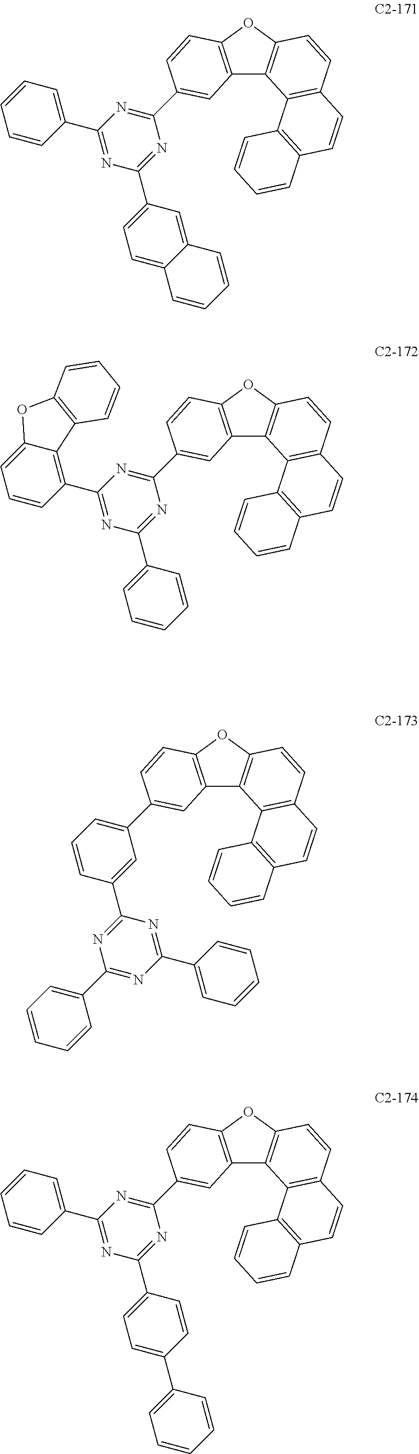 [SMILES: c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1 ; c1ccc(-c2nc(-c3ccccc3)nc(-c3cccc(-c4ccc5oc6ccc7ccc8ccccc8c7c6c5c4)c3)n2)cc1 ; c1ccc(-c2ccc(-c3nc(-c4ccccc4)nc(-c4ccc5oc6ccc7ccc8ccccc8c7c6c5c4)n3)cc2)cc1]

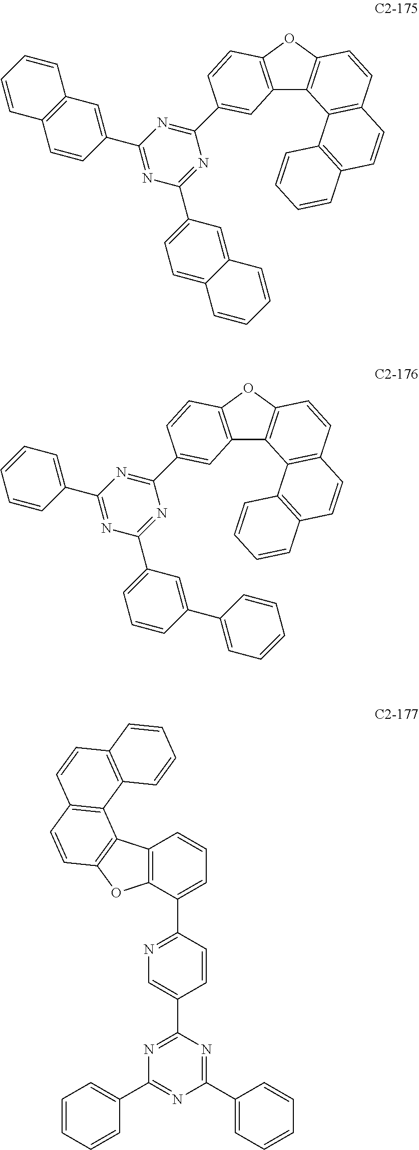 [SMILES: c1ccc2cc(-c3nc(-c4ccc5ccccc5c4)nc(-c4ccc5oc6ccc7ccc8ccccc8c7c6c5c4)n3)ccc2c1 ; c1ccc(-c2cccc(-c3nc(-c4ccccc4)nc(-c4ccc5oc6ccc7ccc8ccccc8c7c6c5c4)n3)c2)cc1 ; c1ccc(-c2nc(-c3ccccc3)nc(-c3ccc(-c4cccc5c4oc4ccc6ccc7ccccc7c6c45)nc3)n2)cc1]

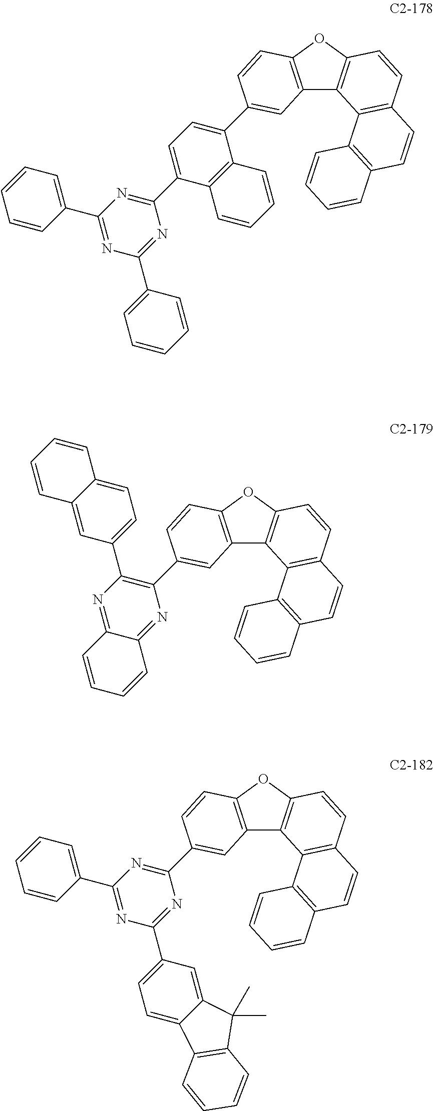 [SMILES: c1ccc(-c2nc(-c3ccccc3)nc(-c3ccc(-c4ccc5oc6ccc7ccc8ccccc8c7c6c5c4)c4ccccc34)n2)cc1 ; c1ccc2cc(-c3nc4ccccc4nc3-c3ccc4oc5ccc6ccc7ccccc7c6c5c4c3)ccc2c1 ; CC1(C)c2ccccc2-c2ccc(-c3nc(-c4ccccc4)nc(-c4ccc5oc6ccc7ccc8ccccc8c7c6c5c4)n3)cc21]

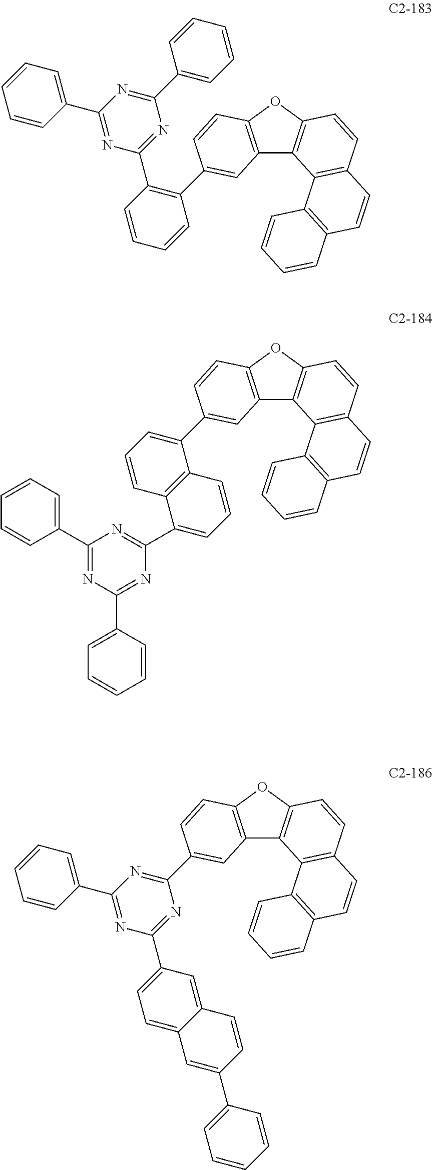 [SMILES: c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1 ; c1ccc(-c2ccc3cc(-c4nc(-c5ccccc5)nc(-c5ccc6oc7ccc8ccc9ccccc9c8c7c6c5)n4)ccc3c2)cc1]

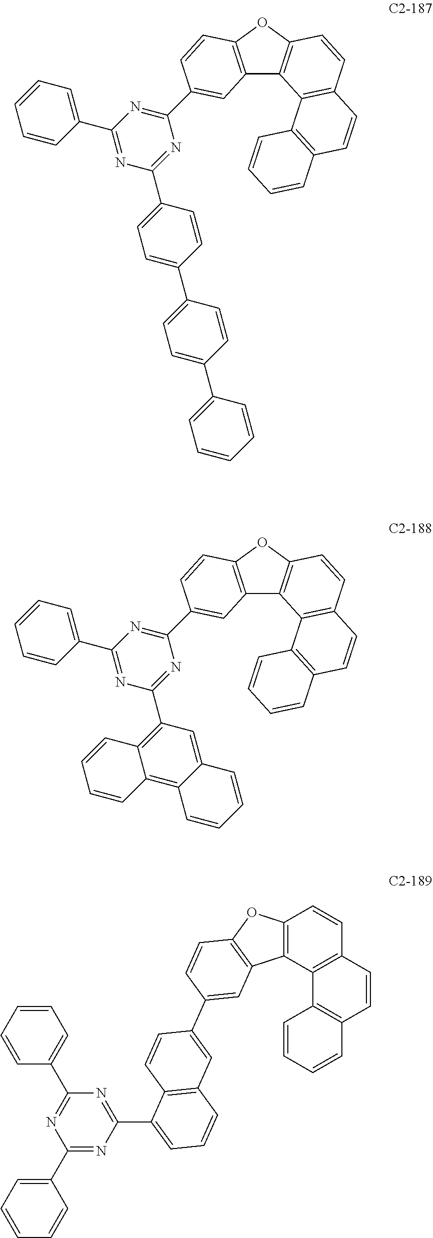 [SMILES: c1ccc(-c2ccc(-c3ccc(-c4nc(-c5ccccc5)nc(-c5ccc6oc7ccc8ccc9ccccc9c8c7c6c5)n4)cc3)cc2)cc1 ; c1ccc(-c2nc(-c3ccc4oc5ccc6ccc7ccccc7c6c5c4c3)nc(-c3cc4ccccc4c4ccccc34)n2)cc1 ; c1ccc(-c2nc(-c3ccccc3)nc(-c3cccc4cc(-c5ccc6oc7ccc8ccc9ccccc9c8c7c6c5)ccc34)n2)cc1]

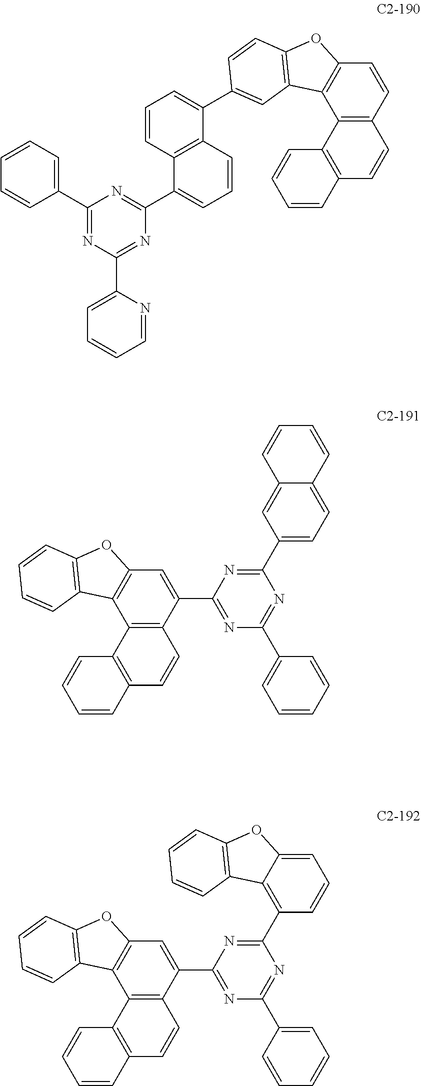 [SMILES: c1ccc(-c2nc(-c3ccccn3)nc(-c3cccc4c(-c5ccc6oc7ccc8ccc9ccccc9c8c7c6c5)cccc34)n2)cc1 ; c1ccc(-c2nc(-c3ccc4ccccc4c3)nc(-c3cc4oc5ccccc5c4c4c3ccc3ccccc34)n2)cc1 ; c1ccc(-c2nc(-c3cc4oc5ccccc5c4c4c3ccc3ccccc34)nc(-c3cccc4oc5ccccc5c34)n2)cc1]

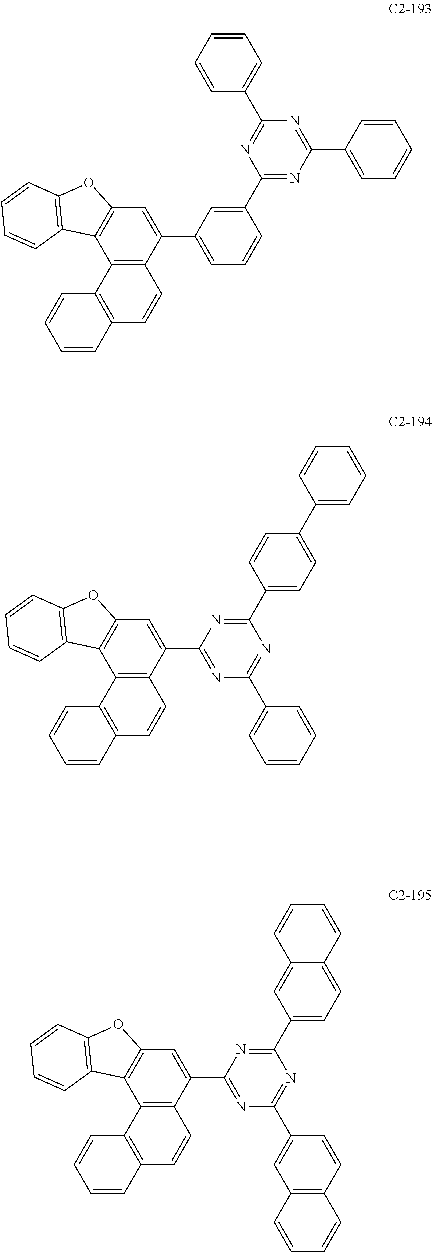 [SMILES: c1ccc(-c2nc(-c3ccccc3)nc(-c3cccc(-c4cc5oc6ccccc6c5c5c4ccc4ccccc45)c3)n2)cc1 ; c1ccc(-c2ccc(-c3nc(-c4ccccc4)nc(-c4cc5oc6ccccc6c5c5c4ccc4ccccc45)n3)cc2)cc1 ; c1ccc2cc(-c3nc(-c4ccc5ccccc5c4)nc(-c4cc5oc6ccccc6c5c5c4ccc4ccccc45)n3)ccc2c1]

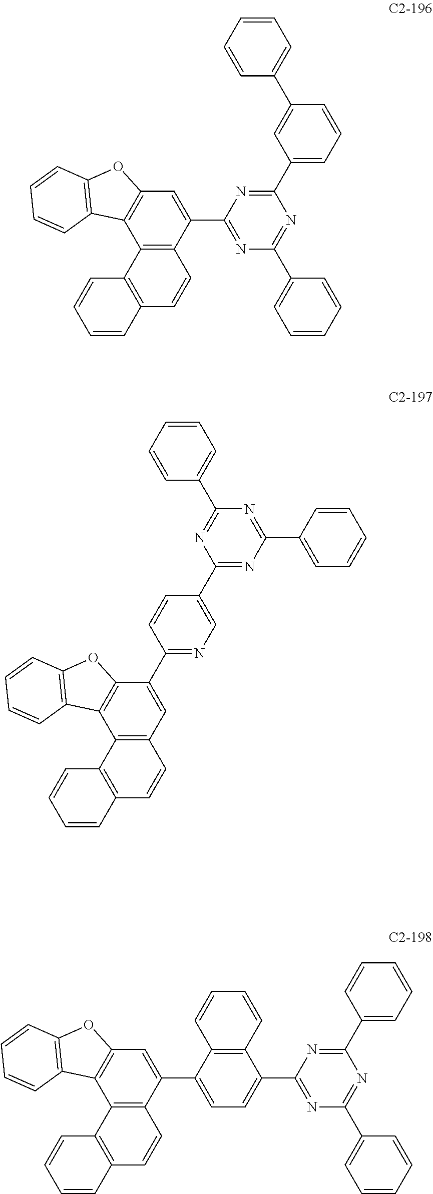 [SMILES: c1ccc(-c2cccc(-c3nc(-c4ccccc4)nc(-c4cc5oc6ccccc6c5c5c4ccc4ccccc45)n3)c2)cc1 ; c1ccc(-c2nc(-c3ccccc3)nc(-c3ccc(-c4cc5ccc6ccccc6c5c5c4oc4ccccc45)nc3)n2)cc1 ; c1ccc(-c2nc(-c3ccccc3)nc(-c3ccc(-c4cc5oc6ccccc6c5c5c4ccc4ccccc45)c4ccccc34)n2)cc1]

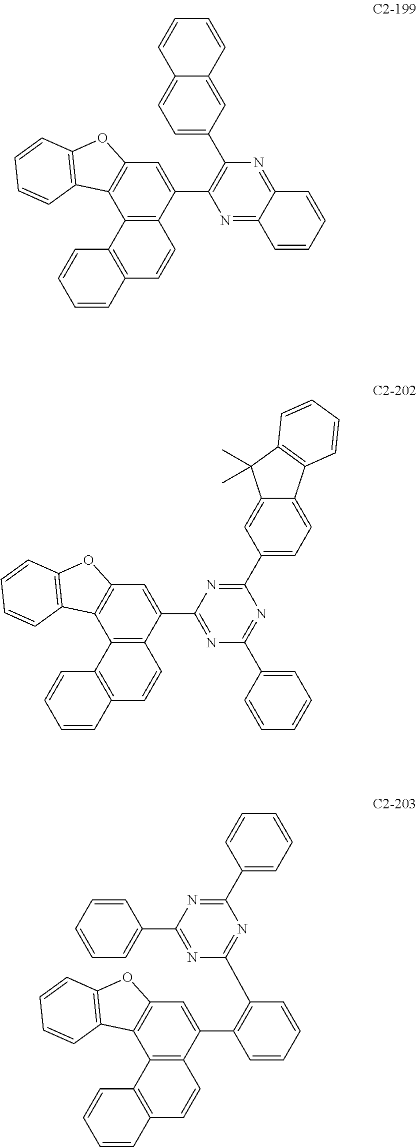 [SMILES: c1ccc2cc(-c3nc4ccccc4nc3-c3cc4oc5ccccc5c4c4c3ccc3ccccc34)ccc2c1 ; CC1(C)c2ccccc2-c2ccc(-c3nc(-c4ccccc4)nc(-c4cc5oc6ccccc6c5c5c4ccc4ccccc45)n3)cc21 ; c1ccc(-c2nc(-c3ccccc3)nc(-c3ccccc3-c3cc4oc5ccccc5c4c4c3ccc3ccccc34)n2)cc1]

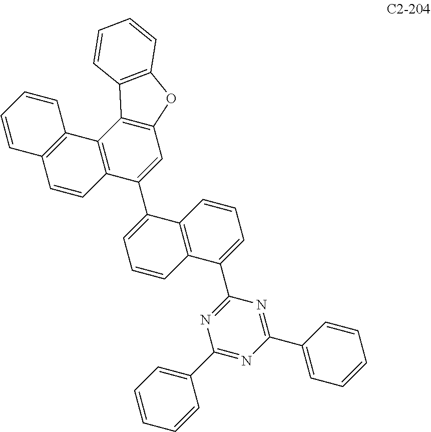 [SMILES: c1ccc(-c2nc(-c3ccccc3)nc(-c3cccc4c(-c5cc6oc7ccccc7c6c6c5ccc5ccccc56)cccc34)n2)cc1]

Hereinafter, the aforementioned plurality of host materials, the organic electroluminescent compound, and/or an organic electroluminescent device being applied to the organic electroluminescent material comprising the same will be described.

The organic electroluminescent device according to one embodiment includes a first electrode; a second electrode; and at least one organic layer interposed between the first electrode and the second electrode. The organic layer may include a light-emitting layer, and the light-emitting layer may comprise a plurality of host materials comprising at least one first host material represented by the above formula 1 and at least one second host material represented by the above formula 2. For example, the light-emitting layer may comprise an organic electroluminescent compound represented by the above formula 2′ and/or an organic electroluminescent compound represented by the above formula 2″. According to another embodiment, an organic electroluminescent compound represented by the above formula 2′ and/or an organic electroluminescent compound represented by the above formula 2″ may be included in a hole transport zone or an electron transport zone of the organic electroluminescent device.

According to one embodiment, the organic electroluminescent material of the present disclosure comprises at least one compound(s) of compounds H1-1 to H1-180 as the first host material represented by the above formula 1 and at least one compound(s) of compounds C2-1 to C2-245 as the second host material represented by the above formula 2, and the plurality of host materials may be included in the same organic layer or may be included in different organic layers, respectively.

According to another embodiment, the organic electroluminescent material of the present disclosure may comprises an organic electroluminescent compound represented by the above formula 2′ and/or an organic electroluminescent compound represented by the above formula 2″ which may be included in the same organic layer or may be included in different organic layers, respectively.

The light-emitting layer is a layer from which light is emitted, and can be a single layer or a multi-layer of which two or more layers are stacked. The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer, in addition to the light-emitting layer.

The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material.

Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

An organic electroluminescent material according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer. In the light-emitting layer, it is preferable that the doping concentration of the dopant compound based on the host compound may be less than 20 wt %, preferably, 17 wt %.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compounds) of a metal atom(s) selected from iridium (Ir), osmium (Os), copper (Cu), and platinum (Pt), more preferably an ortho-metallated complex compound(s) of a metal atoms) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following formula 101, but is not limited thereto,

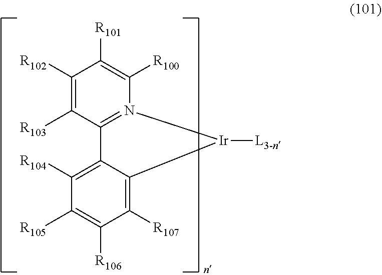

in formula 101,

L is selected from any one of the following structures 1 to 3;

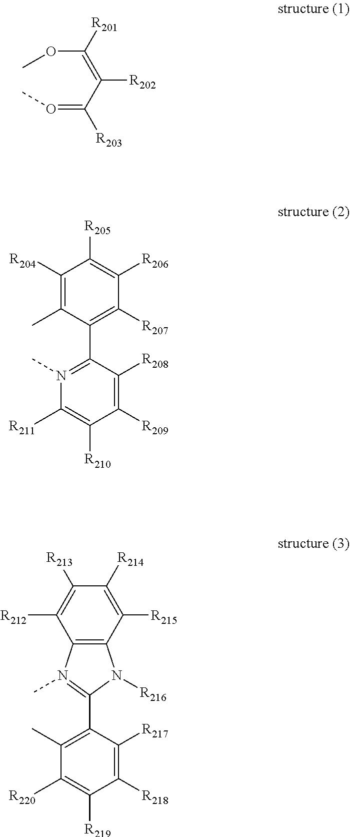

in structures 1 to 3,

R₁₀₀ to R₁₀₃ each independently represent, hydrogen, deuterium, halogen, deuterium- and/or halogen-substituted or unsubstituted (C1-C30)alkyl, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to an adjacent substituent(s) to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent, hydrogen, deuterium, halogen, deuterium- and/or halogen-substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to an adjacent substituent(s) to form a ring(s), for example, to form a ring(s) with a benzene, e.g., a substituted or unsubstituted naphthalene, a substituted or unsubstituted fluoren, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₂₀ each independently represent, hydrogen, deuterium, halogen, deuterium- and/or halogen-substituted or unsubstituted (C1-C30)alkyl, a substituted or unsubstituted (C3-C30)cycloalkyl, or a substituted or unsubstituted (C6-C30)aryl; or may be linked to an adjacent substituent(s) to form a ring(s); and

n′ represent an integer of 1 to 3.

Specifically, the specific examples of the dopant compound include the following, but are not limited thereto.

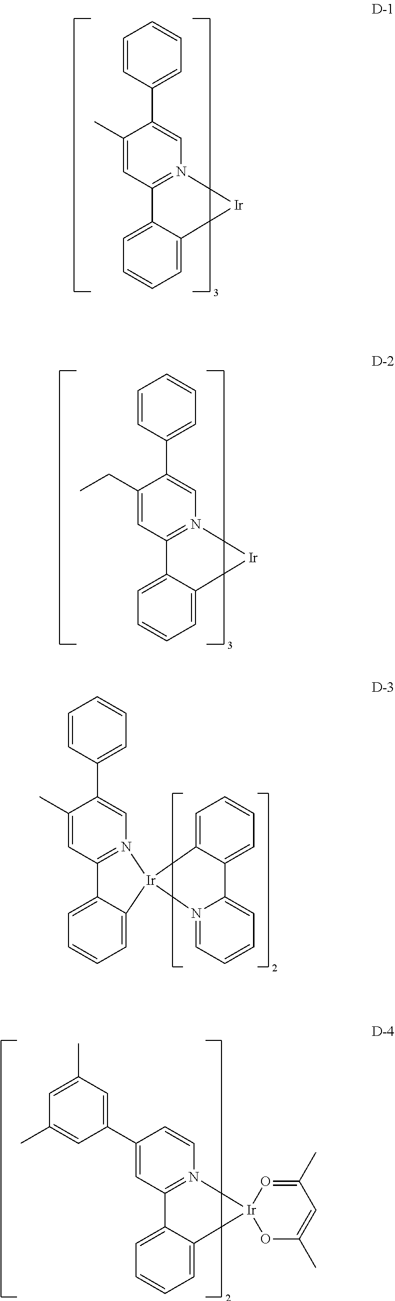

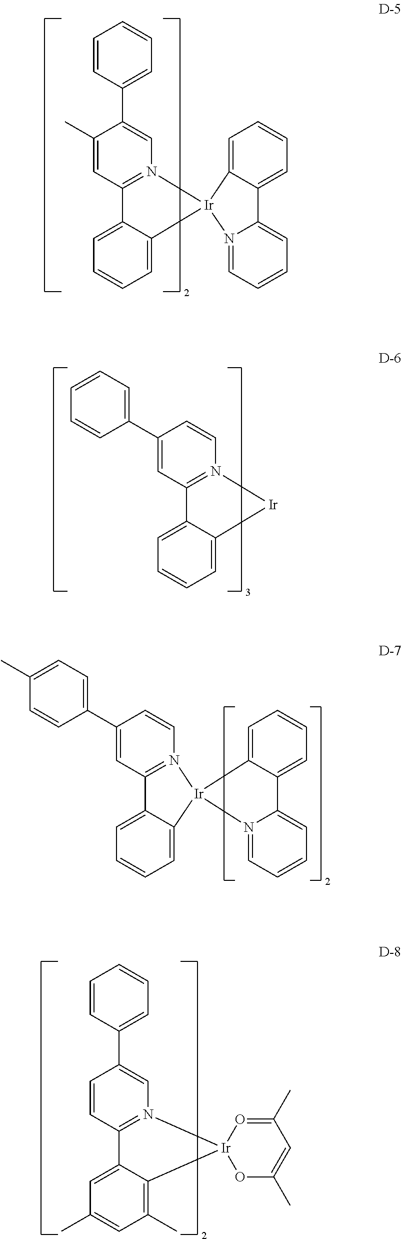

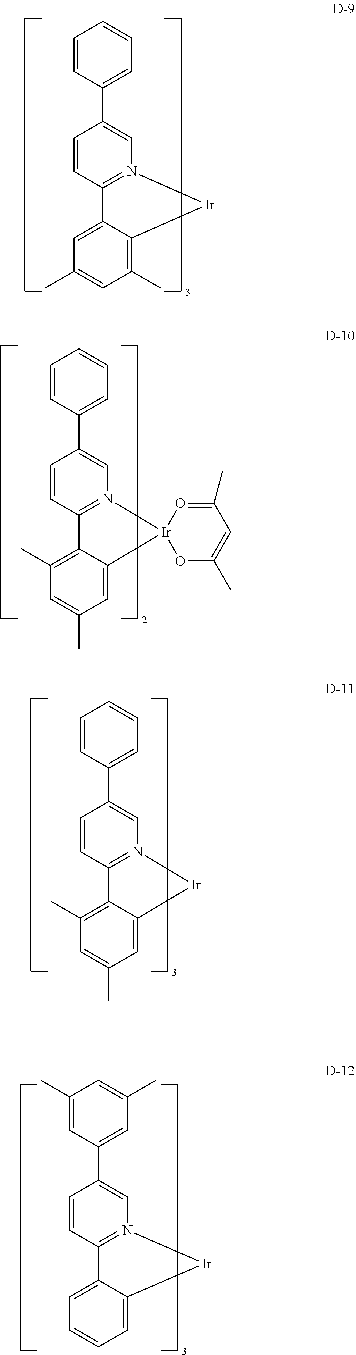

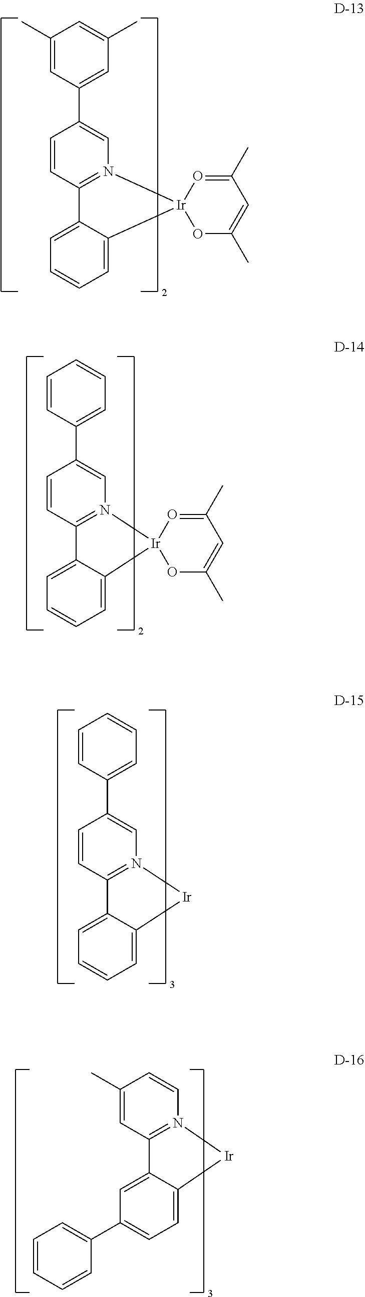

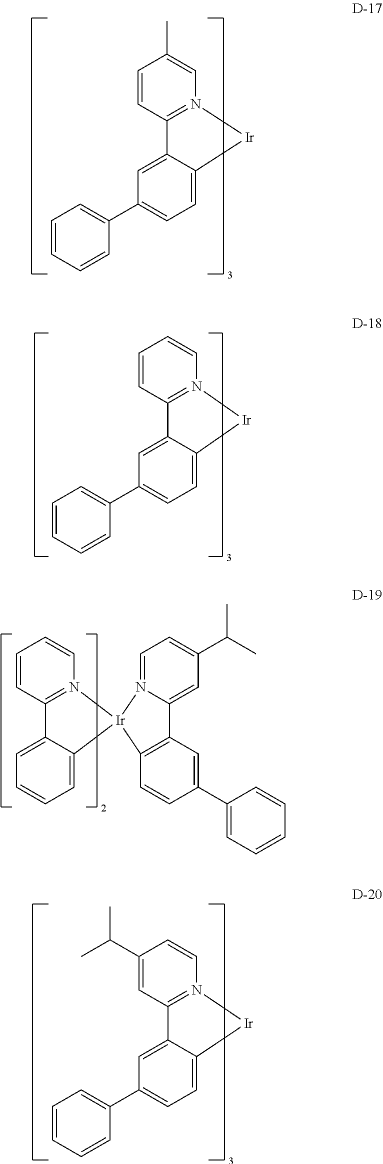

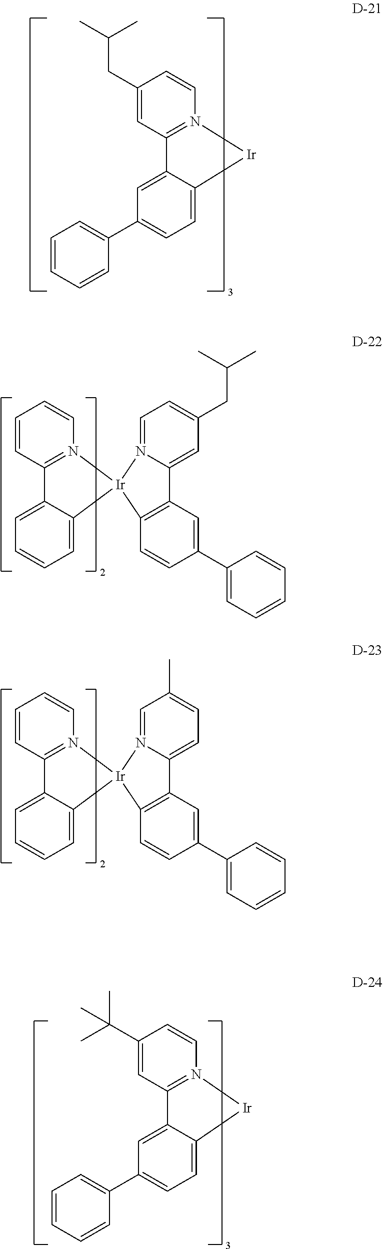

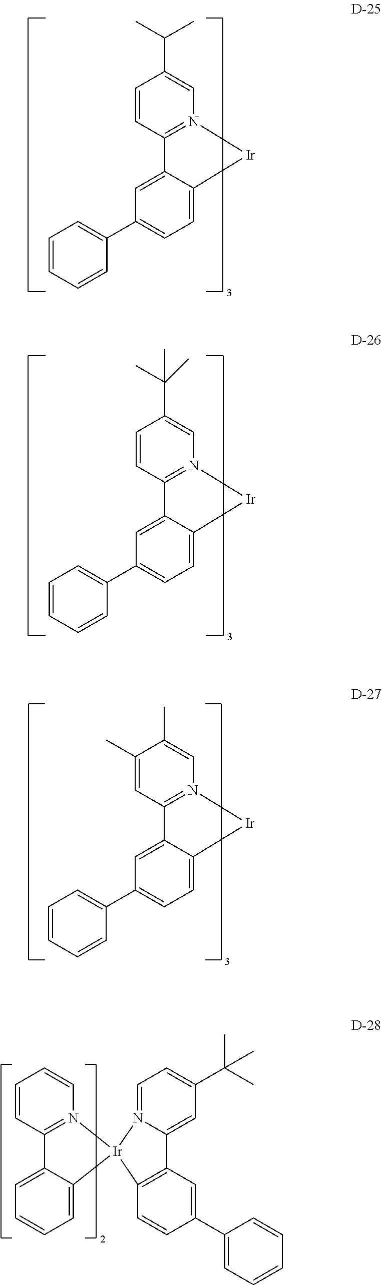

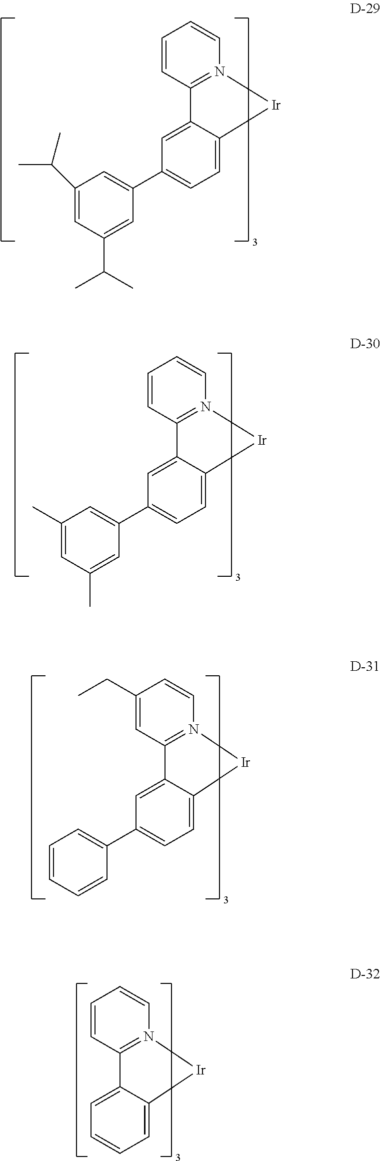

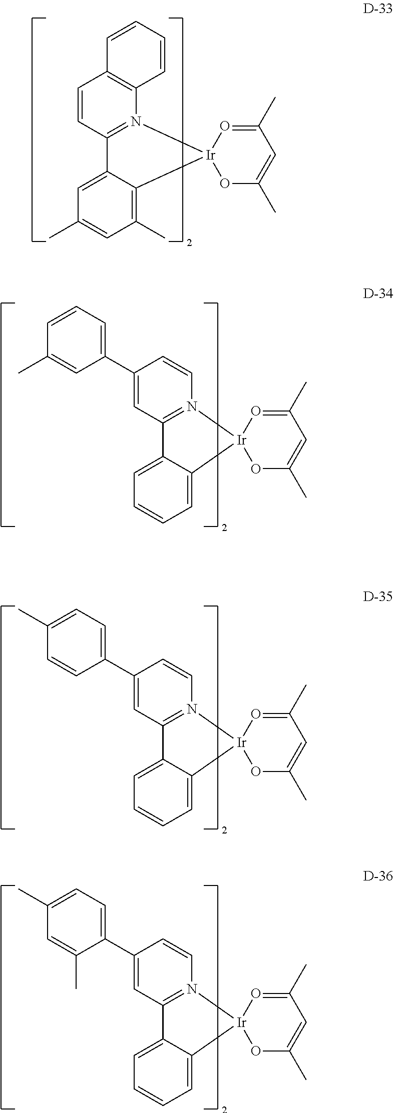

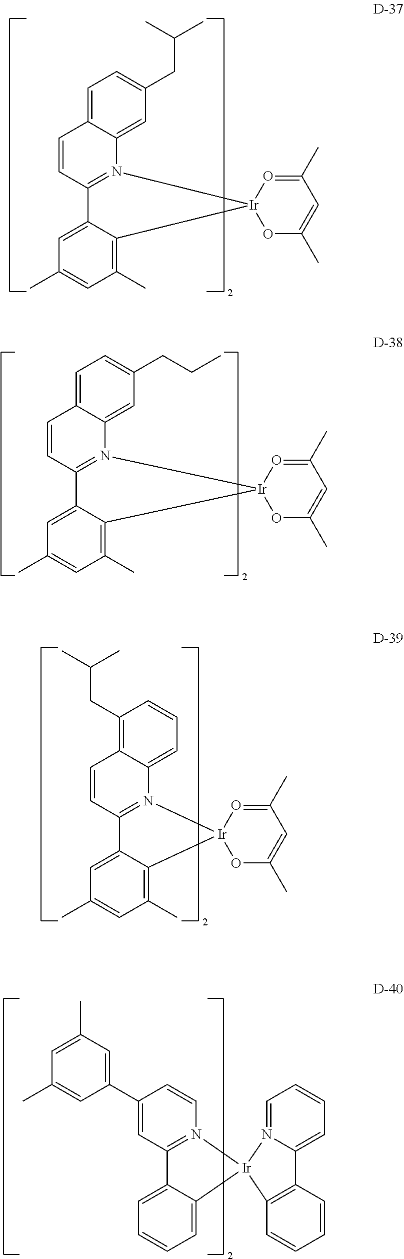

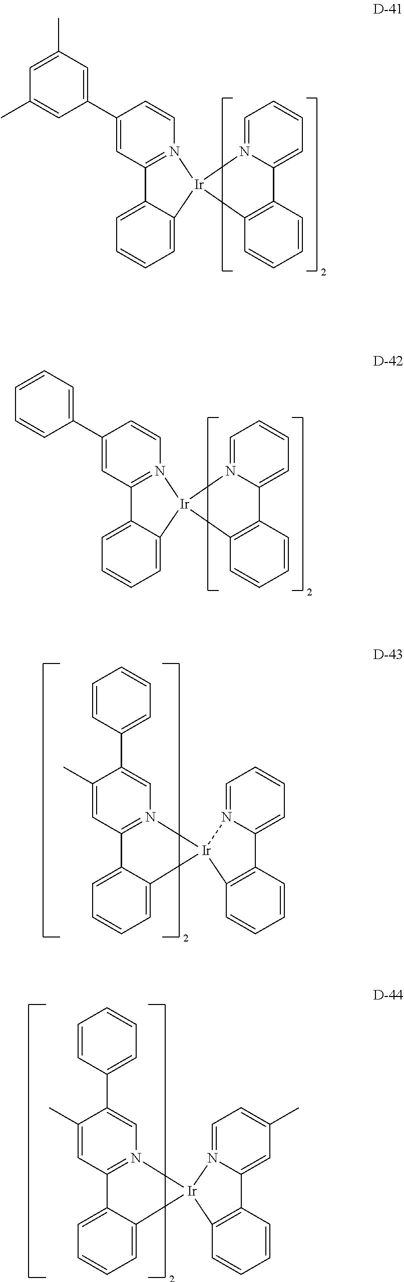

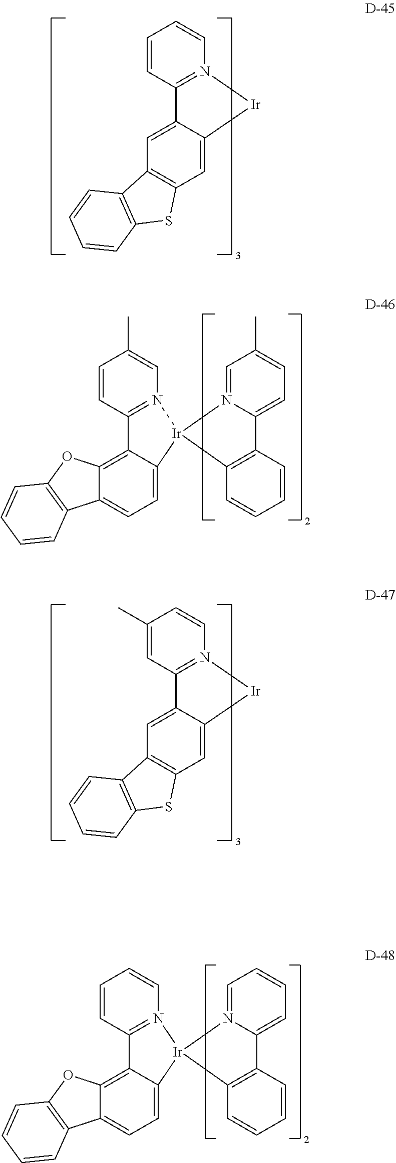

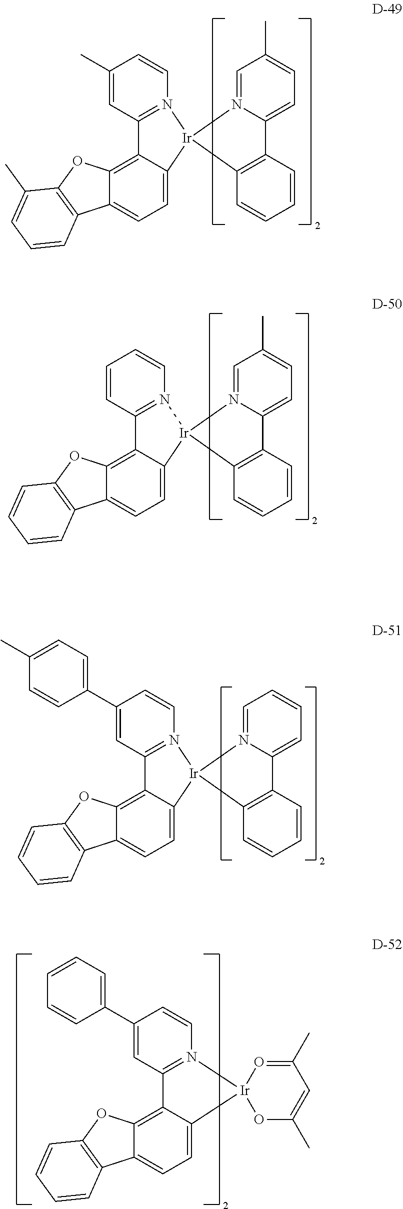

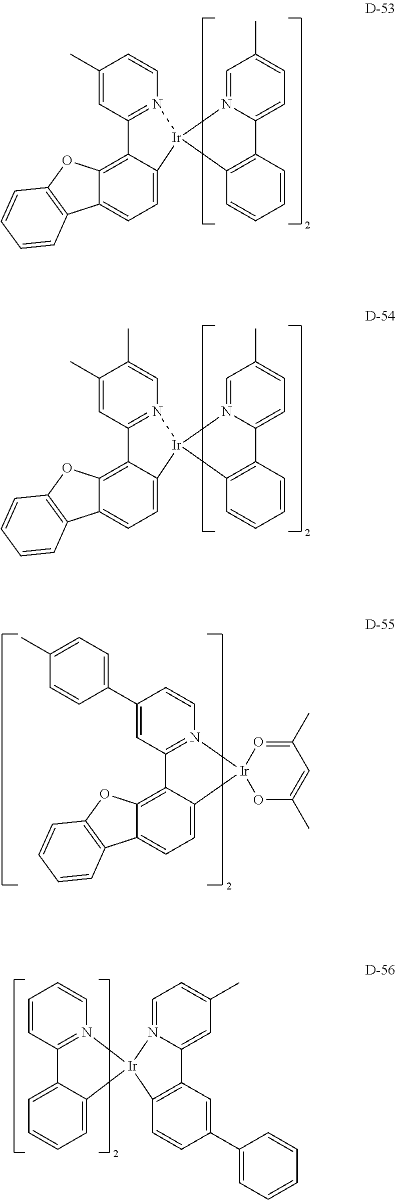

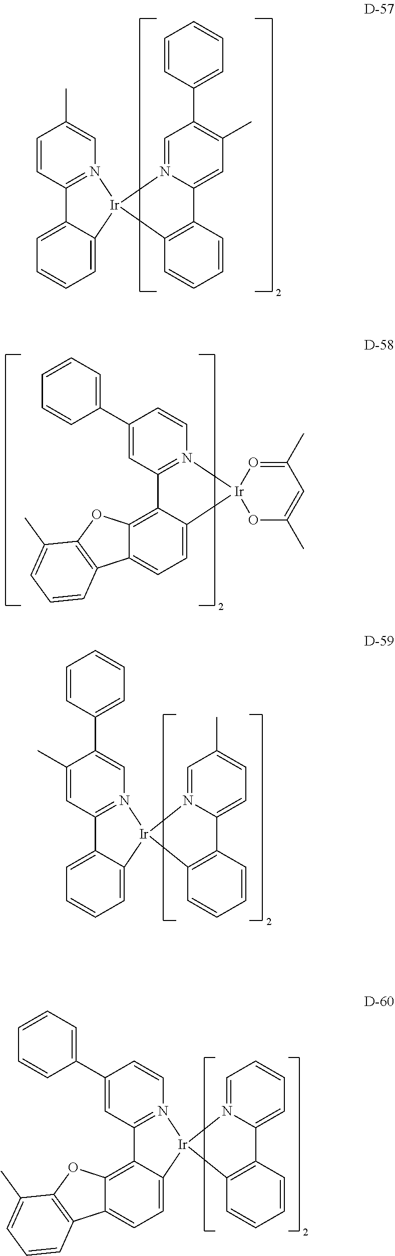

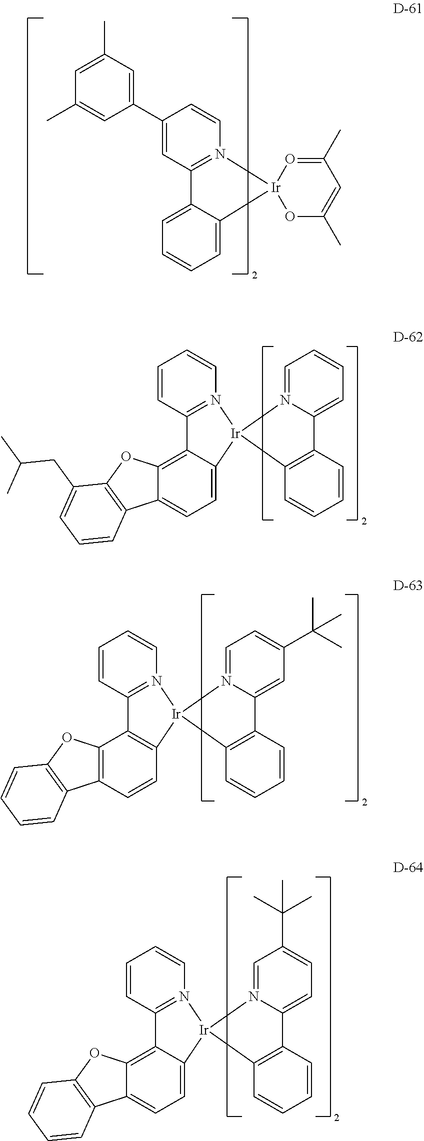

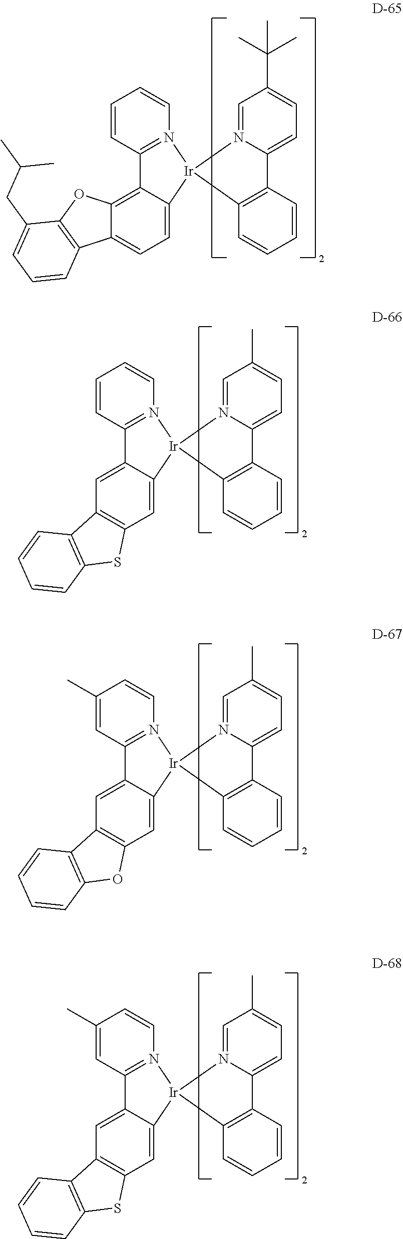

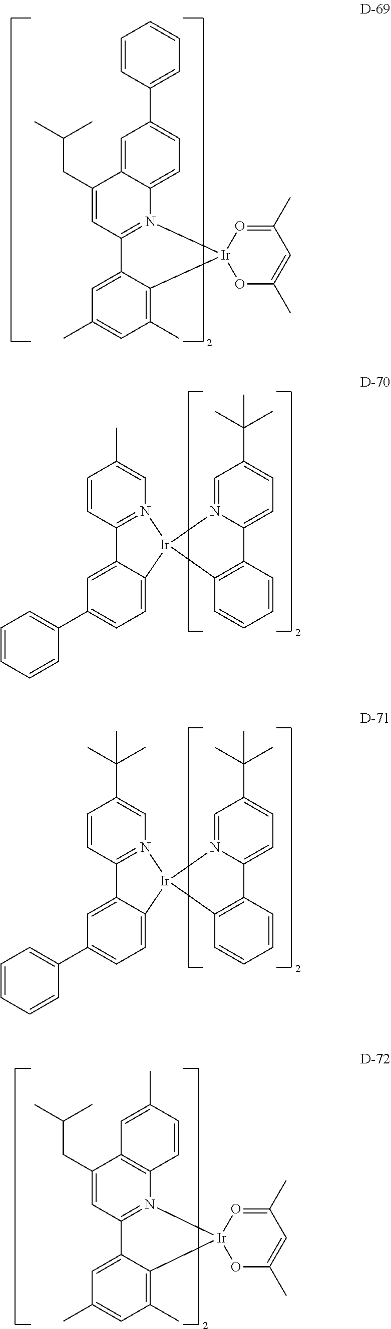

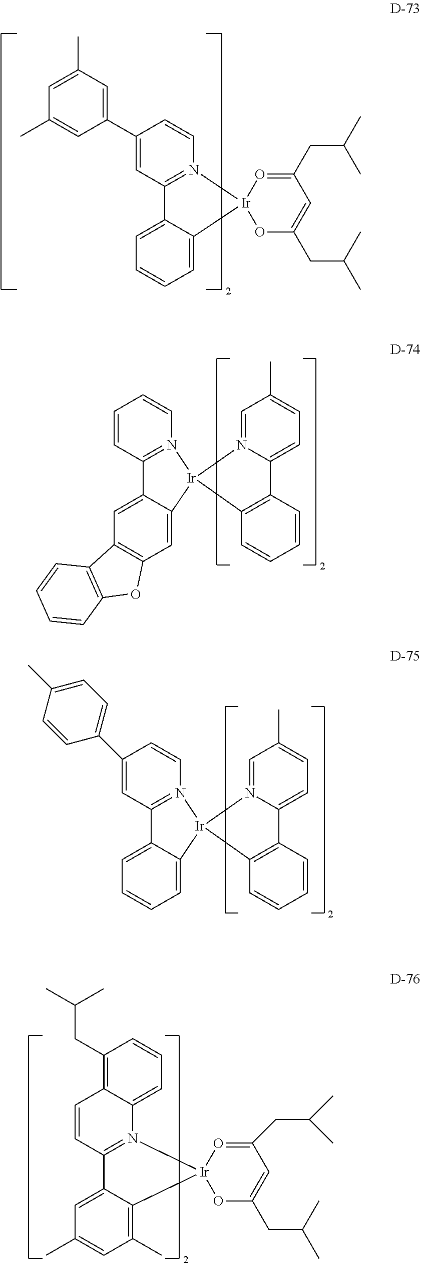

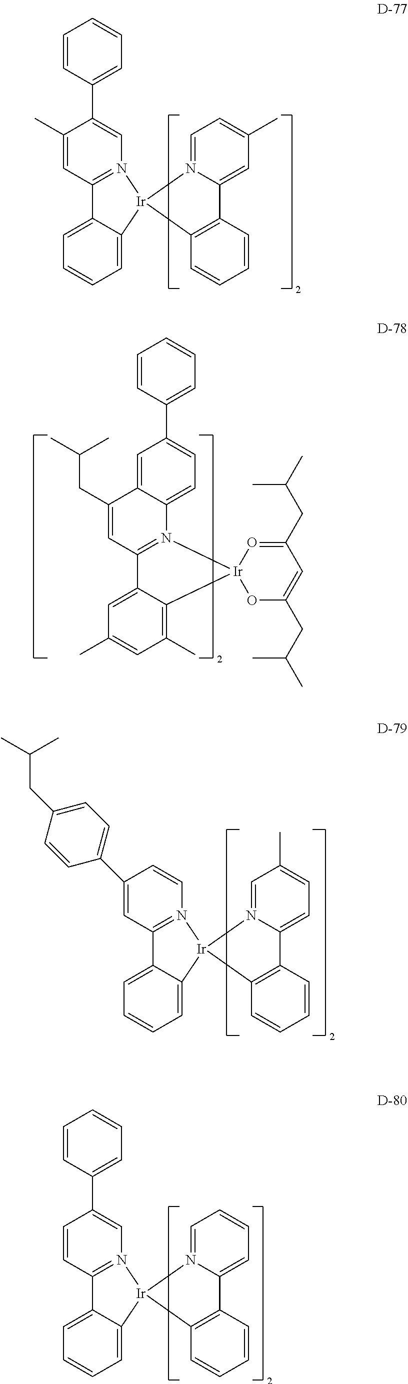

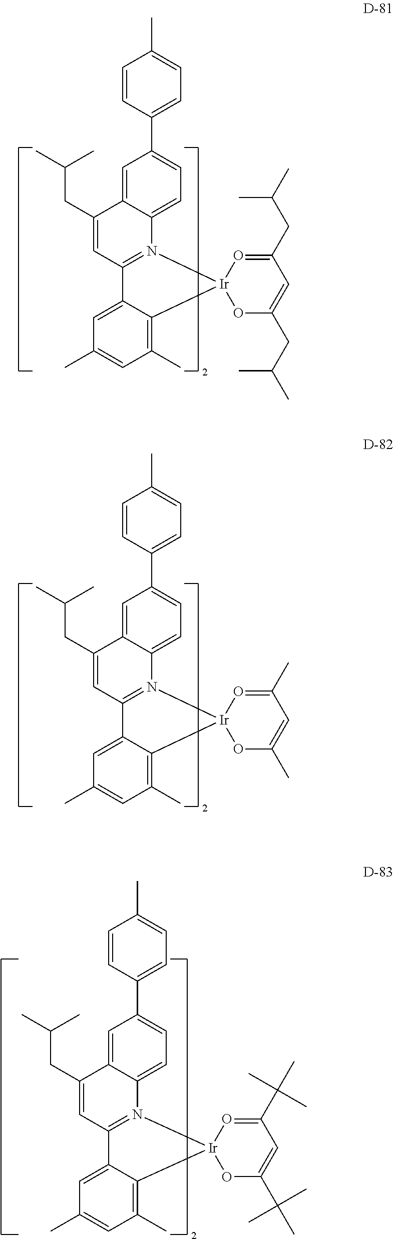

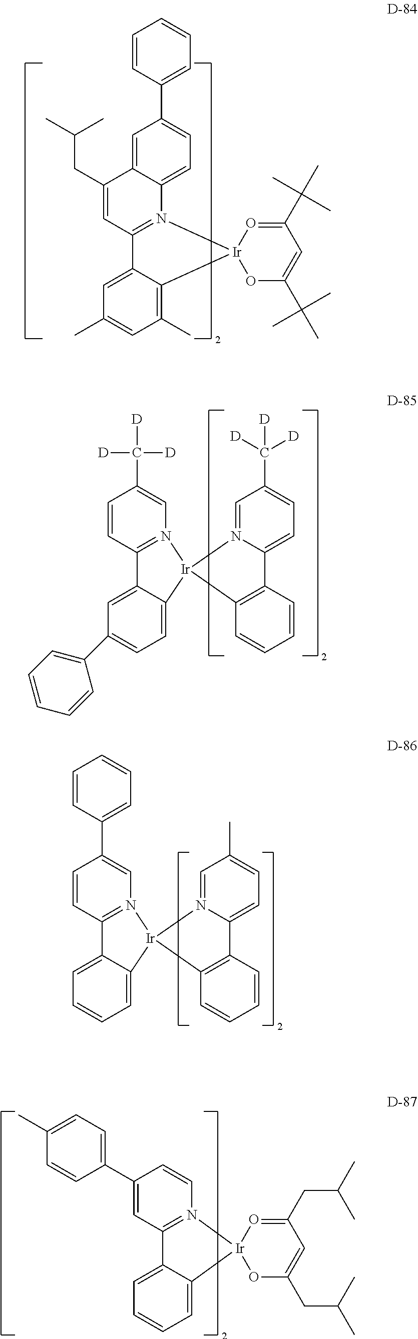

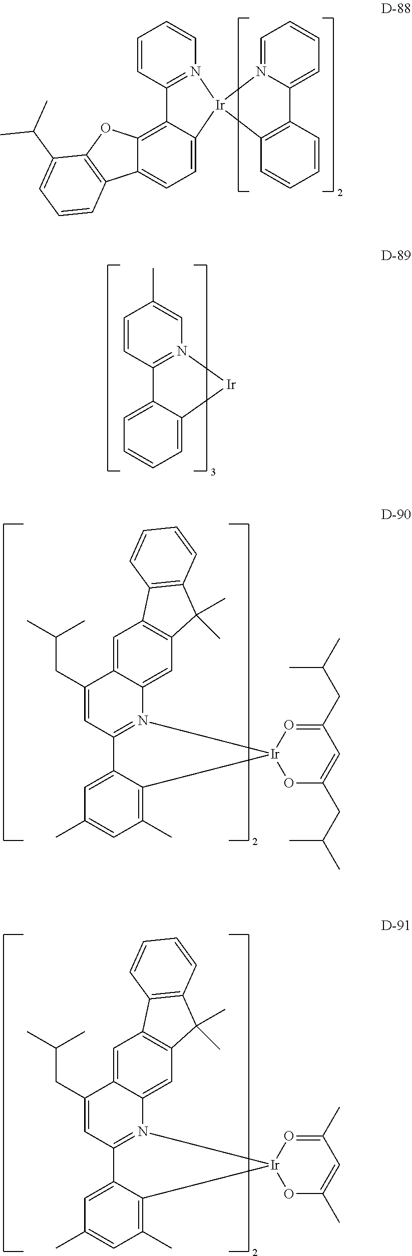

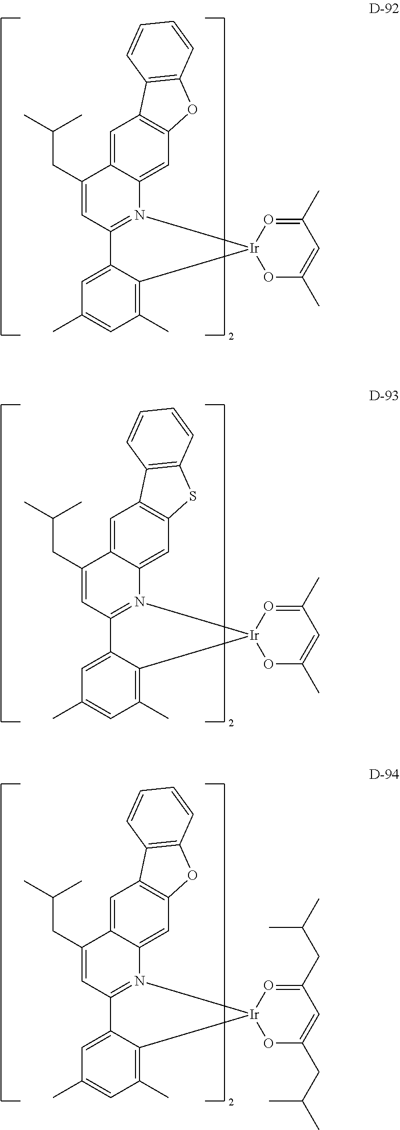

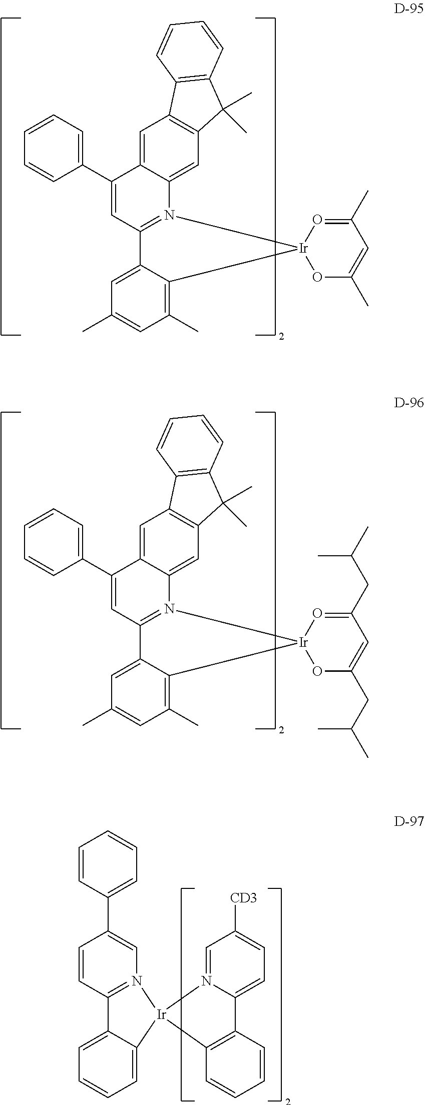

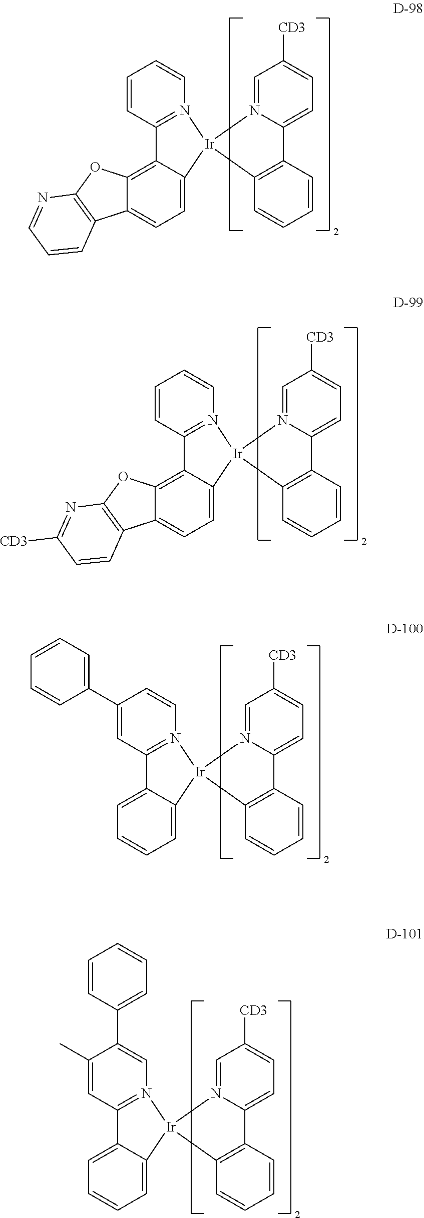

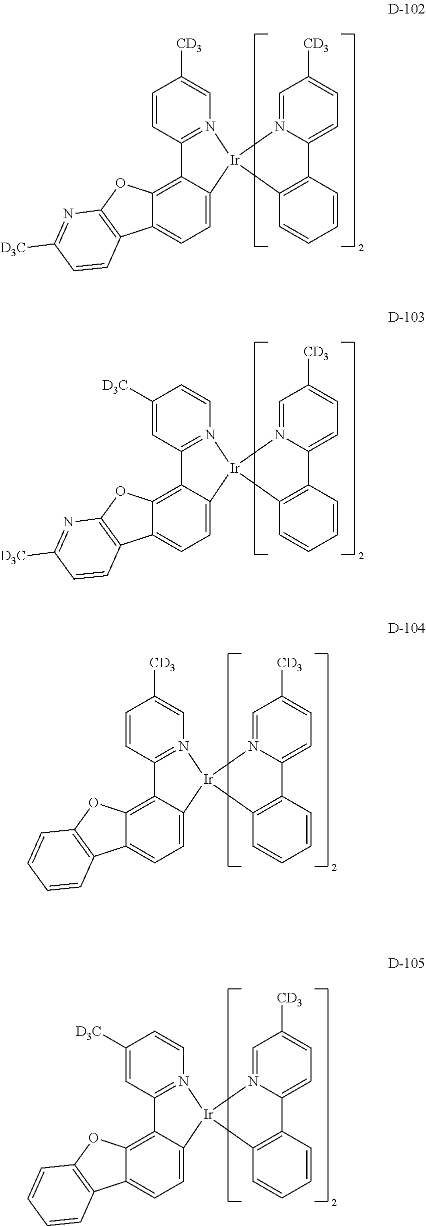

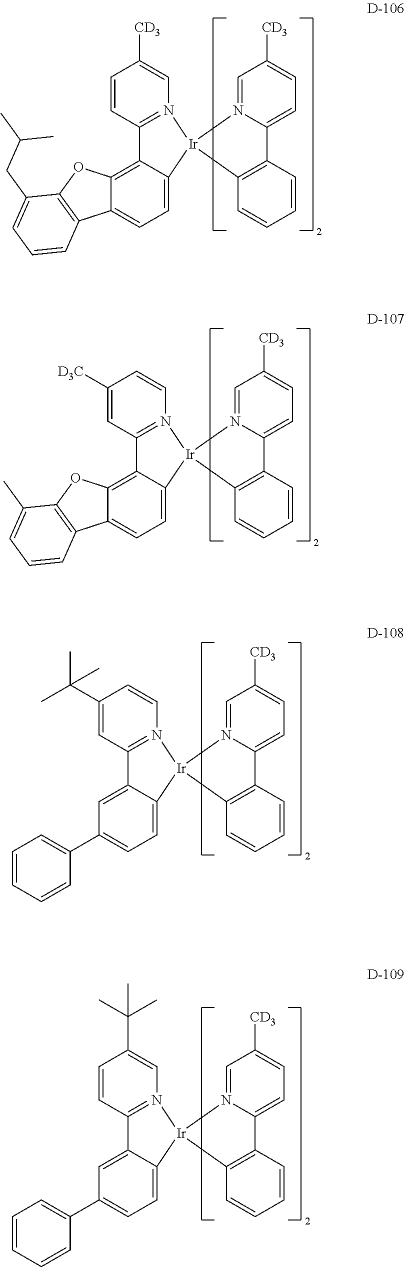

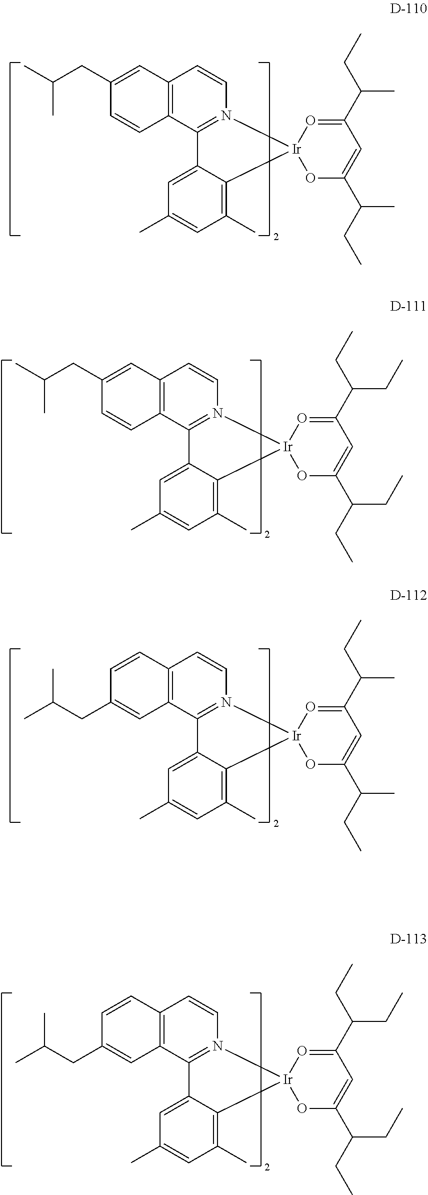

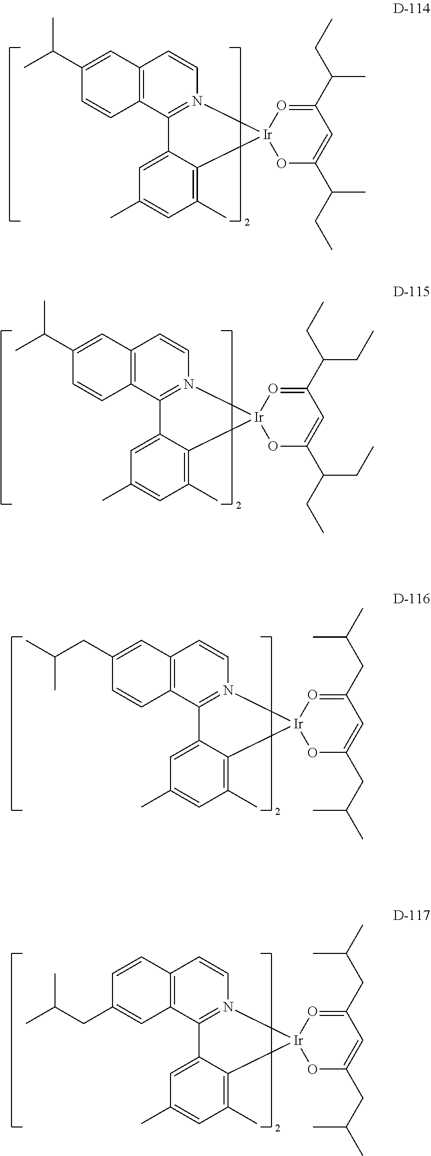

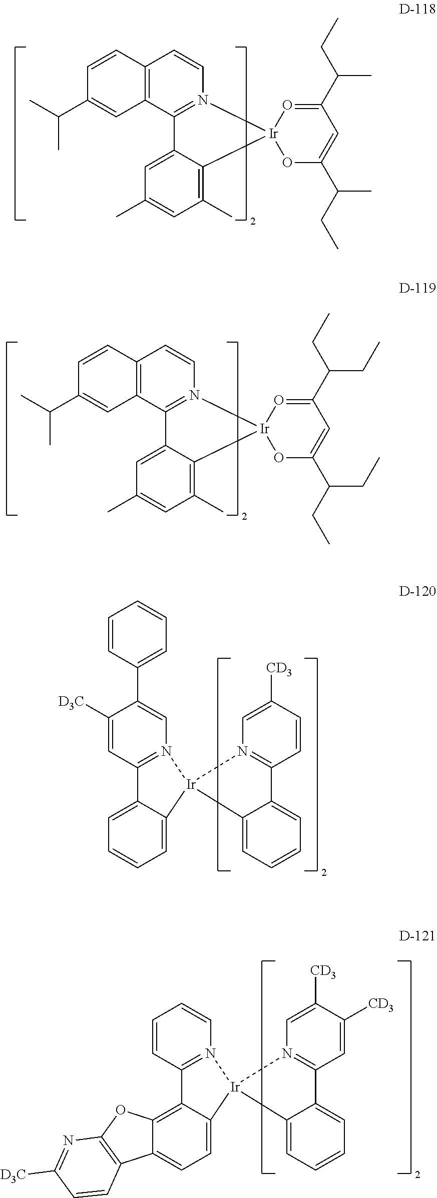

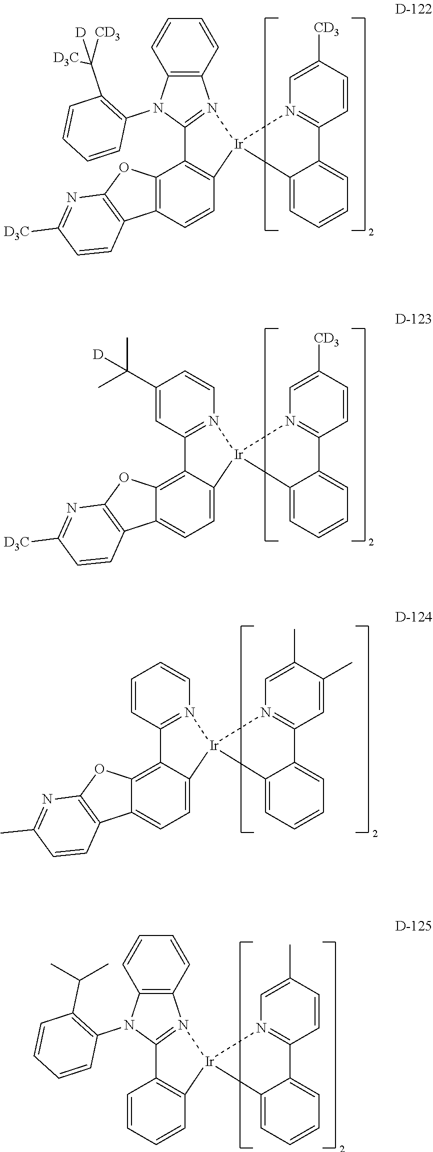

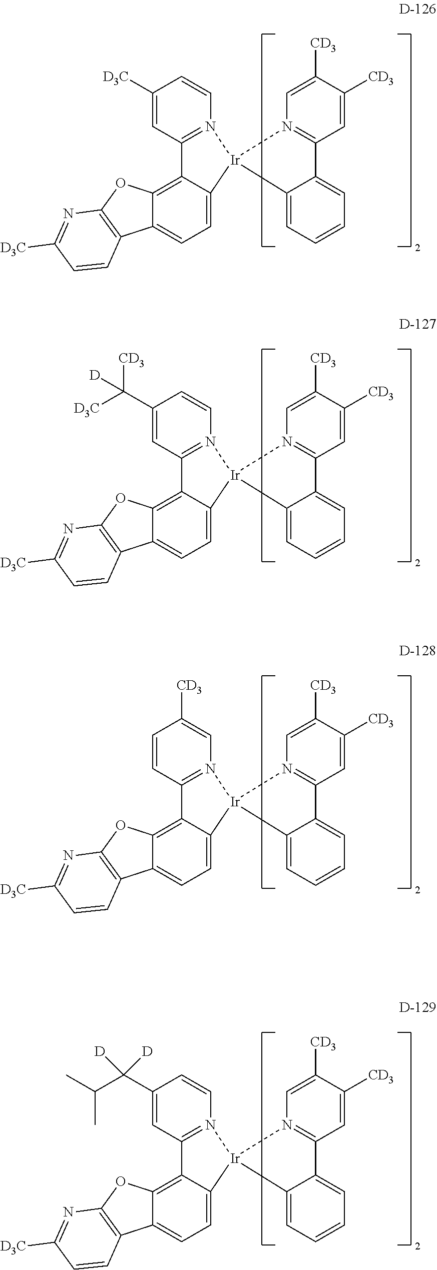

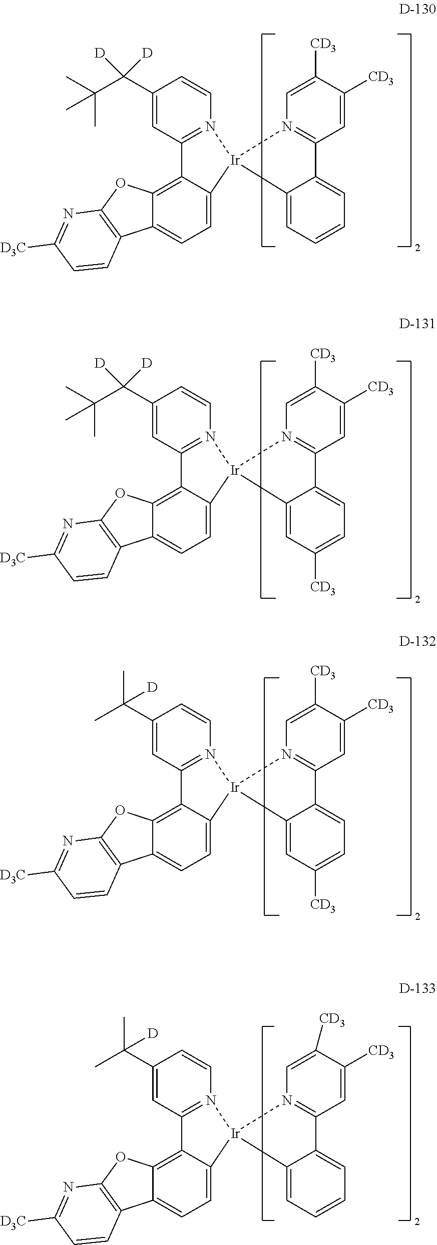

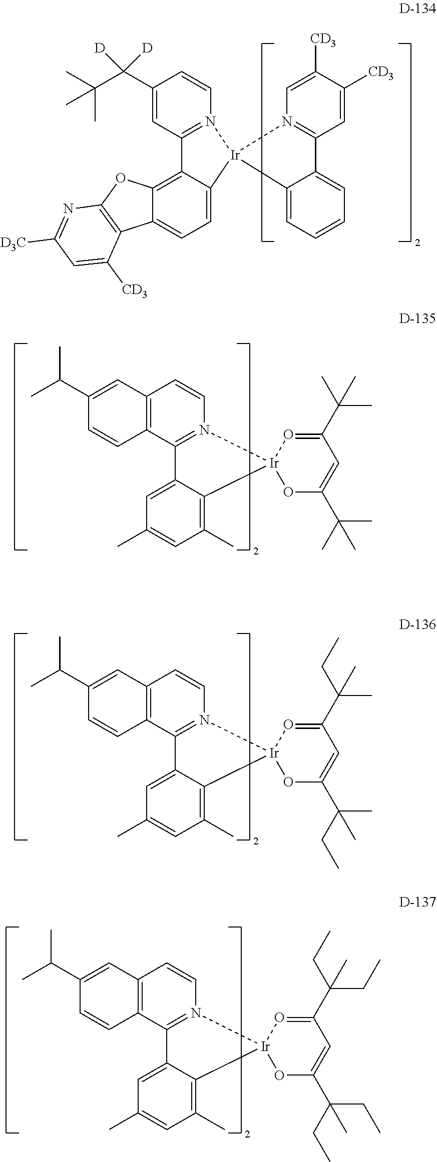

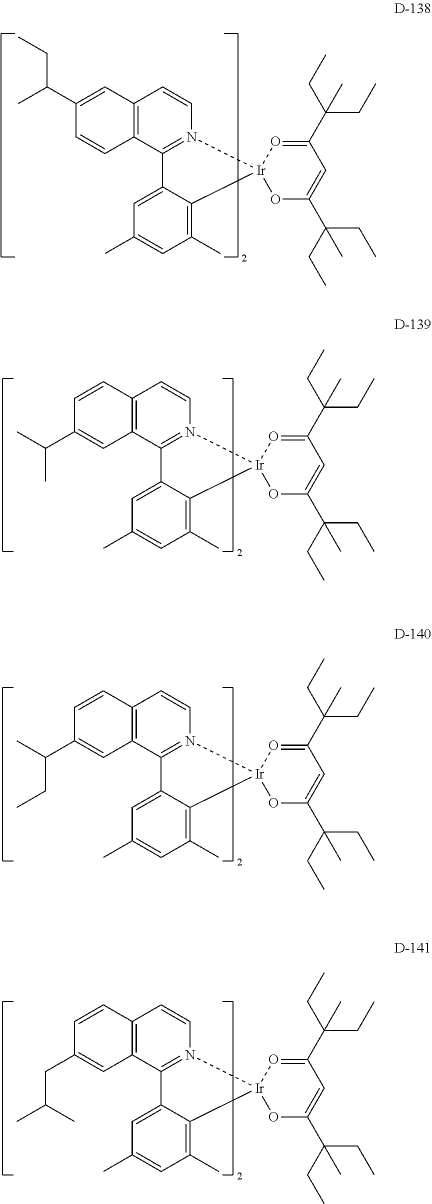

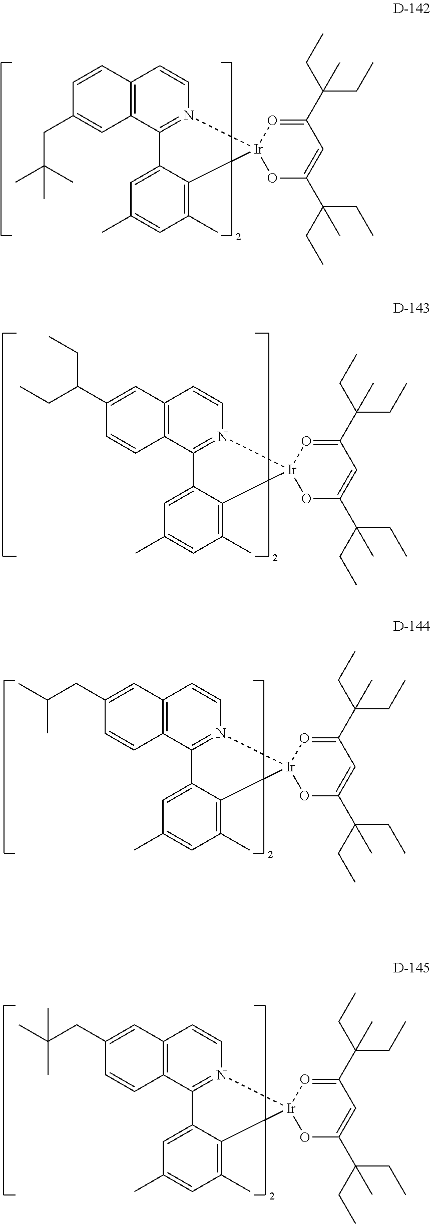

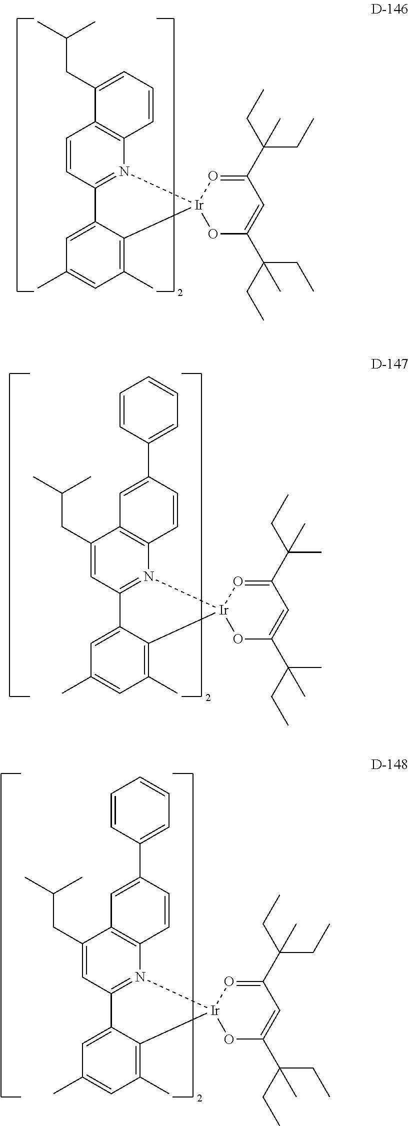

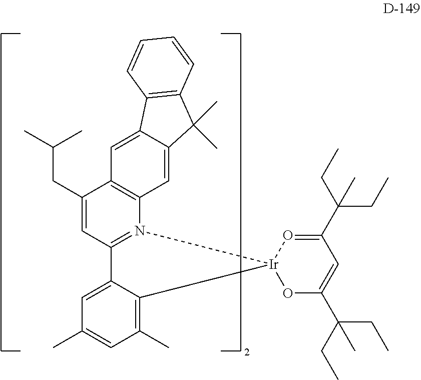

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host material and the second host material according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, when the first host material and the second host material exist in the same layer or different layers in the organic electroluminescent device, the layers by the two host compounds may be separately formed. For example, after depositing the first host material, a second host material may be deposited.

According to one embodiment, the present disclosure can provide display devices comprising a plurality of host materials including a first host material represented by the formula 1 and a second host material represented by the formula 2. In addition, it is possible to manufacture a display device or a lighting device using the organic electroluminescent device of the present disclosure. Specifically, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of compounds according to the present disclosure, and the properties thereof will be explained with reference to the synthesis method of a representative compound in order to understand the present disclosure in detail.

### [Example 1] Synthesis of Compound H1-147

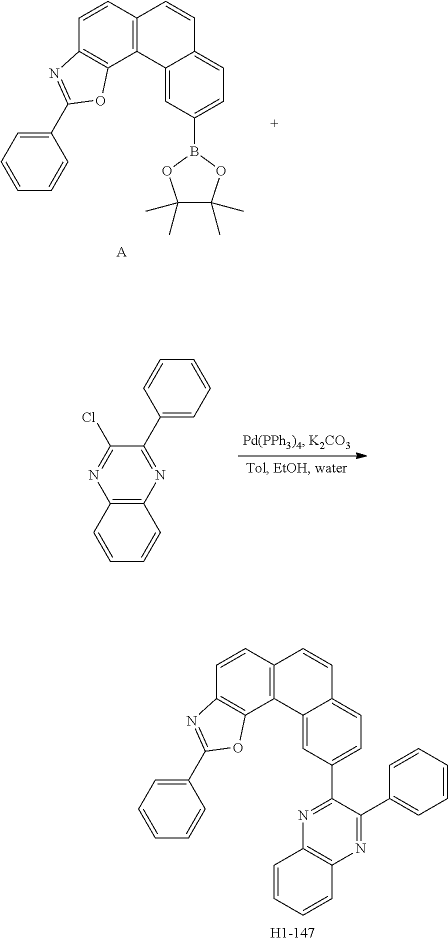

Compound A (CAS: 2085325-18-2, 4.0 g, 9.5 mmol), 2-chloro-3-phenylquinoxaline (2.8 g, 11.4 mmol), tetrakis(triphenylphosphine)palladium(0) (Pd(PPh₃)₄) (0.5 g, 0.5 mmol), potassium carbonate (K₂CO₃) (2.0 g, 19 mmol), toluene (30 mL), EtOH (7 mL), and water (10 mL) were added into a reaction vessel and refluxed for one day. After completion of the reaction, followed by cooling at room temperature, the reaction mixture was filtered with celite filter with methylene chloride (MC) and then distilled under reduced pressure. Thereafter, separated by column chromatography with methylene chloride/hexane (MC/Hex), compound H1-147 was obtained (2.7 g, yield: 57%).

| Compound | MW | M.P |
|---|---|---|
| H1-147 | 499.6 | 266° C. |

### [Example 2] Synthesis of Compound H1-146

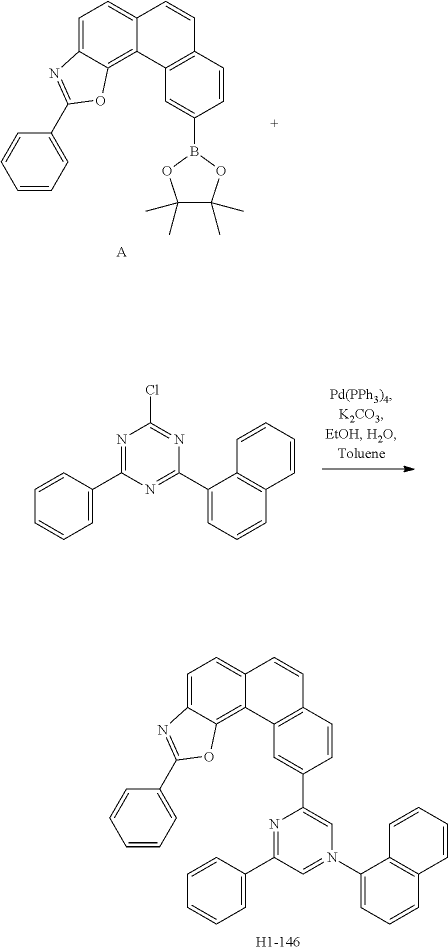

Compound A (23.8 g, 56.6 mmol), 2-chloro-4-(naphthalen-1-yl)-6-phenyl-1,3,5-triazine (15.0 g, 47.2 mmol), Pd(PPh₃)₄ (2.72 g, 2.36 mmol), K₂CO₃ (16.3 g, 118 mmol), toluene (240 mL), EtOH (60 mL), and purified water (60 mL) were added into a reaction vessel and refluxed for 2 hours. After completion of the reaction, the reaction mixture was cooled to room temperature and the organic layer was separated by a silica filter. The organic layer was distilled under reduced pressure, and recrystallized from toluene to obtain compound H1-146 (13.8 g, yield: 51%).

| Compound | MW | M.P |
|---|---|---|
| H1-146 | 576.6 | 231° C. |

### [Example 3] Synthesis of Compound H1-157

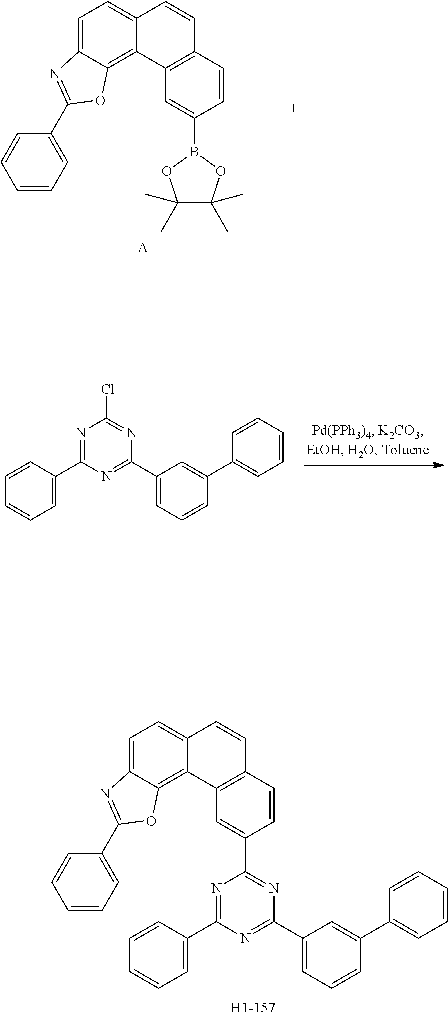

Compound A (4.0 g, 9.5 mmol), 2-([1,1-biphenyl]-3-yl)-4-chloro-6-phenyl-1,3,5-triazine (3.9 g, 11.4 mmol), Pd(PPh₃)₄ (0.5 g, 0.5 mmol), K₂CO₃ (2.6 g, 19 mmol), toluene (30 mL), EtOH (7 mL), and purified water (10 mL) were added into a reaction vessel and refluxed for 6 hours. After completion of the reaction, followed by cooling to room temperature, the reaction mixture was stirred at room temperature, and then methanol (MeOH) was added thereto. The resulting solid was filtered under reduced pressure, and separated by column chromatography with MC to obtain compound H1-157 (4.6 g, yield: 80%).

| Compound | MW | M.P |
|---|---|---|
| H1-157 | 602.7 | 227° C. |

### [Example 4] Synthesis of Compound H1-145

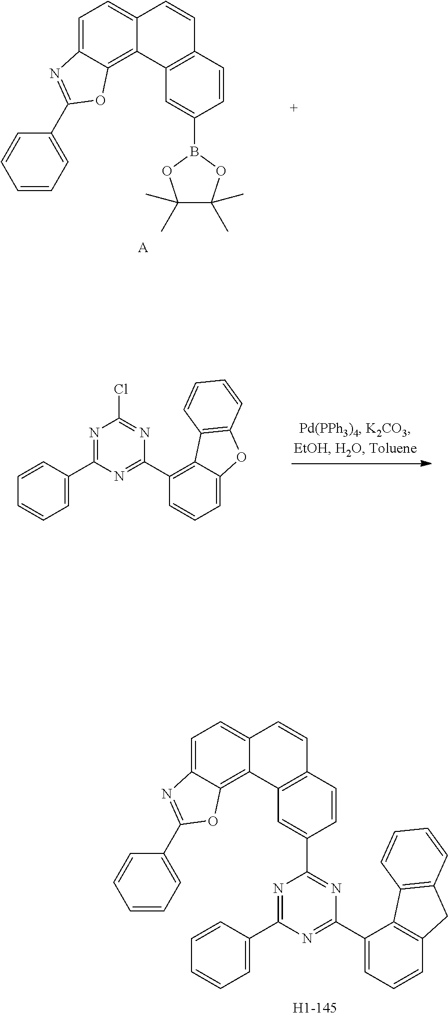

Compound A (3.0 g, 7.1 mmol), 2-chloro-4-(dibenzo[b,d]furan-1-yl)-6-phenyl-1,3,5-triazine (3.4 g, 9.26 mmol), Pd(PPh₃)₄ (0.4 g, 0.36 mmol), K₂CO₃ (2.0 g, 14 mmol), toluene (36 mL), EtOH (8 mL), and purified water (12 mL) were added into a reaction vessel and refluxed for 6 hours. After completion of the reaction, followed by cooling to room temperature, the reaction mixture was stirred at room temperature, and then methanol (MeOH) was added thereto. The resulting solid was filtered under reduced pressure, and separated by column chromatography with MC to obtain compound H1-145 (3.3 g, yield: 75%).

| Compound | MW | M.P |
|---|---|---|
| H1-145 | 616.7 | 282° C. |

### [Example 5] Synthesis of Compound H1-156

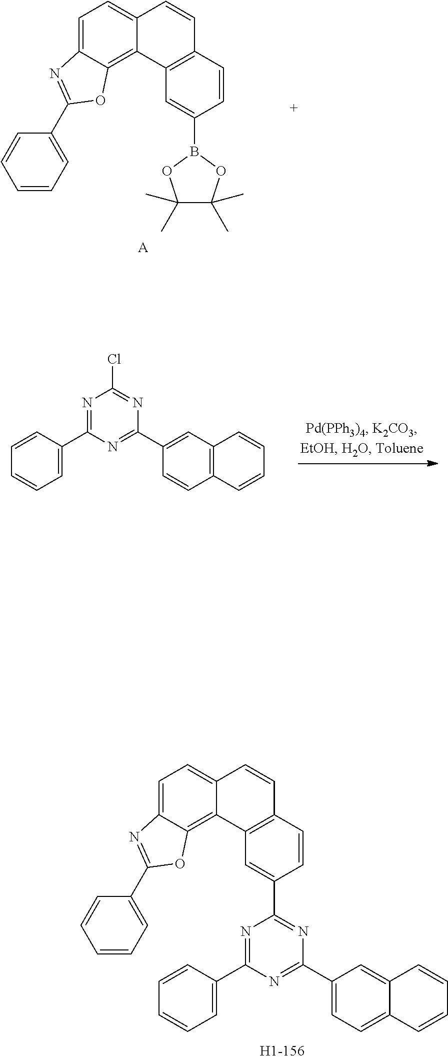

Compound A (4.0 g, 9.5 mmol), 2-chloro-4-(naphthalen-2-yl)-6-phenyl-1,3,5-triazine (3.6 g, 11.4 mmol), Pd(PPh₃)₄ (0.5 g, 0.5 mmol), K₂CO₃ (2.6 g, 19 mmol), toluene (30 mL), EtOH (7 mL), and purified water (10 mL) were added into a reaction vessel and refluxed for 4 hours. After completion of the reaction, followed by cooling to a room temperature, the reaction mixture was stirred at room temperature, and then methanol (MeOH) was added thereto. The resulting solid was filtered under reduced pressure, and separated by column chromatography with MC to obtain compound H1-156 (3.45 g, yield: 63%).

| Compound | MW | M.P |
|---|---|---|
| H1-156 | 576.6 | 268° C. |

### [Example 6] Synthesis of Compound H1-51

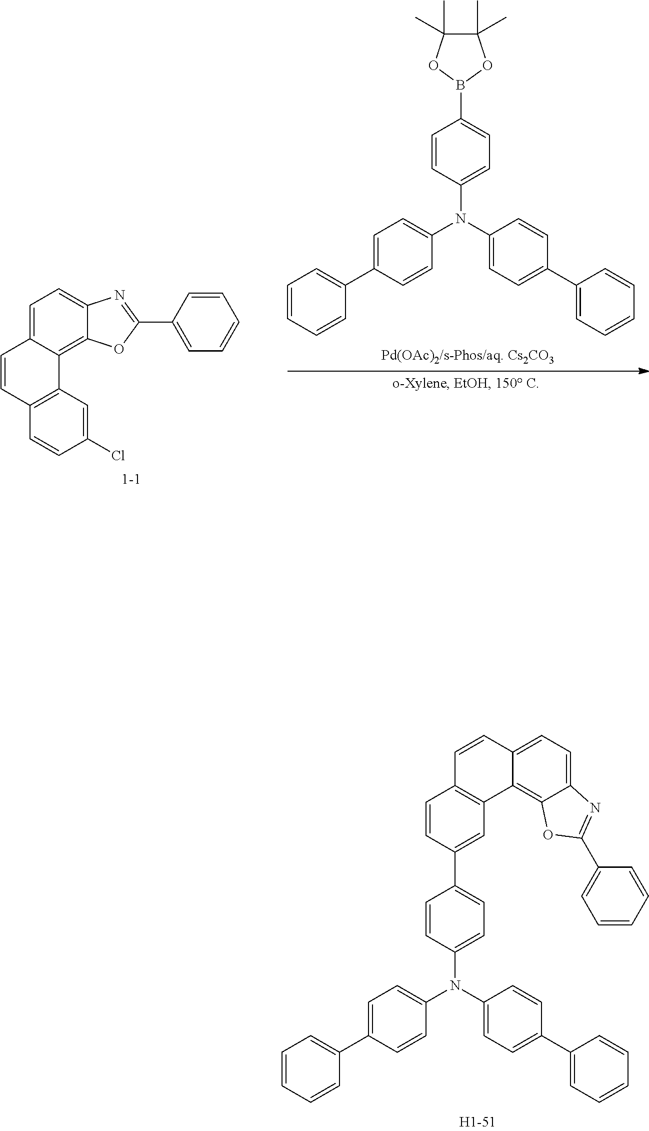 [SMILES: Clc1ccc2ccc3ccc4nc(-c5ccccc5)oc4c3c2c1 ; CC1(C)OB(c2ccc(N(c3ccc(-c4ccccc4)cc3)c3ccc(-c4ccccc4)cc3)cc2)OC1(C)C ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

Compound 1-1 (4 g, 12 mmol), bis(biphenyl-4-yl)[4-(4,4,5,5-tetramethyl-[1,3,2]-dioxaborolan-2-yl)phenyl]amine (6.8 g, 13 mmol), palladium(11) acetate(Pd(OAc)₂) (0.3 g, 1 mmol), 2-dicyclohexylphosphino-2′,6′-dimethoxybiphenyl(s-Phos) (0.9 g, 2 mmol), cesium carbonate(Cs₂CO₃) (11.5 g, 35 mmol), o-xylene (60 mL), ethanol(EtOH) (15 mL), and distilled water (15 mL) were added into a reaction vessel and refluxed for 3 hours at 150° C. After completion of the reaction, the reaction mixture was washed with distilled water and then the organic layer was extracted with ethyl acetate. After drying the extracted organic layer with magnesium sulfate, the solvent was removed with a rotary evaporator and then purified with column chromatography to obtain compound H1-51 (2.2 g, yield: 27%).

| Compound | MW | UV | PL | M.P |
|---|---|---|---|---|
| H1-51 | 690.85 | 406 nm | 427 m | 271° C. |

### [Example 7] Synthesis of Compound H1-80

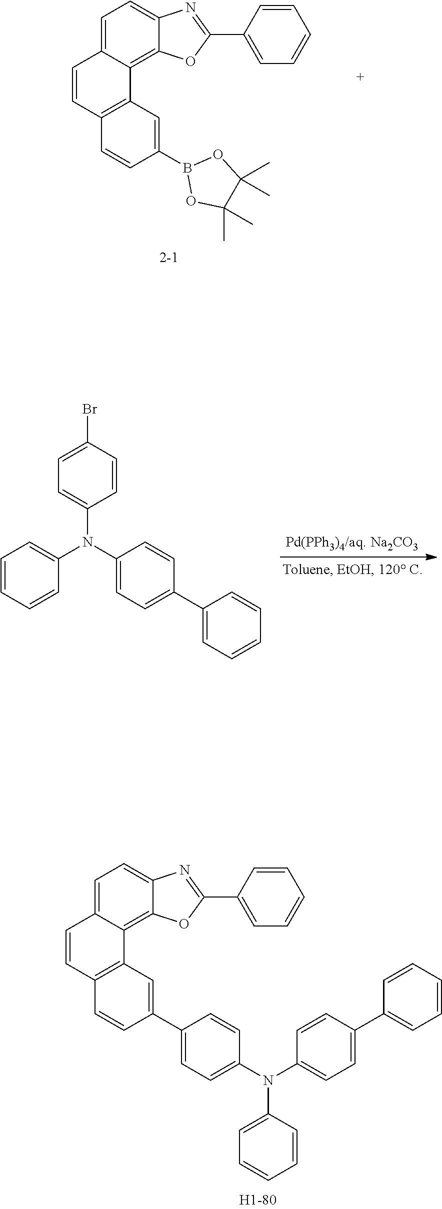 [SMILES: CC1(C)OB(c2ccc3ccc4ccc5nc(-c6ccccc6)oc5c4c3c2)OC1(C)C ; Brc1ccc(N(c2ccccc2)c2ccc(-c3ccccc3)cc2)cc1 ; c1ccc(-c2ccc(N(c3ccccc3)c3ccc(-c4ccc5ccc6ccc7nc(-c8ccccc8)oc7c6c5c4)cc3)cc2)cc1]

Compound 2-1 (4.8 g, 11.34 mmol), N-(4-bromophenyl)-N-phenyl-[1,1′-biphenyl]-4-amine (5 g, 12.47 mmol), Pd(PPh₃)₄ (0.4 g, 0.34 mmol), sodium carbonate (Na₂CO₃) (3.0 g, 28.35 mmol), toluene (57 mL), EtOH (14 mL), and distilled water (14 mL) were added into a reaction vessel and refluxed for 4 hours at 120° C. After completion of the reaction, the mixture was added dropwise to methanol, and then the resulting solid was filtered. The resulting solid was purified by recrystallization by column chromatography to obtain compound H1-80 (1.4 g, yield: 20.0%),

| Compound | MW | MP |
|---|---|---|
| H1-80 | 614.73 | 230° C. |

### [Example 8] Synthesis of Compound H1-158

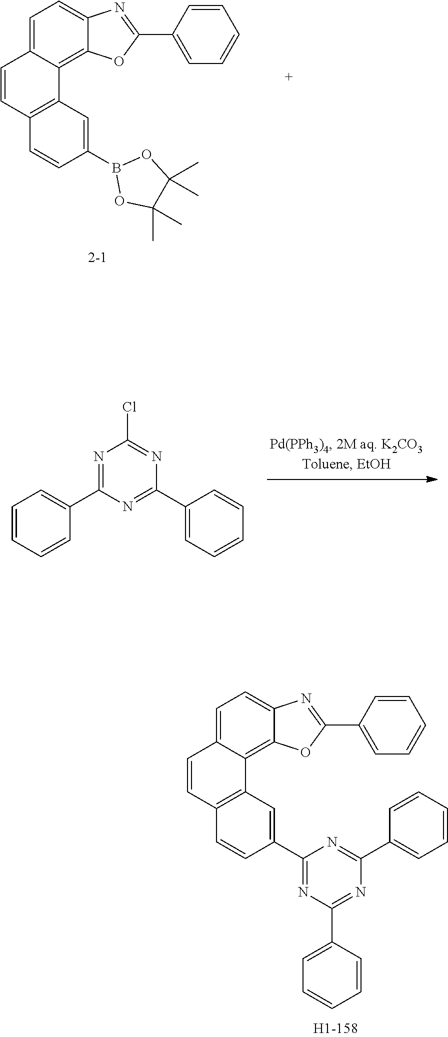 [SMILES: CC1(C)OB(c2ccc3ccc4ccc5nc(-c6ccccc6)oc5c4c3c2)OC1(C)C ; Clc1nc(-c2ccccc2)nc(-c2ccccc2)n1 ; c1ccc(-c2nc(-c3ccccc3)nc(-c3ccc4ccc5ccc6nc(-c7ccccc7)oc6c5c4c3)n2)cc1]

Compound 2-1 (10 g, 23.7 mmol), 2-chloro-4,6-diphenyltriazine (CAS: 3842-55-5, 5.8 g, 21.6 mmol), Pd(PPh₃)₄ (1.2 g, 1.0 mmol), potassium carbonate (K₂CO₃) (7.5 g, 59 mmol), toluene (90 mL), ethanol (30 mL), and distilled water (30 mL) were added into a reaction vessel and refluxed for 4 hours at 120° C., After completion of the reaction, the mixture was added dropwise to methanol, and then the resulting solid was filtered. The resulting solid was purified by recrystallization by column chromatography to obtain compound H1-158 (5.7 g, yield: 50%).

| Compound | MW | UV | PL | MP |
|---|---|---|---|---|
| H1-158 | 526.18 | 290 nm | 427 nm | 291° C. |

### [Example 9] Synthesis of Compound H1-102

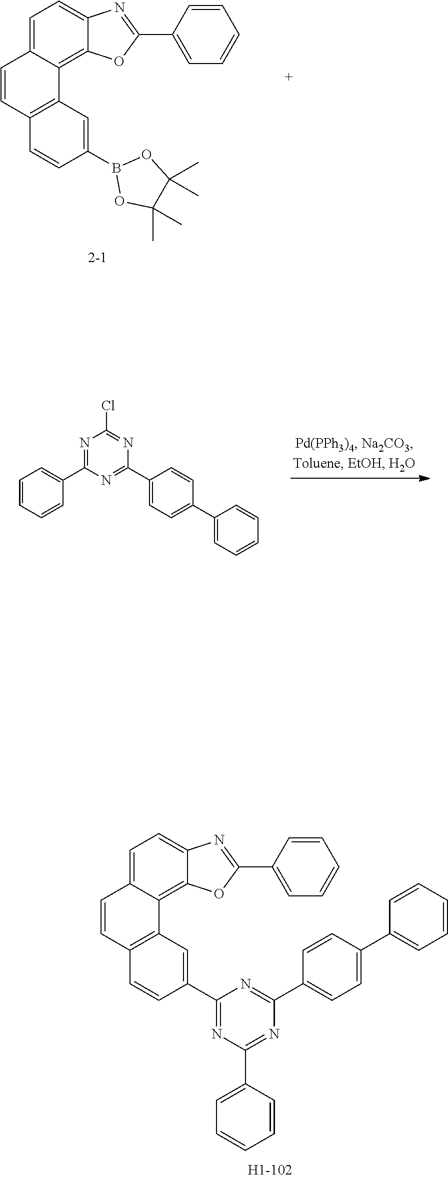 [SMILES: CC1(C)OB(c2ccc3ccc4ccc5nc(-c6ccccc6)oc5c4c3c2)OC1(C)C ; Clc1nc(-c2ccccc2)nc(-c2ccc(-c3ccccc3)cc2)n1 ; c1ccc(-c2ccc(-c3nc(-c4ccccc4)nc(-c4ccc5ccc6ccc7nc(-c8ccccc8)oc7c6c5c4)n3)cc2)cc1]

Compound 2-1 (3.48 g, 8.3 mmol), 2-([1,1′-biphenyl]-4-yl)-4-chloro-6-phenyl-1,3,5-triazine (CAS: 1472062-94-4, 3.53 g, 9.1 mmol), Pd(PPh₃)₄ (0.48 g, 0.41 mmol), sodium carbonate (2.2 g, 20.7 mmol), toluene (28 mL), ethanol (7 mL), and distilled water (7 mL) were added into a reaction vessel and refluxed for 5 hours at 120° C. After completion of the reaction, the mixture was added dropwise to methanol, and then the resulting solid was filtered. The resulting solid was purified by recrystallization by column chromatography to obtain compound H1-102 (3.7 g, yield: 74%).

| Compound | MW | UV | PL | M.P |
|---|---|---|---|---|
| H1-102 | 602.21 | 324 nm | 429 nm | 299° C. |

### [Example 10] Synthesis of Compound C2-153

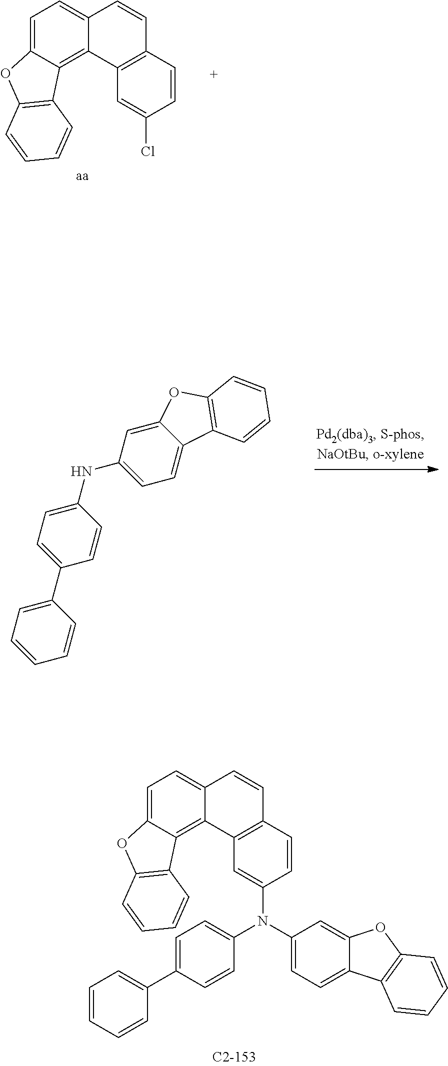 [SMILES: Clc1ccc2ccc3ccc4oc5ccccc5c4c3c2c1 ; c1ccc(-c2ccc(Nc3ccc4c(c3)oc3ccccc34)cc2)cc1 ; c1ccc(-c2ccc(N(c3ccc4c(c3)oc3ccccc34)c3ccc4ccc5ccc6oc7ccccc7c6c5c4c3)cc2)cc1]

Compound aa (8.0 g, 26.4 mmol), N-([1,1′-biphenyl]-4-yl)dibenzo[b,d]furan-3-amine) (8.85 g, 26.4 mmol), Pd₂(dba)₃ (1.21 g, 1.32 mmol), 3-phos (1.08 g, 2.64 mmol), NaOtBu (3.81 g, 39.6 mmol), and o-xylene (140 mL) were added into a reaction vessel and refluxed for 5 hours. After completion of the reaction, followed by cooling at a room temperature, the reaction mixture was separated with silica filter and then the organic layer was distilled under reduced pressure followed by recrystallization with toluene to obtain compound C2-153 (5.0 g, yield: 31%).

| Compound | MW | M.P |
|---|---|---|
| C2-153 | 601.7 | 250° C. |

### [Example 11] Synthesis of Compound C2-154

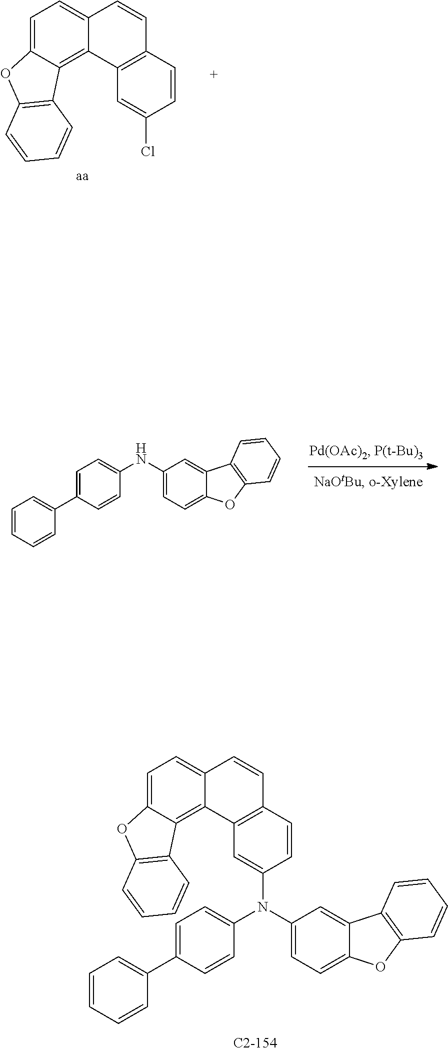 [SMILES: Clc1ccc2ccc3ccc4oc5ccccc5c4c3c2c1 ; c1ccc(-c2ccc(Nc3ccc4oc5ccccc5c4c3)cc2)cc1 ; c1ccc(-c2ccc(N(c3ccc4oc5ccccc5c4c3)c3ccc4ccc5ccc6oc7ccccc7c6c5c4c3)cc2)cc1]

Compound aa (7 g, 23.15 mmol), N-([1,1-biphenyl]-4-yl)dibenzo[b,d]furan-2-amine (7.8 g, 23.15 mmol), Pd(OAc)₂ (0.26 g, 1.158 mmol), P(t-Bu)₃ (0.47 g, 2.3 mmol), sodium tert-butoxide (NaOtBu) (4.4 g, 46.3 mmol), and o-xylene (115 mL) were added into a reaction vessel and dissolved followed by refluxing for 2 hours. After completion of the reaction, the organic layer was extracted with ethyl acetate and then separated with column chromatography to obtain compound C2-154 (3 g, yield: 21%),

| Compound | MW | M.P |
|---|---|---|
| C2-154 | 601.69 | 215° C. |

### [Example 12] Synthesis of Compound C2-146

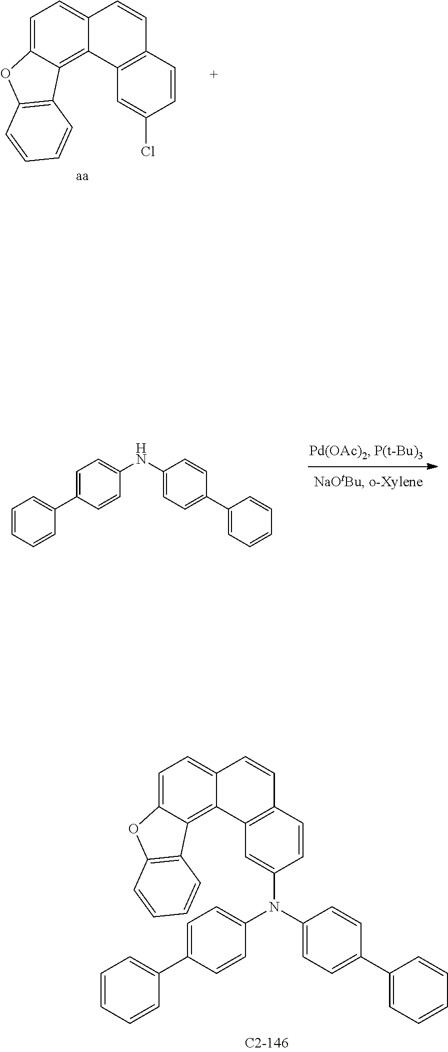 [SMILES: Clc1ccc2ccc3ccc4oc5ccccc5c4c3c2c1 ; c1ccc(-c2ccc(Nc3ccc(-c4ccccc4)cc3)cc2)cc1 ; c1ccc(-c2ccc(N(c3ccc(-c4ccccc4)cc3)c3ccc4ccc5ccc6oc7ccccc7c6c5c4c3)cc2)cc1]

Compound aa (5.0 g, 16.5 mmol), di([1,1-biphenyl]-4-yl)amine (5.3 g, 16.5 mmol), Pd(OAc)₂ (0.19 g, 0.83 mmol), P(t-Bu)₃ (0.82 mL, 1.65 mmol), NaOtBu (3.2 g, 33.0 mmol), and o-xylene (83 mL) were added into a flask and dissolved followed by refluxing for 3 hours. After completion of the reaction, the organic layer was extracted with ethyl acetate and then separated with column chromatography to obtain compound C2-146 (3 g, yield: 30%).

| Compound | MW | M.P |
|---|---|---|
| C2-146 | 587.71 | 237° C. |

### [Example 13] Synthesis of Compound C2-9

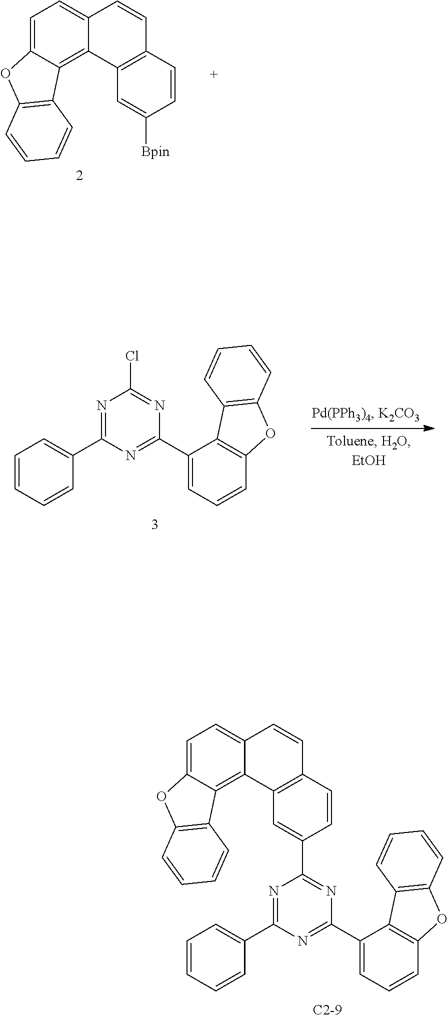

Compound 2 (5.0 g, 12.7 mmol), compound 3 (5.5 g, 15.3 mmol), K₂CO₃ (3.5 g, 25.4 mmol), and Pd(PPh₃)₄ (0.73 g, 0.63 mmol) were added into a flask and dissolved with toluene (39 mL), ethanol (10 mL), and water (13 mL) followed by refluxing for 6 hours at 130° C. After completion of the reaction, the organic layer was extracted with ethyl acetate and the residual water was removed with magnesium sulfate and then dried. Thereafter, by using column chromatography, compound C2-9 was obtained (4.4 g, yield: 20%).

| Compound | MW | M.P |
|---|---|---|
| C2-9 | 589.65 | 318° C. |

### [Example 14] Synthesis of Compound C2-2

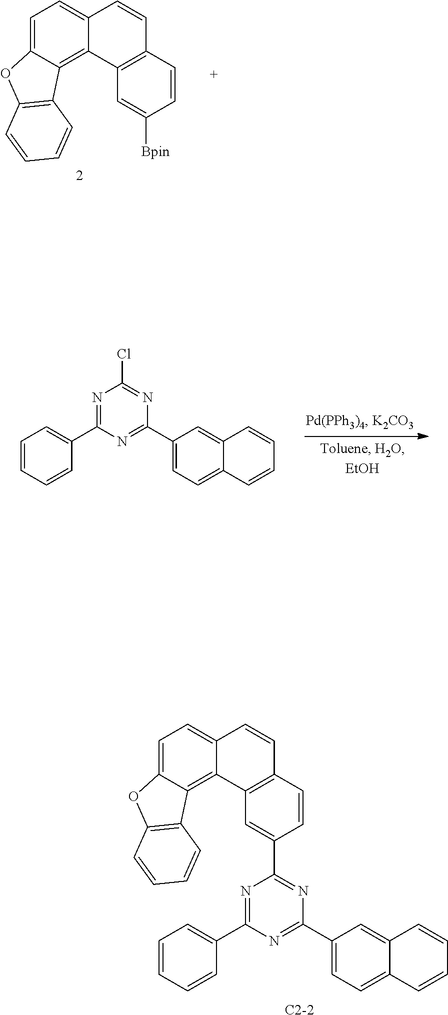

Compound 2 (5.0 g, 12.7 mmol), 2-chloro-4-(naphthalen-2-yl)-6-phenyl-1,3,5-triazine (4.8 g, 15.2 mmol), K₂CO₃ (3.5 g, 25.4 mmol), and Pd(PPh₃)₄ (0.73 g, 0.63 mmol) were added into a flask and dissolved with toluene (39 mL), ethanol (10 mL), and water (13 mL) followed by refluxing for 6 hours at 130° C. After completion of the reaction, the organic layer was extracted with ethyl acetate and the residual water was removed with magnesium sulfate and then dried. Thereafter, by using column chromatography, compound C2-2 was obtained (4.4 g, yield: 20%).

| Compound | MW | M.P |
|---|---|---|
| C2-2 | 549.62 | 229° C. |

### [Example 15] Synthesis of Compound C2-191

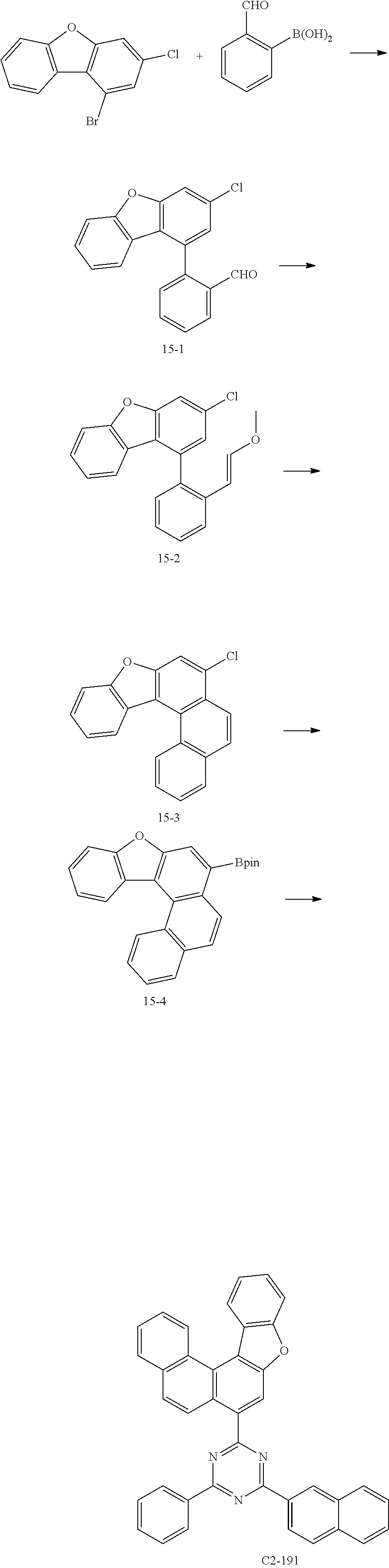

1) Synthesis of Compound 15-1

1-bromo-3-chlorodibenzo[b,d]furan (39.2 g, 139.3 mmol), 2-formylphenyl)boronic acid (52.2 g, 348.1 mmol), Pd(PPh₃)₄ (16.1 g, 13.9 mmol), Cs₂CO₃ (136.1 g, 418 mmol), toluene (840 mL), ethanol (160 mL), and distilled water (210 mL) were added into a reaction vessel and stirred for 5 hours at 140° C. After completion of the reaction, the reaction mixture was cooled to a room temperature and then the organic layer was extracted with ethyl acetate. After drying the extracted organic layer with magnesium sulfate, the solvent was removed with a rotary evaporator. Thereafter, it was purified with column chromatography to obtain compound 15-1 (32.1 g, yield: 75%).

2) Synthesis of Compound 15-2

Compound 15-1 (31.6 g, 103 mmol), (methoxymethyl)triphenylphosphonium chloride (45.9 g, 133.9 mmol), and tetrahydrofuran (THF) (515 mL) were added into a reaction vessel and stirred for 10 minutes followed by adding slowly dropwise potassium tert-butoxide (1M of THF, 150 mL) at 0° C. Thereafter, the temperature was slowly raised and then the reaction mixture was stirred at room temperature for 3 hours. After completion of the reaction by adding distilled water into the reaction solution, the organic layer was extracted with ethyl acetate. After drying the extracted organic layer with magnesium sulfate, the solvent was removed with a rotary evaporator. Thereafter, it was purified with column chromatography to obtain compound 15-2 (31.2 g, yield: 90%).

3) Synthesis of Compound 15-3

Compound 15-2 (29.8 g, 89.0 mmol), boron trifluoride etherate (22.4 mL) and methylene chloride (MC) (890 mL) were added into a reaction vessel and stirred for 3 hours. After completion of the reaction, the organic layer was extracted with MC together with water. After drying the extracted organic layer with magnesium sulfate, the solvent was removed with a rotary evaporator. Thereafter, it was purified with column chromatography to obtain compound 15-3 (24.2 g, yield: 90%).

4) Synthesis of Compound 15-4

Compound 15-3 (18.0 g, 59.5 mmol), bis(pinacollato)diborane (19.7 g, 77.3 mmol), tris(dibenzylideneacetone)dipalladium (Pd₂(dba)₃) (2.8 g, 2.9 mmol), 2-dicyclohexylphosphino-2′,6′-dimethoxybiphenyl(s-phos) (2.4 g, 5.9 mmol), potassium acetate (KOAC) (17.5 g, 178.5 mmol) and 1,4-dioxane (300 mL) were added into a reaction vessel and stirred for 6 hours at 150° C. After completion of the reaction, the reaction mixture was cooled to a room temperature and then the organic layer was extracted with ethyl acetate. After drying the extracted organic layer with magnesium sulfate, the solvent was removed with a rotary evaporator. Thereafter, it was purified with column chromatography to obtain compound 15-4 (18.4 g, yield: 78%).

5) Synthesis of Compound C2-191

Compound 15-4 (4.0 g, 10.1 mmol), 2-chloro-4-(naphthalen-2-yl)-6-phenyl-1,3,5-triazine (3.9 g, 12.2 mmol), Pd(PPh₃)₄ (0.6 g, 0.51 mmol), potassium carbonate (2.8 g, 20.2 mmol), toluene (30 mL), ethanol (7 mL) and distilled water (10 mL) were added into a reaction vessel and stirred for 6 hours at 130° C. After completion of the reaction, the mixture was added dropwise to methanol, and then the resulting solid was filtered. The resulting solid was purified by column chromatography to obtain compound C2-191 (4.5 g, yield: 81%),

| Compound | MW | M.P |
|---|---|---|
| C2-191 | 547.6 | 228° C. |

### [Example 16] Synthesis of Compound C2-192

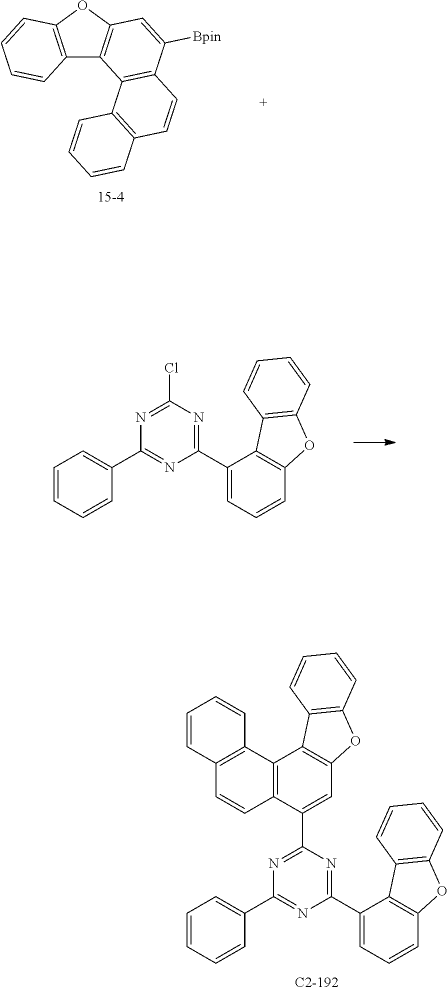

Compound 15-4 (4.0 g, 10.1 mmol), 2-chloro-4-(dibenzo[b,d]furan-1-yl)-6-phenyl-1,3,5-triazine (4.4 g, 12.2 mmol), Pd(PPh₃)₄ (0.6 g, 0.5 mmol), potassium carbonate (2.8 g, 20.2 mmol), toluene (30 mL), ethanol (7 mL), and distilled water (10 mL) were added into a reaction vessel and stirred for 6 hours at 130° C., After completion of the reaction, the mixture was added dropwise to methanol, and then the resulting solid was filtered. The resulting solid was purified by column chromatography to obtain compound C-192 (3.13 g, yield 53%).

| Compound | MW | M.P |
|---|---|---|
| C2-192 | 589.6 | 250° C. |

### [Example 17] Synthesis of Compound C2-242

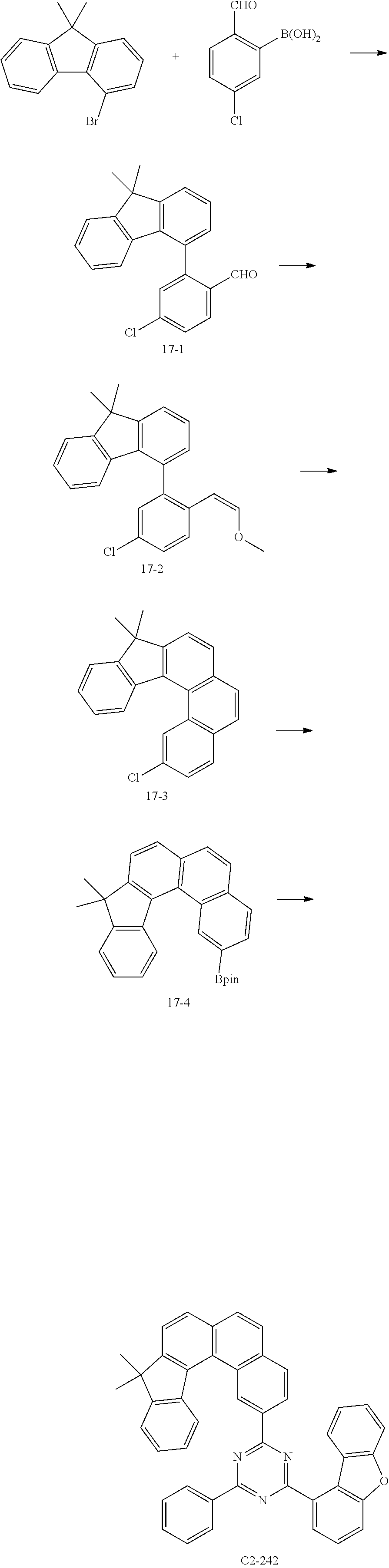

1) Synthesis of Compound 17-1

4-bromo-9,9-dimethyl-91-1-fluorene (50 g, 183 mmol), (5-chloro-2-formylphenyl)boronic acid (40.5 g, 219 mmol), Pd(PPh₃)₄ (10.6 g, 9.15 mmol), potassium carbonate (63 g, 457 mmol), toluene (690 mL), ethanol (180 mL), and distilled water (230 mL) were added into a reaction vessel and stirred for 5 hours at 140° C., After completion of the reaction, the reaction mixture was cooled to a room temperature and then the organic layer was extracted with ethyl acetate. After drying the extracted organic layer with magnesium sulfate, the solvent was removed with a rotary evaporator. Thereafter, it was purified with column chromatography to obtain compound 17-1 (40.3 g, yield: 66%).

2) Synthesis of Compound 17-2

Compound 17-1 (40.3 g, 121 mmol), (methoxymethyl)triphenylphosphonium chloride (53.9 g, 157.4 mmol) and THF (600 mL) were added into a reaction vessel and stirred for 10 minutes followed by adding slowly dropwise potassium tert-butoxide (1M of THF, 162 mL) at 0° C. The temperature was slowly raised and then the reaction mixture was stirred at room temperature for 3 hours. After completion of the reaction by adding distilled water to the reaction solution, the organic layer was extracted with ethyl acetate. After drying the extracted organic layer with magnesium sulfate, the solvent was removed with a rotary evaporator. Thereafter, it was purified with column chromatography to obtain compound 17-2 (39 g, yield: 89%).

3) Synthesis of Compound 17-3

Compound 17-2 (38 g, 105.3 mmol), boron trifluoride etherate (26.5 mL) and methylene chloride (MC) (1,000 mL) were added into a reaction vessel and stirred for 3 hours. After completion of the reaction, the organic layer was extracted with methylene chloride (MC) together with water. After drying the extracted organic layer with magnesium sulfate, the solvent was removed with a rotary evaporator. Thereafter, it was purified with column chromatography to obtain compound 17-3 (23.2 g, yield: 67%).

4) Synthesis of Compound 17-4

Compound 17-3 (19.1 g, 58.1 mmol), bis(pinacolato)diborane (19.1 g, 75.5 mmol), Pd₂(dba)₃ (2.7 g, 2.9 mmol), 2-dicyclohexylphosphino-2′,6′-dimethoxybiphenyl(s-phos) (2.4 g, 5.81 mmol), potassium acetate (17.1 g, 174.3 mmol), and 1,4-dioxane (290 mL) were added into a reaction vessel and stirred for 6 hours at 150c′C. After completion of the reaction, the reaction mixture was cooled to room temperature and then the organic layer was extracted with ethyl acetate. After drying the extracted organic layer with magnesium sulfate, the solvent was removed with a rotary evaporator. Thereafter, it was purified with column chromatography to obtain compound 17-4 (12.7 g, yield: 52%).

5) Synthesis of Compound C2-242

Compound 17-4 (4 g, 9.5 mmol), 2-chloro-4-(dibenzo[b,d]furan-1-yl)-6-phenyl-1,3,5-triazine (4.1 g, 11.4 mmol), Pd(PPh₃)₄ (0.55 g, 0.48 mmol), potassium carbonate (2.6 g, 19.0 mmol), toluene (30 mL), ethanol (7 mL), and distilled water (10 mL) were added into a reaction vessel and stirred for 6 hours at 130″C. After completion of the reaction, the mixture was added dropwise to methanol, and then the resulting solid was filtered. The resulting solid was purified by column chromatography to obtain compound C-242 (4.73 g, yield 80%).

| Compound | MW | M.P |
|---|---|---|
| C-242 | 615.7 | 237° C. |

### [Example 18] Synthesis of Compound C2-241

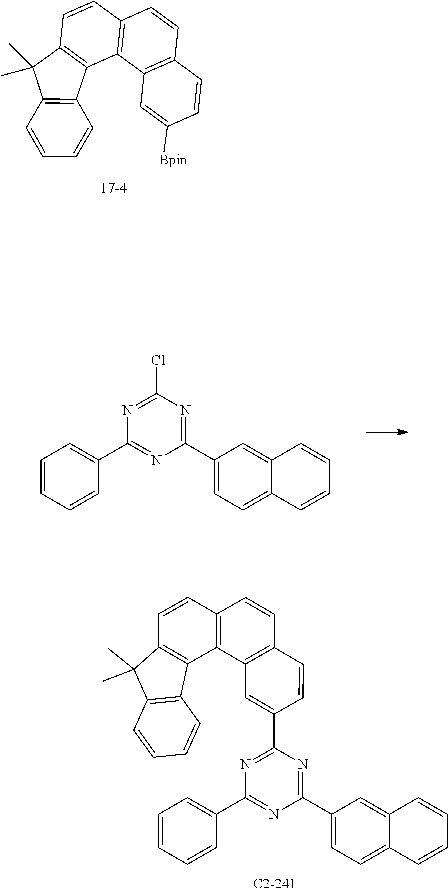

Compound 17-4 (5.0 g, 11.9 mmol), 2-chloro-4-(naphthalen-2-yl)-6-phenyl-1,3,5-triazine (4.5 g, 14.3 mmol), Pd(PPh₃))₄ (0.7 g, 0.6 mmol), potassium carbonate (3.3 g, 23.8 mmol), toluene (36 mL), ethanol (10 mL) and distilled water (12 mL) were added into a reaction vessel and stirred for 6 hours at 130° C. After completion of the reaction, the reaction mixture was added dropwise to methanol, and then the resulting solid was filtered. The resulting solid was purified by column chromatography to obtain compound C2-241 (3.64 g, yield: 53%).

| Compound | MW | M.P |
|---|---|---|
| C2-241 | 575.7 | 209° C. |

### [Example 19] Synthesis of Compound C2-57

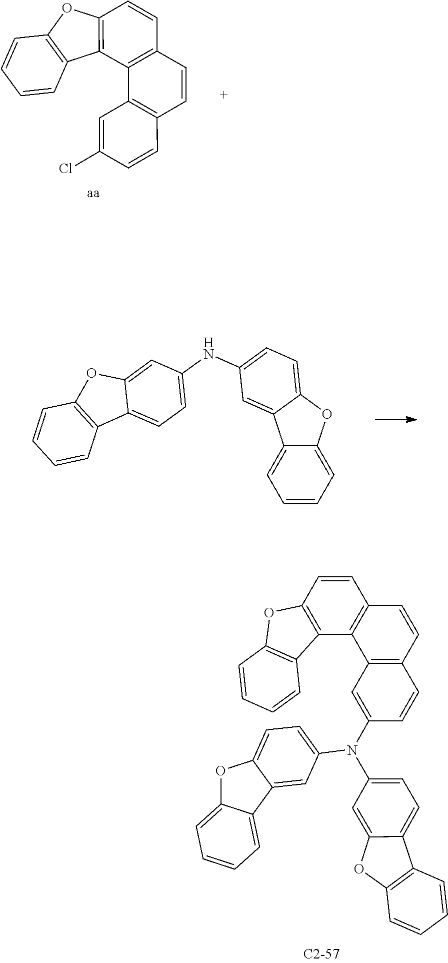 [SMILES: Clc1ccc2ccc3ccc4oc5ccccc5c4c3c2c1 ; c1ccc2c(c1)oc1cc(Nc3ccc4oc5ccccc5c4c3)ccc12 ; c1ccc2c(c1)oc1cc(N(c3ccc4oc5ccccc5c4c3)c3ccc4ccc5ccc6oc7ccccc7c6c5c4c3)ccc12]

Compound aa (5.0 g, 16.5 mmol), N-(dibenzo[b,d]furan-3-yl)dibenzo[b,d]furan-2-amine (5.7 g, 16.5 mmol). Pd(OAc)₂ (0.19 g, 0.82 mmol), P(t-Bu)₃ (0.82 mL, 1.65 mmol), NaOtBu (3.2 g, 33.0 mmol), and o-xylene (83 mL) were added into a flask and refluxed for 2 hours. After completion of the reaction, the organic layer was extracted with EA/H₂O and separated by column chromatography to obtain compound C2-57 (4.46 g, yield: 43%).

| Compound | MW | M.P |
|---|---|---|
| C2-57 | 615.69 | 239° C. |

### [Example 20] Synthesis of Compound C2-52

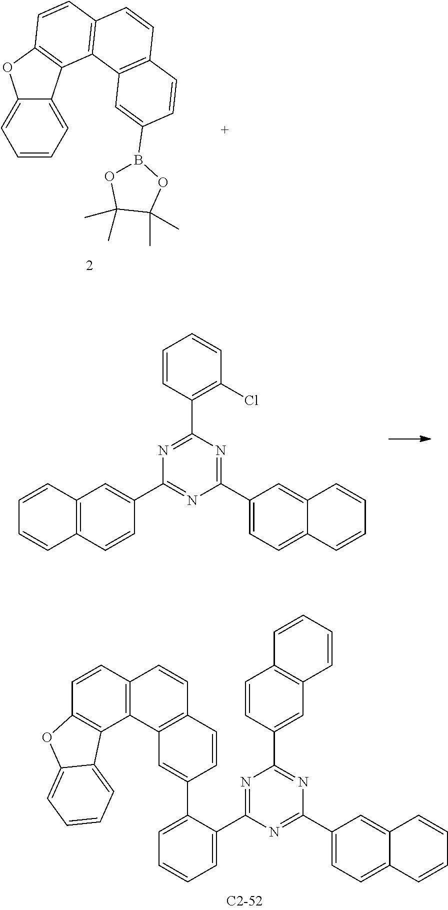 [SMILES: CC1(C)OB(c2ccc3ccc4ccc5oc6ccccc6c5c4c3c2)OC1(C)C ; Clc1ccccc1-c1nc(-c2ccc3ccccc3c2)nc(-c2ccc3ccccc3c2)n1 ; c1ccc(-c2nc(-c3ccc4ccccc4c3)nc(-c3ccc4ccccc4c3)n2)c(-c2ccc3ccc4ccc5oc6ccccc6c5c4c3c2)c1]

Compound 2 (4 g, 10.14 mmol), 2-(2-chlorophenyl)-4,6-di(naphthalen-2-yl)-1,3,5-triazine (4.3 g, 10.14 mmol), Pd(PPh₃)₄ (586 mg, 0.507 mmol), K₂CO₃ (2.8 g, 20.29 mmol), toluene (50 mL), EtOH (12 mL), and H₂O (13 mL) were added into a flask and refluxed for 6 hours at 140° C. After completion of the reaction, followed by cooling at room temperature, the resulting solid was filtered under reduced pressure. The resulting solid was dissolved in CHCl₃ and filtered by SiO₂ filter with CHCl₃ and then recrystallized with o-xylene and o-DCB to obtain compound C2-52 (5.8 g, yield: 65%).

| Compound | MW | M.P |
|---|---|---|
| C2-52 | 675.7 | 270.8° C. |

### [Example 21] Compound C2-245

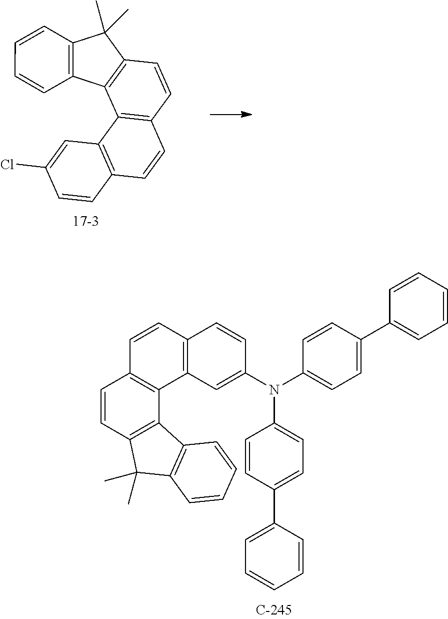 [SMILES: CC1(C)c2ccccc2-c2c1ccc1ccc3ccc(Cl)cc3c21 ; CC1(C)c2ccccc2-c2c1ccc1ccc3ccc(N(c4ccc(-c5ccccc5)cc4)c4ccc(-c5ccccc5)cc4)cc3c21]

Compound 17-3 (3.3 g, 10.04 mmol), di([1,1″-biphenyl]-4-yl)amine (3.2 g, 10.04 mmol), Pd₂(dba)₃ (0.5 g, 0.50 mmol), P(t-Bu)₃ (0.5 mL, 1.04 mmol), NaOtBu (1.5 g, 15.06 mmol), and toluene (50 mL) were added into a reaction vessel and stirred for 4 hours at 150° C. After completion of the reaction, followed by cooling the reaction mixture to room temperature, the resulting solid was filtered and washed with ethyl acetate. The filtrate was distilled under reduced pressure and purified by column chromatography to obtain compound C2-245 (3.2 g, yield: 52%).

| Compound | MW | M.P |
|---|---|---|
| C2-245 | 613.79 | 213° C. |

### [Example 22] Synthesis of Compound C2-230

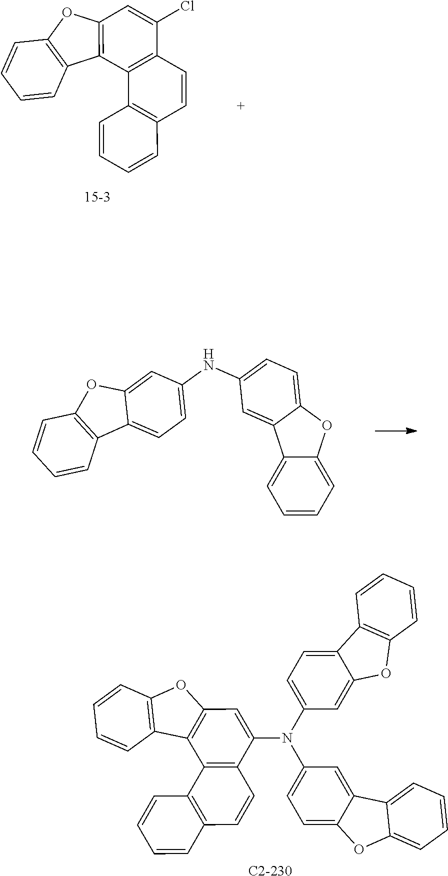 [SMILES: Clc1cc2oc3ccccc3c2c2c1ccc1ccccc12 ; c1ccc2c(c1)oc1cc(Nc3ccc4oc5ccccc5c4c3)ccc12 ; c1ccc2c(c1)ccc1c(N(c3ccc4c(c3)oc3ccccc34)c3ccc4oc5ccccc5c4c3)cc3oc4ccccc4c3c12 ; "C2-230"]

Compound 15-3 (2.14 g, 7.1 mmol), N-(dibenzo[b,d]furan-3-yl)dibenzo[b,d]furan-2-amine (2.5 g, 7.1 mmol), Pd(OAc)₂ (0.08 g, 0.36 mmol), P(tert-Bu)₃ (0.35 mL, 0.71 mmol), sodium tert-butoxide (1.4 g, 14.2 mmol), and xylene (36 mL) were added into a reaction vessel and stirred for 6 hours at 165° C. After completion of the reaction, the reaction mixture was cooled to room temperature and then the organic layer was extracted with ethyl acetate. After drying the extracted organic layer with magnesium sulfate, the solvent was removed with a rotary evaporator. Thereafter, it was purified with column chromatography to obtain compound C2-230 (2.0 g, yield: 12%).

| Compound | MW |
|---|---|
| C2-230 | 615.69 |

Hereinafter, the preparation method of an organic elect ° luminescent device (OLED) according to the present disclosure and the luminous efficiency and the lifespan characteristics thereof will be explained in order to understand the present disclosure in detail. However, it is only to describe the characteristics of the OLED according to the present application, and is not limited to the following examples.

### [Device Examples 1-1 to 1-3] Producing OLEDs in which the First Host Compound and the Second Compound According to the Present Disclosure are Co-Deposited

OLEDs according to the present disclosure were produced. First, a transparent electrode indium tin oxide (ITO) thin film (10 Ω/sq) on a glass substrate for an OLED device (GEOMATEC CO., LTD., Japan) was subject to an ultrasonic washing with acetone and isopropyl alcohol, sequentially, and then was stored in isopropanol. Next, the ITO substrate was then mounted on a substrate holder of a vacuum vapor deposition apparatus. Compound HI-1 as a first hole injection compound was introduced into a cell of the vacuum vapor deposition apparatus and compound HT-1 as a first hole transport compound was introduced into another cell of the vacuum vapor deposition apparatus, and then the two materials were evaporated at different rates to deposit a first hole injection layer having a thickness of 10 nm by doping the first hole injection compound in an amount of 3 wt % based on the total amount of the first hole injection compound and the first hole transport compound. Next, compound HT-1 was deposited as a first hole transport layer having a thickness of 80 nm on the first hole injection layer. Compound HT-2 was then introduced into another cell of the vacuum vapor deposition apparatus, and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The first host compound and the second host compound of the following Table 1 were introduced into one cell of the vacuum vapor depositing apparatus as hosts, and compound D-39 was introduced into another cell as a dopant. The two host materials were evaporated at a rate of 1:1 and simultaneously, the dopant was deposited in a doping amount of 3 wt % to form a light-emitting layer having a thickness of 40 nm on the second hole transport layer. Next, compounds ET-1 and EI-1 were evaporated at a rate of 1:1, and were deposited to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound was purified by vacuum sublimation under 10⁻⁶ torr and then used.

### [Comparative Example 1-1] Producing an OLED Comprising a Single Host Compound

An OLED was produced in the same manner as in Device Example 1-1, except that a first host compound shown in the following Table 1 was used alone as a host of the light-emitting layer.

The results of the driving voltage, the luminous efficiency, and the luminous color at a luminance of 5,000 nits, and the time taken to reduce from 100% to 95% at a luminance of 5,500 nits (lifespan; T95) of the organic electroluminescent device according to Device Examples and Comparative Example produced as described above, are shown in the following Table 1.

TABLE 1

| | First | Second | | | | Life- |
|---|---|---|---|---|---|---|
| | Host | Host | Driving | Luminous | Lu- | span |
| | Com- | Com- | Voltage | Efficiency | minous | (T95) |
| | pound | pound | [VA] | [cd/A] | Color | [hr] |
| Device | H1-145 | C2-146 | 4.2 | 30.9 | Red | 526 |
| Example 1-1 | | | | | | |
| Device | H1-145 | C2-154 | 4.2 | 32.0 | Red | 475 |
| Example 1-2 | | | | | | |
| Device | H1-145 | C2-153 | 4.5 | 29.7 | Red | 438 |
| Example 1-3 | | | | | | |
| Comparative | H1-145 | — | 4.4 | 25.4 | Red | 58 |
| Example 1-1 | | | | | | |

From the Table 1 above, by comprising the compounds of a specific combination according to the present disclosure as host materials, an organic electroluminescent device having a significantly improved luminous efficiency and significantly improved lifespan can be provided.

### [Comparative Example 2-1] Preparation of OLED Comprising the Comparative Compound as a Second Hole Transport Material

An OLED was produced in the same manner as in Device Example 1-1, except that the compound shown in the following Table 2 was used as a second hole transport material and compound H-1 was used alone as a host of the light-emitting layer.

### [Device Example 2-1] Preparation of OLED Comprising the Organic Electroluminescent Compound According to the Present Disclosure

An OLED was produced in the same manner as in Comparative Example 2-1, except that the compound shown in the following Table 2 was used as a second hole transport material.

The results of the luminous efficiency and the luminous color at a luminance of 1,000 nits, and the time taken to reduce from 100% to 95% at a luminance of 5,000 nits (lifespan; T95) of the organic electroluminescent device according to Device Example 2-1 and Comparative Example 2-2 produced as described above, are shown in the following Table 2.

TABLE 2

| | | Luminous | | |
|---|---|---|---|---|
| | Second Hole | Efficiency | Luminous | Lifespan |
| | Transport Layer | [cd/A] | Color | (T95) [hr] |
| Comparative | Compound X | 26.5 | Red | 13 |
| Example 2-1 | | | | |
| Device Example 2-1 | C2-57 | 31.9 | Red | 690 |

From the Table 2 above, it was confirmed that an organic electroluminescent device exhibiting improved luminous efficiency and particularly considerably improved lifespan characteristics can be provided by comprising the organic electroluminescent compounds according to the present disclosure as a hole transport material.

The compounds used in Device Examples and Comparative Examples above are shown in the following Table 3.

TABLE 3

| | |
|---|---|
| Hole Injection Layer/ Hole Transport Layer | 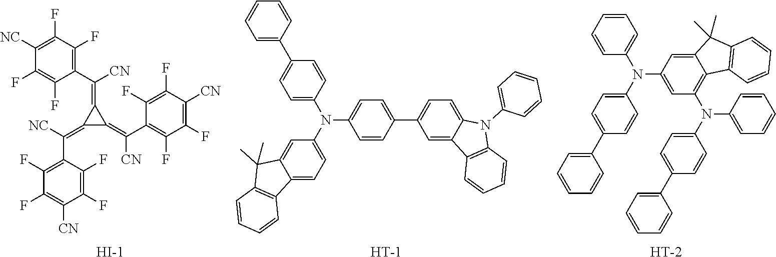 |
| | 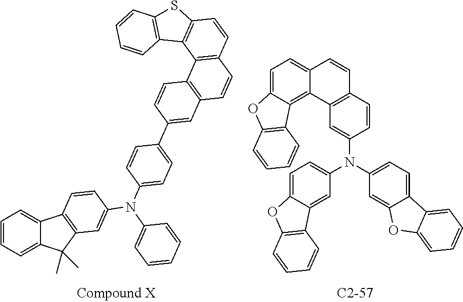 |
| Light-Emitting Layer | 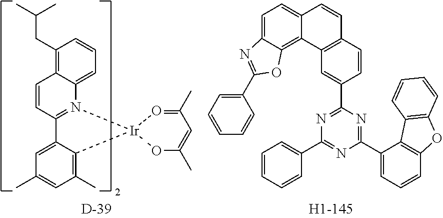 |
| | 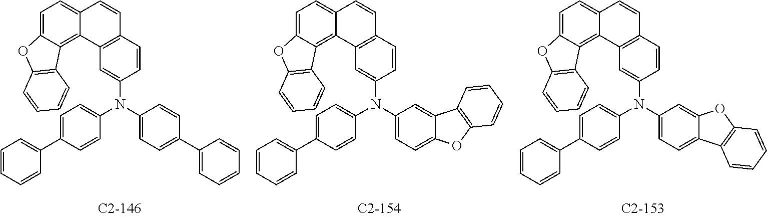 |
| | 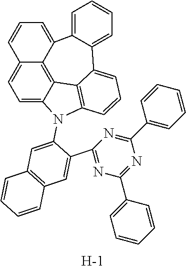 |
| Electron Transport Layer/ Electron Injection Layer | 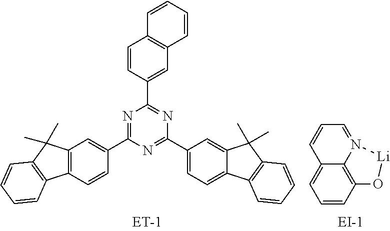 |

1. A plurality of host materials comprising a first host material and a second host material, wherein the first host material comprises a compound represented by the following formula 1 and the second host material comprises a compound represented by the following formula 2: 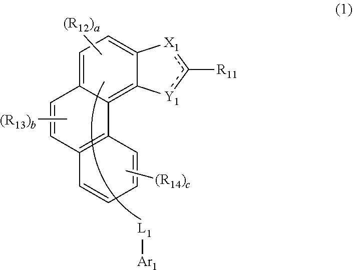
wherein,
X₁ and Y₁ each independently represent, —N═, —NR₅—, —O—, or —S—, provided that any one of X₁ and Y₁ is —N═, and other of X₁ and Y₁ is —NR₅—, —O— or —S—;
L₁ represents a single bond or a substituted or unsubstituted (C6-C30)arylene;
Ar₁ represents a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, or a substituted or unsubstituted (C6-C30)aryl(3- to 30-membered)heteroarylamino;
R₁₁ represents a substituted or unsubstituted (C6-C30)aryl or a substituted or unsubstituted (3- to 30-membered)heteroaryl;
R₁₂ to R₁₄ and R₅ each independently represent, hydrogen, deuterium, halogen, cyano, a substituted or unsubstituted (C1-C30)alkyl, a substituted or unsubstituted (C6-C30)aryl; a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl; a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to the adjacent substituent(s) to form a ring(s); and
a and b each independently represent an integer of 1 or 2, c represents an integer of 1 to 4, and when a to c are an integer of 2 or more, each of R₁₂ to R₁₄ may be the same or different; 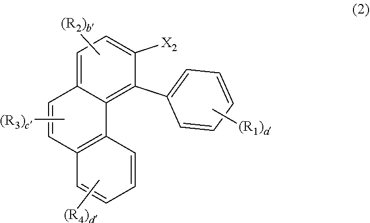
wherein,
X₂ represents O, S, or CR₇R₈;
R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a fused ring of a substituted or unsubstituted (C3-C30) aliphatic ring and a (C6-C30) aromatic ring, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered) heteroarylamino;
at least one of R₁ to R₄ is a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino, with the proviso that the case where any one of R₁ to R₄ is triphenylene is excluded;
R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s); and
a′ and d′ each independently represent, an integer of 1 to 4, b′ and c′ each independently represent an integer of 1 or 2, and when a′ to d′ represent an integer of 2 or more, each of R₁ to R₄ may be the same or different.

2. The plurality of host materials according to claim 1, wherein the compound represented by the formula 1 is represented by any one of the following formulas 1-1 to 1-3: 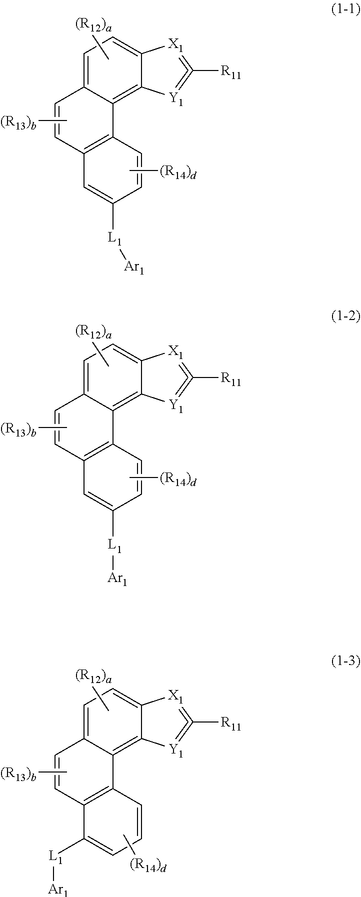
wherein,
X₁, Y₁, Ar₁, L₁, R₁₁ to R₁₄, a, and b are as defined in claim 1; and
d represents an integer of 1 to 3, and when d is an integer of 2 or more, each of R₁₄ may be the same or different.

3. The plurality of host materials according to claim 1, wherein the compound represented by the formula 2 is represented by the following formula 2-1 or 2-2: 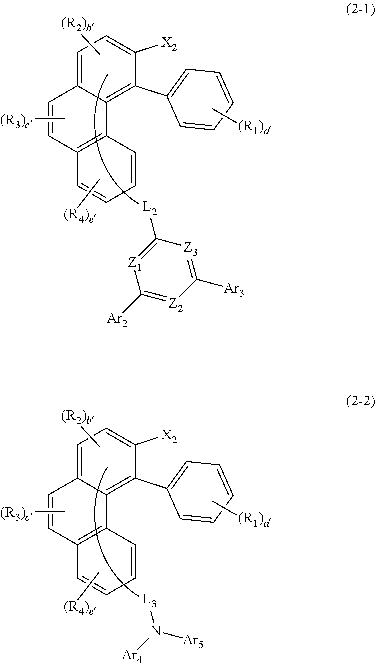
wherein,
X₂, R₁ to R₄, and a′ to c′ are as defined in claim 1;
L₂ and L₃ each independently represent, a single bond, a substituted or unsubstituted (C6-C30)arylene, or a substituted or unsubstituted (3- to 30-membered)heteroarylene;
Z₁ to Z₃ each independently represent, N or CH, provided that at least one of Z₁ to Z₃ is N;
Ar₂ to Ar₅ each independently represent, a substituted or unsubstituted (C6-C30)aryl or a substituted or unsubstituted (3- to 30-membered)heteroaryl; and
e′ represents an integer of 1 to 4, and when e′ is an integer of 2 or more, each of R₄ may be the same or different.

4. The plurality of host materials according to claim 1, wherein Ar₁ in the formula 1 represents a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, a substituted or unsubstituted phenanthrenyl, a substituted or unsubstituted benzophenanthrenyl, a substituted or unsubstituted fluoranthenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted spiro[fluoren-benzofluoren]yl, a substituted or unsubstituted pyrimidyl, a substituted or unsubstituted triazinyl, a substituted or unsubstituted quinazolinyl, a substituted or unsubstituted quinoxalinyl, a substituted or unsubstituted benzofuropyrimidinyl, or an amino substituted with at least one substituent selected from the group consisting of substituted or unsubstituted phenyl, naphthyl, o-biphenyl, m-biphenyl, p-biphenyl, terphenyl, phenanthrenyl, substituted or unsubstituted fluorenyl, a substituted or unsubstituted carbazolyl, dibenzofuranyl, and dibenzothiophenyl.

5. The plurality of host materials according to claim 1, wherein substituents in the substituted (C1-C30)alkyl, the substituted (C6-C30)aryl(ene), the substituted (3- to 30-membered)heteroaryl(ene), the substituted (C1-C30)alkoxy, the fused ring of the substituted (C3-C30) aliphatic ring and the (C6-C30) aromatic ring, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C2-C30)alkenylamino, the substituted (C1-C30)alkyl(C2-C30)alkenylamino, the substituted mono- or di-(C6-C30)arylamino, the substituted (C1-C30)alkyl(C6-C30)arylamino, the substituted mono- or di-(3- to 30-membered)heteroarylamino, the substituted (C1-C30)alkyl(3- to 30-membered)heteroarylamino, the substituted (C2-C30)alkenyl(C6-C30)arylamino, the substituted (C2-C30)alkenyl(3- to 30-membered)heteroarylamino, the substituted (C6-C30)aryl(3- to 30-membered)heteroarylamino, and the substituted ring, each independently represent at least one selected from the group consisting of deuterium, halogen, cyano, carboxyl, nitro, hydroxy, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, a (5- to 30-membered)heteroaryl unsubstituted or substituted with (C6-C30)aryl, an (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, a mono- or di-(C6-C30)arylamino unsubstituted or substituted with (C1-C30)alkyl, (C1-C30)alkyl(C6-C30)arylamino, (C1-C30)alkylcarbonyl, (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl.

6. The plurality of host materials according to claim 1, wherein the compound represented by the formula 1 is selected from the following compounds:

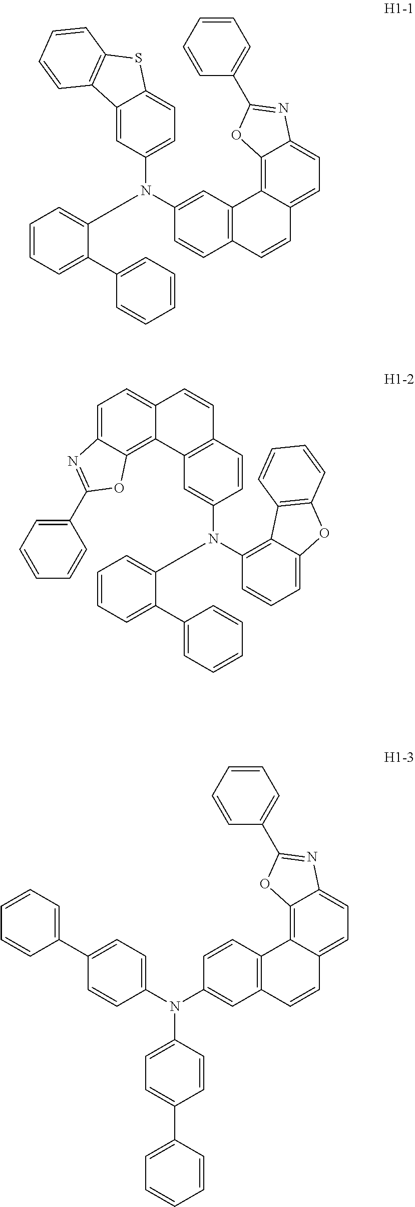 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

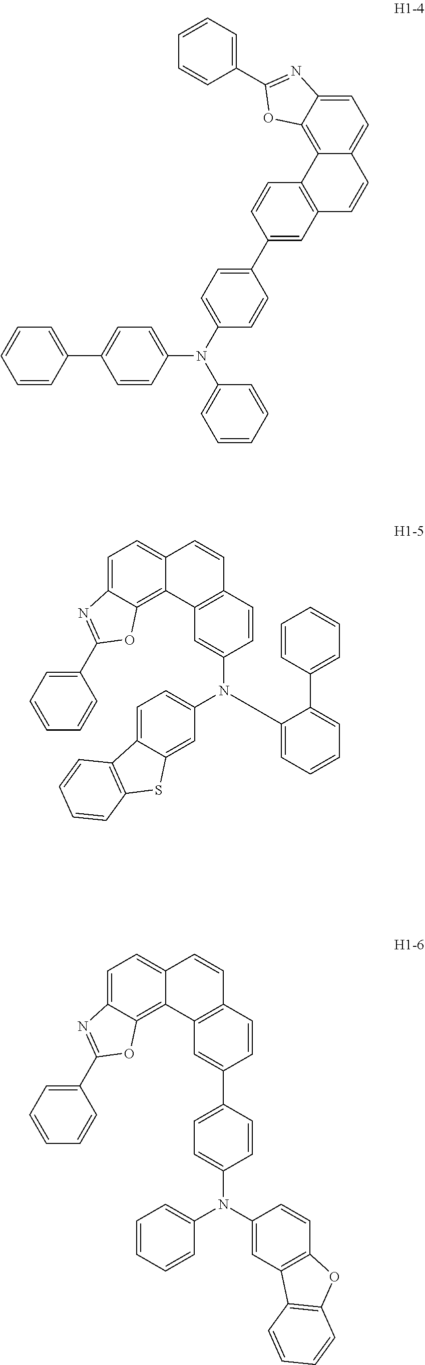 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

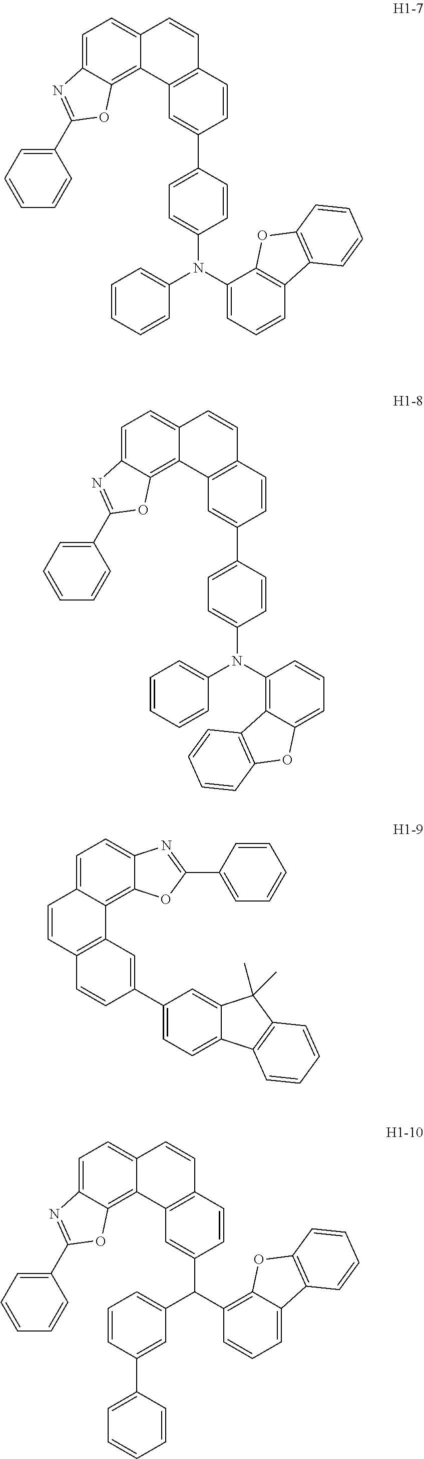 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; CC1(C)c2ccccc2-c2ccc(-c3ccc4ccc5ccc6nc(-c7ccccc7)oc6c5c4c3)cc21 ; c1ccc(-c2cccc(C(c3ccc4ccc5ccc6nc(-c7ccccc7)oc6c5c4c3)c3cccc4c3oc3ccccc34)c2)cc1]

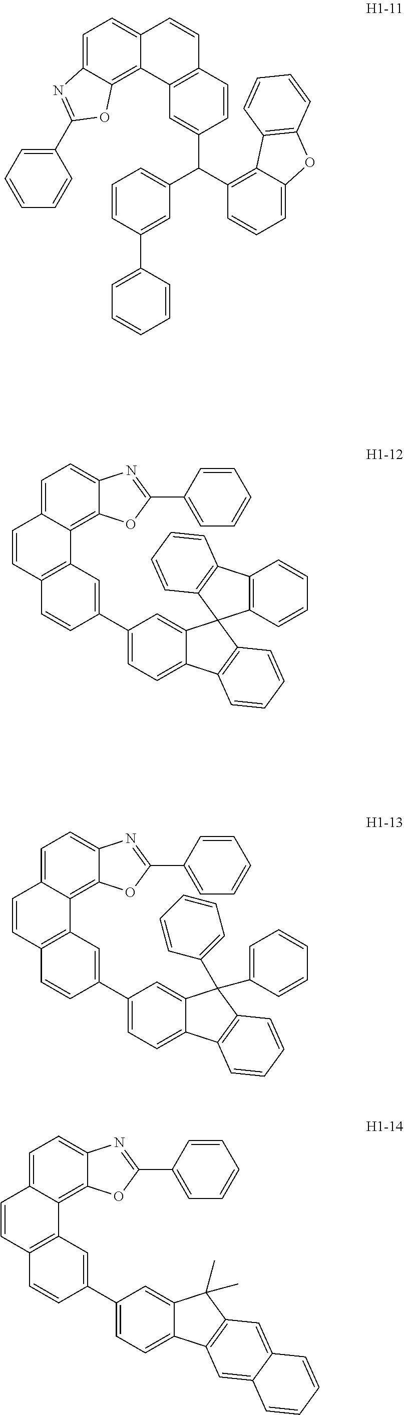 [SMILES: c1ccc(-c2cccc(C(c3ccc4ccc5ccc6nc(-c7ccccc7)oc6c5c4c3)c3cccc4oc5ccccc5c34)c2)cc1 ; c1ccc(-c2nc3ccc4ccc5ccc(-c6ccc7c(c6)C6(c8ccccc8-c8ccccc86)c6ccccc6-7)cc5c4c3o2)cc1 ; c1ccc(-c2nc3ccc4ccc5ccc(-c6ccc7c(c6)C(c6ccccc6)(c6ccccc6)c6ccccc6-7)cc5c4c3o2)cc1 ; CC1(C)c2cc(-c3ccc4ccc5ccc6nc(-c7ccccc7)oc6c5c4c3)ccc2-c2cc3ccccc3cc21]

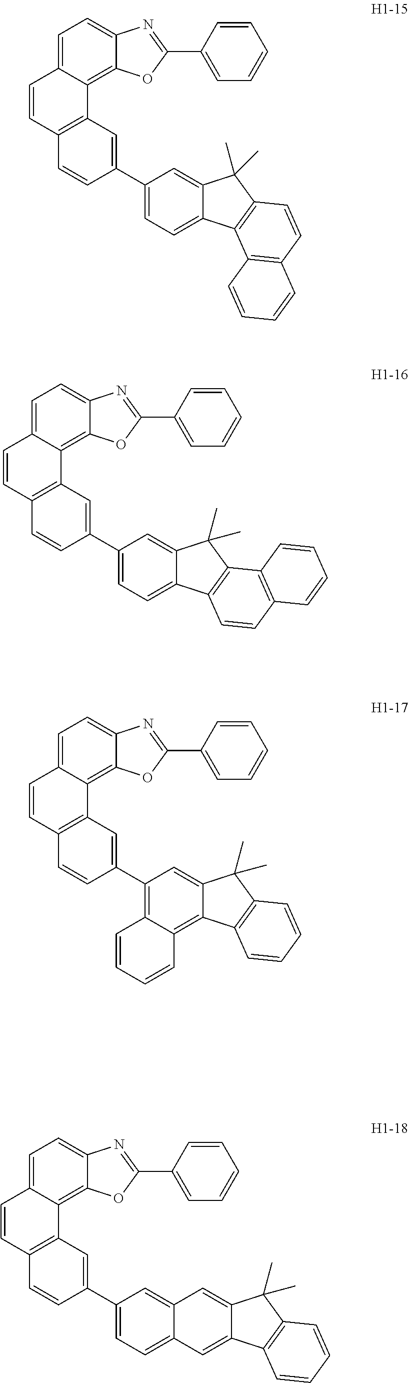 [SMILES: CC1(C)c2cc(-c3ccc4ccc5ccc6nc(-c7ccccc7)oc6c5c4c3)ccc2-c2c1ccc1ccccc21 ; CC1(C)c2cc(-c3ccc4ccc5ccc6nc(-c7ccccc7)oc6c5c4c3)ccc2-c2ccc3ccccc3c21 ; CC1(C)c2ccccc2-c2c1cc(-c1ccc3ccc4ccc5nc(-c6ccccc6)oc5c4c3c1)c1ccccc21 ; CC1(C)c2ccccc2-c2cc3ccc(-c4ccc5ccc6ccc7nc(-c8ccccc8)oc7c6c5c4)cc3cc21]

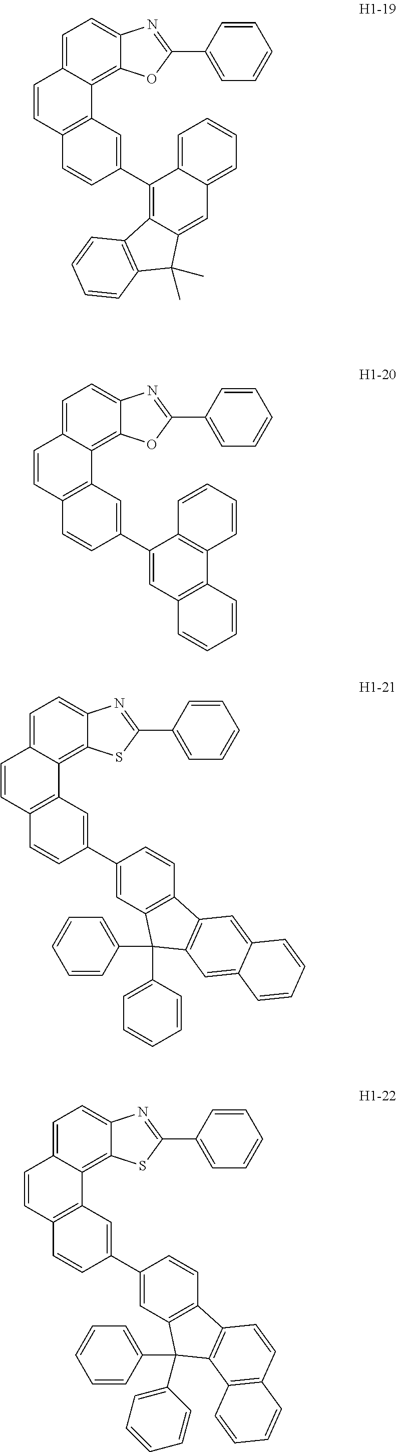 [SMILES: CC1(C)c2ccccc2-c2c1cc1ccccc1c2-c1ccc2ccc3ccc4nc(-c5ccccc5)oc4c3c2c1 ; c1ccc(-c2nc3ccc4ccc5ccc(-c6cc7ccccc7c7ccccc67)cc5c4c3o2)cc1 ; c1ccc(-c2nc3ccc4ccc5ccc(-c6ccc7c(c6)C(c6ccccc6)(c6ccccc6)c6cc8ccccc8cc6-7)cc5c4c3s2)cc1 ; c1ccc(-c2nc3ccc4ccc5ccc(-c6ccc7c(c6)C(c6ccccc6)(c6ccccc6)c6c-7ccc7ccccc67)cc5c4c3s2)cc1]

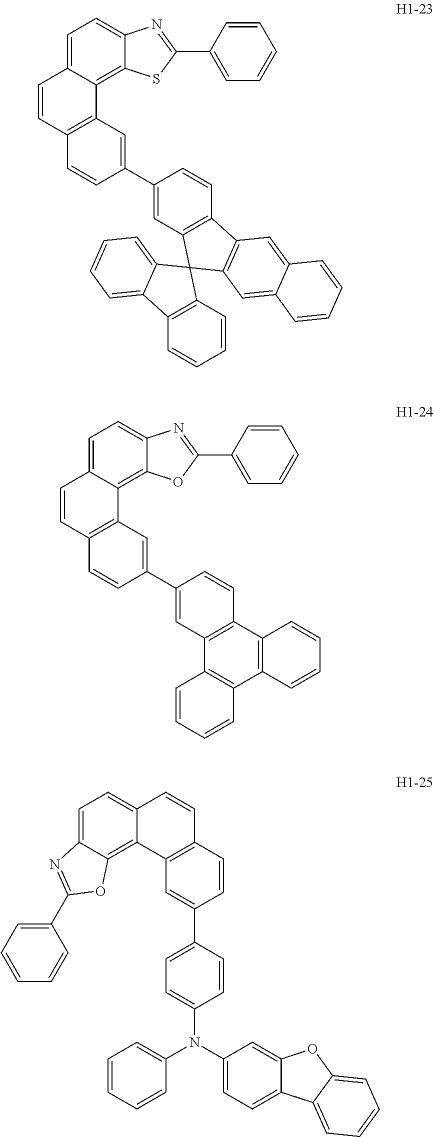 [SMILES: c1ccc(-c2nc3ccc4ccc5ccc(-c6ccc7c(c6)C6(c8ccccc8-c8ccccc86)c6cc8ccccc8cc6-7)cc5c4c3s2)cc1 ; c1ccc(-c2nc3ccc4ccc5ccc(-c6ccc7c8ccccc8c8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1]

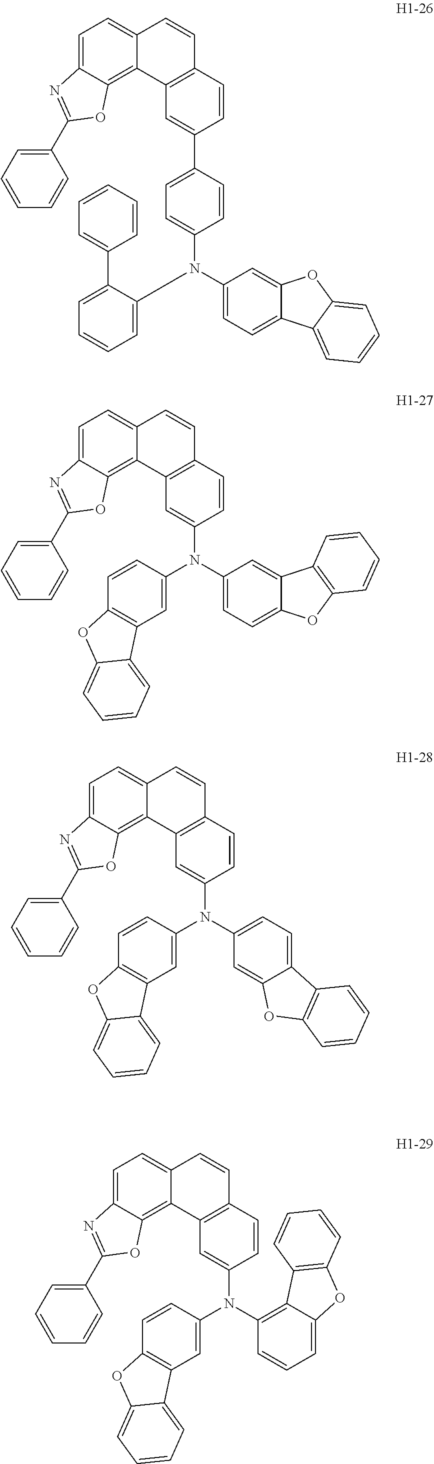 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

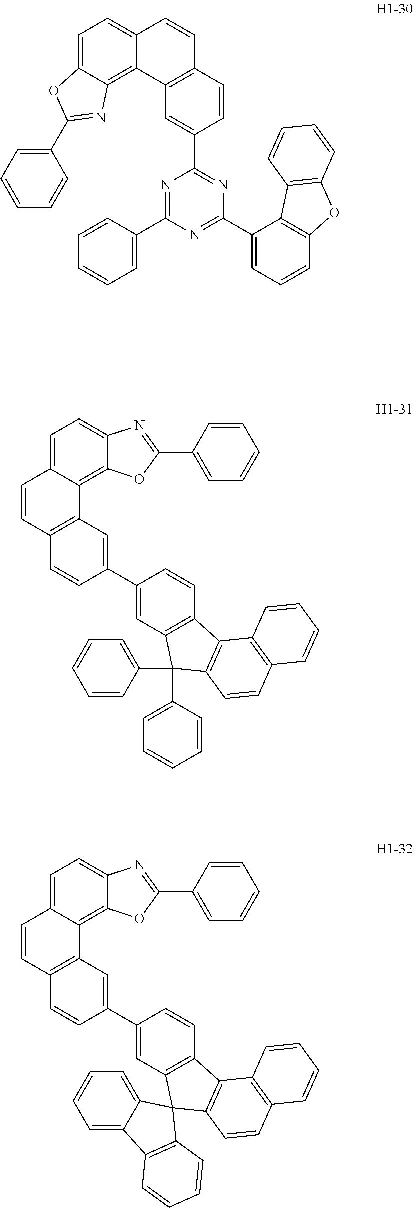 [SMILES: c1ccc(-c2nc(-c3ccc4ccc5ccc6oc(-c7ccccc7)nc6c5c4c3)nc(-c3cccc4oc5ccccc5c34)n2)cc1 ; c1ccc(-c2nc3ccc4ccc5ccc(-c6ccc7c(c6)C(c6ccccc6)(c6ccccc6)c6ccc8ccccc8c6-7)cc5c4c3o2)cc1 ; c1ccc(-c2nc3ccc4ccc5ccc(-c6ccc7c(c6)C6(c8ccccc8-c8ccccc86)c6ccc8ccccc8c6-7)cc5c4c3o2)cc1]

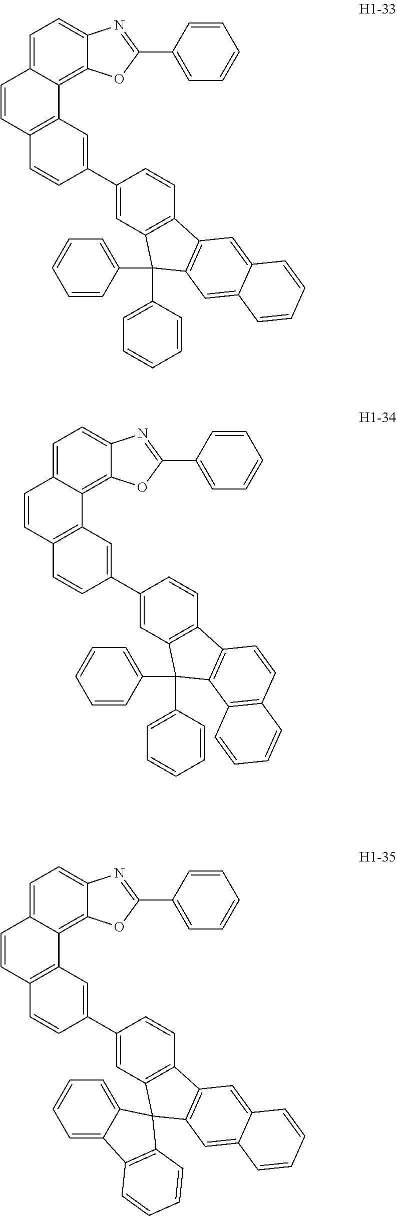 [SMILES: c1ccc(-c2nc3ccc4ccc5ccc(-c6ccc7c(c6)C(c6ccccc6)(c6ccccc6)c6cc8ccccc8cc6-7)cc5c4c3o2)cc1 ; c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

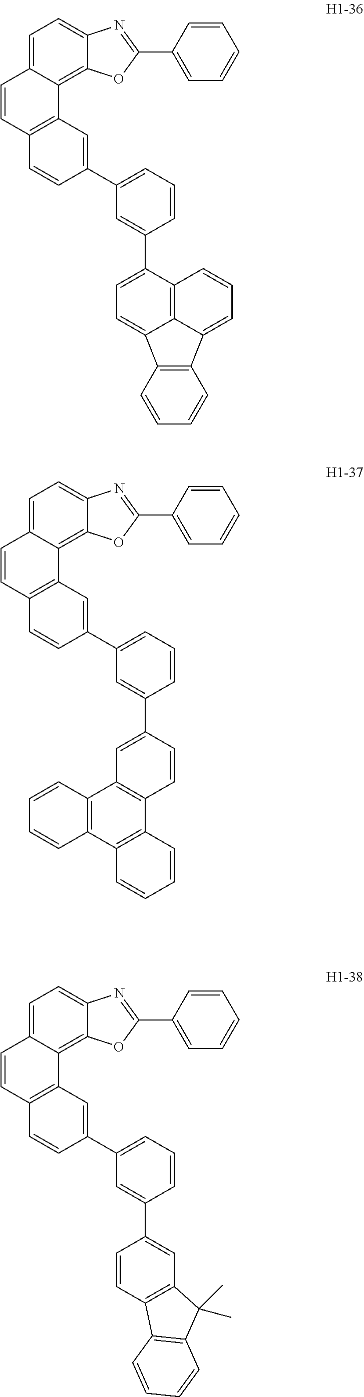 [SMILES: c1ccc(-c2nc3ccc4ccc5ccc(-c6cccc(-c7ccc8c9c(cccc79)-c7ccccc7-8)c6)cc5c4c3o2)cc1 ; c1ccc(-c2nc3ccc4ccc5ccc(-c6cccc(-c7ccc8c9ccccc9c9ccccc9c8c7)c6)cc5c4c3o2)cc1 ; CC1(C)c2ccccc2-c2ccc(-c3cccc(-c4ccc5ccc6ccc7nc(-c8ccccc8)oc7c6c5c4)c3)cc21]

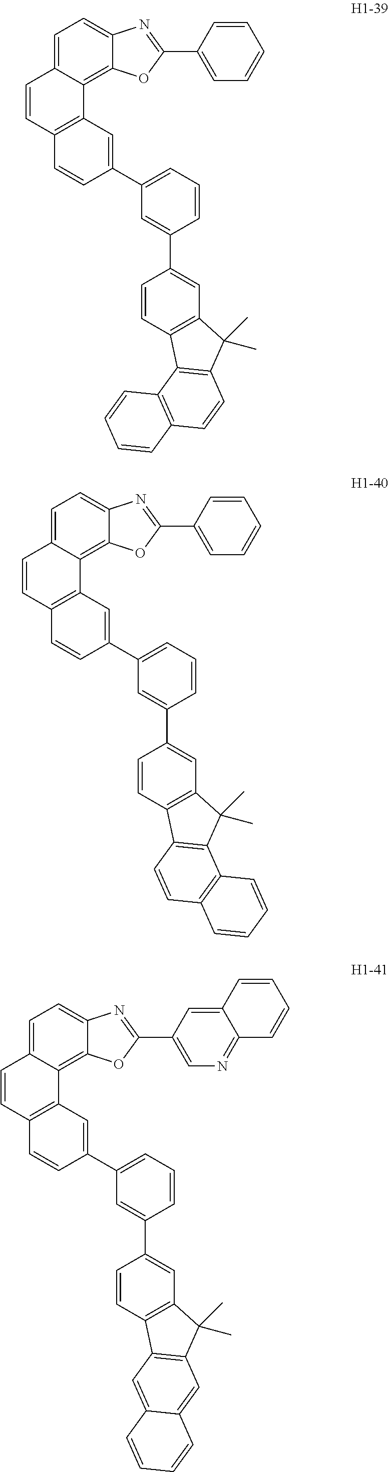

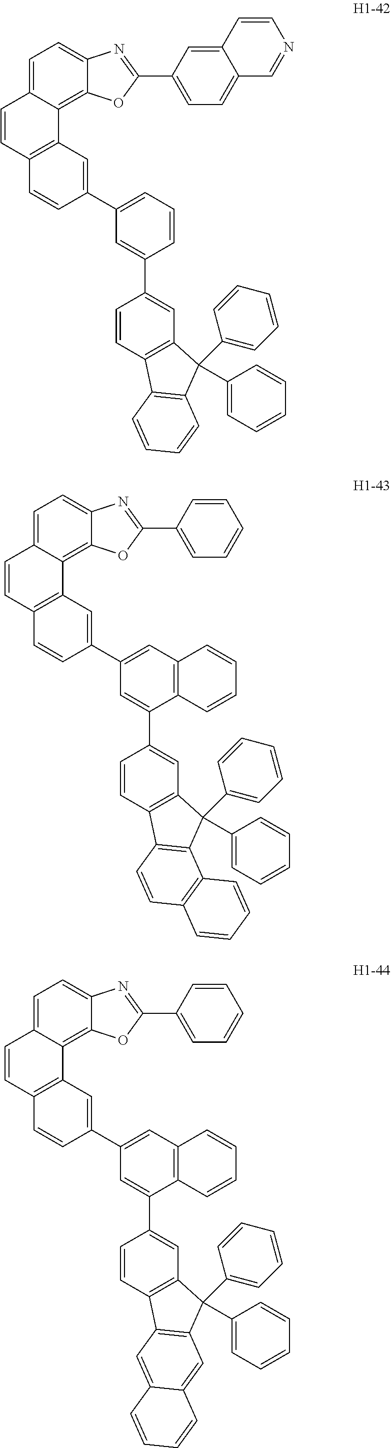

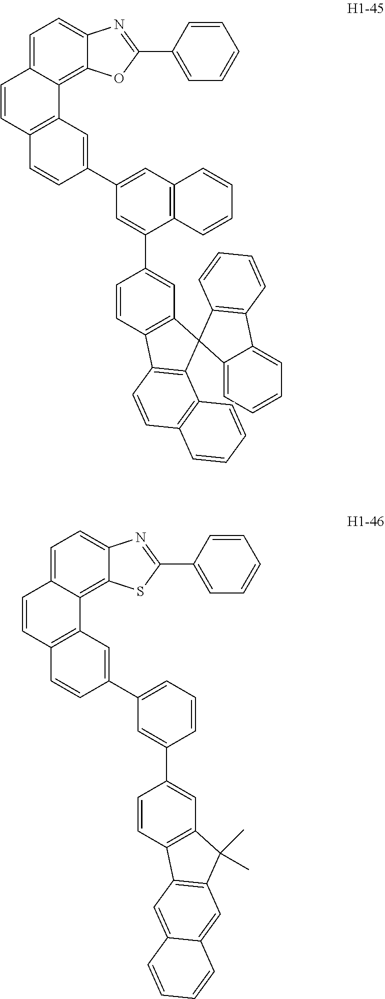 [SMILES: c1ccc(-c2nc3ccc4ccc5ccc(-c6cc(-c7ccc8c(c7)C7(c9ccccc9-c9ccccc97)c7c-8ccc8ccccc78)c7ccccc7c6)cc5c4c3o2)cc1 ; CC1(C)c2cc(-c3cccc(-c4ccc5ccc6ccc7nc(-c8ccccc8)sc7c6c5c4)c3)ccc2-c2cc3ccccc3cc21]

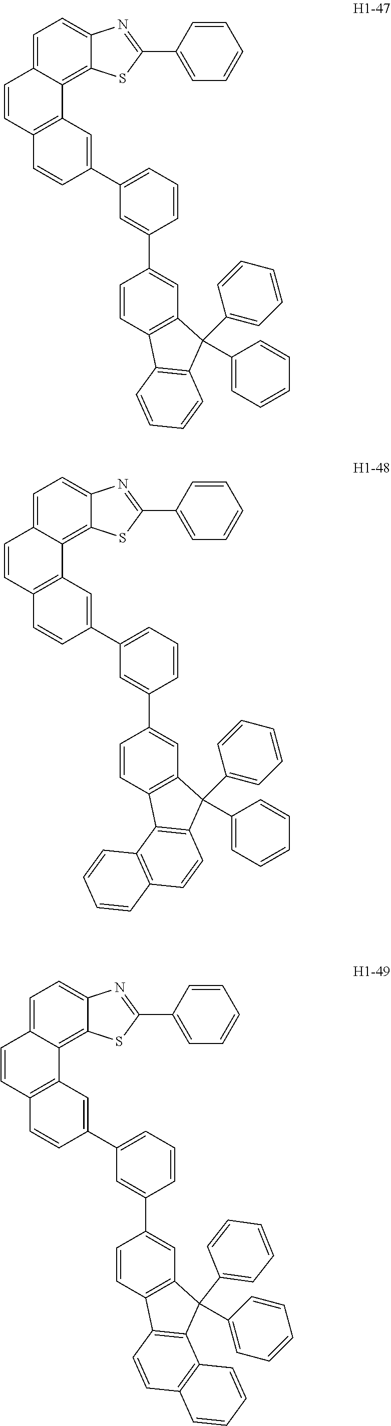 [SMILES: c1ccc(-c2nc3ccc4ccc5ccc(-c6cccc(-c7ccc8c(c7)C(c7ccccc7)(c7ccccc7)c7ccccc7-8)c6)cc5c4c3s2)cc1 ; c1ccc(-c2nc3ccc4ccc5ccc(-c6cccc(-c7ccc8c(c7)C(c7ccccc7)(c7ccccc7)c7ccc9ccccc9c7-8)c6)cc5c4c3s2)cc1 ; c1ccc(-c2nc3ccc4ccc5ccc(-c6cccc(-c7ccc8c(c7)C(c7ccccc7)(c7ccccc7)c7c-8ccc8ccccc78)c6)cc5c4c3s2)cc1]

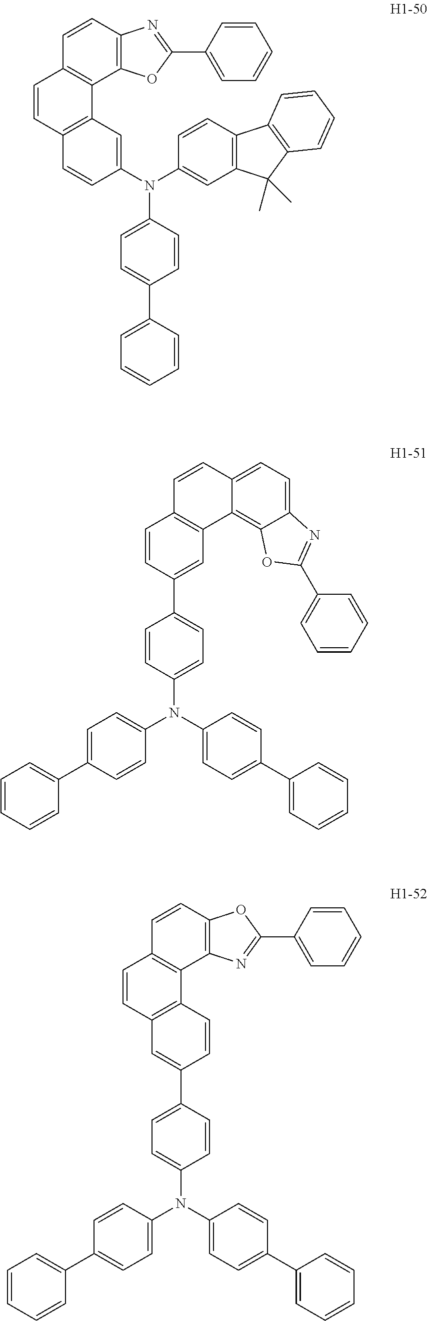 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1]

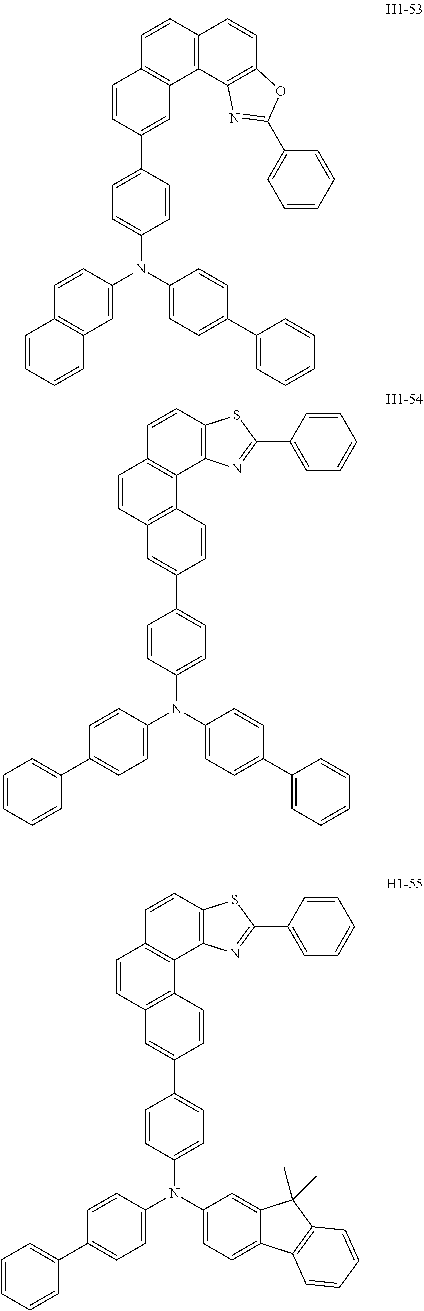

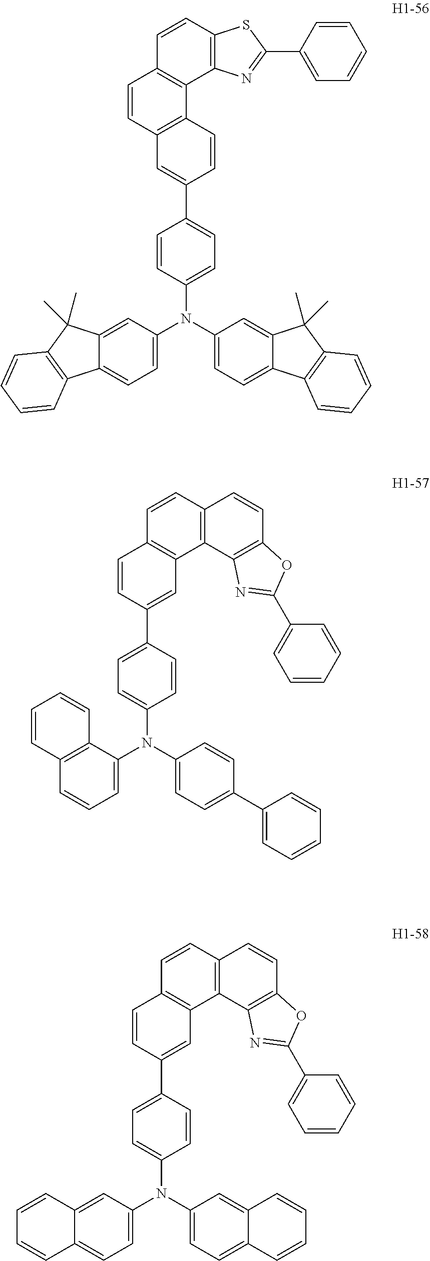 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1]

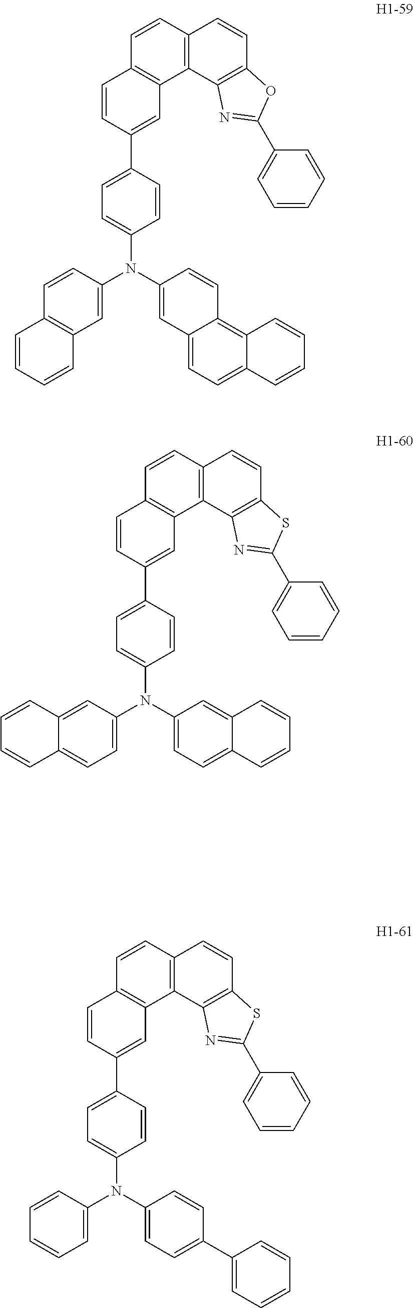 [SMILES: c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1]

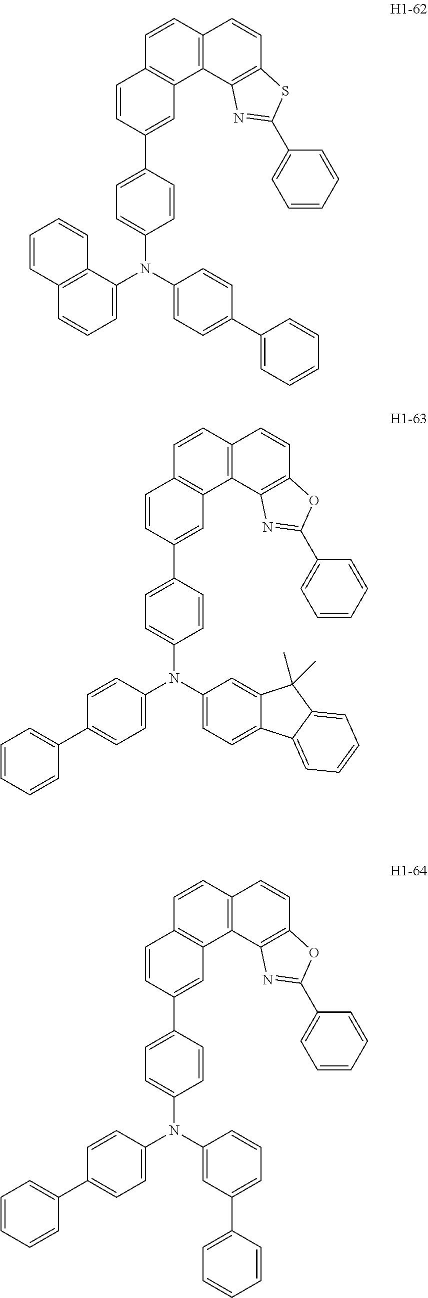 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

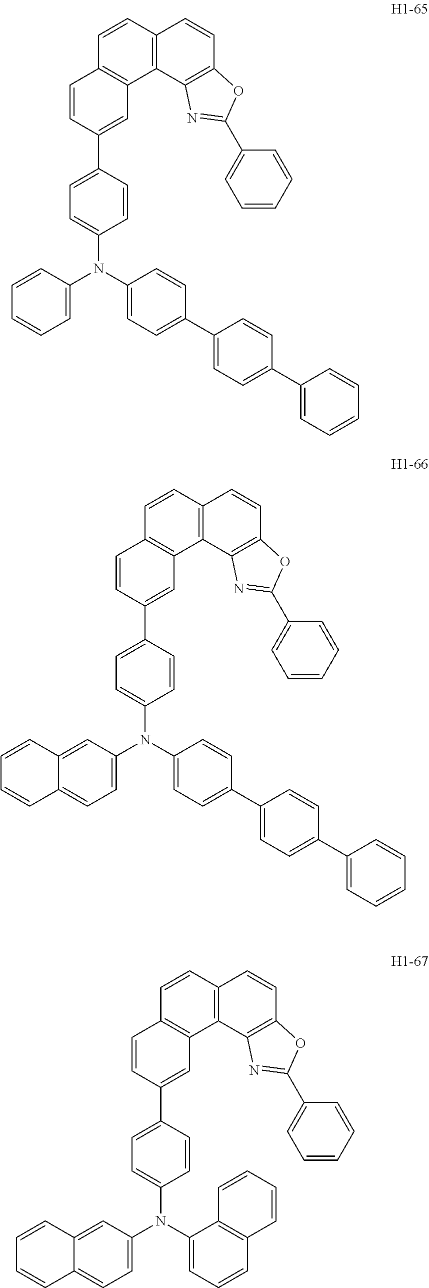 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

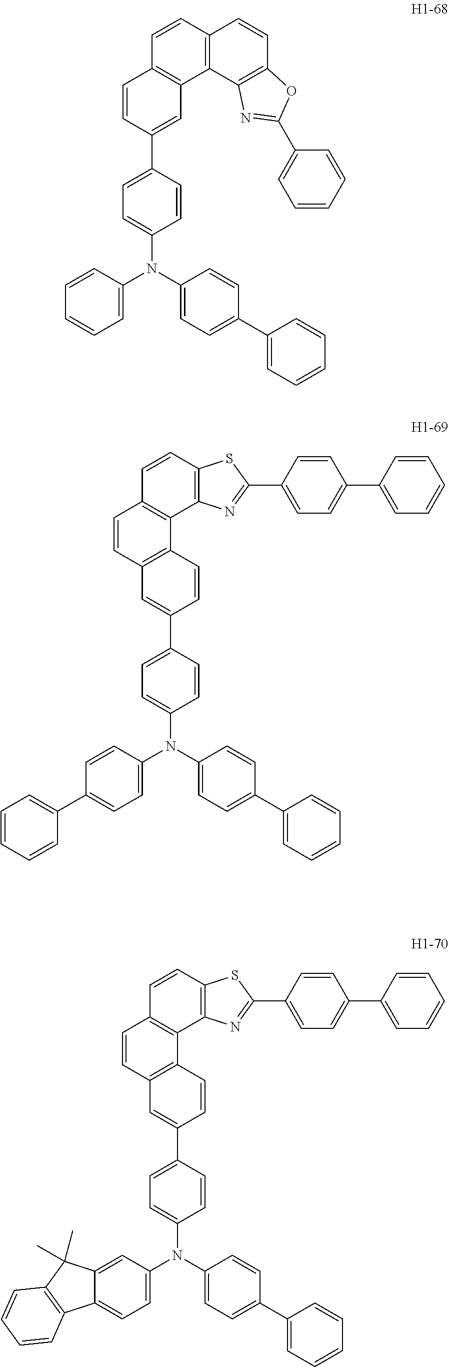 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

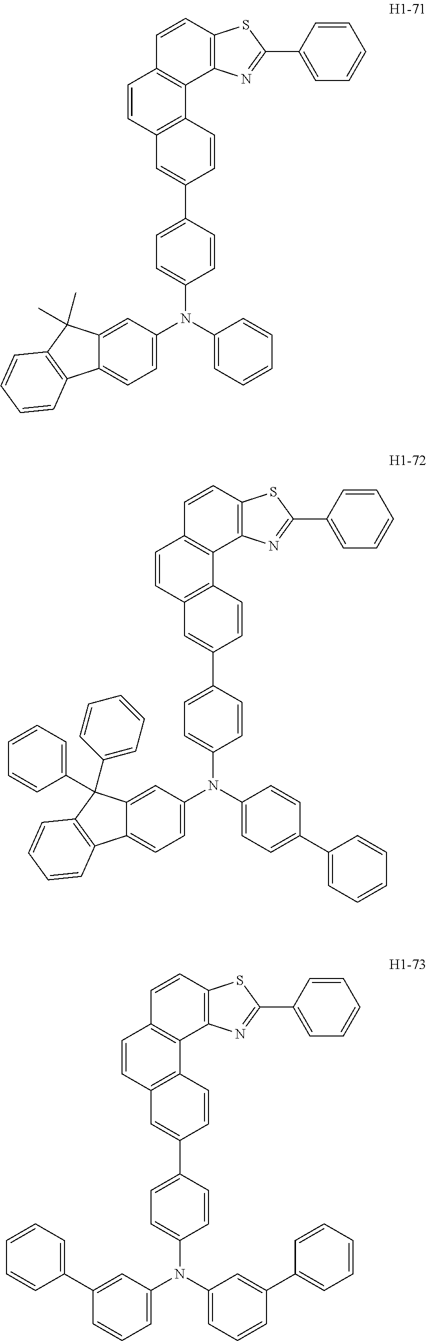 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

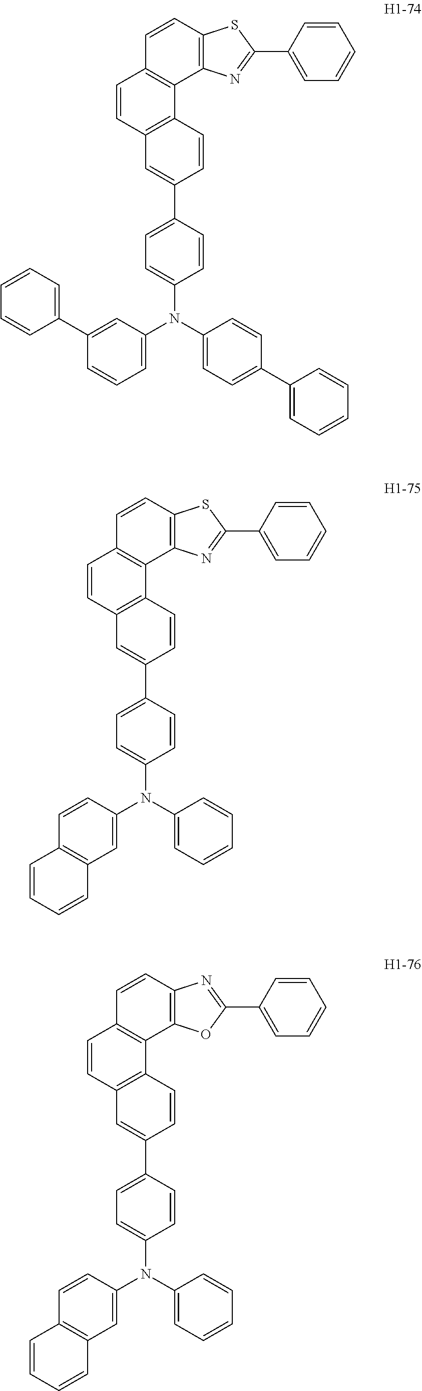 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1]

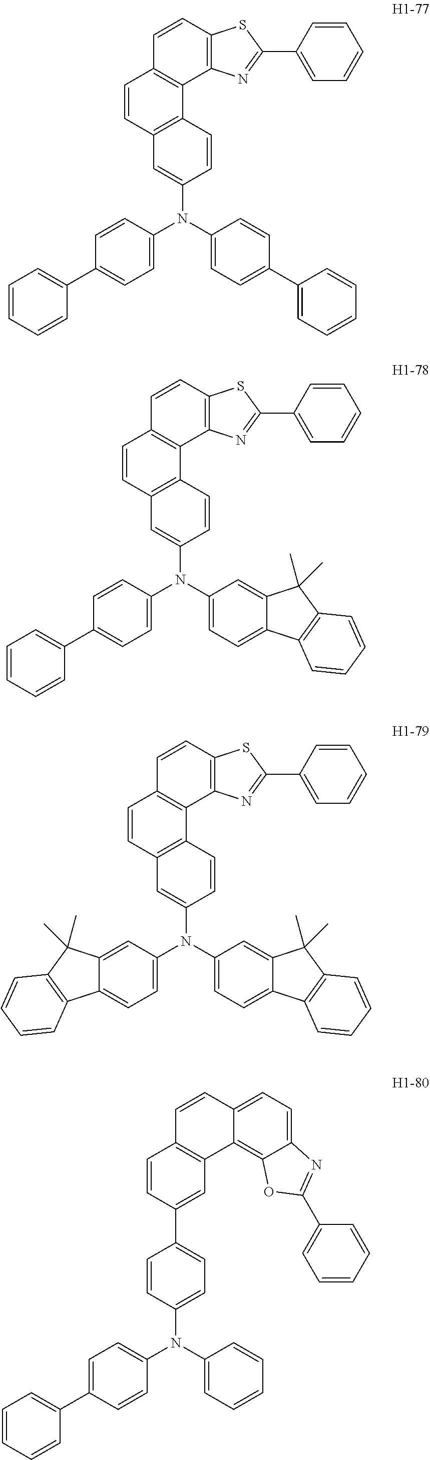 [SMILES: c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

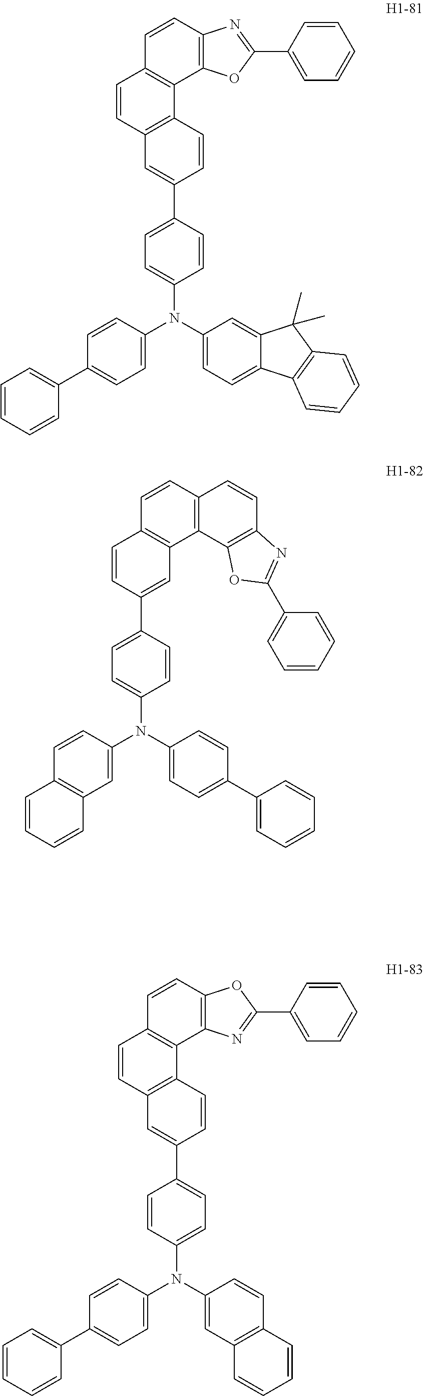 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

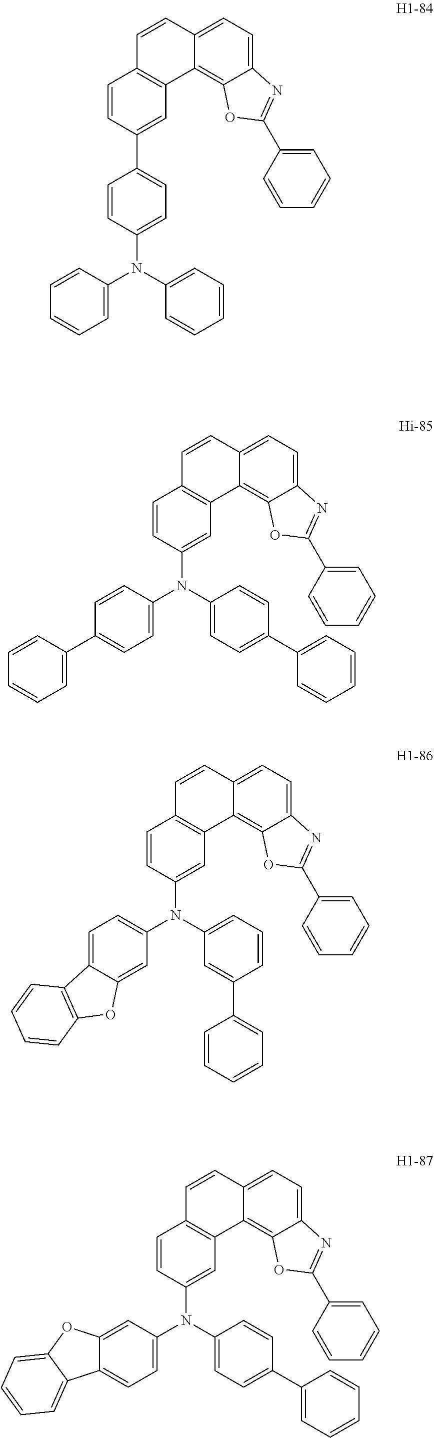 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1]

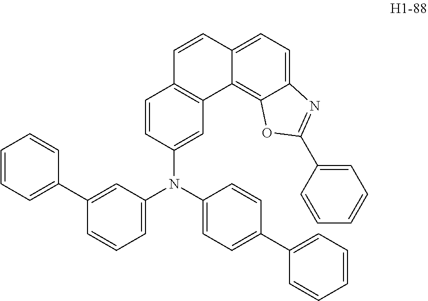 [SMILES: c1ccc(-c2ccc(N(c3cccc(-c4ccccc4)c3)c3ccc4ccc5ccc6nc(-c7ccccc7)oc6c5c4c3)cc2)cc1]

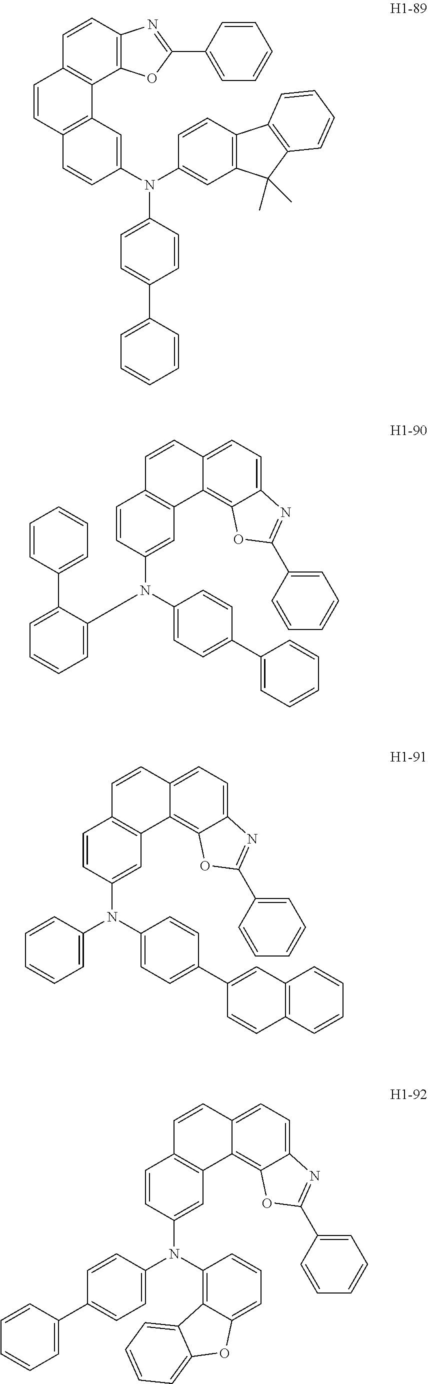 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1]

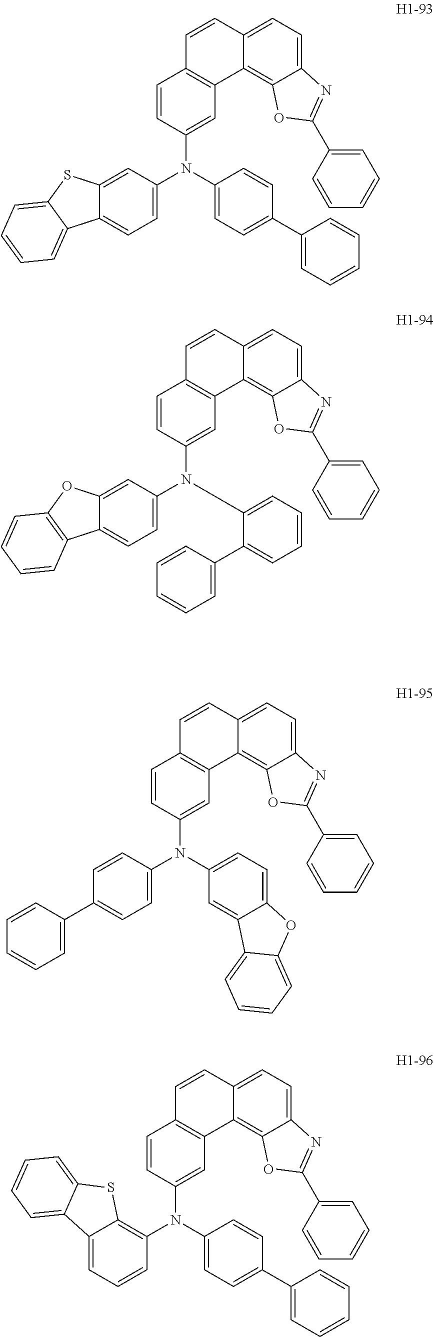 [SMILES: c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4c3sc3ccccc34)cc2)cc1]

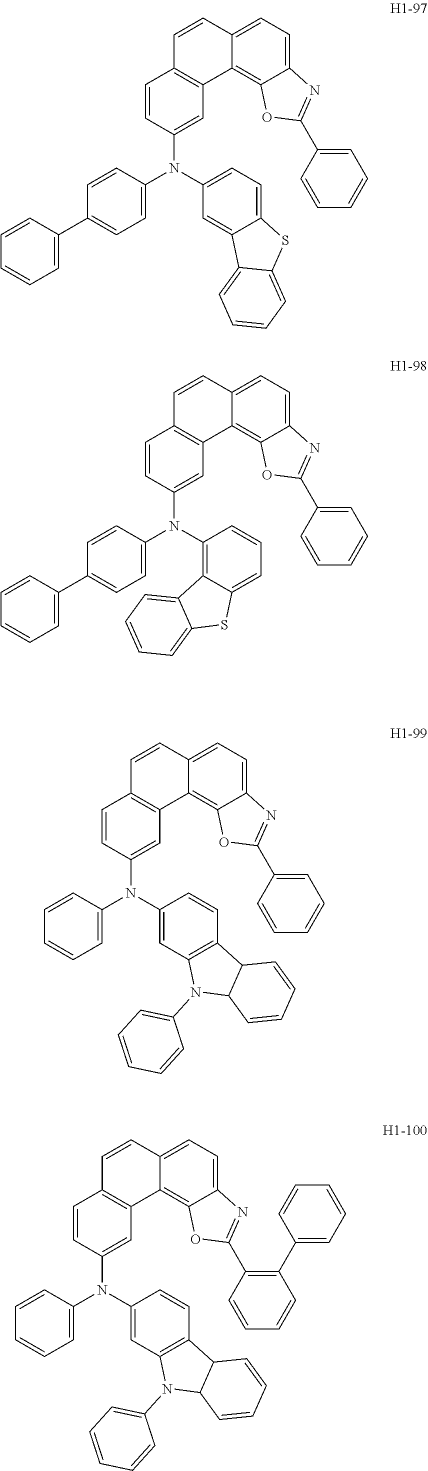 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

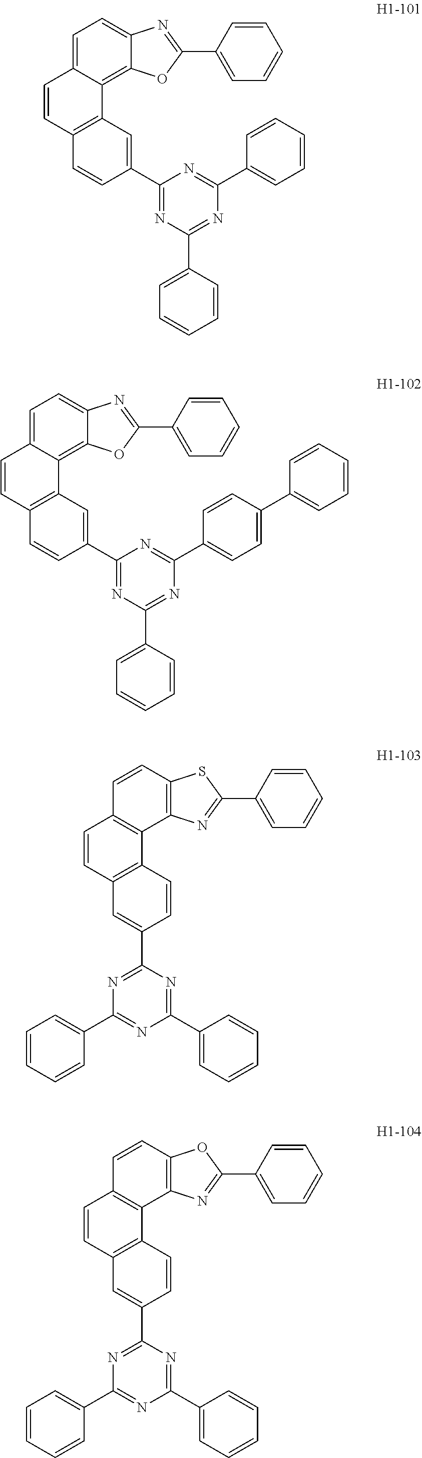 [SMILES: c1ccc(-c2nc(-c3ccccc3)nc(-c3ccc4ccc5ccc6nc(-c7ccccc7)oc6c5c4c3)n2)cc1 ; c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6oc(-c7ccccc7)nc6c54)c3)n2)cc1]

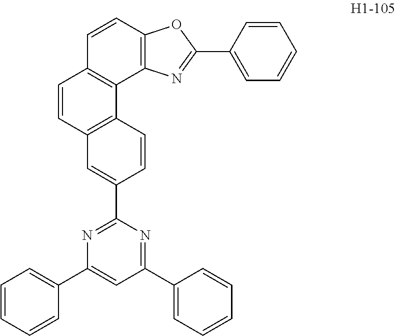 [SMILES: c1ccc(-c2cc(-c3ccccc3)nc(-c3ccc4c(ccc5ccc6oc(-c7ccccc7)nc6c54)c3)n2)cc1]

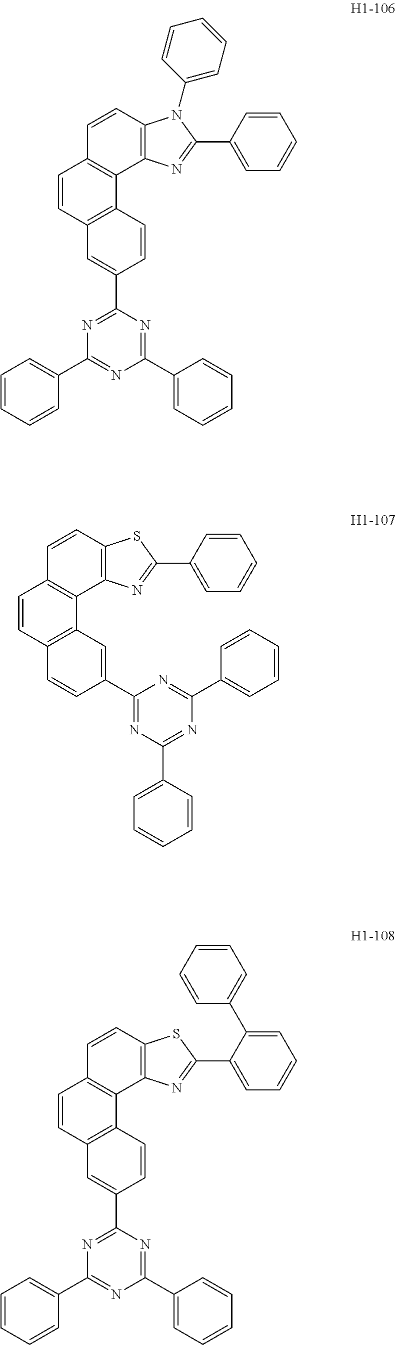 [SMILES: c1ccc(-c2nc(-c3ccccc3)nc(-c3ccc4c(ccc5ccc6c(nc(-c7ccccc7)n6-c6ccccc6)c54)c3)n2)cc1 ; c1ccc(-c2nc(-c3ccccc3)nc(-c3ccc4ccc5ccc6sc(-c7ccccc7)nc6c5c4c3)n2)cc1 ; c1ccc(-c2nc(-c3ccccc3)nc(-c3ccc4c(ccc5ccc6sc(-c7ccccc7-c7ccccc7)nc6c54)c3)n2)cc1]

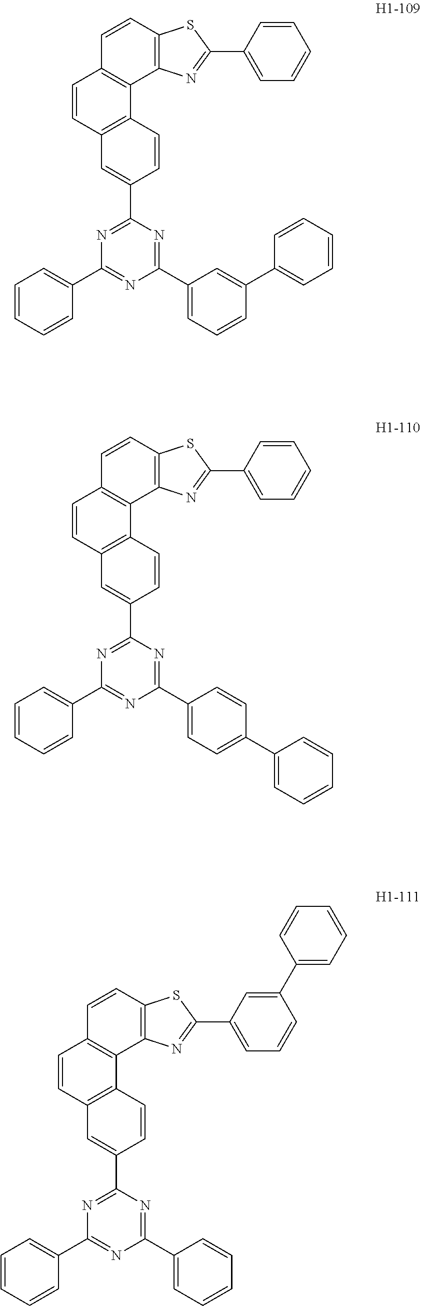 [SMILES: c1ccc(-c2cccc(-c3nc(-c4ccccc4)nc(-c4ccc5c(ccc6ccc7sc(-c8ccccc8)nc7c65)c4)n3)c2)cc1 ; c1ccc(-c2ccc(-c3nc(-c4ccccc4)nc(-c4ccc5c(ccc6ccc7sc(-c8ccccc8)nc7c65)c4)n3)cc2)cc1 ; c1ccc(-c2cccc(-c3nc4c(ccc5ccc6cc(-c7nc(-c8ccccc8)nc(-c8ccccc8)n7)ccc6c54)s3)c2)cc1]

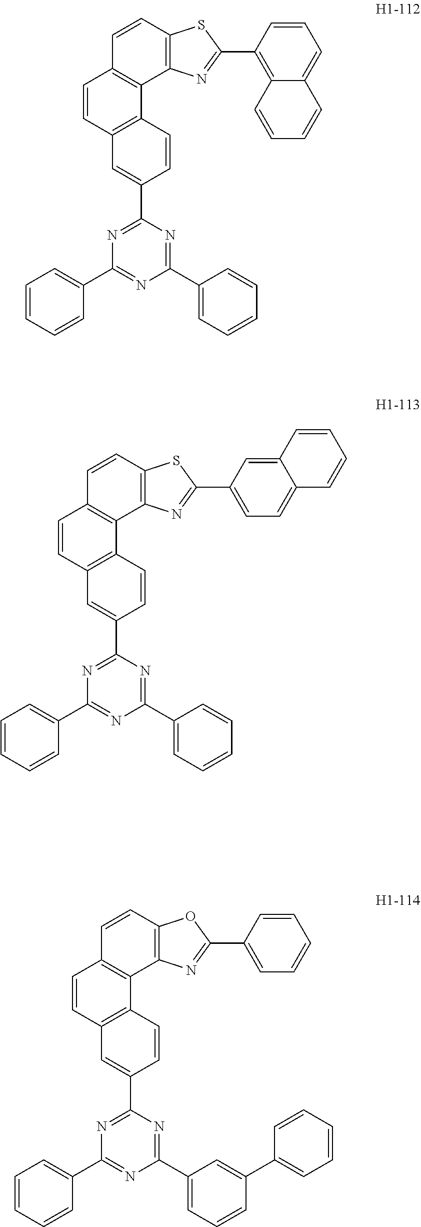 [SMILES: c1ccc(-c2nc(-c3ccccc3)nc(-c3ccc4c(ccc5ccc6sc(-c7cccc8ccccc78)nc6c54)c3)n2)cc1 ; c1ccc(-c2nc(-c3ccccc3)nc(-c3ccc4c(ccc5ccc6sc(-c7ccc8ccccc8c7)nc6c54)c3)n2)cc1 ; c1ccc(-c2cccc(-c3nc(-c4ccccc4)nc(-c4ccc5c(ccc6ccc7oc(-c8ccccc8)nc7c65)c4)n3)c2)cc1]

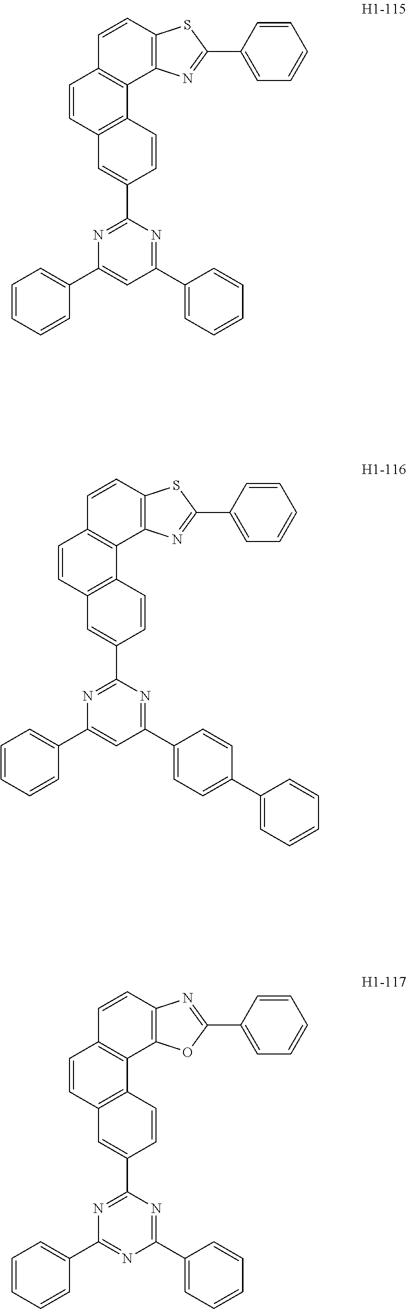 [SMILES: c1ccc(-c2cc(-c3ccccc3)nc(-c3ccc4c(ccc5ccc6sc(-c7ccccc7)nc6c54)c3)n2)cc1 ; c1ccc(-c2ccc(-c3cc(-c4ccccc4)nc(-c4ccc5c(ccc6ccc7sc(-c8ccccc8)nc7c65)c4)n3)cc2)cc1 ; c1ccc(-c2nc(-c3ccccc3)nc(-c3ccc4c(ccc5ccc6nc(-c7ccccc7)oc6c54)c3)n2)cc1]

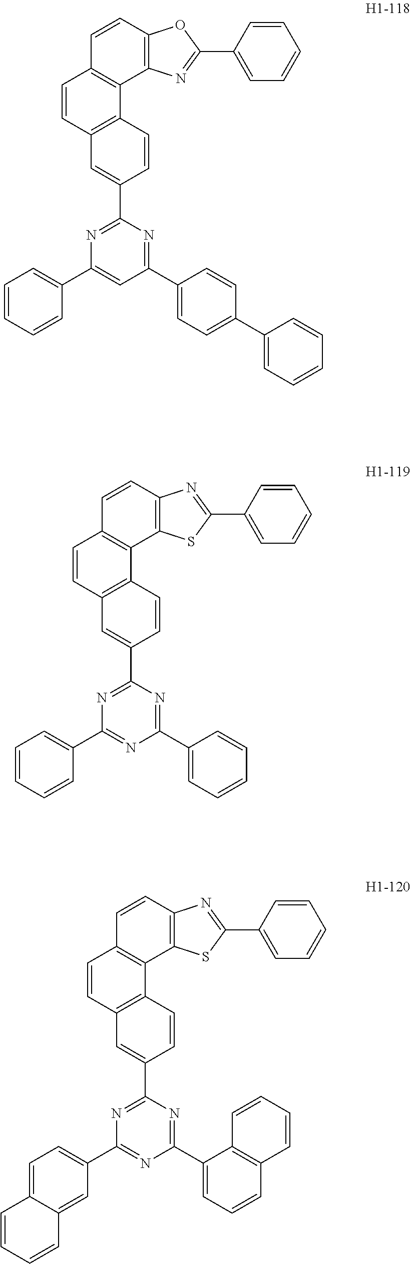 [SMILES: c1ccc(-c2ccc(-c3cc(-c4ccccc4)nc(-c4ccc5c(ccc6ccc7oc(-c8ccccc8)nc7c65)c4)n3)cc2)cc1 ; c1ccc(-c2nc(-c3ccccc3)nc(-c3ccc4c(ccc5ccc6nc(-c7ccccc7)sc6c54)c3)n2)cc1 ; c1ccc(-c2nc3ccc4ccc5cc(-c6nc(-c7ccc8ccccc8c7)nc(-c7cccc8ccccc78)n6)ccc5c4c3s2)cc1]

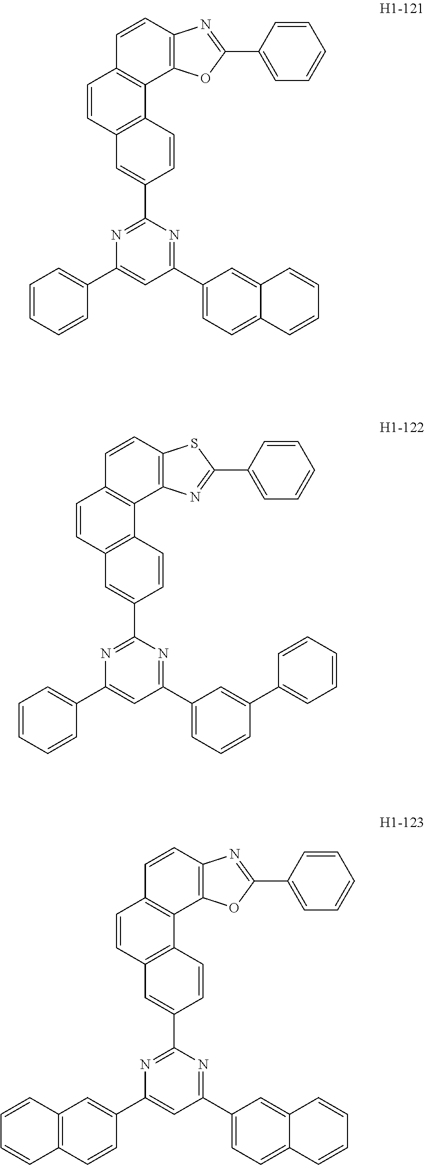 [SMILES: c1ccc(-c2cc(-c3ccc4ccccc4c3)nc(-c3ccc4c(ccc5ccc6nc(-c7ccccc7)oc6c54)c3)n2)cc1 ; c1ccc(-c2cccc(-c3cc(-c4ccccc4)nc(-c4ccc5c(ccc6ccc7sc(-c8ccccc8)nc7c65)c4)n3)c2)cc1 ; c1ccc(-c2nc3ccc4ccc5cc(-c6nc(-c7ccc8ccccc8c7)cc(-c7ccc8ccccc8c7)n6)ccc5c4c3o2)cc1]

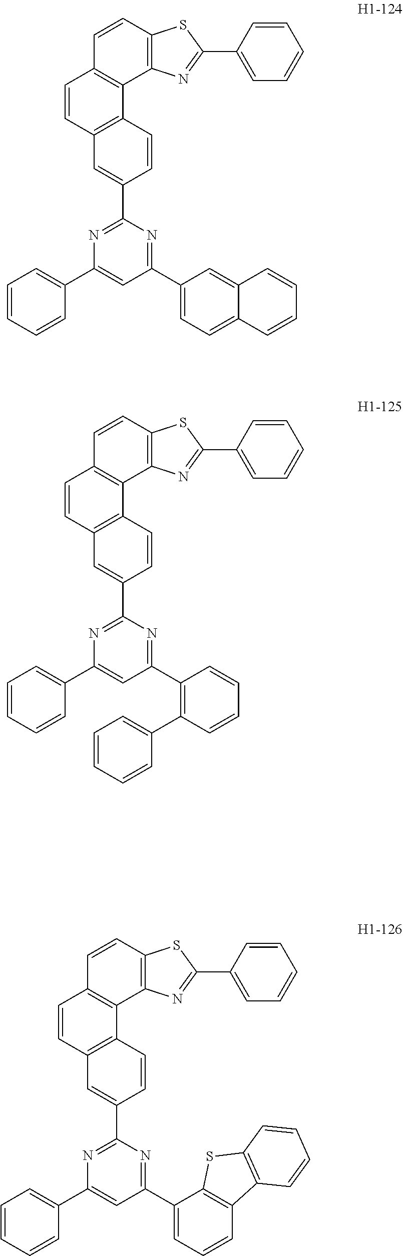 [SMILES: c1ccc(-c2cc(-c3ccc4ccccc4c3)nc(-c3ccc4c(ccc5ccc6sc(-c7ccccc7)nc6c54)c3)n2)cc1 ; c1ccc(-c2cc(-c3ccccc3-c3ccccc3)nc(-c3ccc4c(ccc5ccc6sc(-c7ccccc7)nc6c54)c3)n2)cc1 ; c1ccc(-c2cc(-c3cccc4c3sc3ccccc34)nc(-c3ccc4c(ccc5ccc6sc(-c7ccccc7)nc6c54)c3)n2)cc1]

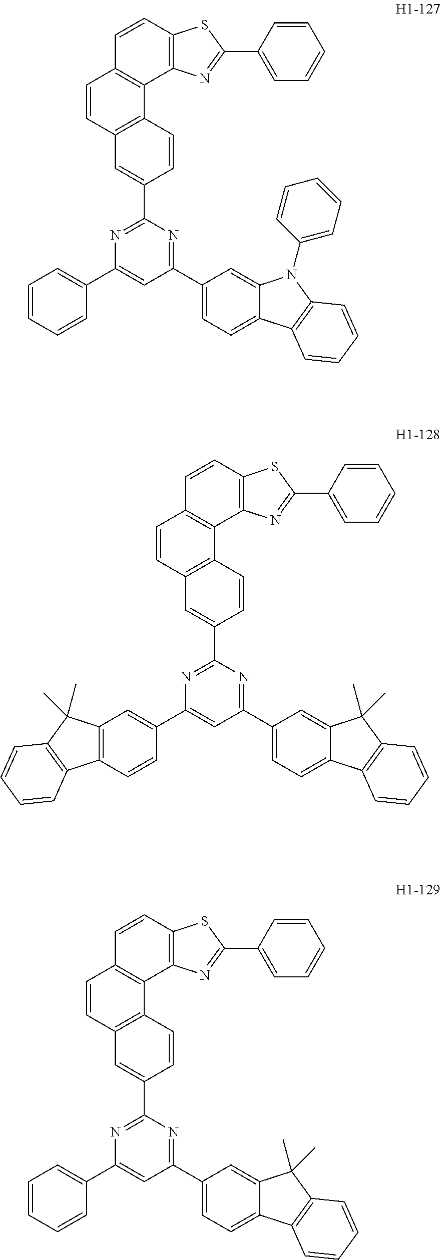 [SMILES: c1ccc(-c2cc(-c3ccc4c5ccccc5n(-c5ccccc5)c4c3)nc(-c3ccc4c(ccc5ccc6sc(-c7ccccc7)nc6c54)c3)n2)cc1 ; CC1(C)c2ccccc2-c2ccc(-c3cc(-c4ccc5c(c4)C(C)(C)c4ccccc4-5)nc(-c4ccc5c(ccc6ccc7sc(-c8ccccc8)nc7c65)c4)n3)cc21 ; CC1(C)c2ccccc2-c2ccc(-c3cc(-c4ccccc4)nc(-c4ccc5c(ccc6ccc7sc(-c8ccccc8)nc7c65)c4)n3)cc21]

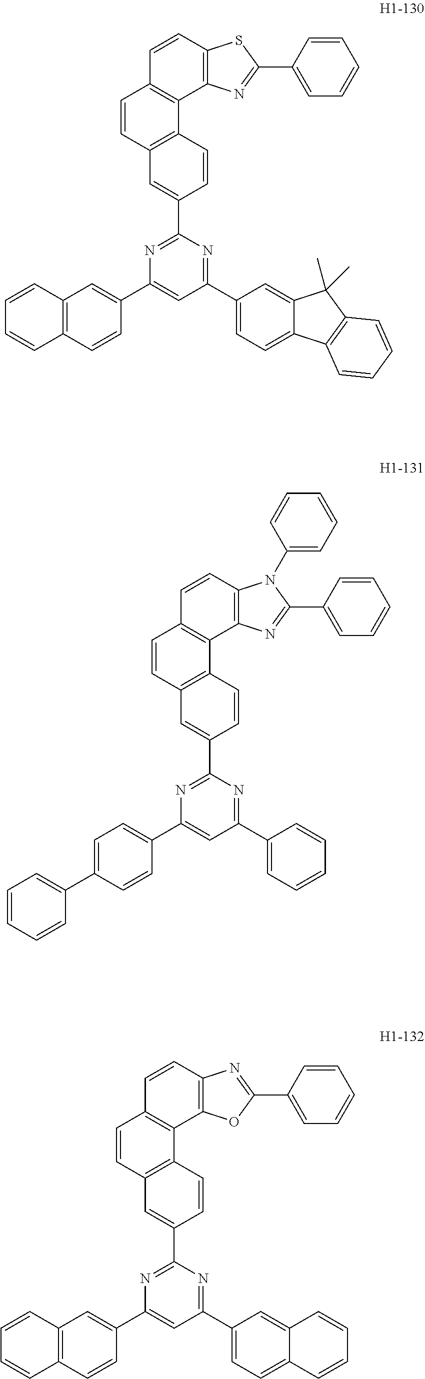 [SMILES: CC1(C)c2ccccc2-c2ccc(-c3cc(-c4ccc5ccccc5c4)nc(-c4ccc5c(ccc6ccc7sc(-c8ccccc8)nc7c65)c4)n3)cc21 ; c1ccc(-c2ccc(-c3cc(-c4ccccc4)nc(-c4ccc5c(ccc6ccc7c(nc(-c8ccccc8)n7-c7ccccc7)c65)c4)n3)cc2)cc1 ; c1ccc(-c2nc3ccc4ccc5cc(-c6nc(-c7ccc8ccccc8c7)cc(-c7ccc8ccccc8c7)n6)ccc5c4c3o2)cc1]

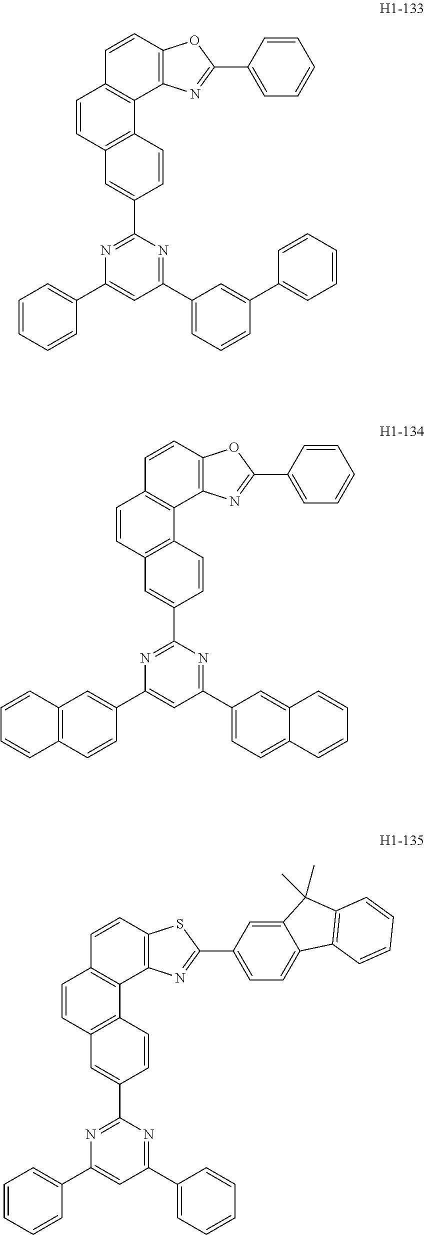 [SMILES: c1ccc(-c2cccc(-c3cc(-c4ccccc4)nc(-c4ccc5c(ccc6ccc7oc(-c8ccccc8)nc7c65)c4)n3)c2)cc1 ; c1ccc(-c2nc3c(ccc4ccc5cc(-c6nc(-c7ccc8ccccc8c7)cc(-c7ccc8ccccc8c7)n6)ccc5c43)o2)cc1 ; CC1(C)c2ccccc2-c2ccc(-c3nc4c(ccc5ccc6cc(-c7nc(-c8ccccc8)cc(-c8ccccc8)n7)ccc6c54)s3)cc21]

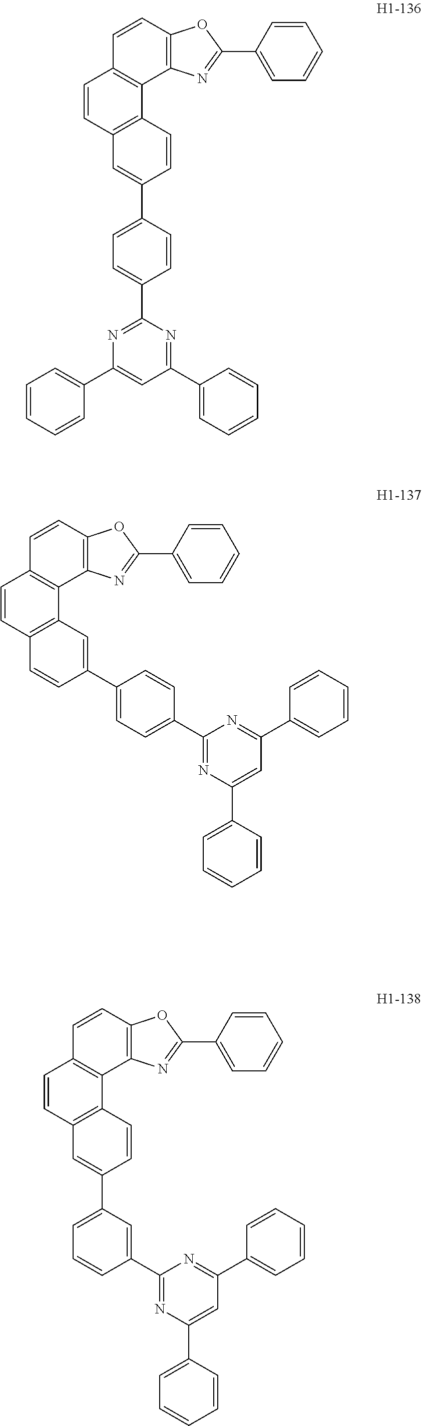 [SMILES: c1ccc(-c2cc(-c3ccccc3)nc(-c3ccc(-c4ccc5c(ccc6ccc7oc(-c8ccccc8)nc7c65)c4)cc3)n2)cc1 ; c1ccc(-c2cc(-c3ccccc3)nc(-c3ccc(-c4ccc5ccc6ccc7oc(-c8ccccc8)nc7c6c5c4)cc3)n2)cc1 ; c1ccc(-c2cc(-c3ccccc3)nc(-c3cccc(-c4ccc5c(ccc6ccc7oc(-c8ccccc8)nc7c65)c4)c3)n2)cc1]

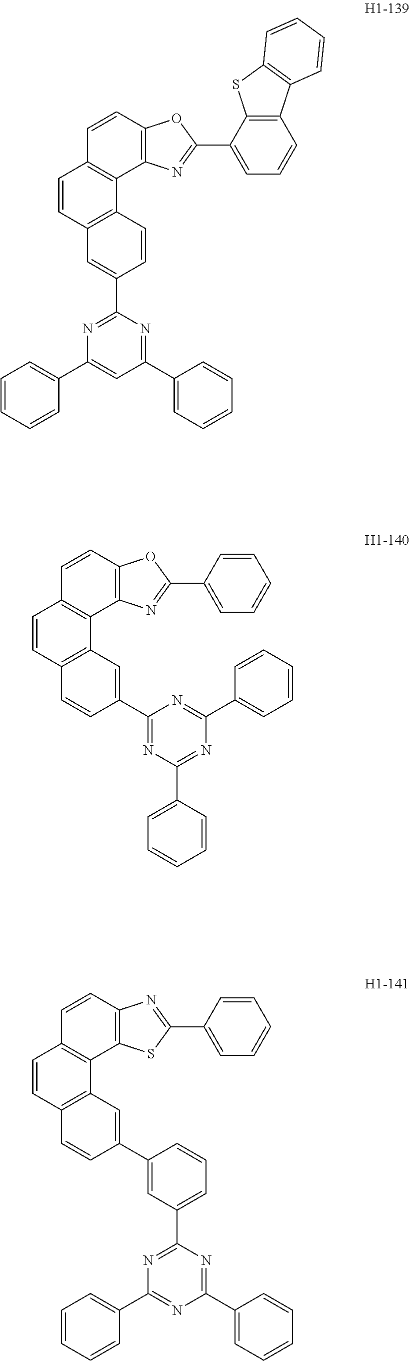 [SMILES: c1ccc(-c2cc(-c3ccccc3)nc(-c3ccc4c(ccc5ccc6oc(-c7cccc8c7sc7ccccc78)nc6c54)c3)n2)cc1 ; c1ccc(-c2nc(-c3ccccc3)nc(-c3ccc4ccc5ccc6oc(-c7ccccc7)nc6c5c4c3)n2)cc1 ; c1ccc(-c2nc(-c3ccccc3)nc(-c3cccc(-c4ccc5ccc6ccc7nc(-c8ccccc8)sc7c6c5c4)c3)n2)cc1]

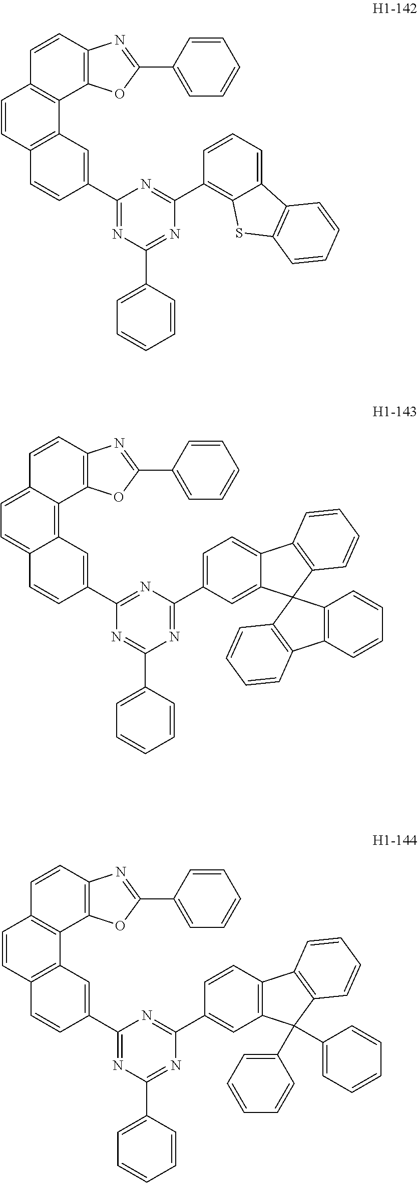 [SMILES: c1ccc(-c2nc(-c3ccc4ccc5ccc6nc(-c7ccccc7)oc6c5c4c3)nc(-c3cccc4c3sc3ccccc34)n2)cc1 ; c1ccc(-c2nc(-c3ccc4c(c3)C3(c5ccccc5-c5ccccc53)c3ccccc3-4)nc(-c3ccc4ccc5ccc6nc(-c7ccccc7)oc6c5c4c3)n2)cc1 ; c1ccc(-c2nc(-c3ccc4c(c3)C(c3ccccc3)(c3ccccc3)c3ccccc3-4)nc(-c3ccc4ccc5ccc6nc(-c7ccccc7)oc6c5c4c3)n2)cc1]

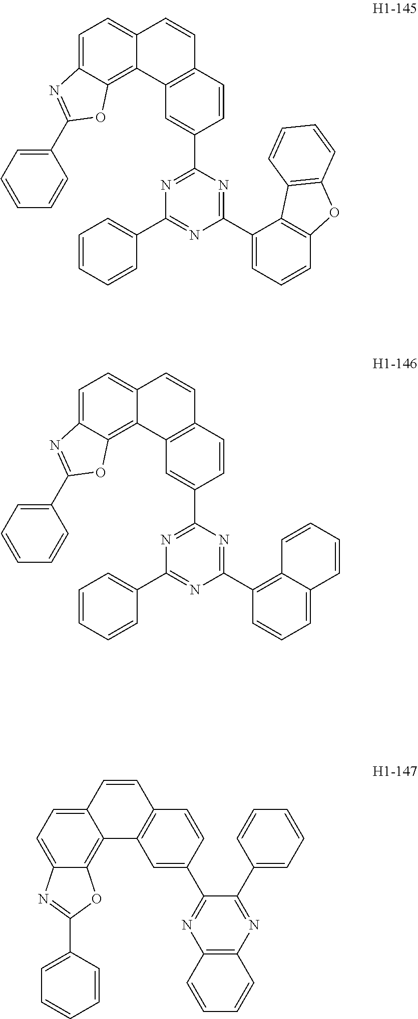 [SMILES: c1ccc(-c2nc(-c3ccc4ccc5ccc6nc(-c7ccccc7)oc6c5c4c3)nc(-c3cccc4oc5ccccc5c34)n2)cc1 ; c1ccc(-c2nc(-c3ccc4ccc5ccc6nc(-c7ccccc7)oc6c5c4c3)nc(-c3cccc4ccccc34)n2)cc1 ; c1ccc(-c2nc3ccc4ccc5ccc(-c6nc7ccccc7nc6-c6ccccc6)cc5c4c3o2)cc1]

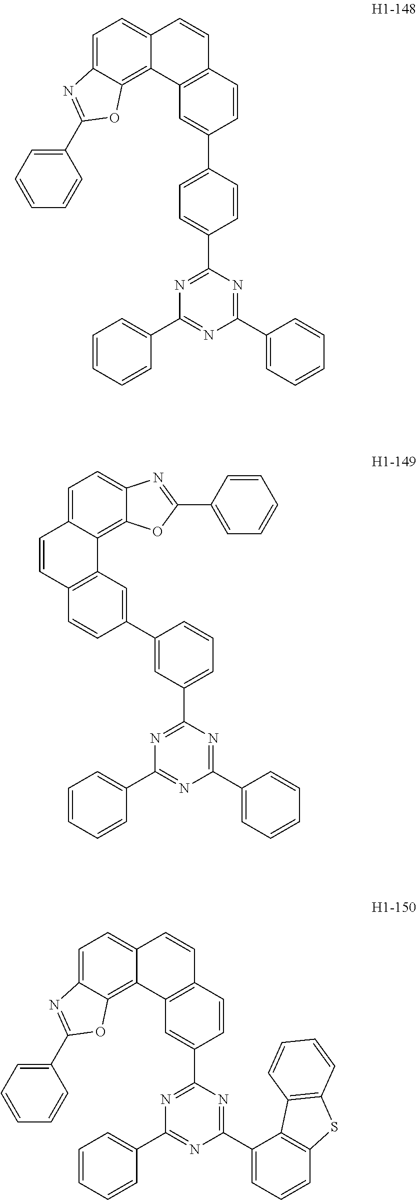 [SMILES: c1ccc(-c2nc(-c3ccccc3)nc(-c3ccc(-c4ccc5ccc6ccc7nc(-c8ccccc8)oc7c6c5c4)cc3)n2)cc1 ; c1ccc(-c2nc(-c3ccccc3)nc(-c3cccc(-c4ccc5ccc6ccc7nc(-c8ccccc8)oc7c6c5c4)c3)n2)cc1 ; c1ccc(-c2nc(-c3ccc4ccc5ccc6nc(-c7ccccc7)oc6c5c4c3)nc(-c3cccc4sc5ccccc5c34)n2)cc1]

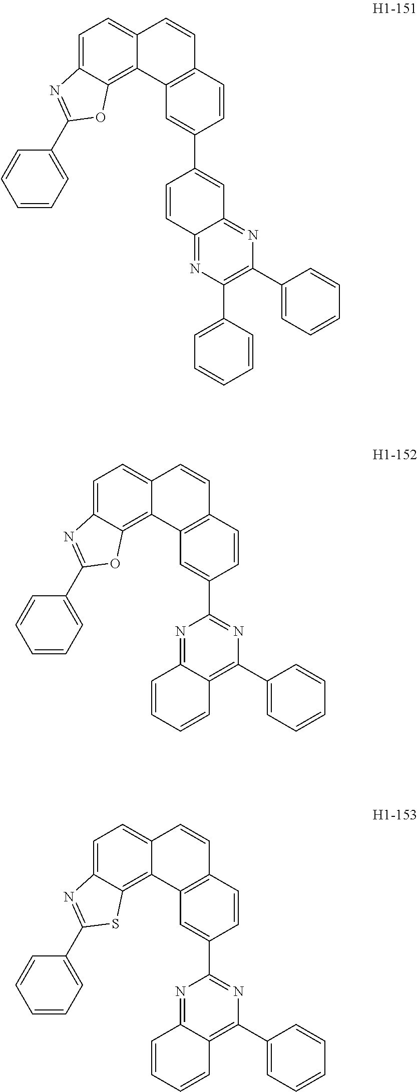 [SMILES: c1ccc(-c2nc3ccc4ccc5ccc(-c6ccc7nc(-c8ccccc8)c(-c8ccccc8)nc7c6)cc5c4c3o2)cc1 ; c1ccc(-c2nc3ccc4ccc5ccc(-c6nc(-c7ccccc7)c7ccccc7n6)cc5c4c3o2)cc1 ; c1ccc(-c2nc3ccc4ccc5ccc(-c6nc(-c7ccccc7)c7ccccc7n6)cc5c4c3s2)cc1]

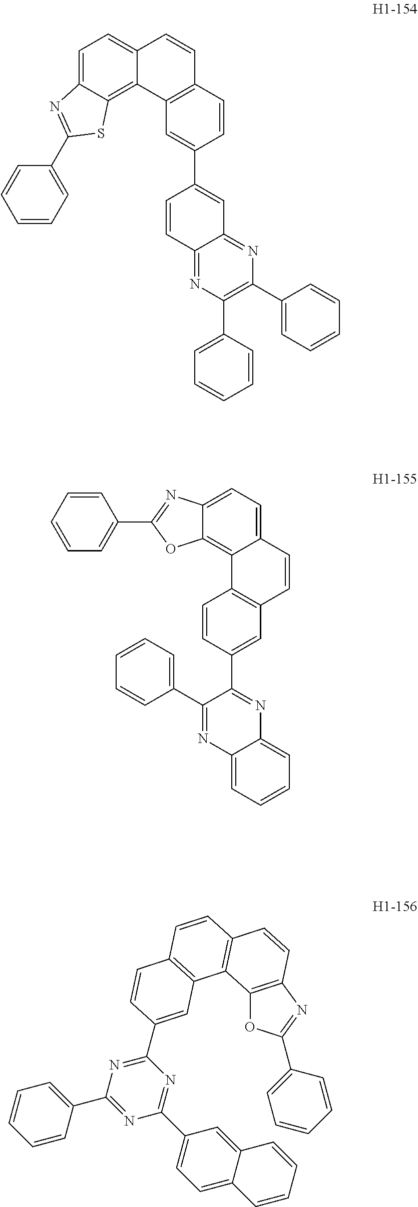 [SMILES: c1ccc(-c2nc3ccc4ccc5ccc(-c6ccc7nc(-c8ccccc8)c(-c8ccccc8)nc7c6)cc5c4c3s2)cc1 ; c1ccc(-c2nc3ccc4ccc5cc(-c6nc7ccccc7nc6-c6ccccc6)ccc5c4c3o2)cc1 ; c1ccc(-c2nc(-c3ccc4ccccc4c3)nc(-c3ccc4ccc5ccc6nc(-c7ccccc7)oc6c5c4c3)n2)cc1]

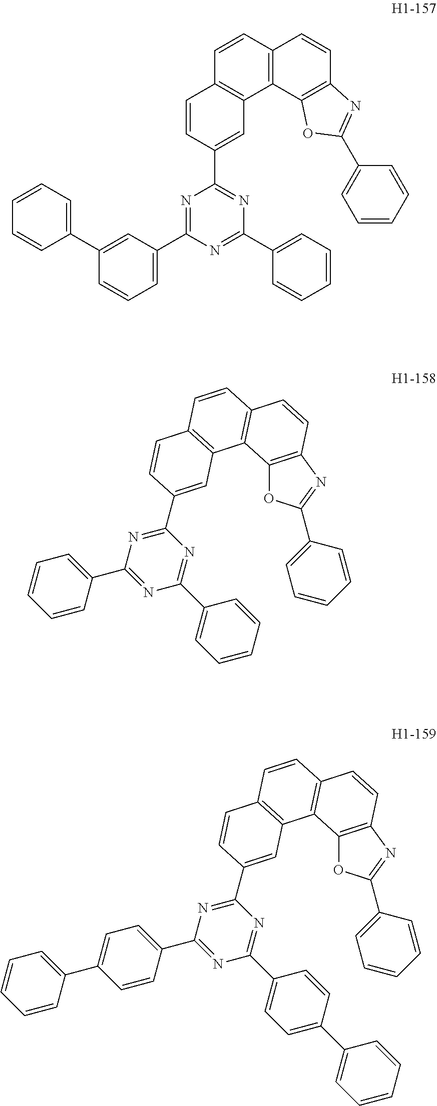 [SMILES: c1ccc(-c2cccc(-c3nc(-c4ccccc4)nc(-c4ccc5ccc6ccc7nc(-c8ccccc8)oc7c6c5c4)n3)c2)cc1 ; c1ccc(-c2nc(-c3ccccc3)nc(-c3ccc4ccc5ccc6nc(-c7ccccc7)oc6c5c4c3)n2)cc1 ; c1ccc(-c2ccc(-c3nc(-c4ccc(-c5ccccc5)cc4)nc(-c4ccc5ccc6ccc7nc(-c8ccccc8)oc7c6c5c4)n3)cc2)cc1]

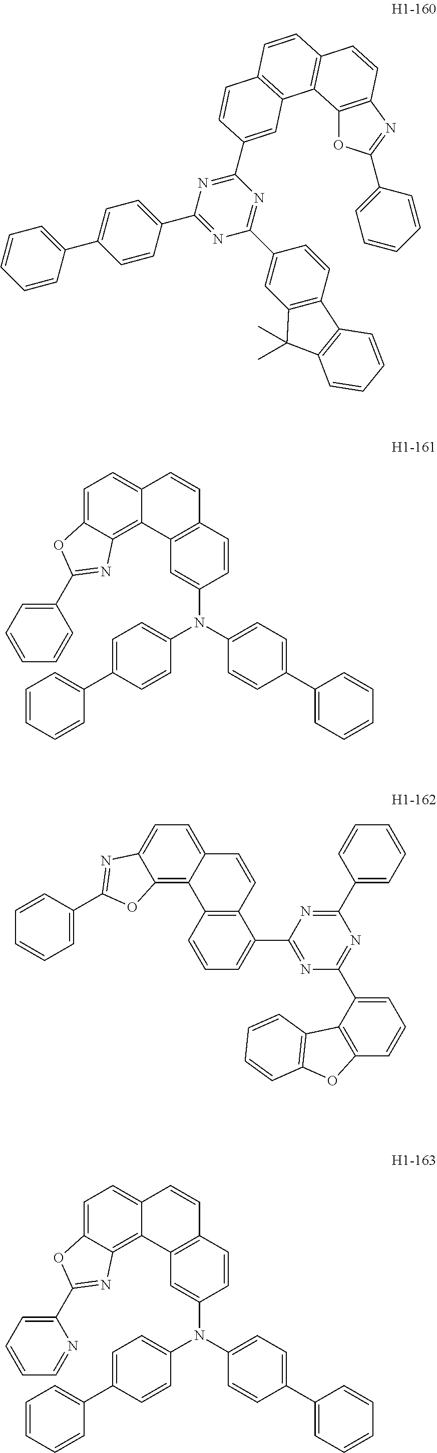 [SMILES: CC1(C)c2ccccc2-c2ccc(-c3nc(-c4ccc(-c5ccccc5)cc4)nc(-c4ccc5ccc6ccc7nc(-c8ccccc8)oc7c6c5c4)n3)cc21 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2nc(-c3cccc4c3ccc3ccc5nc(-c6ccccc6)oc5c34)nc(-c3cccc4oc5ccccc5c34)n2)cc1 ; c1ccc(-c2ccc(N(c3ccc(-c4ccccc4)cc3)c3ccc4ccc5ccc6oc(-c7ccccn7)nc6c5c4c3)cc2)cc1]

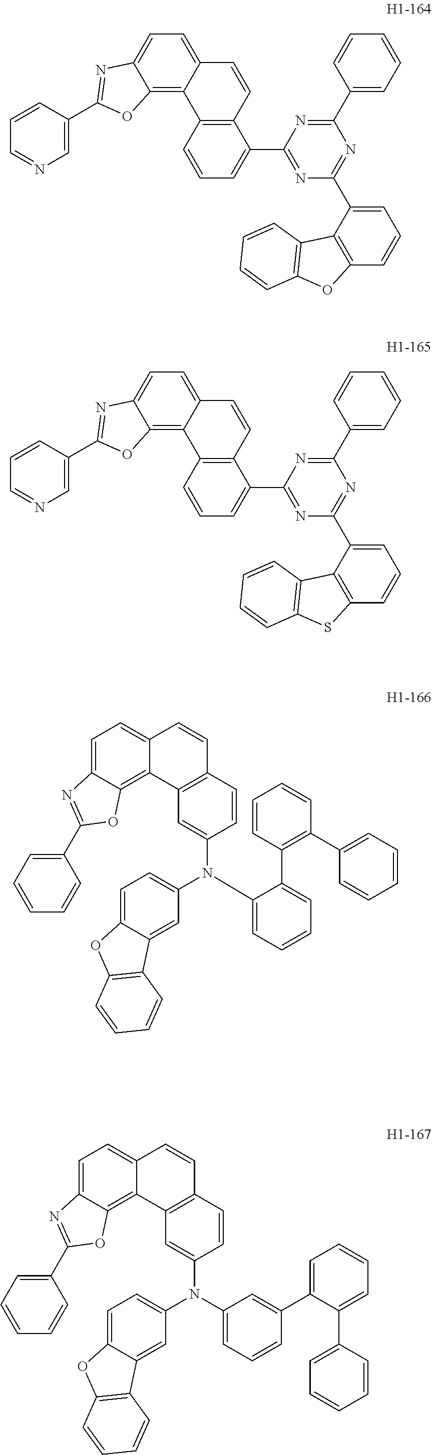 [SMILES: c1ccc(-c2nc(-c3cccc4c3ccc3ccc5nc(-c6cccnc6)oc5c34)nc(-c3cccc4oc5ccccc5c34)n2)cc1 ; c1ccc(-c2nc(-c3cccc4c3ccc3ccc5nc(-c6cccnc6)oc5c34)nc(-c3cccc4sc5ccccc5c34)n2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

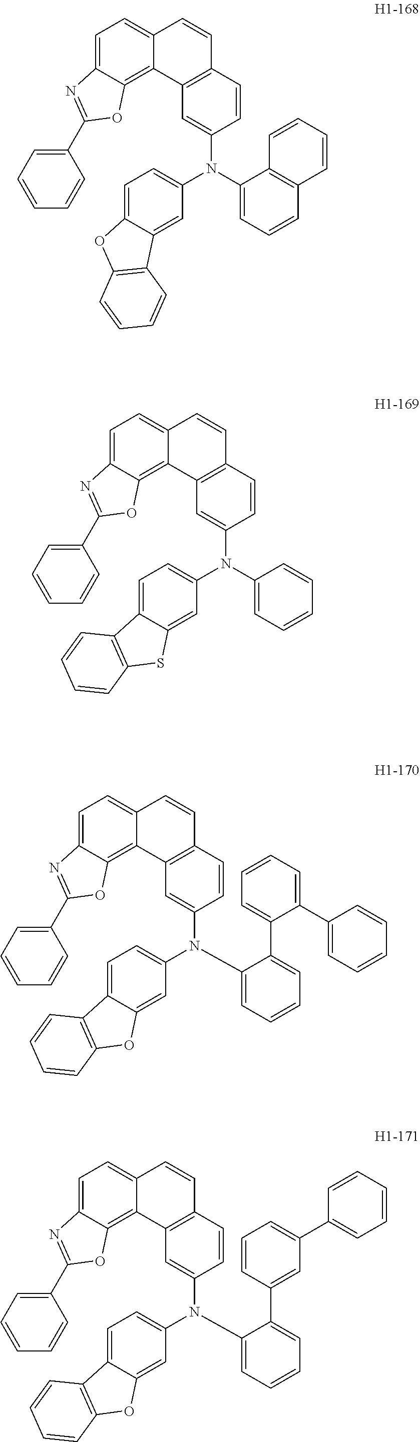 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccccc6)c6ccc7c(c6)sc6ccccc67)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

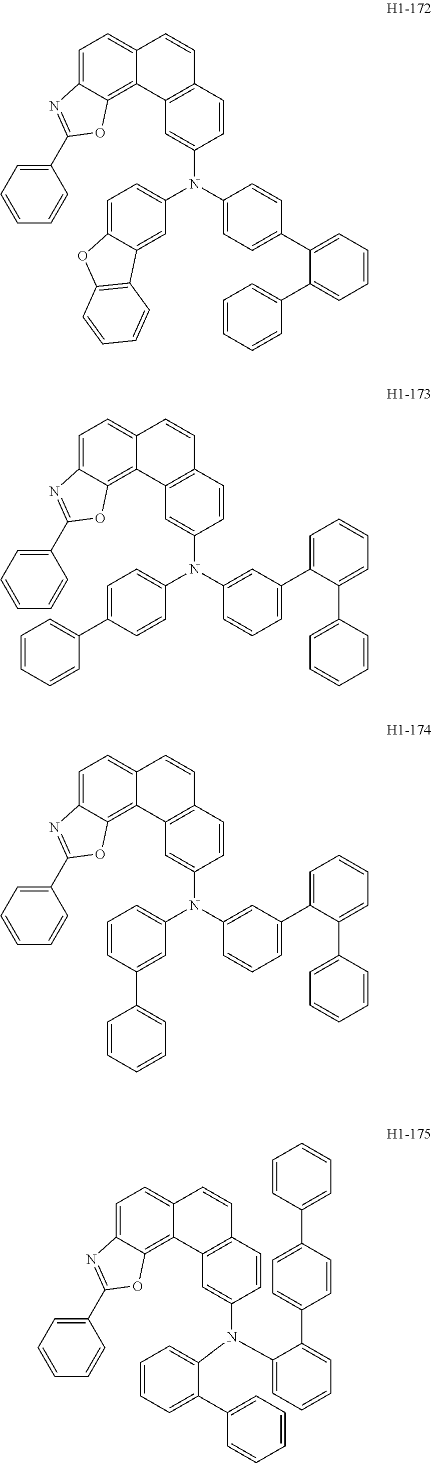 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2ccc(-c3ccccc3N(c3ccc4ccc5ccc6nc(-c7ccccc7)oc6c5c4c3)c3ccccc3-c3ccccc3)cc2)cc1]

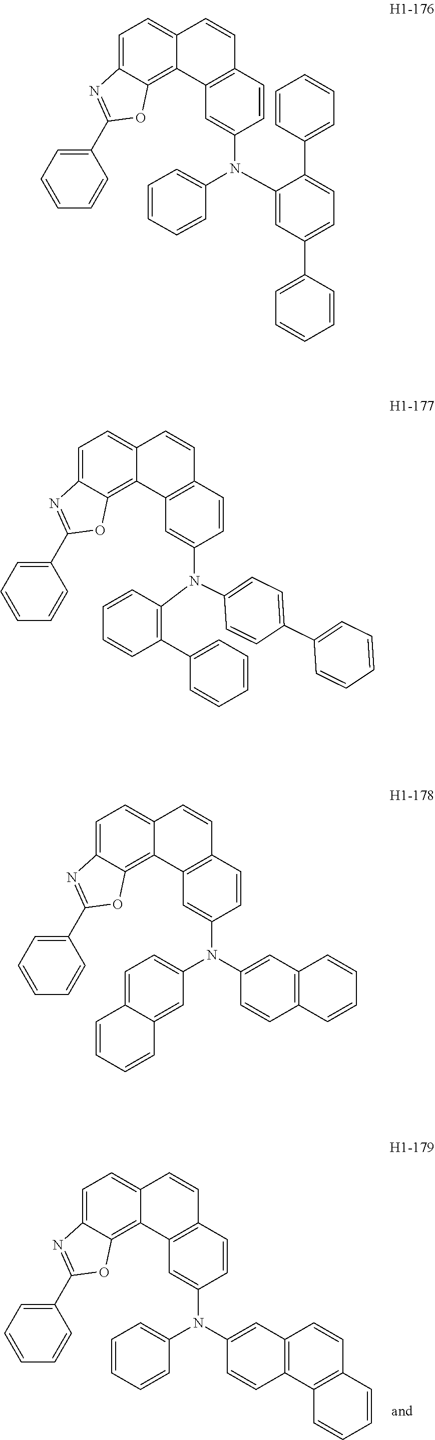 [SMILES: c1ccc(-c2ccc(-c3ccccc3)c(N(c3ccccc3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccc7ccccc7c6)c6ccc7ccccc7c6)cc5c4c3o2)cc1 ; c1ccc(-c2nc3ccc4ccc5ccc(N(c6ccccc6)c6ccc7c(ccc8ccccc87)c6)cc5c4c3o2)cc1]

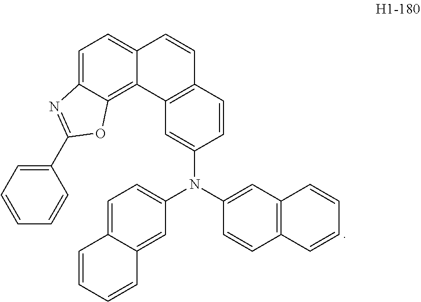 [SMILES: c1ccc(-c2nc3ccc4ccc5ccc(N(c6ccc7ccccc7c6)c6ccc7ccccc7c6)cc5c4c3o2)cc1]

7. The plurality of host materials according to claim 1, wherein the compound represented by the formula 2 is selected from the following compounds:

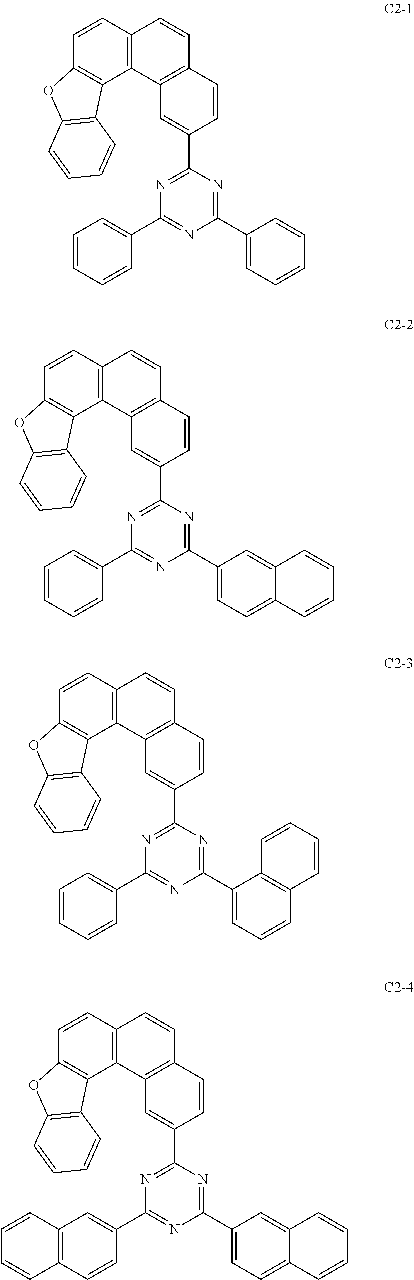 [SMILES: c1ccc(-c2nc(-c3ccccc3)nc(-c3ccc4ccc5ccc6oc7ccccc7c6c5c4c3)n2)cc1 ; c1ccc(-c2nc(-c3ccc4ccccc4c3)nc(-c3ccc4ccc5ccc6oc7ccccc7c6c5c4c3)n2)cc1 ; c1ccc(-c2nc(-c3ccc4ccc5ccc6oc7ccccc7c6c5c4c3)nc(-c3cccc4ccccc34)n2)cc1 ; c1ccc2cc(-c3nc(-c4ccc5ccccc5c4)nc(-c4ccc5ccc6ccc7oc8ccccc8c7c6c5c4)n3)ccc2c1]

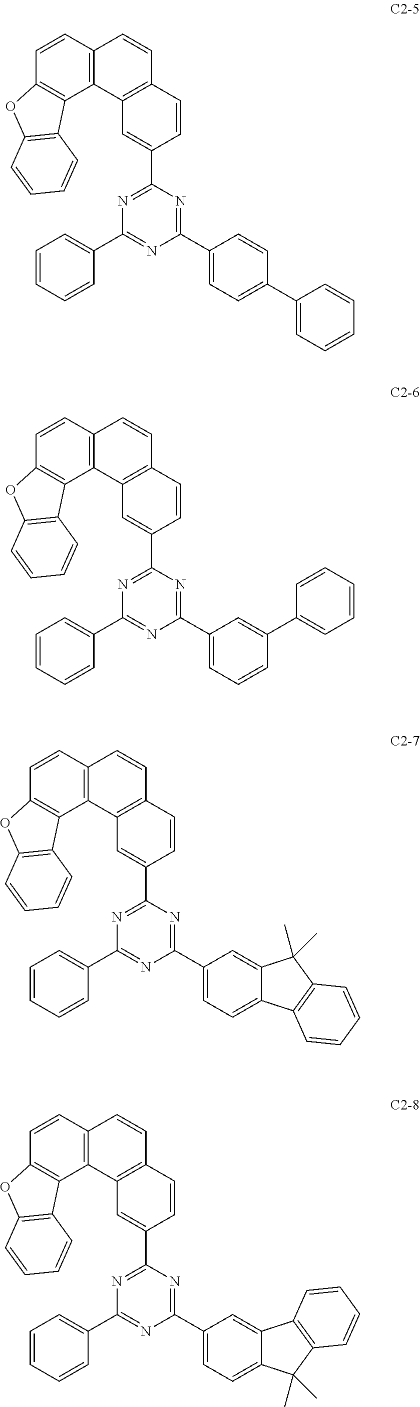 [SMILES: c1ccc(-c2ccc(-c3nc(-c4ccccc4)nc(-c4ccc5ccc6ccc7oc8ccccc8c7c6c5c4)n3)cc2)cc1 ; c1ccc(-c2cccc(-c3nc(-c4ccccc4)nc(-c4ccc5ccc6ccc7oc8ccccc8c7c6c5c4)n3)c2)cc1 ; CC1(C)c2ccccc2-c2ccc(-c3nc(-c4ccccc4)nc(-c4ccc5ccc6ccc7oc8ccccc8c7c6c5c4)n3)cc21 ; CC1(C)c2ccccc2-c2cc(-c3nc(-c4ccccc4)nc(-c4ccc5ccc6ccc7oc8ccccc8c7c6c5c4)n3)ccc21]

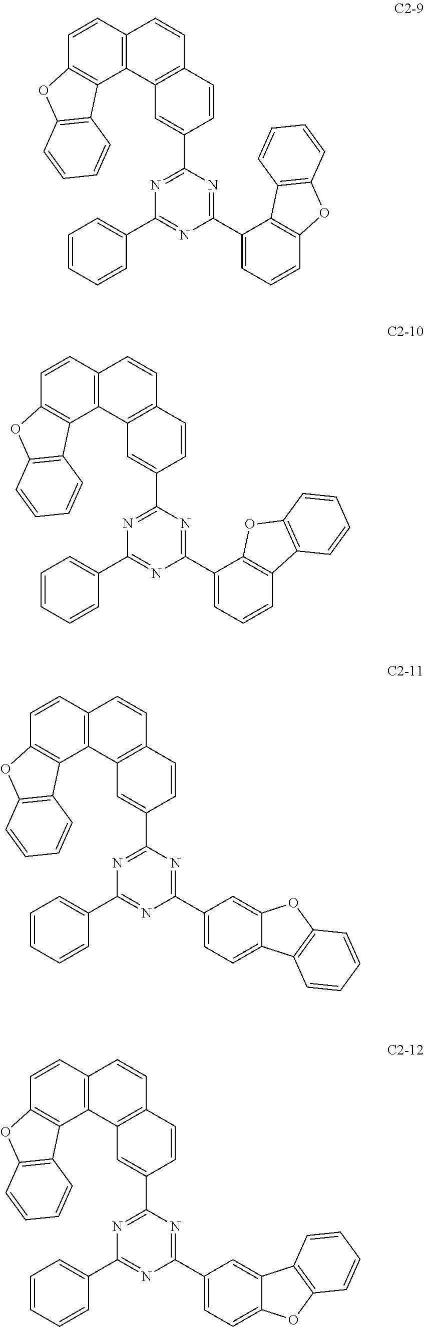 [SMILES: c1ccc(-c2nc(-c3ccc4ccc5ccc6oc7ccccc7c6c5c4c3)nc(-c3cccc4oc5ccccc5c34)n2)cc1 ; c1ccc(-c2nc(-c3ccc4ccc5ccc6oc7ccccc7c6c5c4c3)nc(-c3cccc4c3oc3ccccc34)n2)cc1 ; c1ccc(-c2nc(-c3ccc4c(c3)oc3ccccc34)nc(-c3ccc4ccc5ccc6oc7ccccc7c6c5c4c3)n2)cc1 ; c1ccc(-c2nc(-c3ccc4oc5ccccc5c4c3)nc(-c3ccc4ccc5ccc6oc7ccccc7c6c5c4c3)n2)cc1]

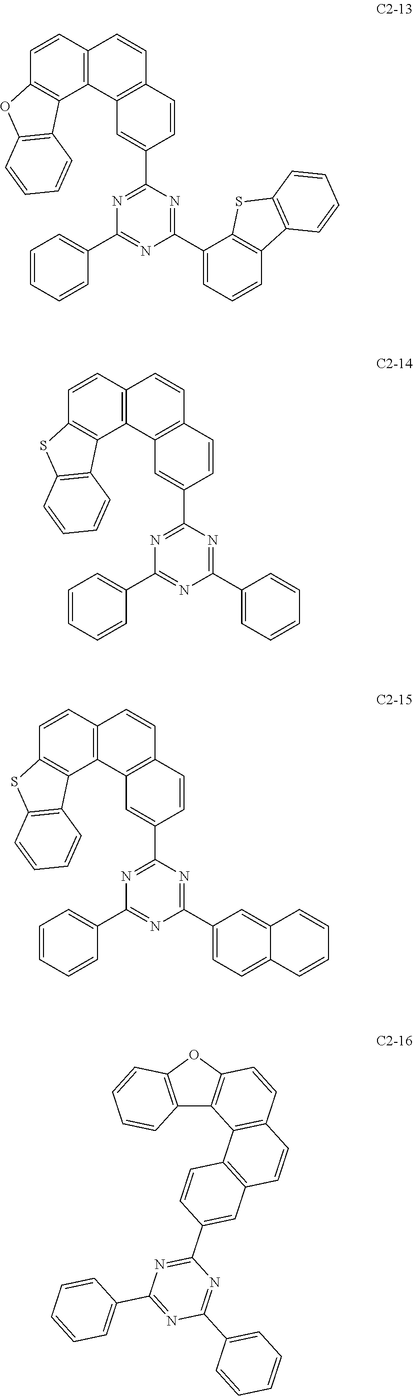 [SMILES: c1ccc(-c2nc(-c3ccc4ccc5ccc6oc7ccccc7c6c5c4c3)nc(-c3cccc4c3sc3ccccc34)n2)cc1 ; c1ccc(-c2nc(-c3ccccc3)nc(-c3ccc4ccc5ccc6sc7ccccc7c6c5c4c3)n2)cc1 ; c1ccc(-c2nc(-c3ccc4ccccc4c3)nc(-c3ccc4ccc5ccc6sc7ccccc7c6c5c4c3)n2)cc1 ; c1ccc(-c2nc(-c3ccccc3)nc(-c3ccc4c(ccc5ccc6oc7ccccc7c6c54)c3)n2)cc1]

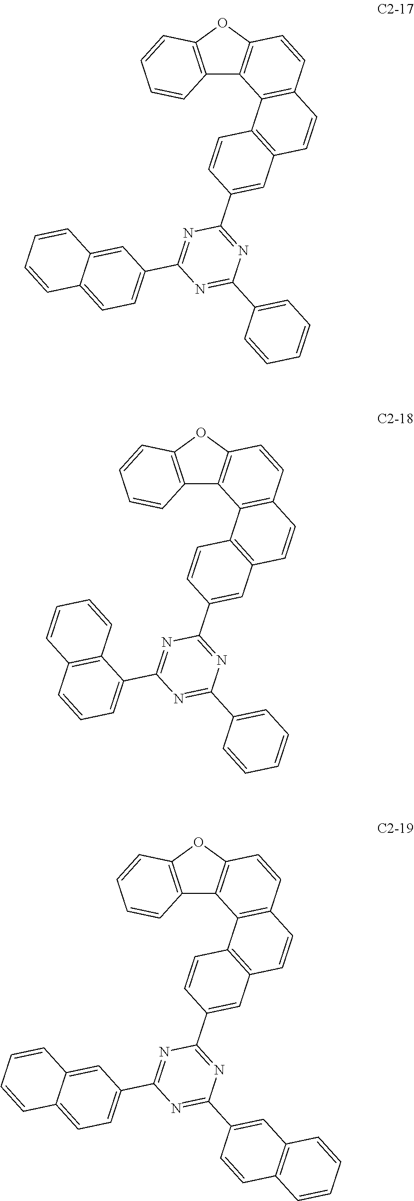 [SMILES: c1ccc(-c2nc(-c3ccc4ccccc4c3)nc(-c3ccc4c(ccc5ccc6oc7ccccc7c6c54)c3)n2)cc1 ; c1ccc(-c2nc(-c3ccc4c(ccc5ccc6oc7ccccc7c6c54)c3)nc(-c3cccc4ccccc34)n2)cc1 ; c1ccc2cc(-c3nc(-c4ccc5ccccc5c4)nc(-c4ccc5c(ccc6ccc7oc8ccccc8c7c65)c4)n3)ccc2c1]

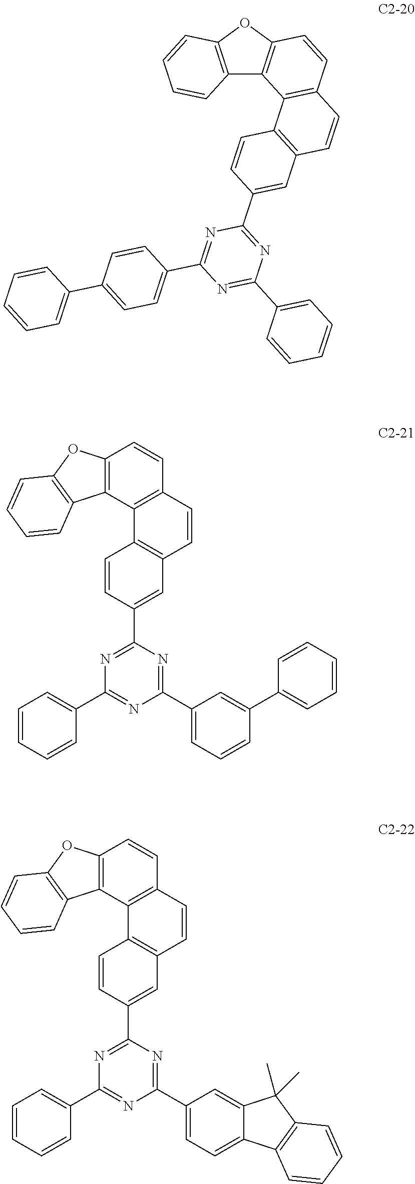 [SMILES: c1ccc(-c2ccc(-c3nc(-c4ccccc4)nc(-c4ccc5c(ccc6ccc7oc8ccccc8c7c65)c4)n3)cc2)cc1 ; c1ccc(-c2cccc(-c3nc(-c4ccccc4)nc(-c4ccc5c(ccc6ccc7oc8ccccc8c7c65)c4)n3)c2)cc1 ; CC1(C)c2ccccc2-c2ccc(-c3nc(-c4ccccc4)nc(-c4ccc5c(ccc6ccc7oc8ccccc8c7c65)c4)n3)cc21]

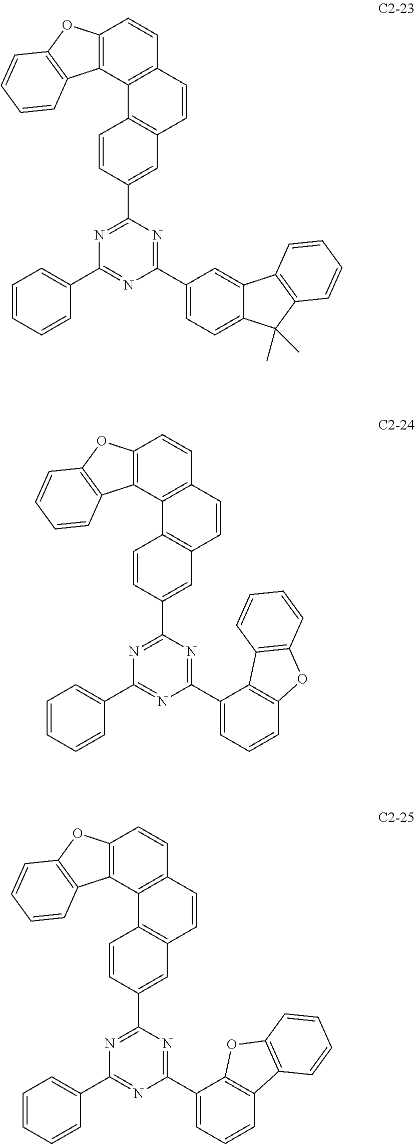 [SMILES: CC1(C)c2ccccc2-c2cc(-c3nc(-c4ccccc4)nc(-c4ccc5c(ccc6ccc7oc8ccccc8c7c65)c4)n3)ccc21 ; c1ccc(-c2nc(-c3ccc4c(ccc5ccc6oc7ccccc7c6c54)c3)nc(-c3cccc4oc5ccccc5c34)n2)cc1 ; c1ccc(-c2nc(-c3ccc4c(ccc5ccc6oc7ccccc7c6c54)c3)nc(-c3cccc4c3oc3ccccc34)n2)cc1]

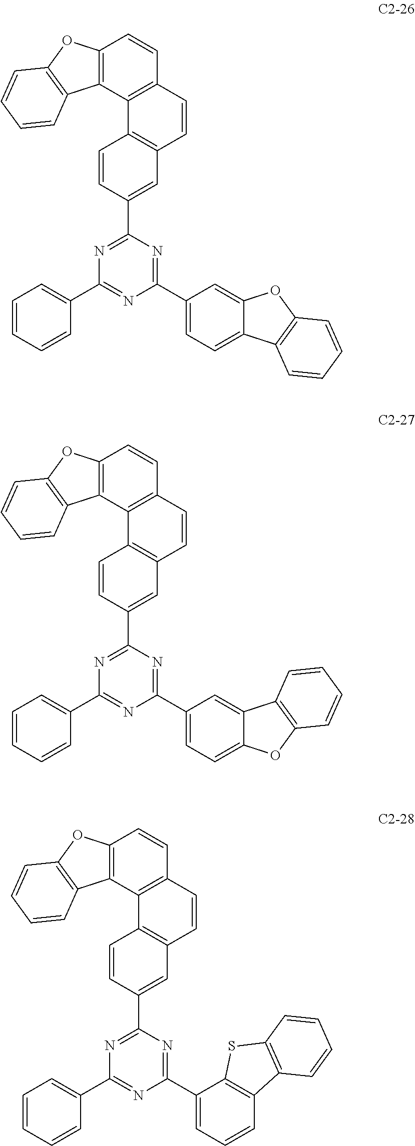 [SMILES: c1ccc(-c2nc(-c3ccc4c(ccc5ccc6oc7ccccc7c6c54)c3)nc(-c3ccc4c(c3)oc3ccccc34)n2)cc1 ; c1ccc(-c2nc(-c3ccc4c(ccc5ccc6oc7ccccc7c6c54)c3)nc(-c3ccc4oc5ccccc5c4c3)n2)cc1 ; c1ccc(-c2nc(-c3ccc4c(ccc5ccc6oc7ccccc7c6c54)c3)nc(-c3cccc4c3sc3ccccc34)n2)cc1]

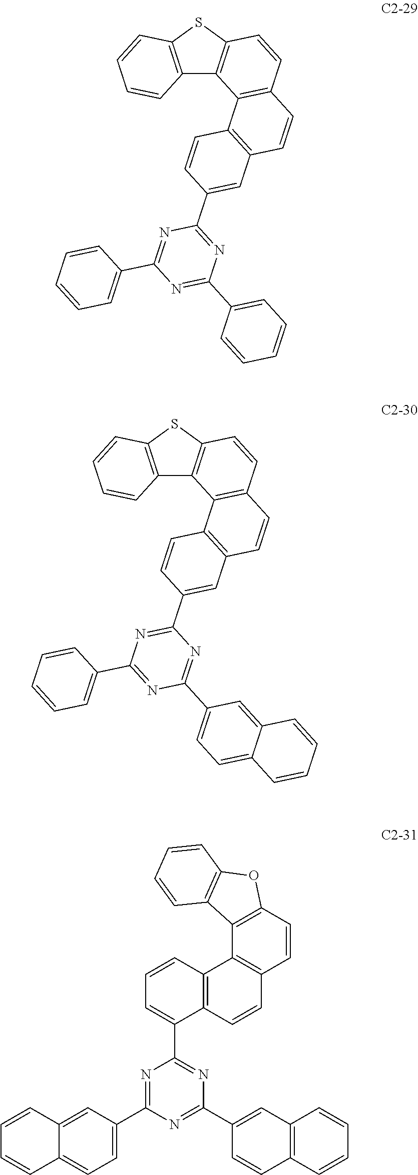 [SMILES: c1ccc(-c2nc(-c3ccccc3)nc(-c3ccc4c(ccc5ccc6sc7ccccc7c6c54)c3)n2)cc1 ; c1ccc(-c2nc(-c3ccc4ccccc4c3)nc(-c3ccc4c(ccc5ccc6sc7ccccc7c6c54)c3)n2)cc1 ; c1ccc2cc(-c3nc(-c4ccc5ccccc5c4)nc(-c4cccc5c4ccc4ccc6oc7ccccc7c6c45)n3)ccc2c1]

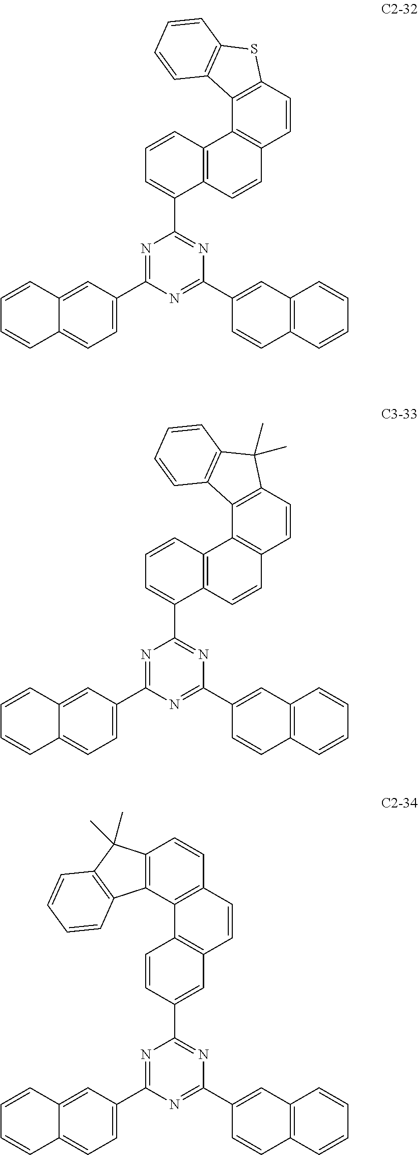 [SMILES: c1ccc2cc(-c3nc(-c4ccc5ccccc5c4)nc(-c4cccc5c4ccc4ccc6sc7ccccc7c6c45)n3)ccc2c1 ; CC1(C)c2ccccc2-c2c1ccc1ccc3c(-c4nc(-c5ccc6ccccc6c5)nc(-c5ccc6ccccc6c5)n4)cccc3c21 ; CC1(C)c2ccccc2-c2c1ccc1ccc3cc(-c4nc(-c5ccc6ccccc6c5)nc(-c5ccc6ccccc6c5)n4)ccc3c21]

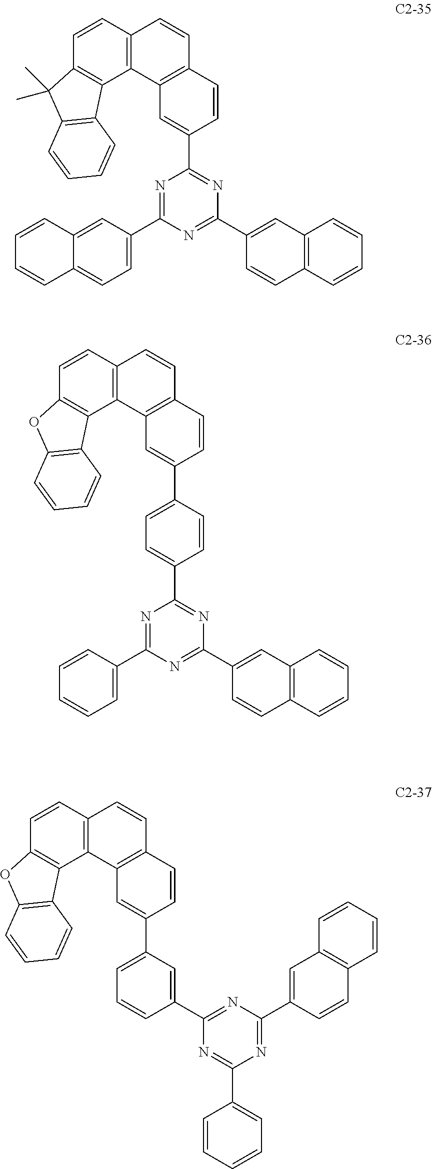 [SMILES: CC1(C)c2ccccc2-c2c1ccc1ccc3ccc(-c4nc(-c5ccc6ccccc6c5)nc(-c5ccc6ccccc6c5)n4)cc3c21 ; c1ccc(-c2nc(-c3ccc(-c4ccc5ccc6ccc7oc8ccccc8c7c6c5c4)cc3)nc(-c3ccc4ccccc4c3)n2)cc1 ; c1ccc(-c2nc(-c3cccc(-c4ccc5ccc6ccc7oc8ccccc8c7c6c5c4)c3)nc(-c3ccc4ccccc4c3)n2)cc1]

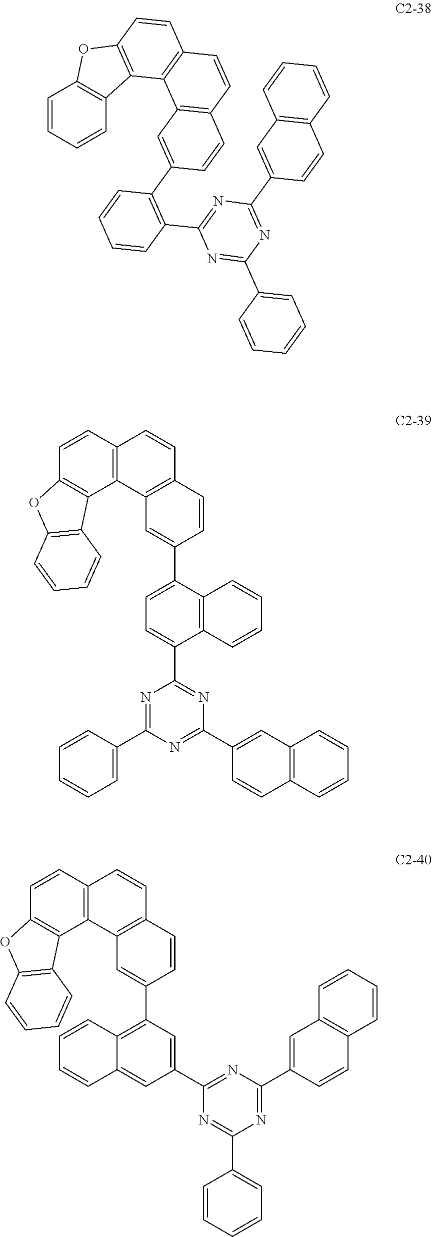 [SMILES: c1ccc(-c2nc(-c3ccc4ccccc4c3)nc(-c3ccccc3-c3ccc4ccc5ccc6oc7ccccc7c6c5c4c3)n2)cc1 ; c1ccc(-c2nc(-c3ccc4ccccc4c3)nc(-c3ccc(-c4ccc5ccc6ccc7oc8ccccc8c7c6c5c4)c4ccccc34)n2)cc1 ; c1ccc(-c2nc(-c3ccc4ccccc4c3)nc(-c3cc(-c4ccc5ccc6ccc7oc8ccccc8c7c6c5c4)c4ccccc4c3)n2)cc1]

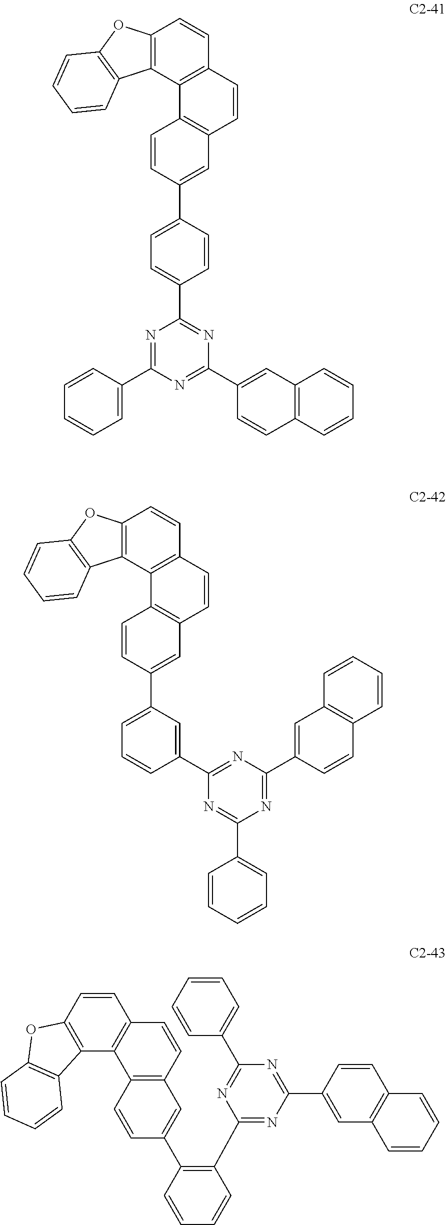 [SMILES: c1ccc(-c2nc(-c3ccc(-c4ccc5c(ccc6ccc7oc8ccccc8c7c65)c4)cc3)nc(-c3ccc4ccccc4c3)n2)cc1 ; c1ccc(-c2nc(-c3cccc(-c4ccc5c(ccc6ccc7oc8ccccc8c7c65)c4)c3)nc(-c3ccc4ccccc4c3)n2)cc1 ; c1ccc(-c2nc(-c3ccc4ccccc4c3)nc(-c3ccccc3-c3ccc4c(ccc5ccc6oc7ccccc7c6c54)c3)n2)cc1]

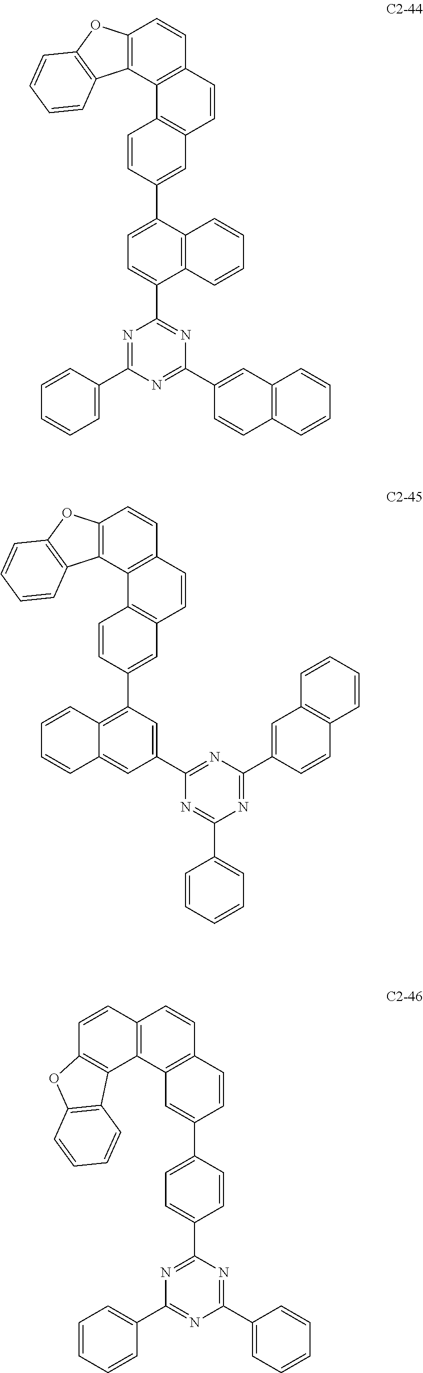 [SMILES: c1ccc(-c2nc(-c3ccc4ccccc4c3)nc(-c3ccc(-c4ccc5c(ccc6ccc7oc8ccccc8c7c65)c4)c4ccccc34)n2)cc1 ; c1ccc(-c2nc(-c3ccc4ccccc4c3)nc(-c3cc(-c4ccc5c(ccc6ccc7oc8ccccc8c7c65)c4)c4ccccc4c3)n2)cc1 ; c1ccc(-c2nc(-c3ccccc3)nc(-c3ccc(-c4ccc5ccc6ccc7oc8ccccc8c7c6c5c4)cc3)n2)cc1]

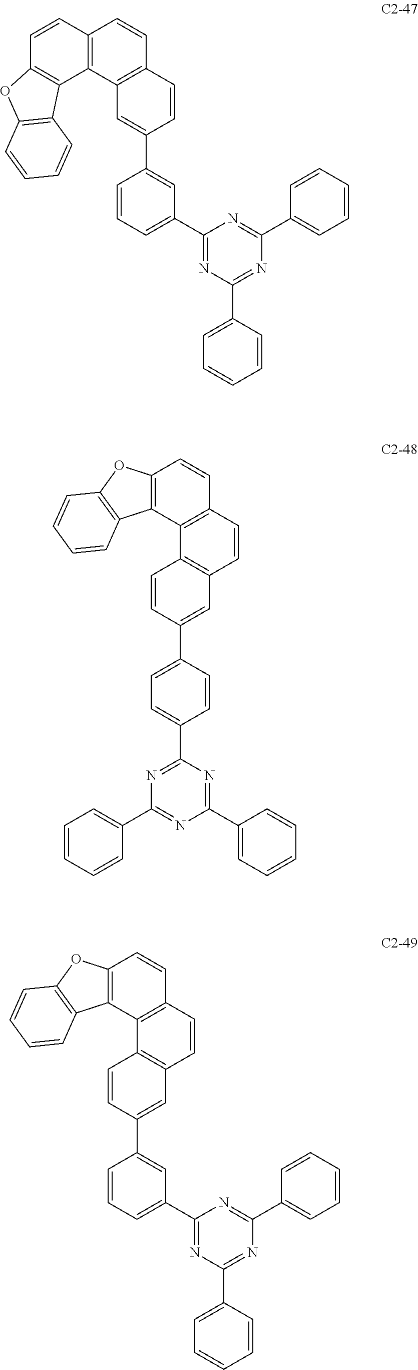 [SMILES: c1ccc(-c2nc(-c3ccccc3)nc(-c3cccc(-c4ccc5ccc6ccc7oc8ccccc8c7c6c5c4)c3)n2)cc1 ; c1ccc(-c2nc(-c3ccccc3)nc(-c3ccc(-c4ccc5c(ccc6ccc7oc8ccccc8c7c65)c4)cc3)n2)cc1 ; c1ccc(-c2nc(-c3ccccc3)nc(-c3cccc(-c4ccc5c(ccc6ccc7oc8ccccc8c7c65)c4)c3)n2)cc1]

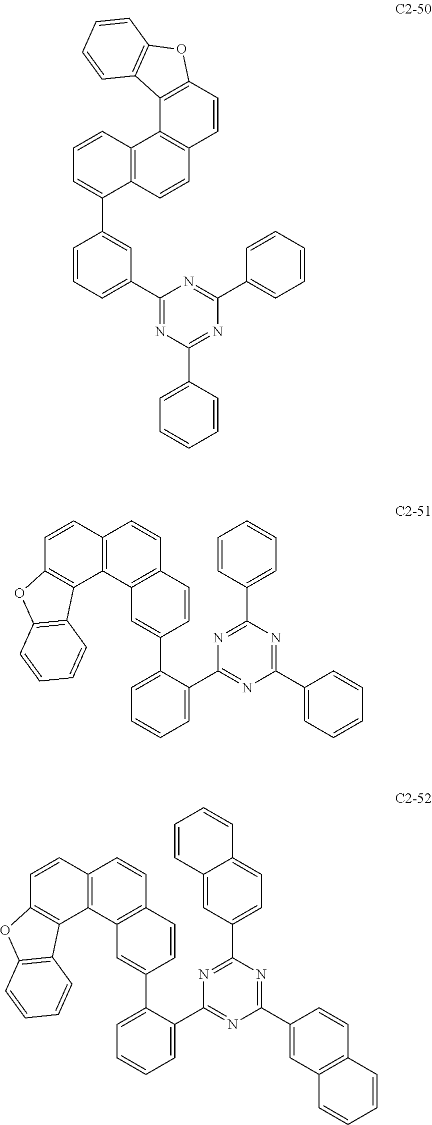 [SMILES: c1ccc(-c2nc(-c3ccccc3)nc(-c3cccc(-c4cccc5c4ccc4ccc6oc7ccccc7c6c45)c3)n2)cc1 ; c1ccc(-c2nc(-c3ccccc3)nc(-c3ccccc3-c3ccc4ccc5ccc6oc7ccccc7c6c5c4c3)n2)cc1 ; c1ccc(-c2nc(-c3ccc4ccccc4c3)nc(-c3ccc4ccccc4c3)n2)c(-c2ccc3ccc4ccc5oc6ccccc6c5c4c3c2)c1]

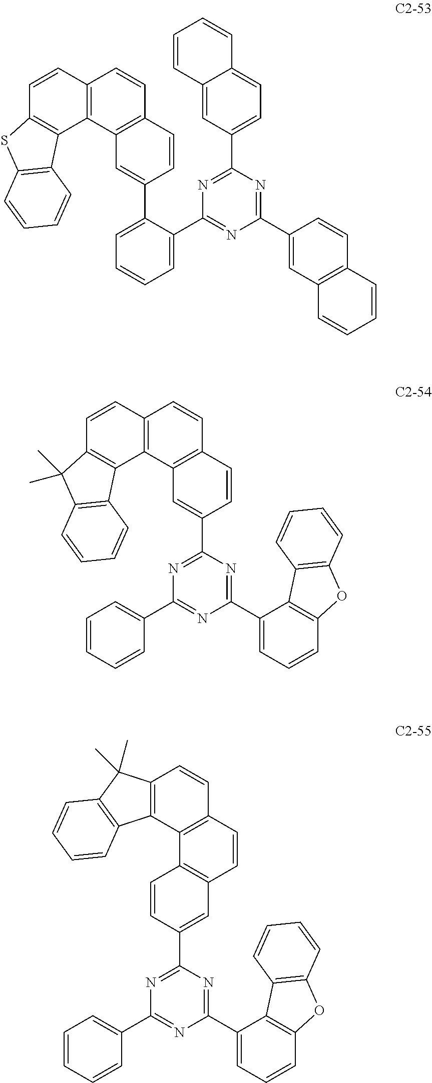 [SMILES: c1ccc(-c2nc(-c3ccc4ccccc4c3)nc(-c3ccc4ccccc4c3)n2)c(-c2ccc3ccc4ccc5sc6ccccc6c5c4c3c2)c1 ; CC1(C)c2ccccc2-c2c1ccc1ccc3ccc(-c4nc(-c5ccccc5)nc(-c5cccc6oc7ccccc7c56)n4)cc3c21 ; CC1(C)c2ccccc2-c2c1ccc1ccc3cc(-c4nc(-c5ccccc5)nc(-c5cccc6oc7ccccc7c56)n4)ccc3c21]

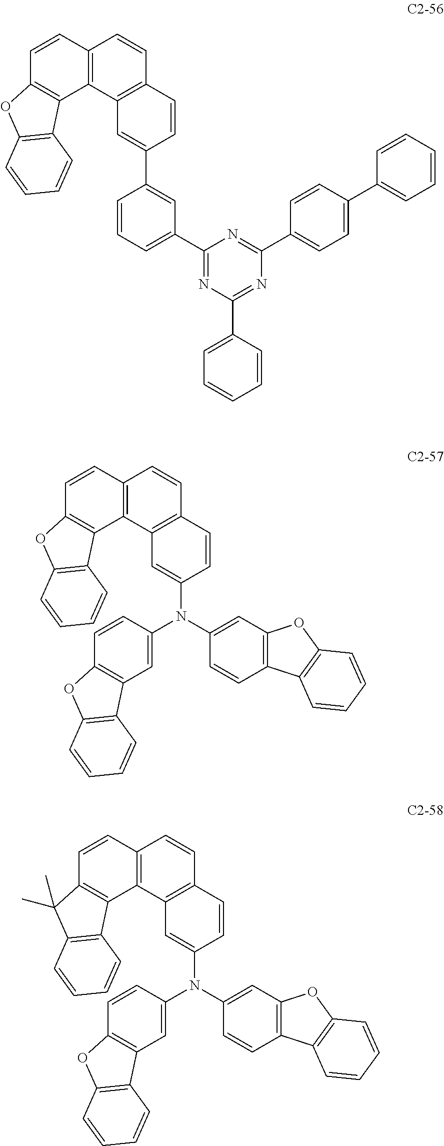 [SMILES: c1ccc(-c2ccc(-c3nc(-c4ccccc4)nc(-c4cccc(-c5ccc6ccc7ccc8oc9ccccc9c8c7c6c5)c4)n3)cc2)cc1 ; c1ccc2c(c1)oc1cc(N(c3ccc4oc5ccccc5c4c3)c3ccc4ccc5ccc6oc7ccccc7c6c5c4c3)ccc12 ; CC1(C)c2ccccc2-c2c1ccc1ccc3ccc(N(c4ccc5c(c4)oc4ccccc45)c4ccc5oc6ccccc6c5c4)cc3c21]

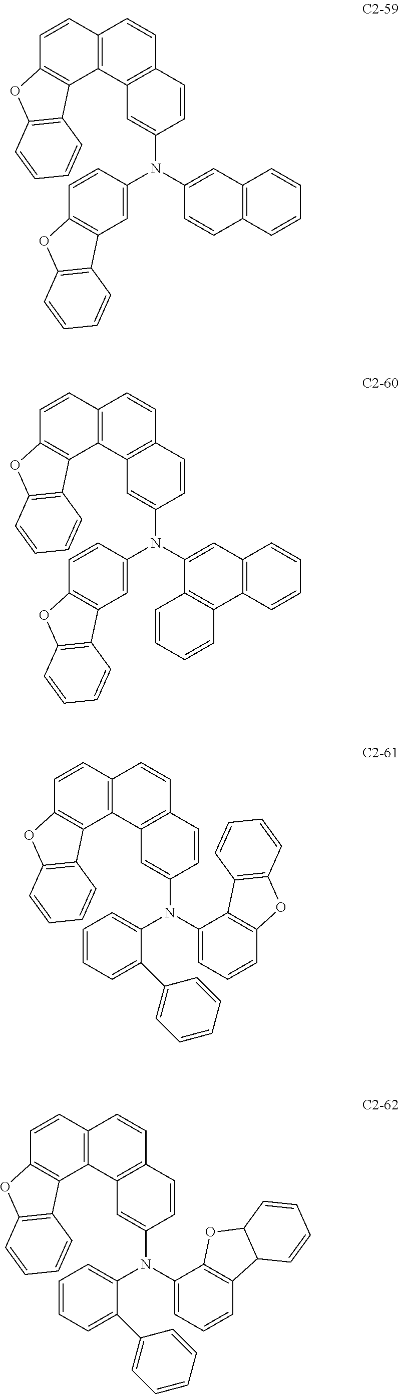 [SMILES: c1ccc2cc(N(c3ccc4oc5ccccc5c4c3)c3ccc4ccc5ccc6oc7ccccc7c6c5c4c3)ccc2c1 ; c1ccc2c(c1)cc(N(c1ccc3oc4ccccc4c3c1)c1ccc3ccc4ccc5oc6ccccc6c5c4c3c1)c1ccccc12 ; c1ccc(-c2ccccc2N(c2ccc3ccc4ccc5oc6ccccc6c5c4c3c2)c2cccc3oc4ccccc4c23)cc1 ; C1=CC2Oc3c(cccc3N(c3ccc4ccc5ccc6oc7ccccc7c6c5c4c3)c3ccccc3-c3ccccc3)C2C=C1]

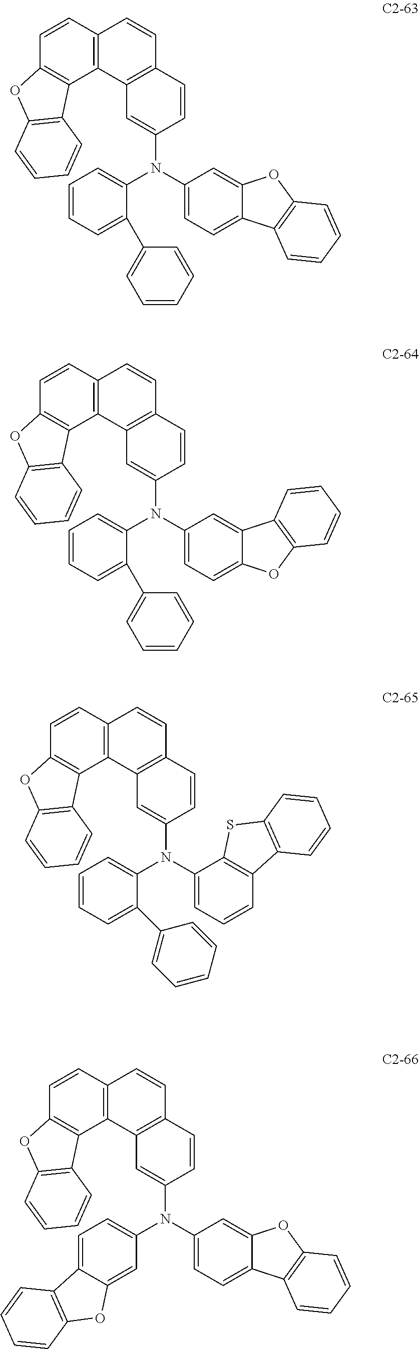 [SMILES: c1ccc(-c2ccccc2N(c2ccc3c(c2)oc2ccccc23)c2ccc3ccc4ccc5oc6ccccc6c5c4c3c2)cc1 ; c1ccc(-c2ccccc2N(c2ccc3oc4ccccc4c3c2)c2ccc3ccc4ccc5oc6ccccc6c5c4c3c2)cc1 ; c1ccc(-c2ccccc2N(c2ccc3ccc4ccc5oc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6oc7ccccc7c6c5c4c3)ccc12]

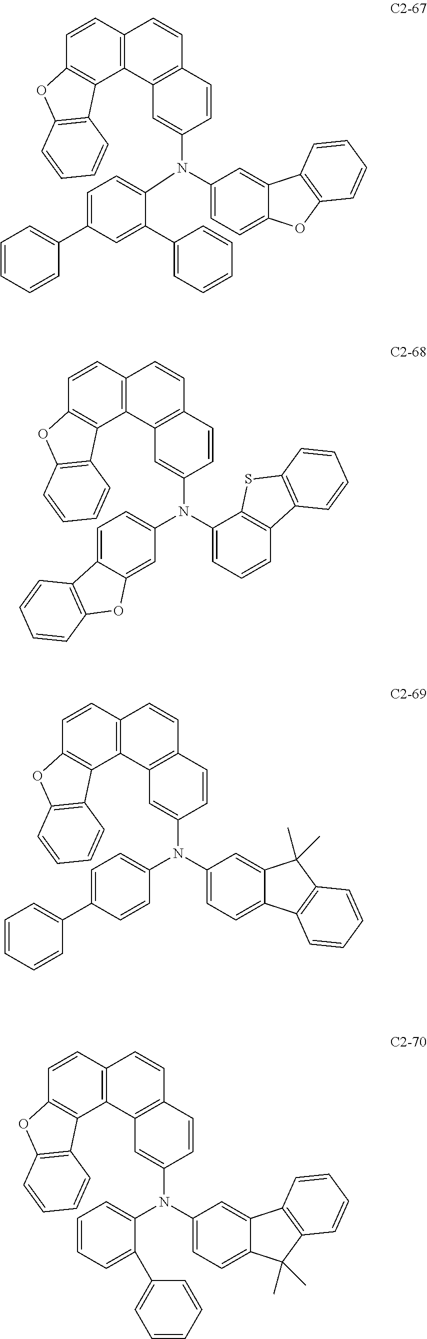 [SMILES: c1ccc(-c2ccc(N(c3ccc4oc5ccccc5c4c3)c3ccc4ccc5ccc6oc7ccccc7c6c5c4c3)c(-c3ccccc3)c2)cc1 ; c1ccc2c(c1)oc1cc(N(c3ccc4ccc5ccc6oc7ccccc7c6c5c4c3)c3cccc4c3sc3ccccc34)ccc12 ; CC1(C)c2ccccc2-c2ccc(N(c3ccc(-c4ccccc4)cc3)c3ccc4ccc5ccc6oc7ccccc7c6c5c4c3)cc21 ; CC1(C)c2ccccc2-c2cc(N(c3ccc4ccc5ccc6oc7ccccc7c6c5c4c3)c3ccccc3-c3ccccc3)ccc21]

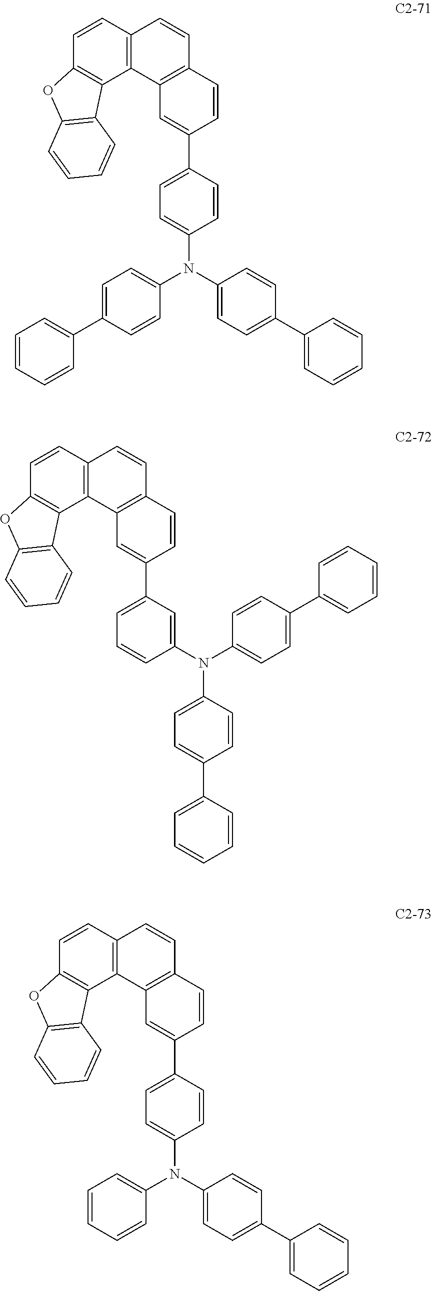 [SMILES: c1ccc(-c2ccc(N(c3ccc(-c4ccccc4)cc3)c3ccc(-c4ccc5ccc6ccc7oc8ccccc8c7c6c5c4)cc3)cc2)cc1 ; c1ccc(-c2ccc(N(c3ccc(-c4ccccc4)cc3)c3cccc(-c4ccc5ccc6ccc7oc8ccccc8c7c6c5c4)c3)cc2)cc1 ; c1ccc(-c2ccc(N(c3ccccc3)c3ccc(-c4ccc5ccc6ccc7oc8ccccc8c7c6c5c4)cc3)cc2)cc1]

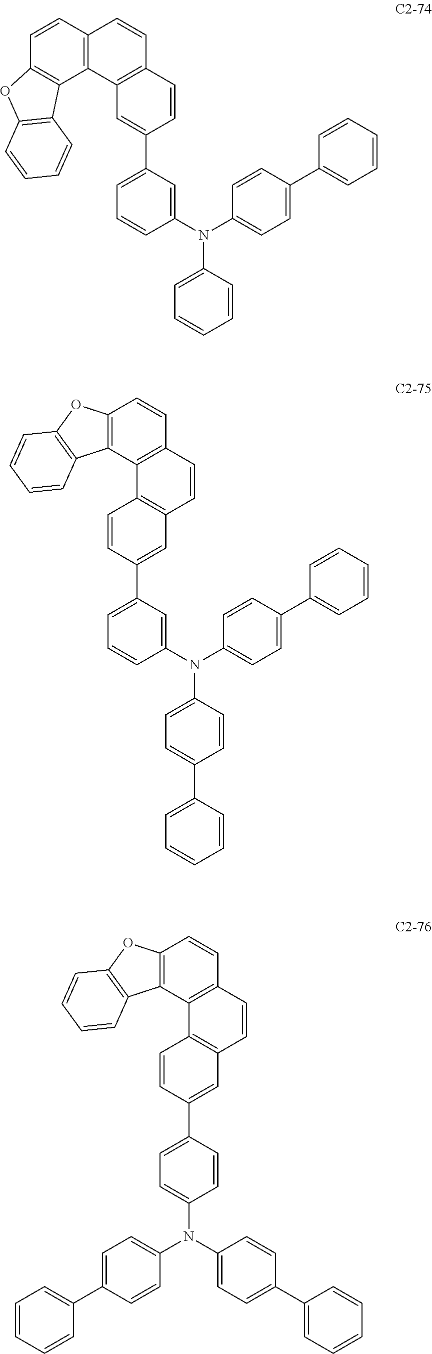 [SMILES: c1ccc(-c2ccc(N(c3ccccc3)c3cccc(-c4ccc5ccc6ccc7oc8ccccc8c7c6c5c4)c3)cc2)cc1 ; c1ccc(-c2ccc(N(c3ccc(-c4ccccc4)cc3)c3cccc(-c4ccc5c(ccc6ccc7oc8ccccc8c7c65)c4)c3)cc2)cc1 ; c1ccc(-c2ccc(N(c3ccc(-c4ccccc4)cc3)c3ccc(-c4ccc5c(ccc6ccc7oc8ccccc8c7c65)c4)cc3)cc2)cc1]

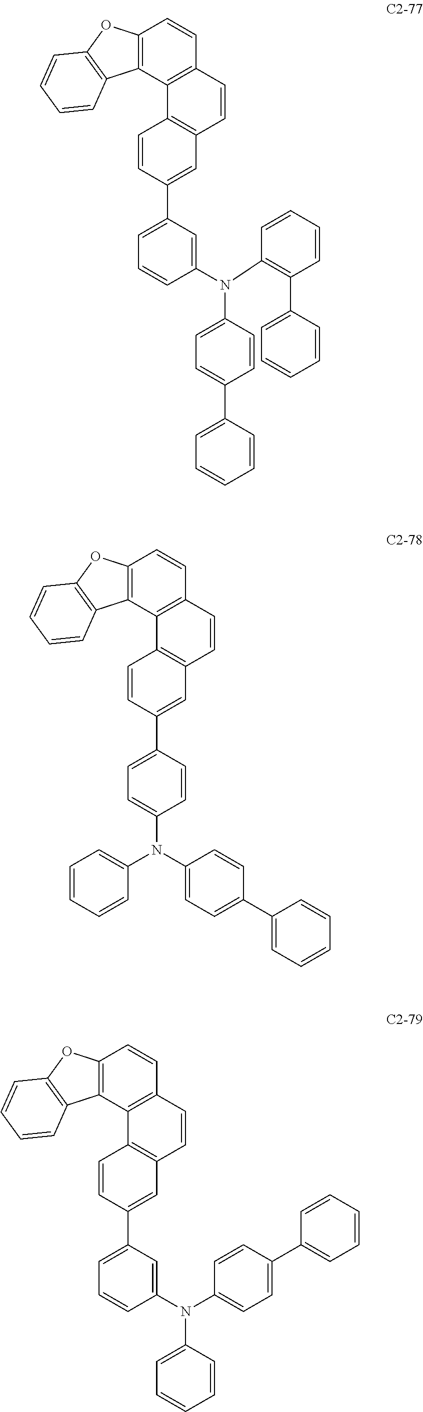 [SMILES: c1ccc(-c2ccc(N(c3cccc(-c4ccc5c(ccc6ccc7oc8ccccc8c7c65)c4)c3)c3ccccc3-c3ccccc3)cc2)cc1 ; c1ccc(-c2ccc(N(c3ccccc3)c3ccc(-c4ccc5c(ccc6ccc7oc8ccccc8c7c65)c4)cc3)cc2)cc1 ; c1ccc(-c2ccc(N(c3ccccc3)c3cccc(-c4ccc5c(ccc6ccc7oc8ccccc8c7c65)c4)c3)cc2)cc1]

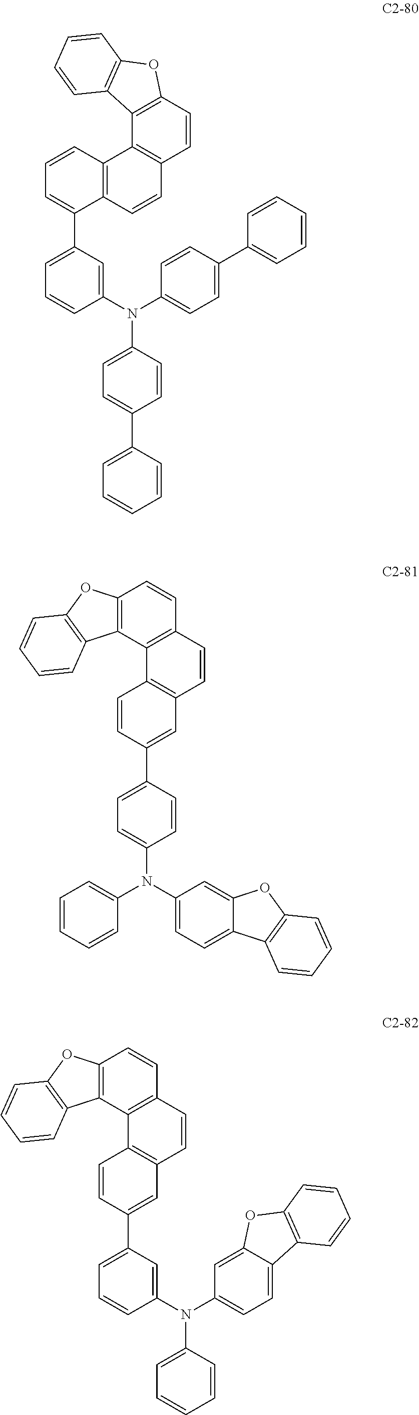 [SMILES: c1ccc(-c2ccc(N(c3ccc(-c4ccccc4)cc3)c3cccc(-c4cccc5c4ccc4ccc6oc7ccccc7c6c45)c3)cc2)cc1 ; c1ccc(N(c2ccc(-c3ccc4c(ccc5ccc6oc7ccccc7c6c54)c3)cc2)c2ccc3c(c2)oc2ccccc23)cc1 ; c1ccc(N(c2cccc(-c3ccc4c(ccc5ccc6oc7ccccc7c6c54)c3)c2)c2ccc3c(c2)oc2ccccc23)cc1]

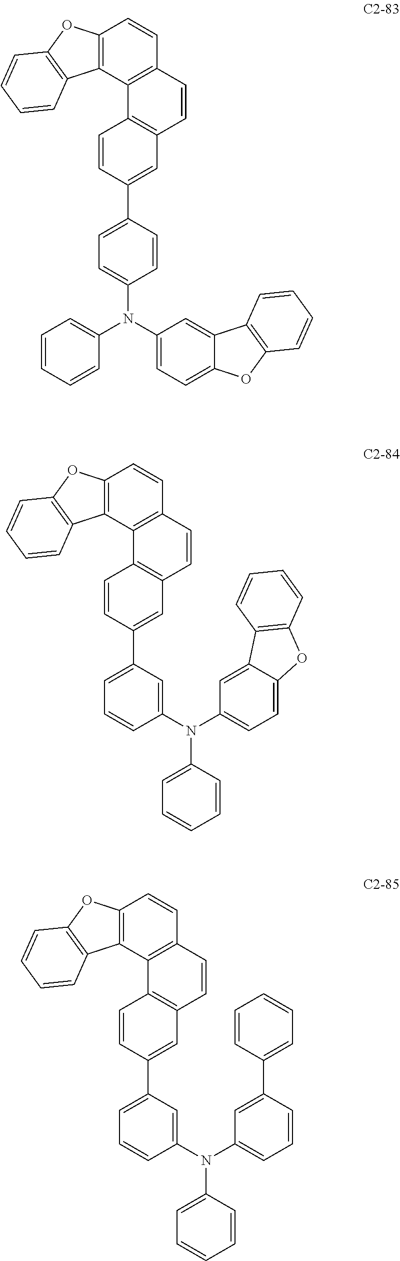 [SMILES: c1ccc(N(c2ccc(-c3ccc4c(ccc5ccc6oc7ccccc7c6c54)c3)cc2)c2ccc3oc4ccccc4c3c2)cc1 ; c1ccc(N(c2cccc(-c3ccc4c(ccc5ccc6oc7ccccc7c6c54)c3)c2)c2ccc3oc4ccccc4c3c2)cc1 ; c1ccc(-c2cccc(N(c3ccccc3)c3cccc(-c4ccc5c(ccc6ccc7oc8ccccc8c7c65)c4)c3)c2)cc1]

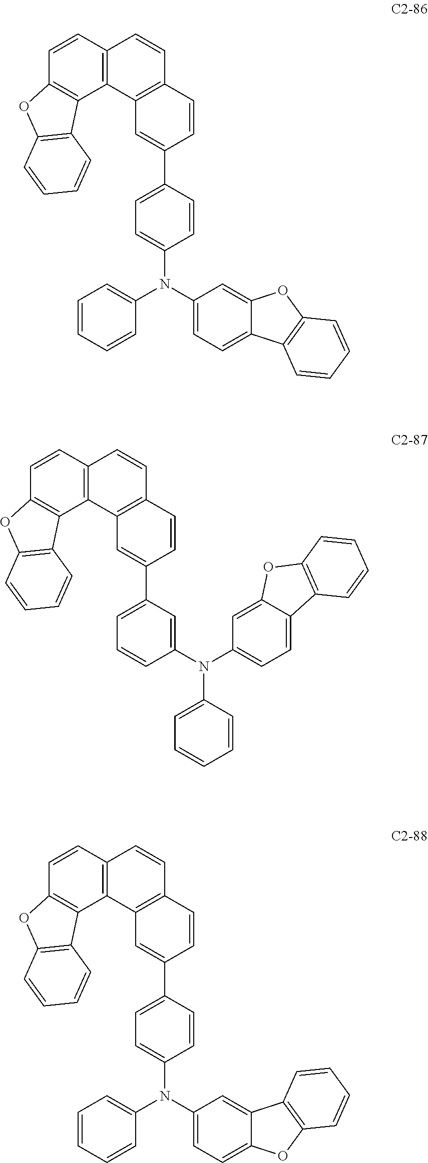 [SMILES: c1ccc(N(c2ccc(-c3ccc4ccc5ccc6oc7ccccc7c6c5c4c3)cc2)c2ccc3c(c2)oc2ccccc23)cc1 ; c1ccc(N(c2cccc(-c3ccc4ccc5ccc6oc7ccccc7c6c5c4c3)c2)c2ccc3c(c2)oc2ccccc23)cc1 ; c1ccc(N(c2ccc(-c3ccc4ccc5ccc6oc7ccccc7c6c5c4c3)cc2)c2ccc3oc4ccccc4c3c2)cc1]

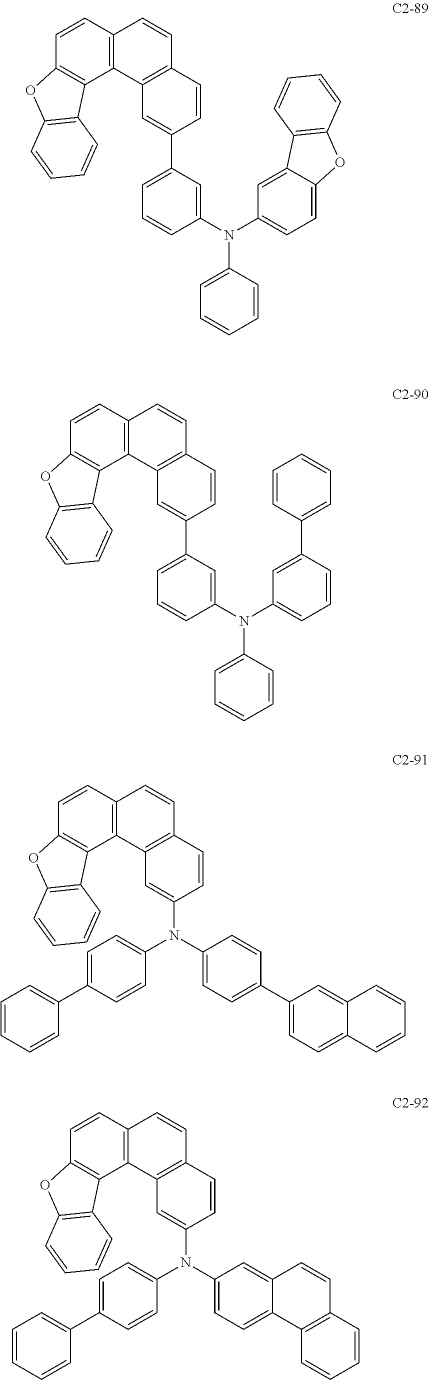 [SMILES: c1ccc(N(c2cccc(-c3ccc4ccc5ccc6oc7ccccc7c6c5c4c3)c2)c2ccc3oc4ccccc4c3c2)cc1 ; c1ccc(-c2cccc(N(c3ccccc3)c3cccc(-c4ccc5ccc6ccc7oc8ccccc8c7c6c5c4)c3)c2)cc1 ; c1ccc(-c2ccc(N(c3ccc(-c4ccc5ccccc5c4)cc3)c3ccc4ccc5ccc6oc7ccccc7c6c5c4c3)cc2)cc1 ; c1ccc(-c2ccc(N(c3ccc4c(ccc5ccccc54)c3)c3ccc4ccc5ccc6oc7ccccc7c6c5c4c3)cc2)cc1]

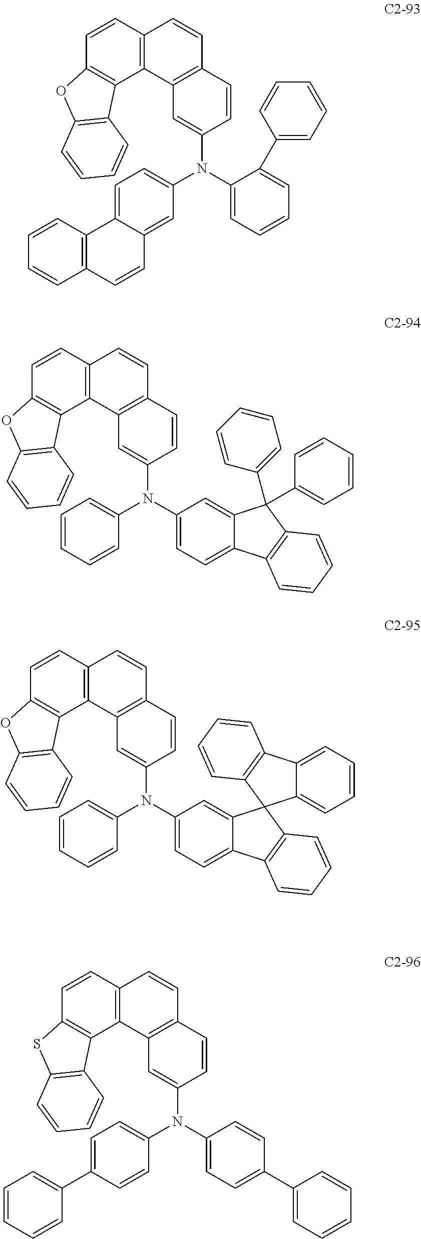 [SMILES: c1ccc(-c2ccccc2N(c2ccc3c(ccc4ccccc43)c2)c2ccc3ccc4ccc5oc6ccccc6c5c4c3c2)cc1 ; c1ccc(N(c2ccc3c(c2)C(c2ccccc2)(c2ccccc2)c2ccccc2-3)c2ccc3ccc4ccc5oc6ccccc6c5c4c3c2)cc1 ; c1ccc(N(c2ccc3c(c2)C2(c4ccccc4-c4ccccc42)c2ccccc2-3)c2ccc3ccc4ccc5oc6ccccc6c5c4c3c2)cc1 ; c1ccc(-c2ccc(N(c3ccc(-c4ccccc4)cc3)c3ccc4ccc5ccc6sc7ccccc7c6c5c4c3)cc2)cc1]

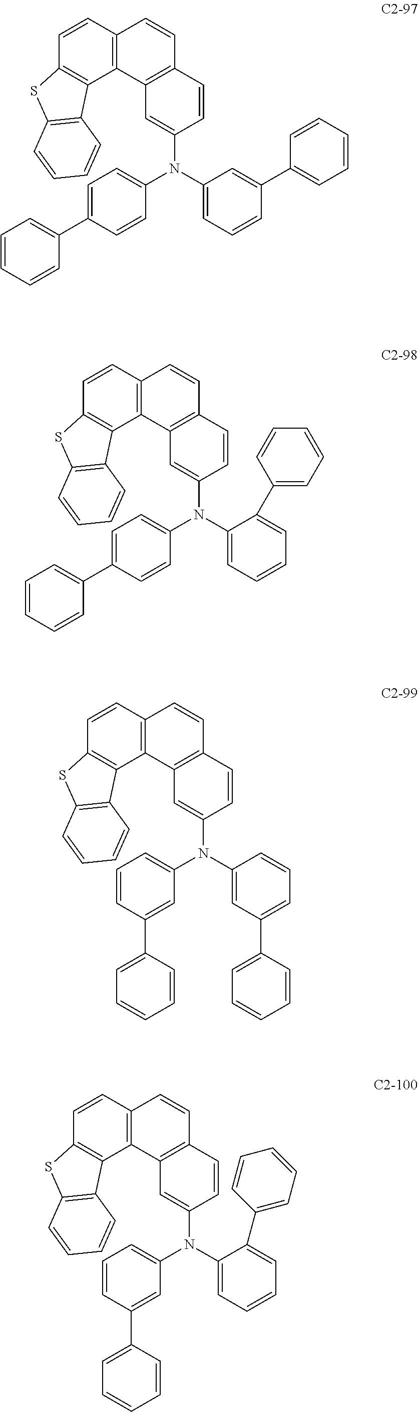 [SMILES: c1ccc(-c2ccc(N(c3cccc(-c4ccccc4)c3)c3ccc4ccc5ccc6sc7ccccc7c6c5c4c3)cc2)cc1 ; c1ccc(-c2ccc(N(c3ccc4ccc5ccc6sc7ccccc7c6c5c4c3)c3ccccc3-c3ccccc3)cc2)cc1 ; c1ccc(-c2cccc(N(c3cccc(-c4ccccc4)c3)c3ccc4ccc5ccc6sc7ccccc7c6c5c4c3)c2)cc1 ; c1ccc(-c2cccc(N(c3ccc4ccc5ccc6sc7ccccc7c6c5c4c3)c3ccccc3-c3ccccc3)c2)cc1]

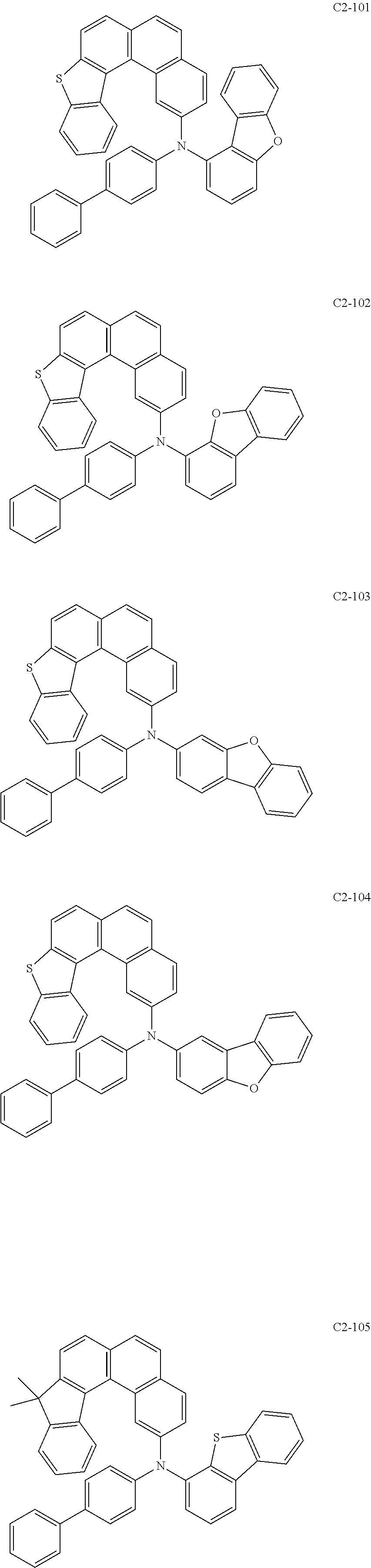 [SMILES: c1ccc(-c2ccc(N(c3ccc4ccc5ccc6sc7ccccc7c6c5c4c3)c3cccc4oc5ccccc5c34)cc2)cc1 ; c1ccc(-c2ccc(N(c3ccc4ccc5ccc6sc7ccccc7c6c5c4c3)c3cccc4c3oc3ccccc34)cc2)cc1 ; c1ccc(-c2ccc(N(c3ccc4c(c3)oc3ccccc34)c3ccc4ccc5ccc6sc7ccccc7c6c5c4c3)cc2)cc1 ; c1ccc(-c2ccc(N(c3ccc4oc5ccccc5c4c3)c3ccc4ccc5ccc6sc7ccccc7c6c5c4c3)cc2)cc1 ; CC1(C)c2ccccc2-c2c1ccc1ccc3ccc(N(c4ccc(-c5ccccc5)cc4)c4cccc5c4sc4ccccc45)cc3c21]

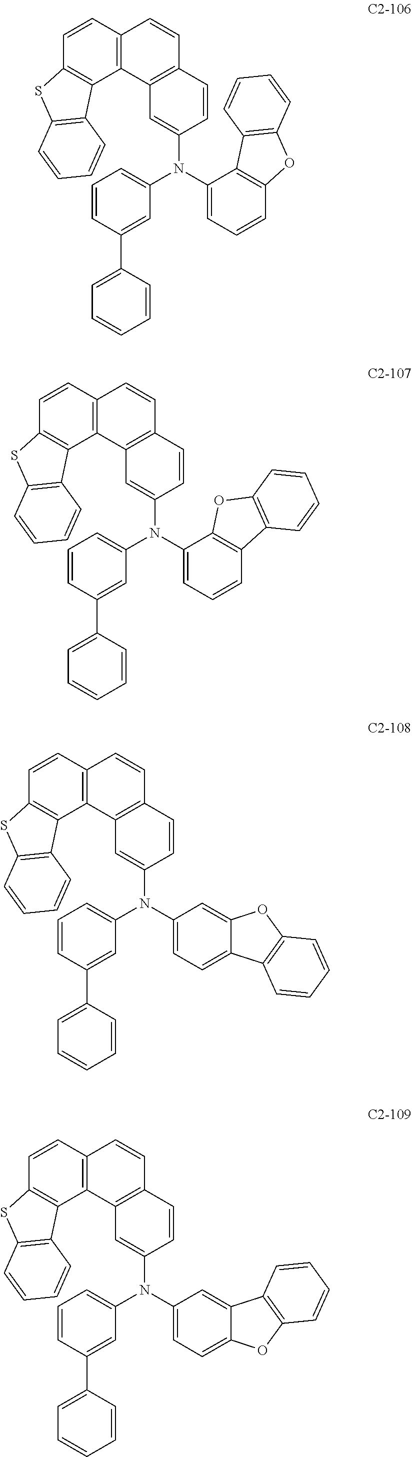 [SMILES: c1ccc(-c2cccc(N(c3ccc4ccc5ccc6sc7ccccc7c6c5c4c3)c3cccc4oc5ccccc5c34)c2)cc1 ; c1ccc(-c2cccc(N(c3ccc4ccc5ccc6sc7ccccc7c6c5c4c3)c3cccc4c3oc3ccccc34)c2)cc1 ; c1ccc(-c2cccc(N(c3ccc4c(c3)oc3ccccc34)c3ccc4ccc5ccc6sc7ccccc7c6c5c4c3)c2)cc1 ; c1ccc(-c2cccc(N(c3ccc4oc5ccccc5c4c3)c3ccc4ccc5ccc6sc7ccccc7c6c5c4c3)c2)cc1]

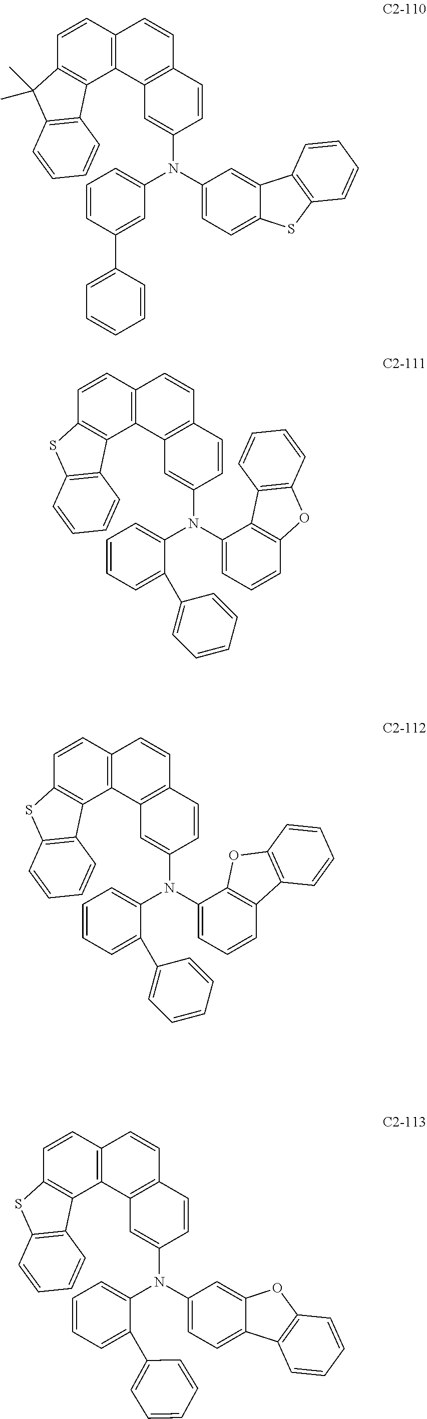

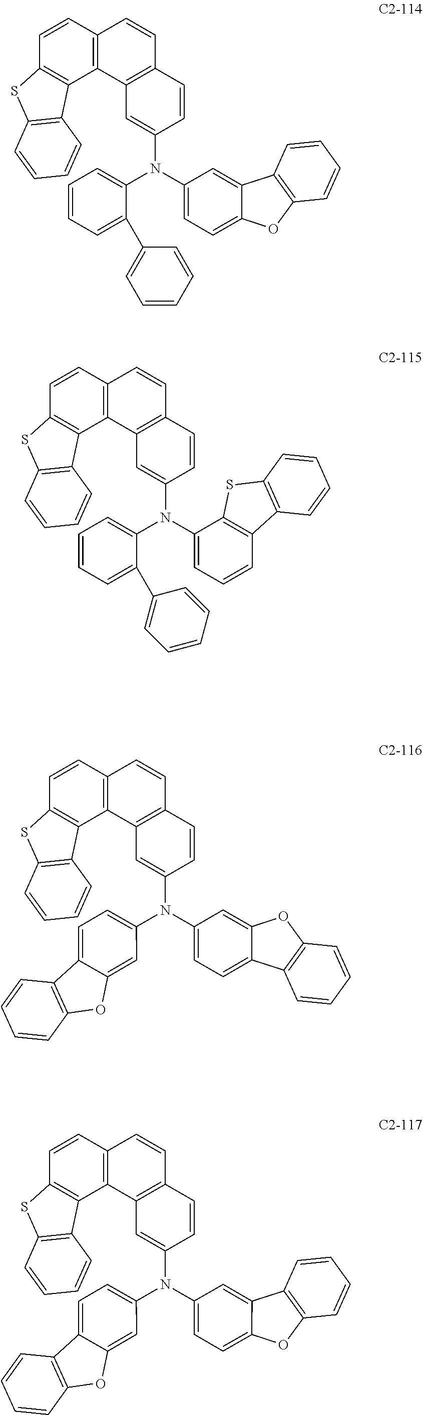 [SMILES: c1ccc(-c2ccccc2N(c2ccc3oc4ccccc4c3c2)c2ccc3ccc4ccc5sc6ccccc6c5c4c3c2)cc1 ; c1ccc(-c2ccccc2N(c2ccc3ccc4ccc5sc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6sc7ccccc7c6c5c4c3)ccc12 ; c1ccc2c(c1)oc1cc(N(c3ccc4oc5ccccc5c4c3)c3ccc4ccc5ccc6sc7ccccc7c6c5c4c3)ccc12]

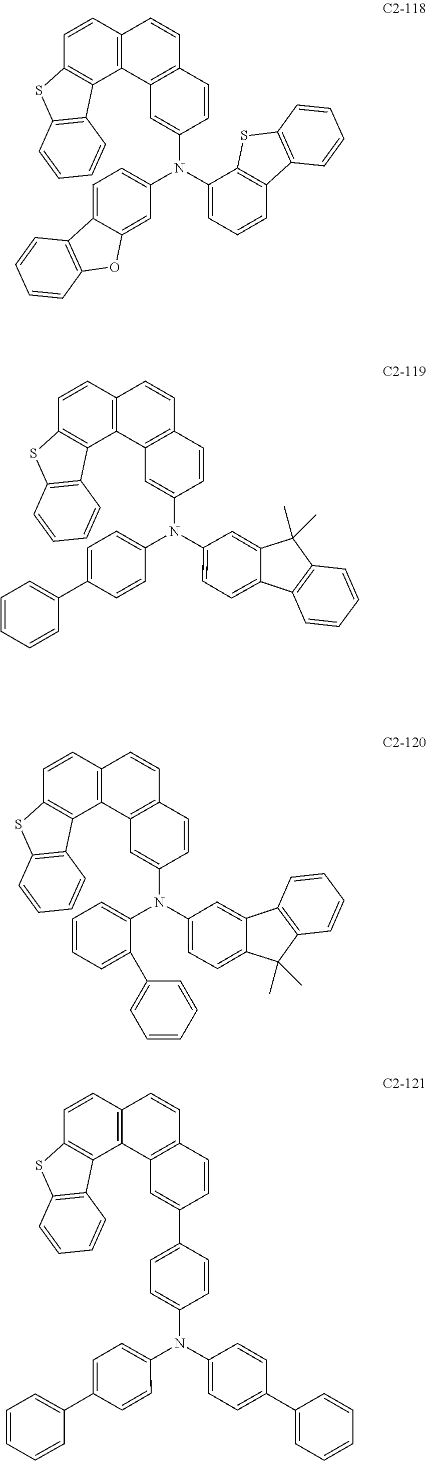 [SMILES: c1ccc2c(c1)oc1cc(N(c3ccc4ccc5ccc6sc7ccccc7c6c5c4c3)c3cccc4c3sc3ccccc34)ccc12 ; CC1(C)c2ccccc2-c2ccc(N(c3ccc(-c4ccccc4)cc3)c3ccc4ccc5ccc6sc7ccccc7c6c5c4c3)cc21 ; CC1(C)c2ccccc2-c2cc(N(c3ccc4ccc5ccc6sc7ccccc7c6c5c4c3)c3ccccc3-c3ccccc3)ccc21 ; c1ccc(-c2ccc(N(c3ccc(-c4ccccc4)cc3)c3ccc(-c4ccc5ccc6ccc7sc8ccccc8c7c6c5c4)cc3)cc2)cc1]

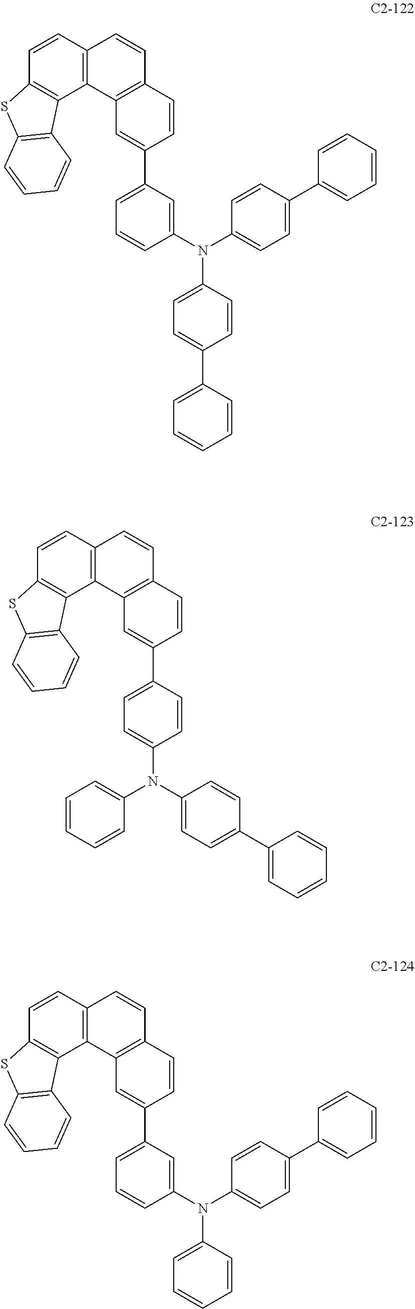 [SMILES: c1ccc(-c2ccc(N(c3ccc(-c4ccccc4)cc3)c3cccc(-c4ccc5ccc6ccc7sc8ccccc8c7c6c5c4)c3)cc2)cc1 ; c1ccc(-c2ccc(N(c3ccccc3)c3ccc(-c4ccc5ccc6ccc7sc8ccccc8c7c6c5c4)cc3)cc2)cc1 ; c1ccc(-c2ccc(N(c3ccccc3)c3cccc(-c4ccc5ccc6ccc7sc8ccccc8c7c6c5c4)c3)cc2)cc1]

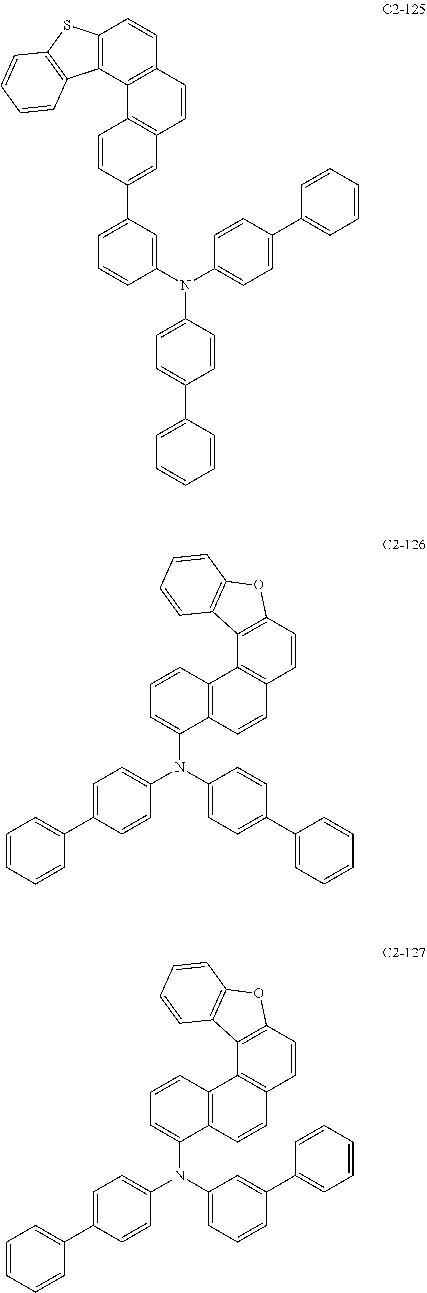 [SMILES: c1ccc(-c2ccc(N(c3ccc(-c4ccccc4)cc3)c3cccc(-c4ccc5c(ccc6ccc7sc8ccccc8c7c65)c4)c3)cc2)cc1 ; c1ccc(-c2ccc(N(c3ccc(-c4ccccc4)cc3)c3cccc4c3ccc3ccc5oc6ccccc6c5c34)cc2)cc1 ; c1ccc(-c2ccc(N(c3cccc(-c4ccccc4)c3)c3cccc4c3ccc3ccc5oc6ccccc6c5c34)cc2)cc1]

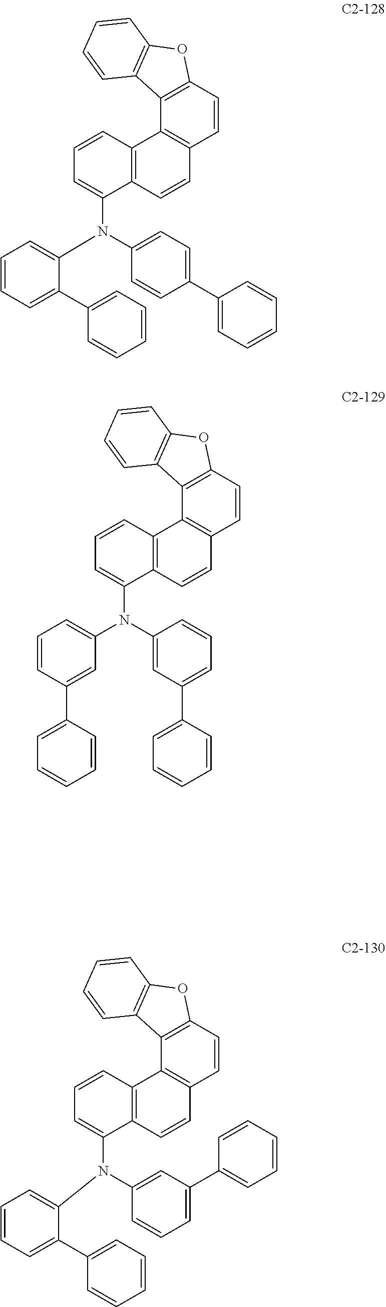 [SMILES: c1ccc(-c2ccc(N(c3ccccc3-c3ccccc3)c3cccc4c3ccc3ccc5oc6ccccc6c5c34)cc2)cc1 ; c1ccc(-c2cccc(N(c3cccc(-c4ccccc4)c3)c3cccc4c3ccc3ccc5oc6ccccc6c5c34)c2)cc1 ; c1ccc(-c2cccc(N(c3ccccc3-c3ccccc3)c3cccc4c3ccc3ccc5oc6ccccc6c5c34)c2)cc1]

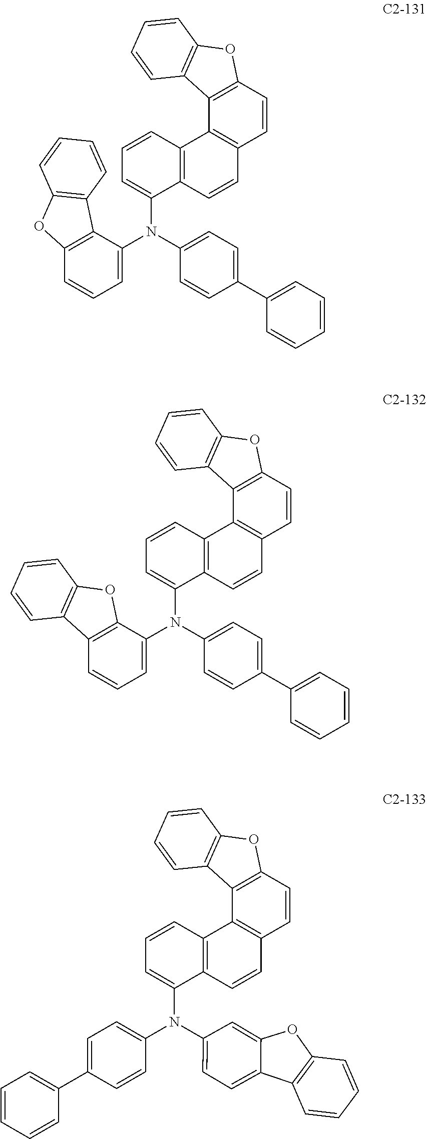 [SMILES: c1ccc(-c2ccc(N(c3cccc4c3ccc3ccc5oc6ccccc6c5c34)c3cccc4oc5ccccc5c34)cc2)cc1 ; c1ccc(-c2ccc(N(c3cccc4c3ccc3ccc5oc6ccccc6c5c34)c3cccc4c3oc3ccccc34)cc2)cc1 ; c1ccc(-c2ccc(N(c3ccc4c(c3)oc3ccccc34)c3cccc4c3ccc3ccc5oc6ccccc6c5c34)cc2)cc1]

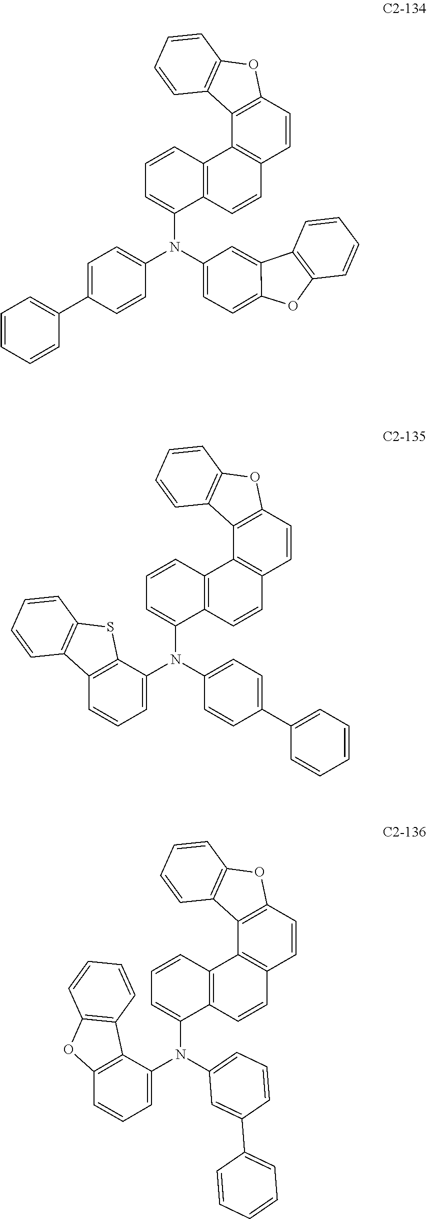 [SMILES: c1ccc(-c2ccc(N(c3ccc4oc5ccccc5c4c3)c3cccc4c3ccc3ccc5oc6ccccc6c5c34)cc2)cc1 ; c1ccc(-c2ccc(N(c3cccc4c3ccc3ccc5oc6ccccc6c5c34)c3cccc4c3sc3ccccc34)cc2)cc1 ; c1ccc(-c2cccc(N(c3cccc4c3ccc3ccc5oc6ccccc6c5c34)c3cccc4oc5ccccc5c34)c2)cc1]

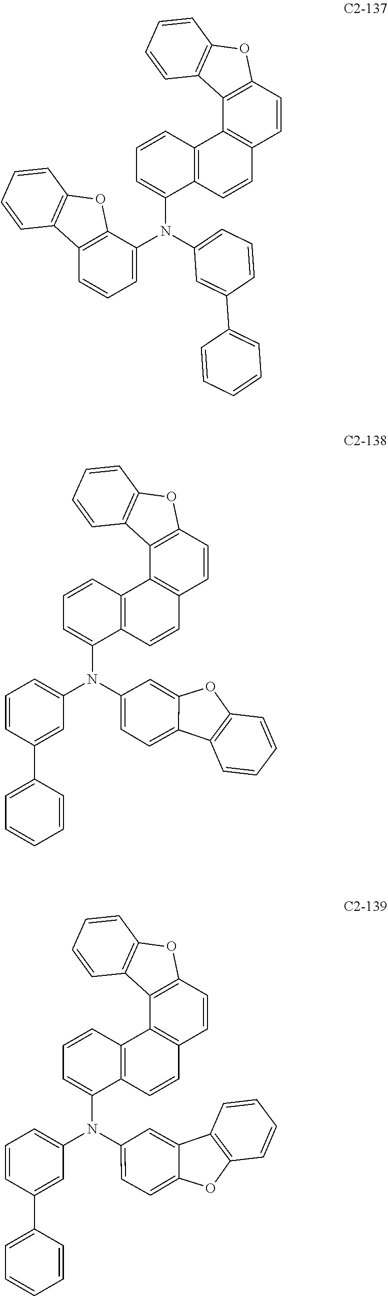 [SMILES: c1ccc(-c2cccc(N(c3cccc4c3ccc3ccc5oc6ccccc6c5c34)c3cccc4c3oc3ccccc34)c2)cc1 ; c1ccc(-c2cccc(N(c3ccc4c(c3)oc3ccccc34)c3cccc4c3ccc3ccc5oc6ccccc6c5c34)c2)cc1 ; c1ccc(-c2cccc(N(c3ccc4oc5ccccc5c4c3)c3cccc4c3ccc3ccc5oc6ccccc6c5c34)c2)cc1]

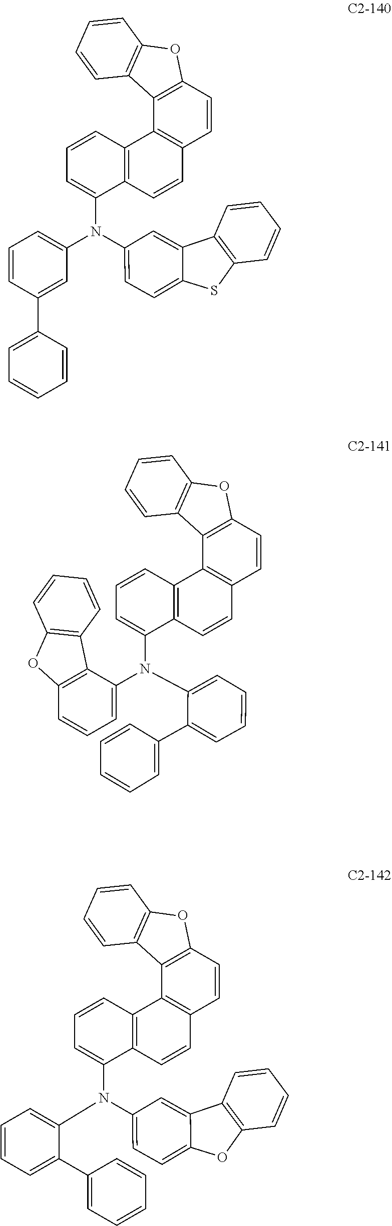 [SMILES: c1ccc(-c2cccc(N(c3ccc4sc5ccccc5c4c3)c3cccc4c3ccc3ccc5oc6ccccc6c5c34)c2)cc1 ; c1ccc(-c2ccccc2N(c2cccc3c2ccc2ccc4oc5ccccc5c4c23)c2cccc3oc4ccccc4c23)cc1 ; c1ccc(-c2ccccc2N(c2ccc3oc4ccccc4c3c2)c2cccc3c2ccc2ccc4oc5ccccc5c4c23)cc1]

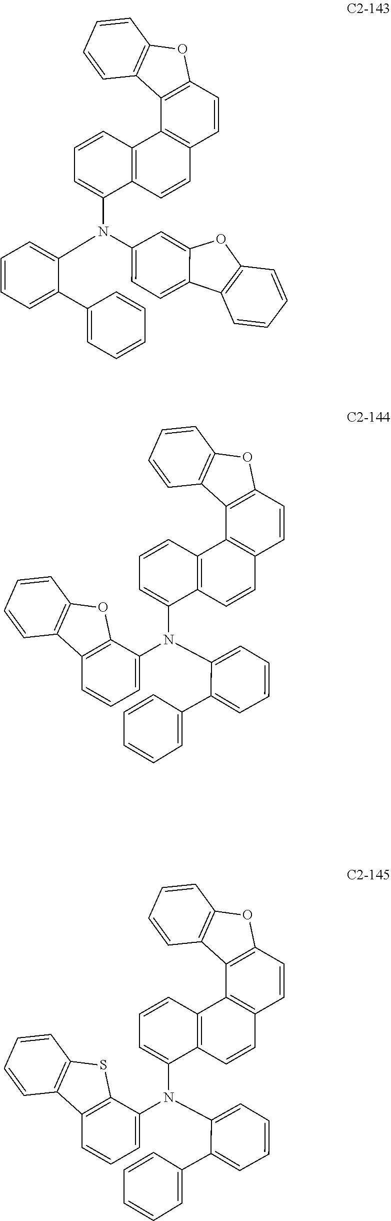 [SMILES: c1ccc(-c2ccccc2N(c2ccc3c(c2)oc2ccccc23)c2cccc3c2ccc2ccc4oc5ccccc5c4c23)cc1 ; c1ccc(-c2ccccc2N(c2cccc3c2ccc2ccc4oc5ccccc5c4c23)c2cccc3c2oc2ccccc23)cc1 ; c1ccc(-c2ccccc2N(c2cccc3c2ccc2ccc4oc5ccccc5c4c23)c2cccc3c2sc2ccccc23)cc1]

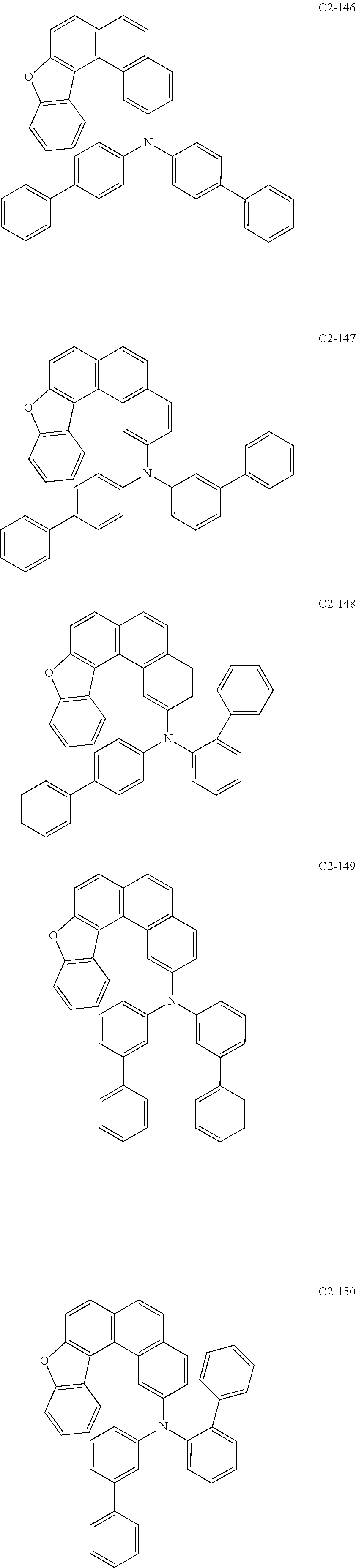 [SMILES: c1ccc(-c2ccc(N(c3ccc(-c4ccccc4)cc3)c3ccc4ccc5ccc6oc7ccccc7c6c5c4c3)cc2)cc1 ; c1ccc(-c2ccc(N(c3cccc(-c4ccccc4)c3)c3ccc4ccc5ccc6oc7ccccc7c6c5c4c3)cc2)cc1 ; c1ccc(-c2ccc(N(c3ccc4ccc5ccc6oc7ccccc7c6c5c4c3)c3ccccc3-c3ccccc3)cc2)cc1 ; c1ccc(-c2cccc(N(c3cccc(-c4ccccc4)c3)c3ccc4ccc5ccc6oc7ccccc7c6c5c4c3)c2)cc1 ; c1ccc(-c2cccc(N(c3ccc4ccc5ccc6oc7ccccc7c6c5c4c3)c3ccccc3-c3ccccc3)c2)cc1]

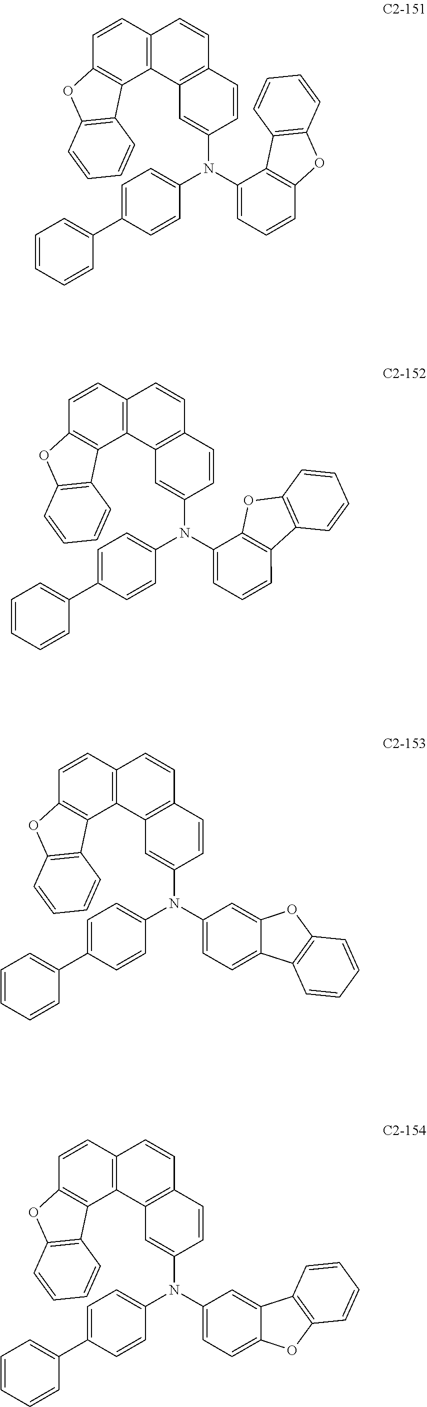 [SMILES: c1ccc(-c2ccc(N(c3ccc4ccc5ccc6oc7ccccc7c6c5c4c3)c3cccc4oc5ccccc5c34)cc2)cc1 ; c1ccc(-c2ccc(N(c3ccc4ccc5ccc6oc7ccccc7c6c5c4c3)c3cccc4c3oc3ccccc34)cc2)cc1 ; c1ccc(-c2ccc(N(c3ccc4c(c3)oc3ccccc34)c3ccc4ccc5ccc6oc7ccccc7c6c5c4c3)cc2)cc1 ; c1ccc(-c2ccc(N(c3ccc4oc5ccccc5c4c3)c3ccc4ccc5ccc6oc7ccccc7c6c5c4c3)cc2)cc1]

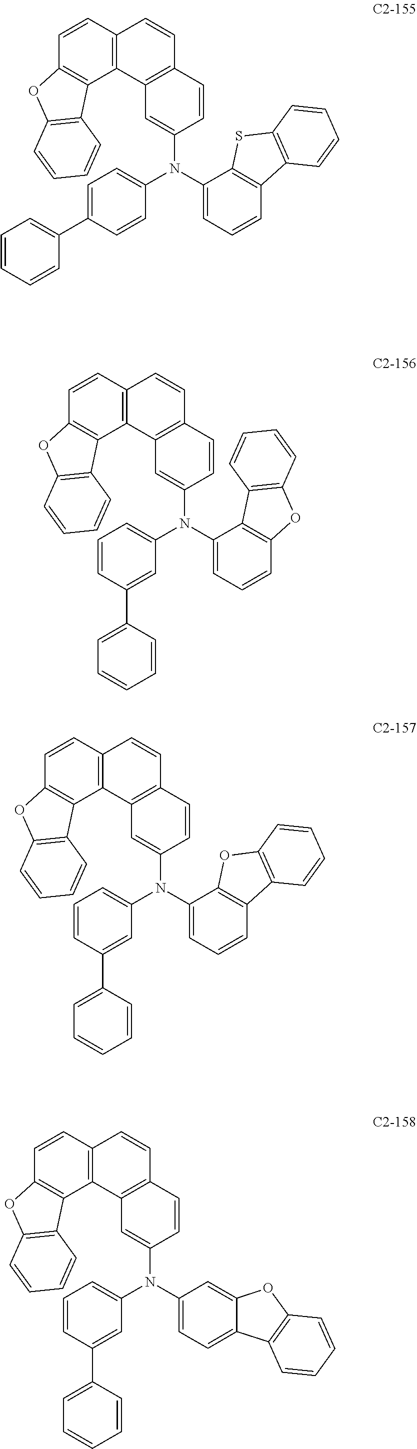 [SMILES: c1ccc(-c2ccc(N(c3ccc4ccc5ccc6oc7ccccc7c6c5c4c3)c3cccc4c3sc3ccccc34)cc2)cc1 ; c1ccc(-c2cccc(N(c3ccc4ccc5ccc6oc7ccccc7c6c5c4c3)c3cccc4oc5ccccc5c34)c2)cc1 ; c1ccc(-c2cccc(N(c3ccc4ccc5ccc6oc7ccccc7c6c5c4c3)c3cccc4c3oc3ccccc34)c2)cc1 ; c1ccc(-c2cccc(N(c3ccc4c(c3)oc3ccccc34)c3ccc4ccc5ccc6oc7ccccc7c6c5c4c3)c2)cc1]

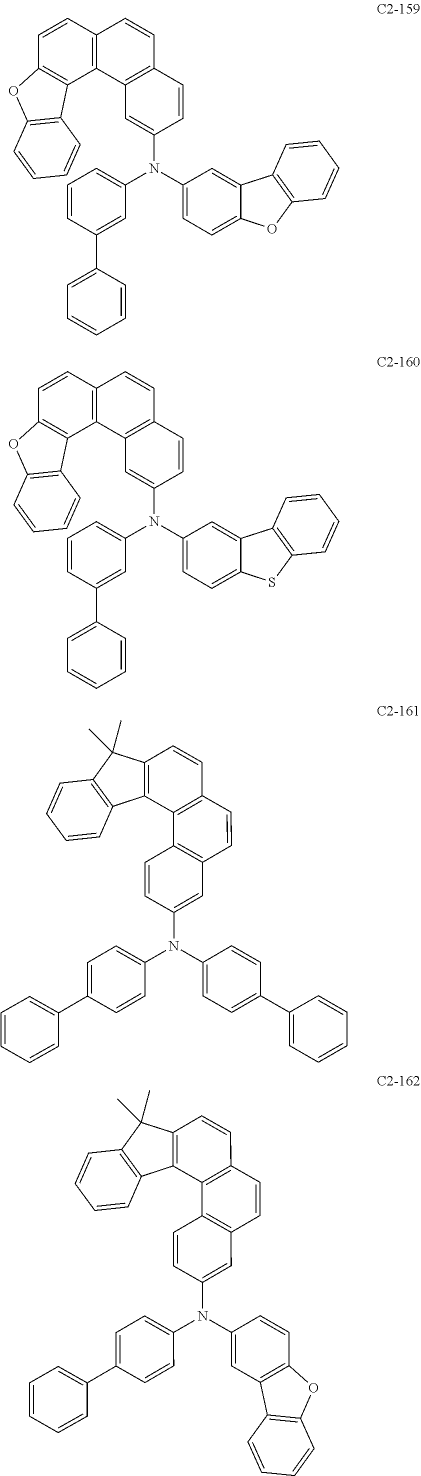

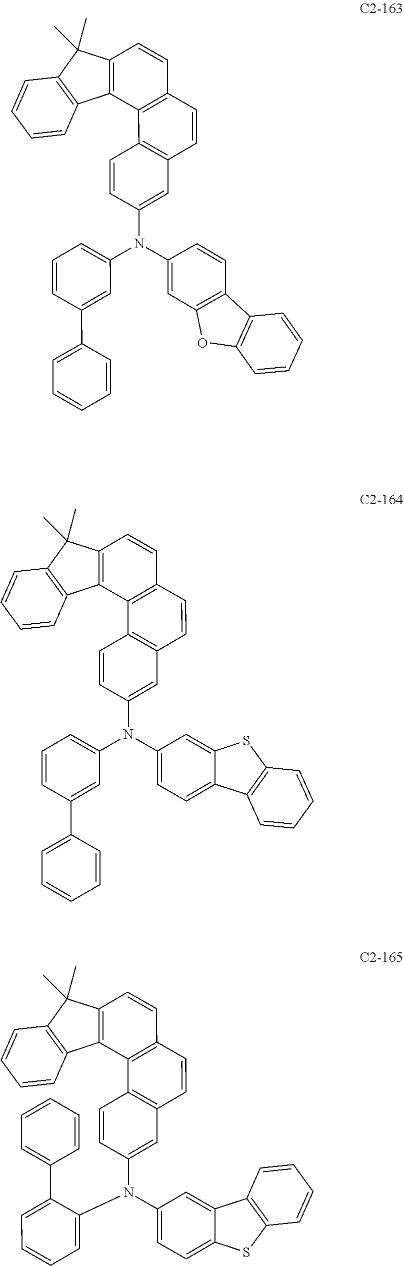 [SMILES: CC1(C)c2ccccc2-c2c1ccc1ccc3cc(N(c4cccc(-c5ccccc5)c4)c4ccc5c(c4)oc4ccccc45)ccc3c21 ; CC1(C)c2ccccc2-c2c1ccc1ccc3cc(N(c4cccc(-c5ccccc5)c4)c4ccc5c(c4)sc4ccccc45)ccc3c21 ; CC1(C)c2ccccc2-c2c1ccc1ccc3cc(N(c4ccc5sc6ccccc6c5c4)c4ccccc4-c4ccccc4)ccc3c21]

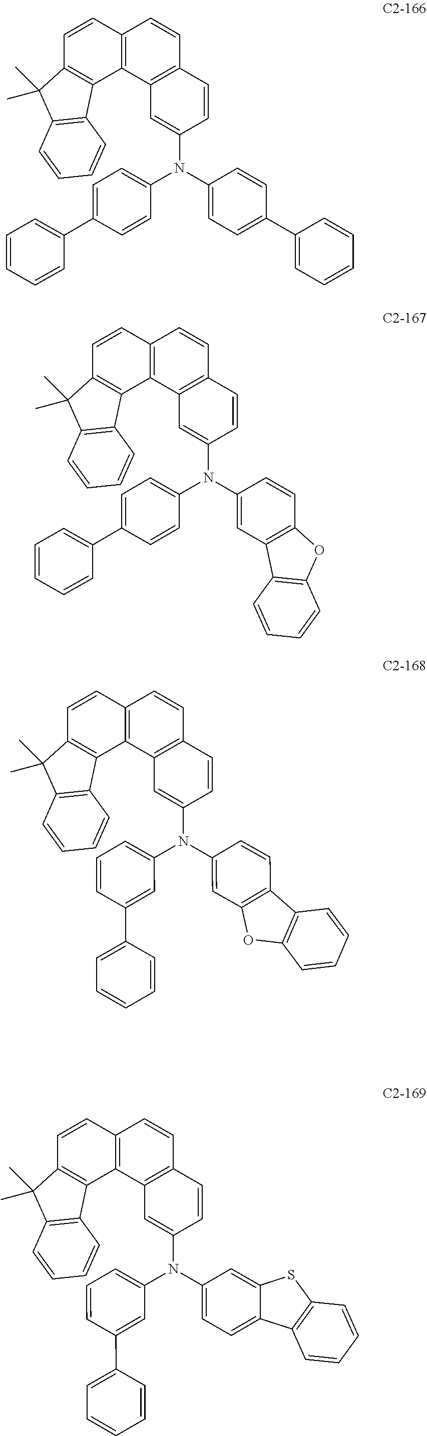 [SMILES: CC1(C)c2ccccc2-c2c1ccc1ccc3ccc(N(c4ccc(-c5ccccc5)cc4)c4ccc(-c5ccccc5)cc4)cc3c21 ; CC1(C)c2ccccc2-c2c1ccc1ccc3ccc(N(c4ccc(-c5ccccc5)cc4)c4ccc5oc6ccccc6c5c4)cc3c21 ; CC1(C)c2ccccc2-c2c1ccc1ccc3ccc(N(c4cccc(-c5ccccc5)c4)c4ccc5c(c4)oc4ccccc45)cc3c21 ; CC1(C)c2ccccc2-c2c1ccc1ccc3ccc(N(c4cccc(-c5ccccc5)c4)c4ccc5c(c4)sc4ccccc45)cc3c21]

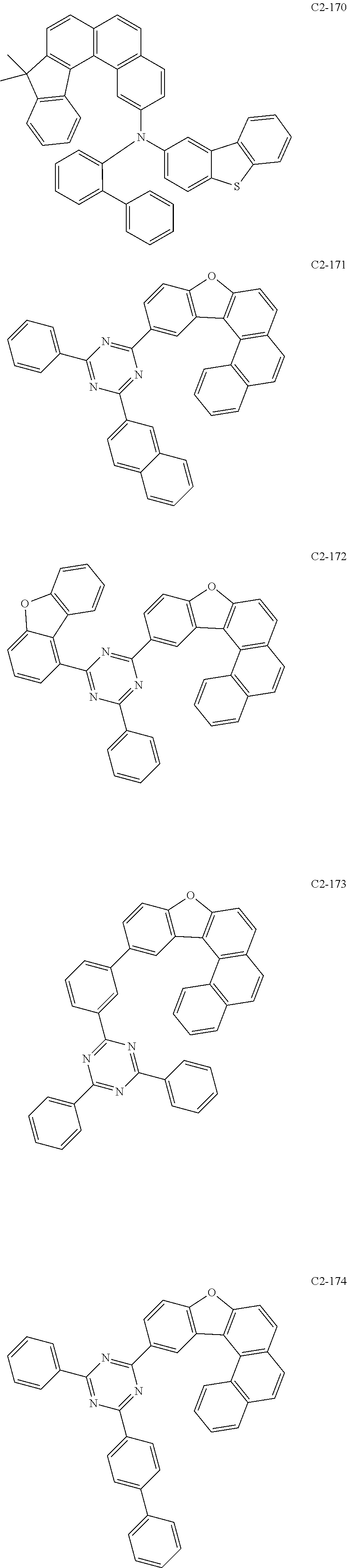 [SMILES: CC1(C)c2ccccc2-c2c1ccc1ccc3ccc(N(c4ccc5sc6ccccc6c5c4)c4ccccc4-c4ccccc4)cc3c21 ; c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1 ; c1ccc(-c2nc(-c3ccccc3)nc(-c3cccc(-c4ccc5oc6ccc7ccc8ccccc8c7c6c5c4)c3)n2)cc1 ; c1ccc(-c2ccc(-c3nc(-c4ccccc4)nc(-c4ccc5oc6ccc7ccc8ccccc8c7c6c5c4)n3)cc2)cc1]

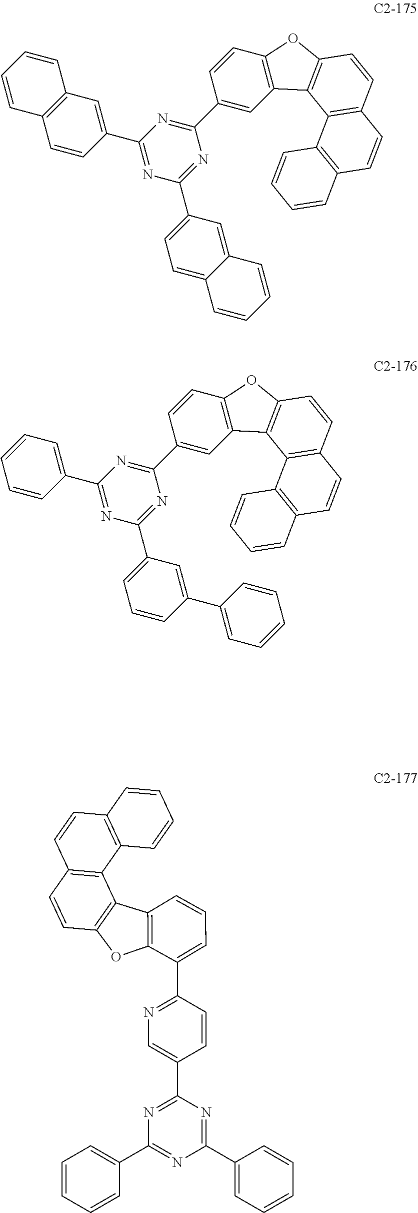 [SMILES: c1ccc2cc(-c3nc(-c4ccc5ccccc5c4)nc(-c4ccc5oc6ccc7ccc8ccccc8c7c6c5c4)n3)ccc2c1 ; c1ccc(-c2cccc(-c3nc(-c4ccccc4)nc(-c4ccc5oc6ccc7ccc8ccccc8c7c6c5c4)n3)c2)cc1 ; c1ccc(-c2nc(-c3ccccc3)nc(-c3ccc(-c4cccc5c4oc4ccc6ccc7ccccc7c6c45)nc3)n2)cc1]

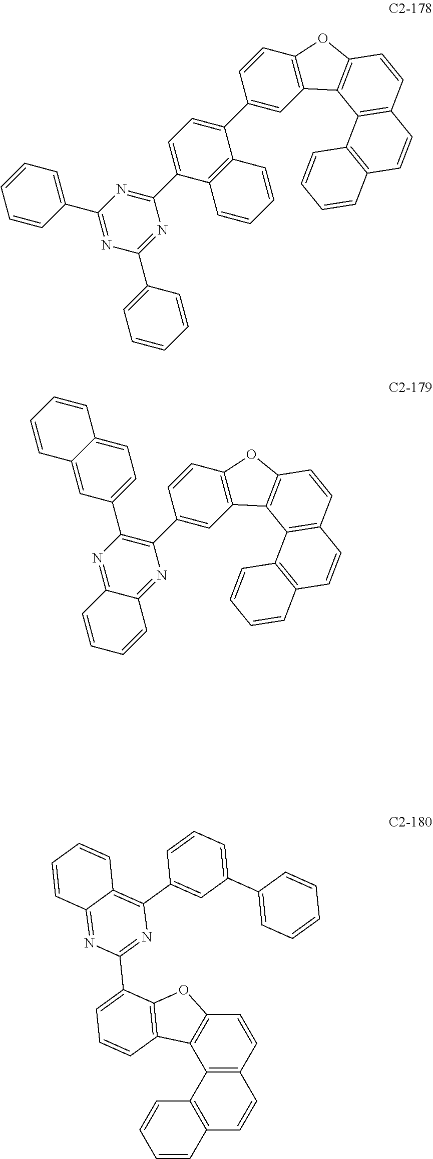 [SMILES: c1ccc(-c2nc(-c3ccccc3)nc(-c3ccc(-c4ccc5oc6ccc7ccc8ccccc8c7c6c5c4)c4ccccc34)n2)cc1 ; c1ccc2cc(-c3nc4ccccc4nc3-c3ccc4oc5ccc6ccc7ccccc7c6c5c4c3)ccc2c1 ; c1ccc(-c2cccc(-c3nc(-c4cccc5c4oc4ccc6ccc7ccccc7c6c45)nc4ccccc34)c2)cc1]

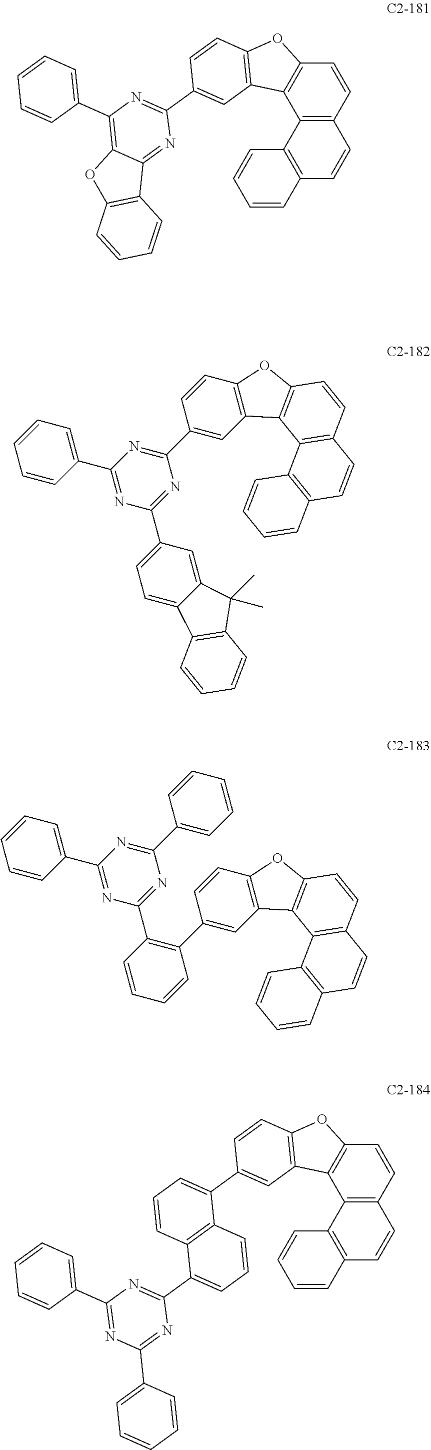 [SMILES: c1ccc(-c2nc(-c3ccc4oc5ccc6ccc7ccccc7c6c5c4c3)nc3c2oc2ccccc23)cc1 ; CC1(C)c2ccccc2-c2ccc(-c3nc(-c4ccccc4)nc(-c4ccc5oc6ccc7ccc8ccccc8c7c6c5c4)n3)cc21 ; c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1]

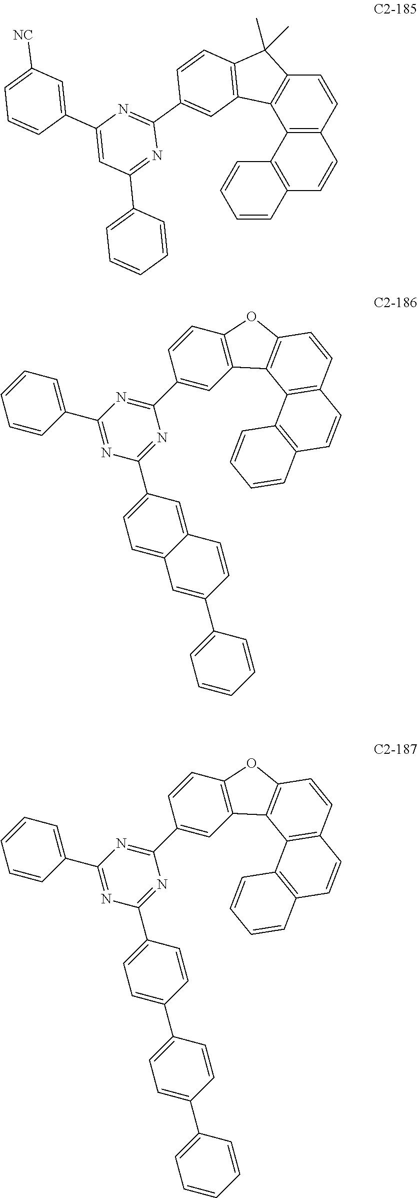 [SMILES: CC1(C)c2ccc(-c3nc(-c4ccccc4)cc(-c4cccc(C#N)c4)n3)cc2-c2c1ccc1ccc3ccccc3c21 ; c1ccc(-c2ccc3cc(-c4nc(-c5ccccc5)nc(-c5ccc6oc7ccc8ccc9ccccc9c8c7c6c5)n4)ccc3c2)cc1 ; c1ccc(-c2ccc(-c3ccc(-c4nc(-c5ccccc5)nc(-c5ccc6oc7ccc8ccc9ccccc9c8c7c6c5)n4)cc3)cc2)cc1]

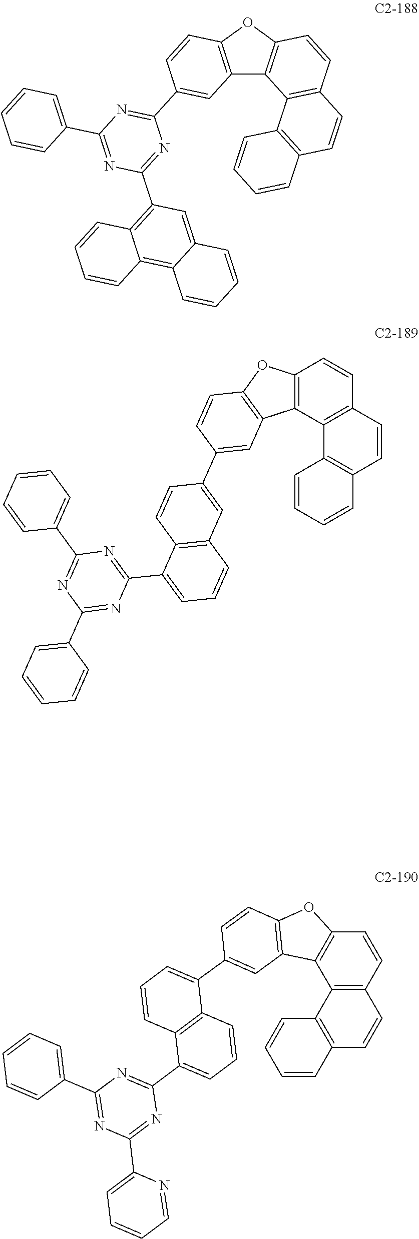 [SMILES: c1ccc(-c2nc(-c3ccc4oc5ccc6ccc7ccccc7c6c5c4c3)nc(-c3cc4ccccc4c4ccccc34)n2)cc1 ; c1ccc(-c2nc(-c3ccccc3)nc(-c3cccc4cc(-c5ccc6oc7ccc8ccc9ccccc9c8c7c6c5)ccc34)n2)cc1 ; c1ccc(-c2nc(-c3ccccn3)nc(-c3cccc4c(-c5ccc6oc7ccc8ccc9ccccc9c8c7c6c5)cccc34)n2)cc1]

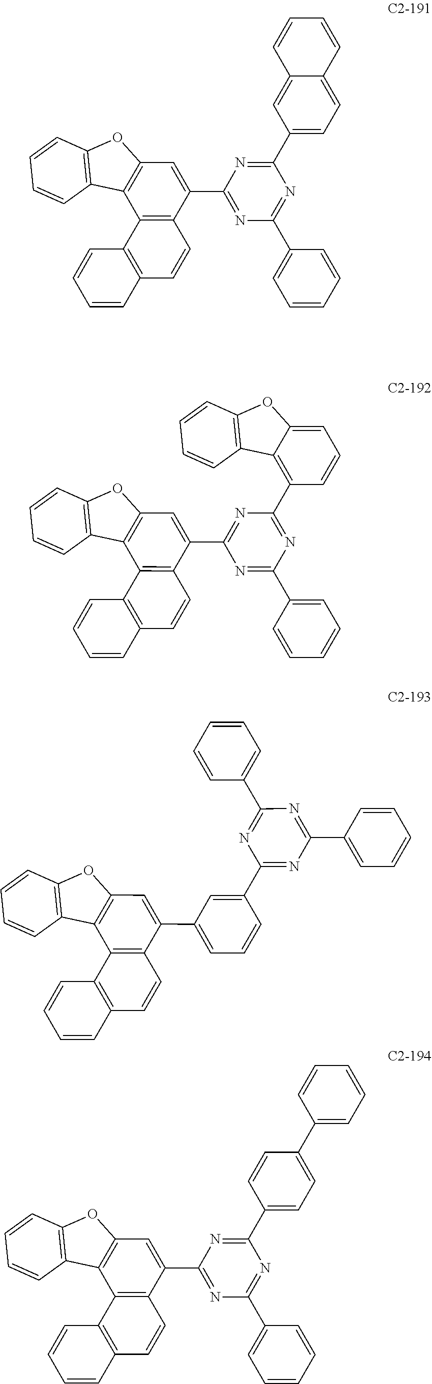 [SMILES: c1ccc(-c2nc(-c3ccc4ccccc4c3)nc(-c3cc4oc5ccccc5c4c4c3ccc3ccccc34)n2)cc1 ; c1ccc(-c2nc(-c3cc4oc5ccccc5c4c4c3ccc3ccccc34)nc(-c3cccc4oc5ccccc5c34)n2)cc1 ; c1ccc(-c2nc(-c3ccccc3)nc(-c3cccc(-c4cc5oc6ccccc6c5c5c4ccc4ccccc45)c3)n2)cc1 ; c1ccc(-c2ccc(-c3nc(-c4ccccc4)nc(-c4cc5oc6ccccc6c5c5c4ccc4ccccc45)n3)cc2)cc1]

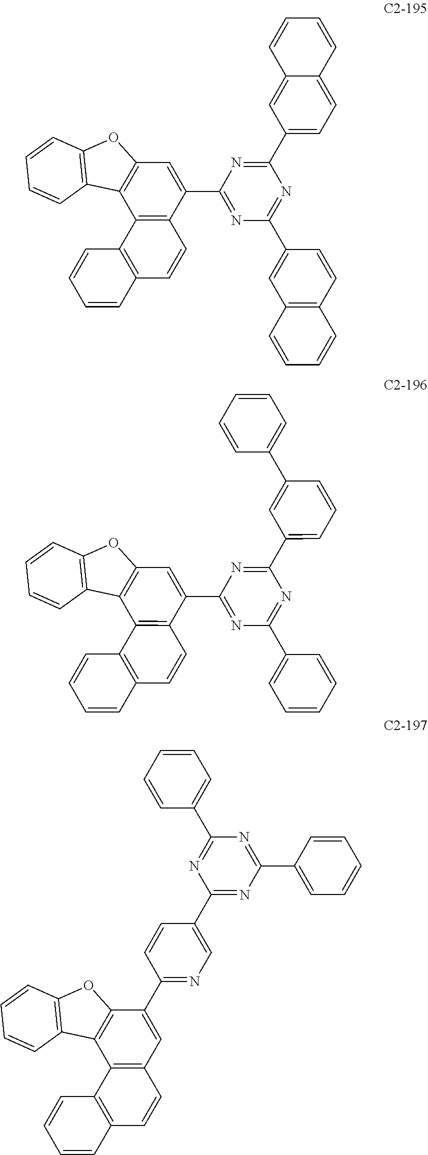

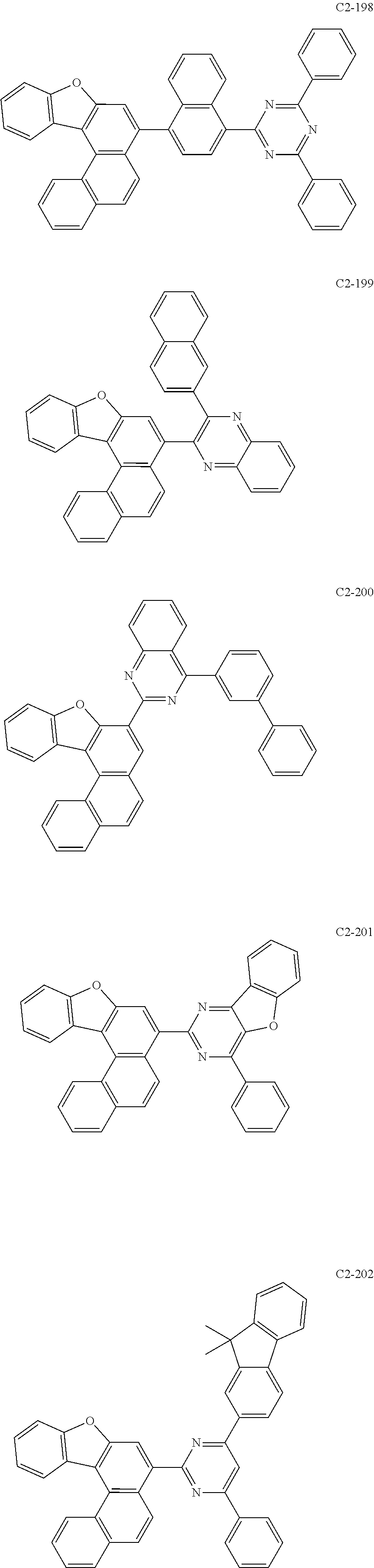 [SMILES: c1ccc(-c2nc(-c3ccccc3)nc(-c3ccc(-c4cc5oc6ccccc6c5c5c4ccc4ccccc45)c4ccccc34)n2)cc1 ; c1ccc2cc(-c3nc4ccccc4nc3-c3cc4oc5ccccc5c4c4c3ccc3ccccc34)ccc2c1 ; c1ccc(-c2cccc(-c3nc(-c4cc5ccc6ccccc6c5c5c4oc4ccccc45)nc4ccccc34)c2)cc1 ; c1ccc(-c2nc(-c3cc4oc5ccccc5c4c4c3ccc3ccccc34)nc3c2oc2ccccc23)cc1 ; CC1(C)c2ccccc2-c2ccc(-c3cc(-c4ccccc4)nc(-c4cc5oc6ccccc6c5c5c4ccc4ccccc45)n3)cc21]

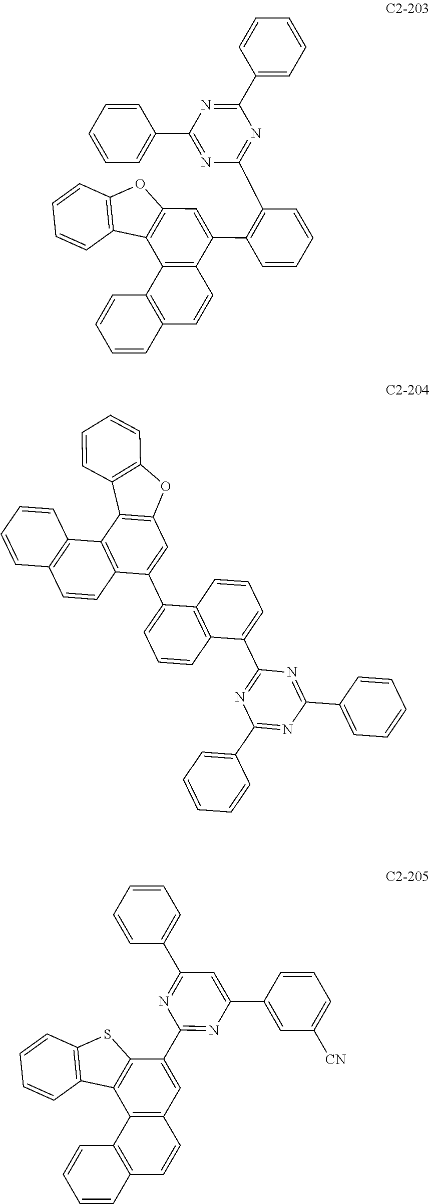 [SMILES: c1ccc(-c2nc(-c3ccccc3)nc(-c3ccccc3-c3cc4oc5ccccc5c4c4c3ccc3ccccc34)n2)cc1 ; c1ccc(-c2nc(-c3ccccc3)nc(-c3cccc4c(-c5cc6oc7ccccc7c6c6c5ccc5ccccc56)cccc34)n2)cc1 ; N#Cc1cccc(-c2cc(-c3ccccc3)nc(-c3cc4ccc5ccccc5c4c4c3sc3ccccc34)n2)c1]

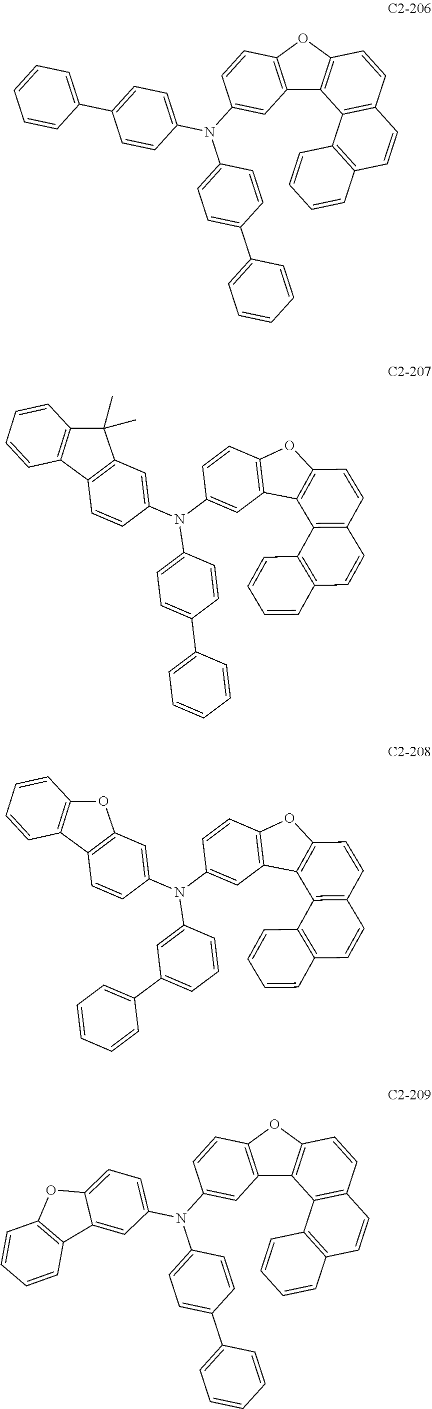 [SMILES: c1ccc(-c2ccc(N(c3ccc(-c4ccccc4)cc3)c3ccc4oc5ccc6ccc7ccccc7c6c5c4c3)cc2)cc1 ; CC1(C)c2ccccc2-c2ccc(N(c3ccc(-c4ccccc4)cc3)c3ccc4oc5ccc6ccc7ccccc7c6c5c4c3)cc21 ; c1ccc(-c2cccc(N(c3ccc4c(c3)oc3ccccc34)c3ccc4oc5ccc6ccc7ccccc7c6c5c4c3)c2)cc1 ; c1ccc(-c2ccc(N(c3ccc4oc5ccccc5c4c3)c3ccc4oc5ccc6ccc7ccccc7c6c5c4c3)cc2)cc1]

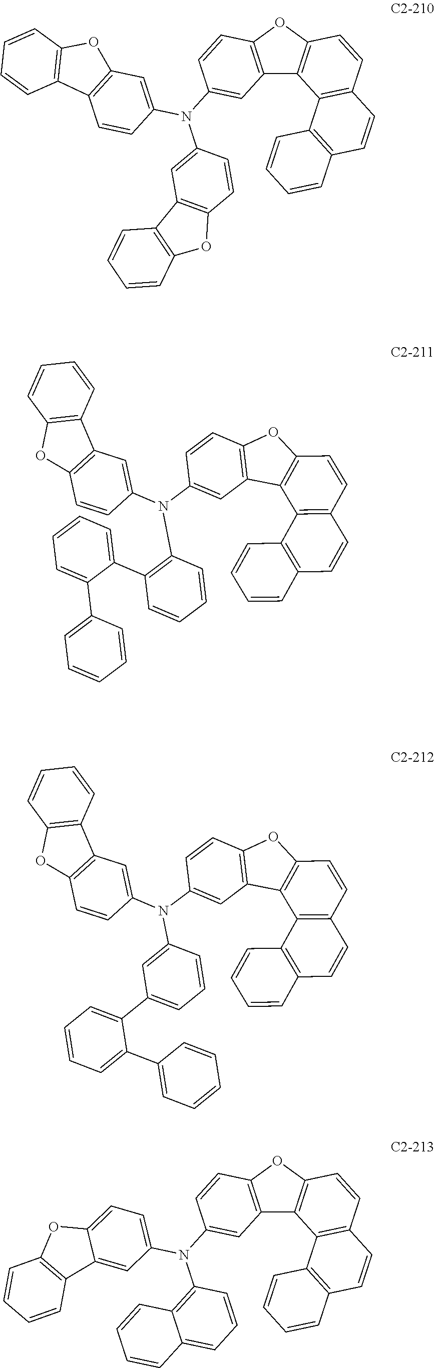 [SMILES: c1ccc2c(c1)ccc1ccc3oc4ccc(N(c5ccc6c(c5)oc5ccccc56)c5ccc6oc7ccccc7c6c5)cc4c3c12 ; c1ccc(-c2ccccc2-c2ccccc2N(c2ccc3oc4ccccc4c3c2)c2ccc3oc4ccc5ccc6ccccc6c5c4c3c2)cc1 ; c1ccc(-c2ccccc2-c2cccc(N(c3ccc4oc5ccccc5c4c3)c3ccc4oc5ccc6ccc7ccccc7c6c5c4c3)c2)cc1 ; c1ccc2c(N(c3ccc4oc5ccccc5c4c3)c3ccc4oc5ccc6ccc7ccccc7c6c5c4c3)cccc2c1]

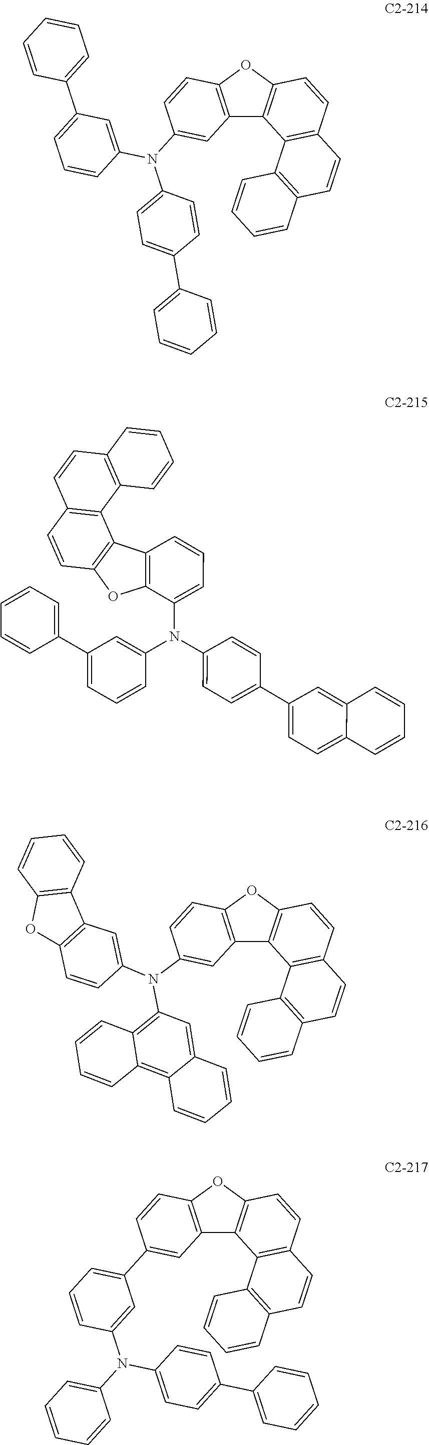 [SMILES: c1ccc(-c2ccc(N(c3cccc(-c4ccccc4)c3)c3ccc4oc5ccc6ccc7ccccc7c6c5c4c3)cc2)cc1 ; c1ccc(-c2cccc(N(c3ccc(-c4ccc5ccccc5c4)cc3)c3cccc4c3oc3ccc5ccc6ccccc6c5c34)c2)cc1 ; c1ccc2c(c1)cc(N(c1ccc3oc4ccccc4c3c1)c1ccc3oc4ccc5ccc6ccccc6c5c4c3c1)c1ccccc12 ; c1ccc(-c2ccc(N(c3ccccc3)c3cccc(-c4ccc5oc6ccc7ccc8ccccc8c7c6c5c4)c3)cc2)cc1]

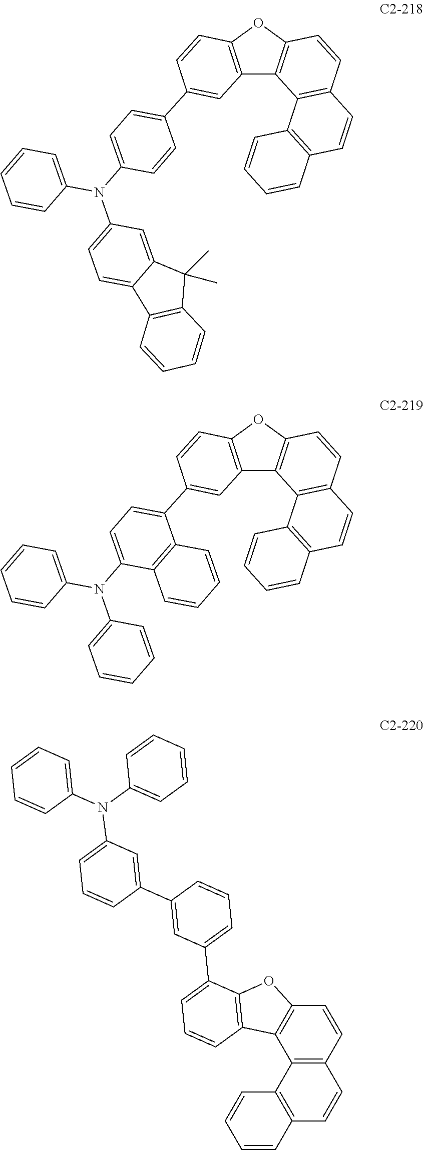 [SMILES: CC1(C)c2ccccc2-c2ccc(N(c3ccccc3)c3ccc(-c4ccc5oc6ccc7ccc8ccccc8c7c6c5c4)cc3)cc21 ; c1ccc(N(c2ccccc2)c2ccc(-c3ccc4oc5ccc6ccc7ccccc7c6c5c4c3)c3ccccc23)cc1 ; c1ccc(N(c2ccccc2)c2cccc(-c3cccc(-c4cccc5c4oc4ccc6ccc7ccccc7c6c45)c3)c2)cc1]

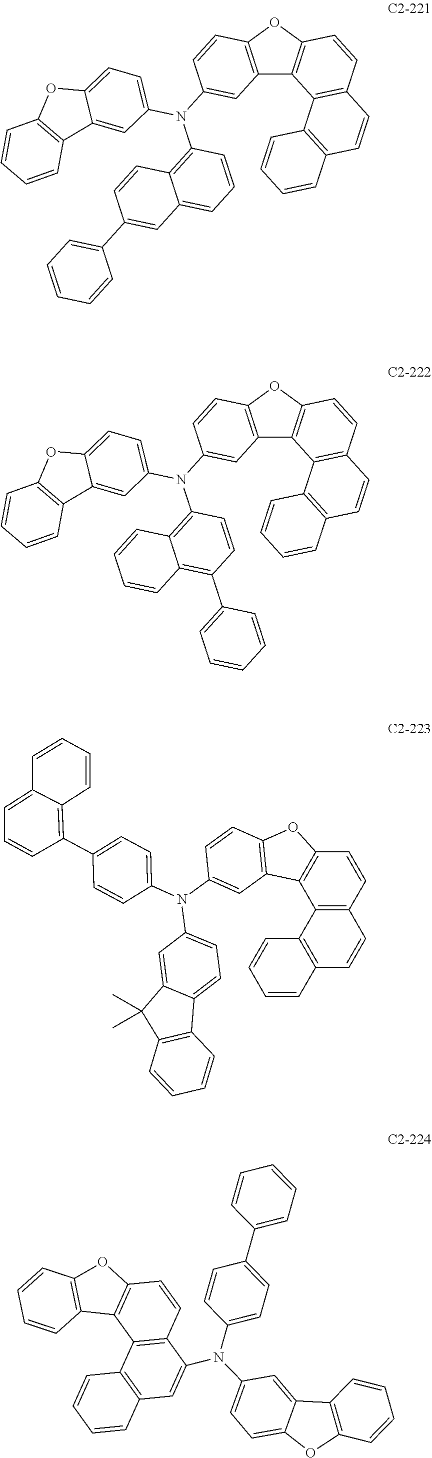 [SMILES: c1ccc(-c2ccc3c(N(c4ccc5oc6ccccc6c5c4)c4ccc5oc6ccc7ccc8ccccc8c7c6c5c4)cccc3c2)cc1 ; c1ccc(-c2ccc(N(c3ccc4oc5ccccc5c4c3)c3ccc4oc5ccc6ccc7ccccc7c6c5c4c3)c3ccccc23)cc1 ; CC1(C)c2ccccc2-c2ccc(N(c3ccc(-c4cccc5ccccc45)cc3)c3ccc4oc5ccc6ccc7ccccc7c6c5c4c3)cc21 ; c1ccc(-c2ccc(N(c3ccc4oc5ccccc5c4c3)c3cc4ccccc4c4c3ccc3oc5ccccc5c34)cc2)cc1]

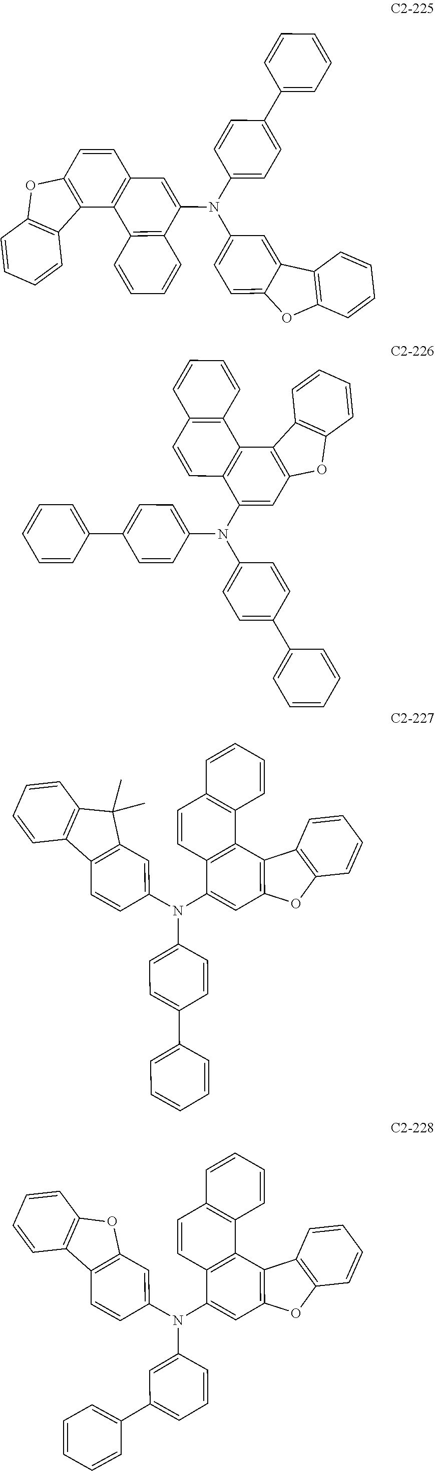 [SMILES: c1ccc(-c2ccc(N(c3ccc4oc5ccccc5c4c3)c3cc4ccc5oc6ccccc6c5c4c4ccccc34)cc2)cc1 ; c1ccc(-c2ccc(N(c3ccc(-c4ccccc4)cc3)c3cc4oc5ccccc5c4c4c3ccc3ccccc34)cc2)cc1 ; CC1(C)c2ccccc2-c2ccc(N(c3ccc(-c4ccccc4)cc3)c3cc4oc5ccccc5c4c4c3ccc3ccccc34)cc21 ; c1ccc(-c2cccc(N(c3ccc4c(c3)oc3ccccc34)c3cc4oc5ccccc5c4c4c3ccc3ccccc34)c2)cc1]

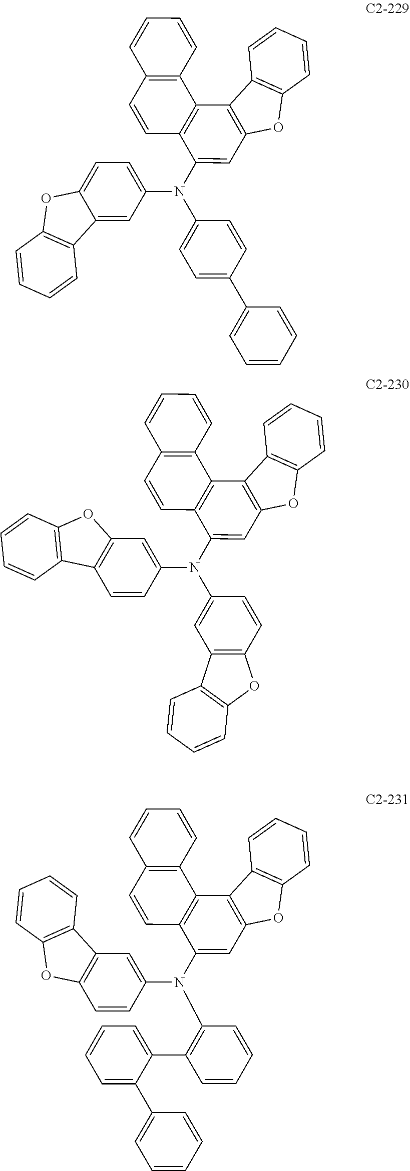

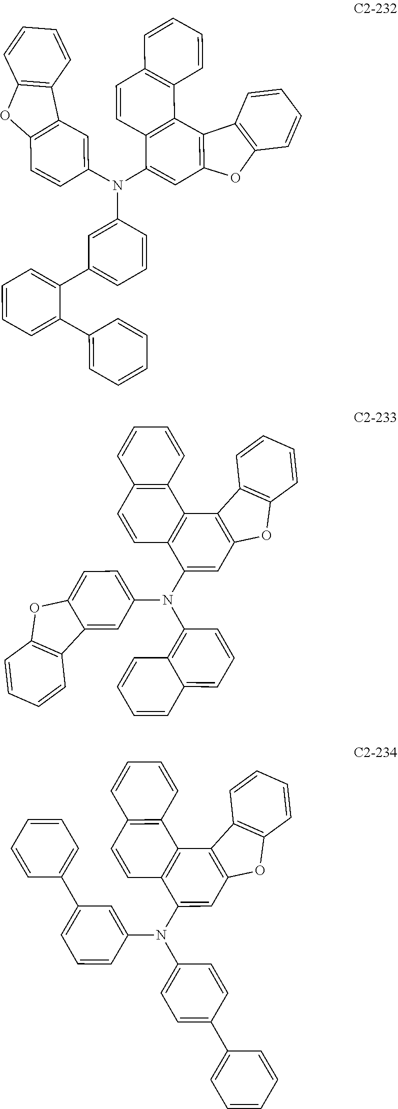

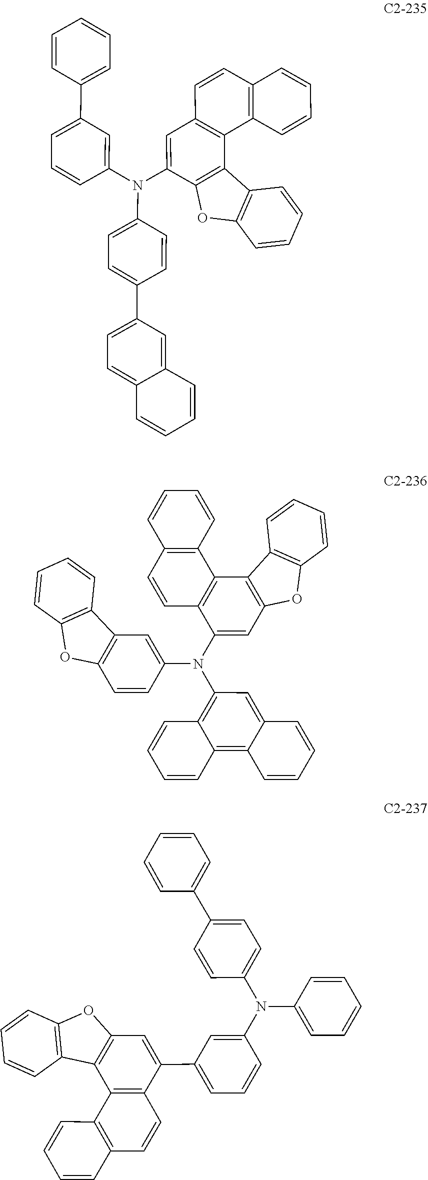 [SMILES: c1ccc(-c2cccc(N(c3ccc(-c4ccc5ccccc5c4)cc3)c3cc4ccc5ccccc5c4c4c3oc3ccccc34)c2)cc1 ; c1ccc2c(c1)cc(N(c1ccc3oc4ccccc4c3c1)c1cc3oc4ccccc4c3c3c1ccc1ccccc13)c1ccccc12 ; c1ccc(-c2ccc(N(c3ccccc3)c3cccc(-c4cc5oc6ccccc6c5c5c4ccc4ccccc45)c3)cc2)cc1]

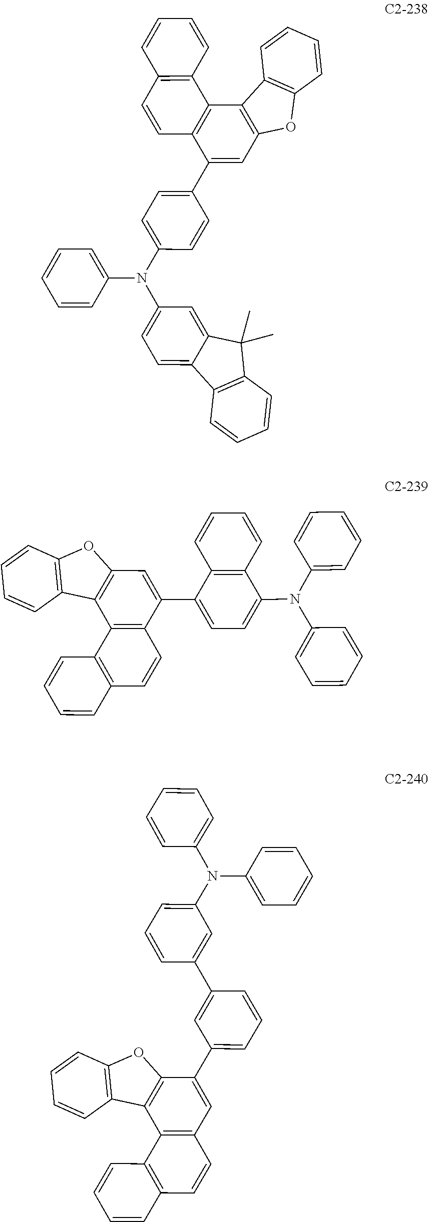 [SMILES: CC1(C)c2ccccc2-c2ccc(N(c3ccccc3)c3ccc(-c4cc5oc6ccccc6c5c5c4ccc4ccccc45)cc3)cc21 ; c1ccc(N(c2ccccc2)c2ccc(-c3cc4oc5ccccc5c4c4c3ccc3ccccc34)c3ccccc23)cc1 ; c1ccc(N(c2ccccc2)c2cccc(-c3cccc(-c4cc5ccc6ccccc6c5c5c4oc4ccccc45)c3)c2)cc1]

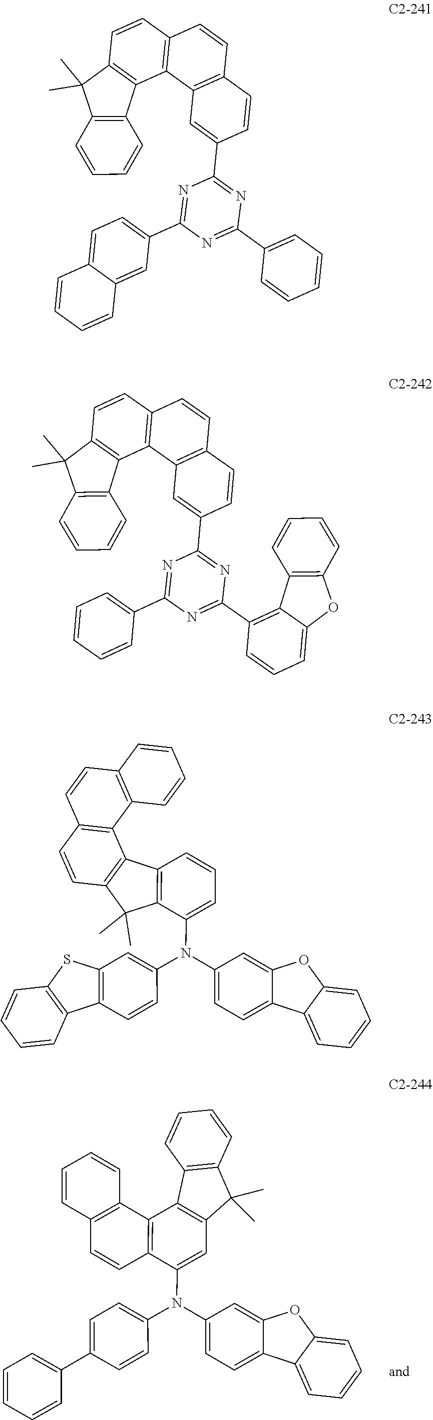 [SMILES: CC1(C)c2ccccc2-c2c1ccc1ccc3ccc(-c4nc(-c5ccccc5)nc(-c5ccc6ccccc6c5)n4)cc3c21 ; CC1(C)c2ccccc2-c2c1ccc1ccc3ccc(-c4nc(-c5ccccc5)nc(-c5cccc6oc7ccccc7c56)n4)cc3c21 ; CC1(C)c2ccc3ccc4ccccc4c3c2-c2cccc(N(c3ccc4c(c3)oc3ccccc34)c3ccc4c(c3)sc3ccccc34)c21 ; CC1(C)c2ccccc2-c2c1cc(N(c1ccc(-c3ccccc3)cc1)c1ccc3c(c1)oc1ccccc13)c1ccc3ccccc3c21]

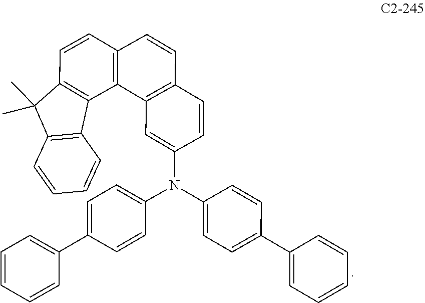 [SMILES: CC1(C)c2ccccc2-c2c1ccc1ccc3ccc(N(c4ccc(-c5ccccc5)cc4)c4ccc(-c5ccccc5)cc4)cc3c21]

8. An organic electroluminescent device comprising: an anode, a cathode, and at least one light-emitting layer between the anode and the cathode, wherein the at least one light-emitting layer comprises the plurality of host materials according to claim 1.

9. An organic electroluminescent compound represented by the following formula 2′: 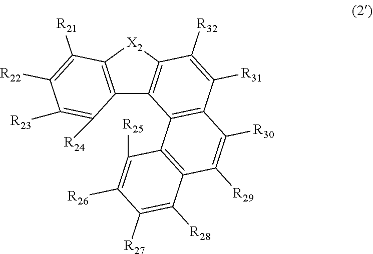
wherein,
X₂ represents O, S, or CR₇R₈;
R₇ and R₈ each independently represent, a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
R₂₁ to R₃₂ each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a fused ring of a substituted or unsubstituted (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-NAr₁Ar₂; provided that at least one of R₂₁ to R₂₂ is *-L₁-NAr₁Ar₂;
L₁ represents a single bond, a substituted or unsubstituted (C10-C30)arylene, or a substituted or unsubstituted (3- to 30-membered)heteroarylene; and
Ar₁ and Ar₂ each independently represent, a substituted or unsubstituted (3- to 30-membered)heteroaryl; provided that the case where all of Ar₁ and Ar₂ are carbazole is excluded.

10. The organic electroluminescent compound according to claim 9, wherein the organic electroluminescent compound represented by the formula 2′ is selected from the following compounds:

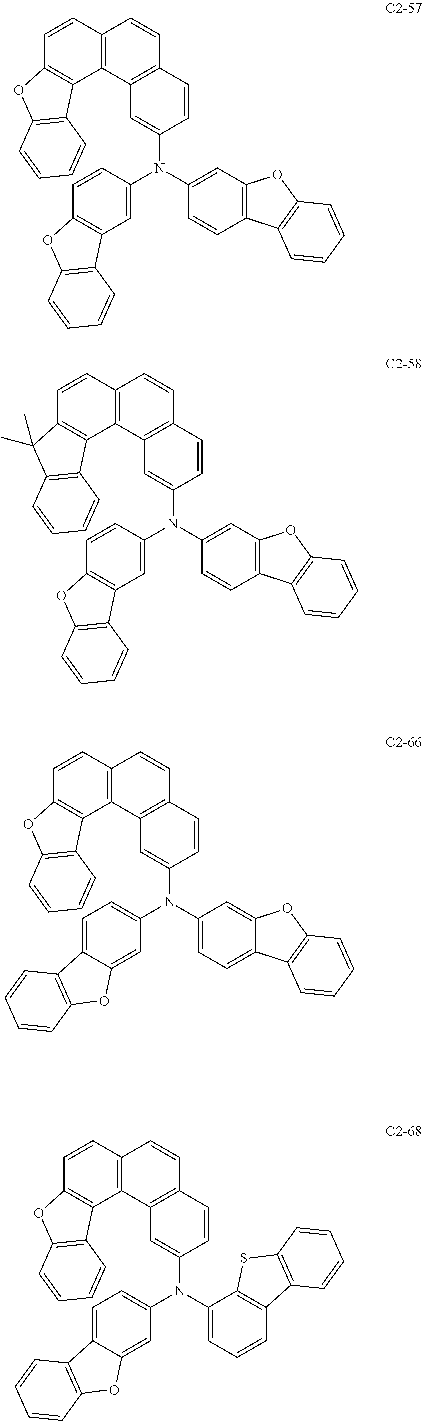 [SMILES: c1ccc2c(c1)oc1cc(N(c3ccc4oc5ccccc5c4c3)c3ccc4ccc5ccc6oc7ccccc7c6c5c4c3)ccc12 ; CC1(C)c2ccccc2-c2c1ccc1ccc3ccc(N(c4ccc5c(c4)oc4ccccc45)c4ccc5oc6ccccc6c5c4)cc3c21 ; c1ccc2c(c1)oc1cc(N(c3ccc4c(c3)oc3ccccc34)c3ccc4ccc5ccc6oc7ccccc7c6c5c4c3)ccc12 ; c1ccc2c(c1)oc1cc(N(c3ccc4ccc5ccc6oc7ccccc7c6c5c4c3)c3cccc4c3sc3ccccc34)ccc12]

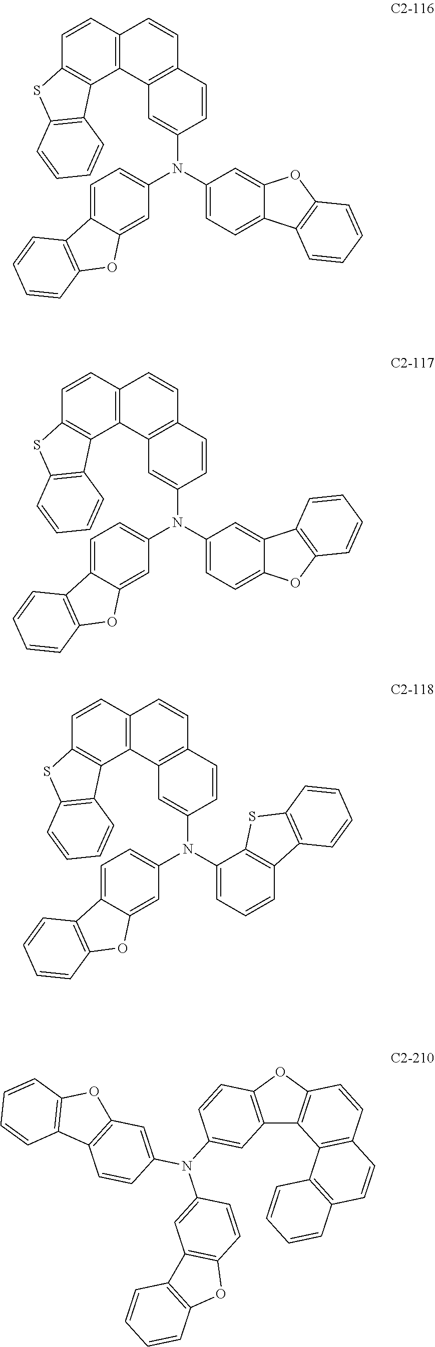 [SMILES: c1ccc2c(c1)oc1cc(N(c3ccc4c(c3)oc3ccccc34)c3ccc4ccc5ccc6sc7ccccc7c6c5c4c3)ccc12 ; c1ccc2c(c1)oc1cc(N(c3ccc4oc5ccccc5c4c3)c3ccc4ccc5ccc6sc7ccccc7c6c5c4c3)ccc12 ; c1ccc2c(c1)oc1cc(N(c3ccc4ccc5ccc6sc7ccccc7c6c5c4c3)c3cccc4c3sc3ccccc34)ccc12 ; c1ccc2c(c1)ccc1ccc3oc4ccc(N(c5ccc6c(c5)oc5ccccc56)c5ccc6oc7ccccc7c6c5)cc4c3c12]

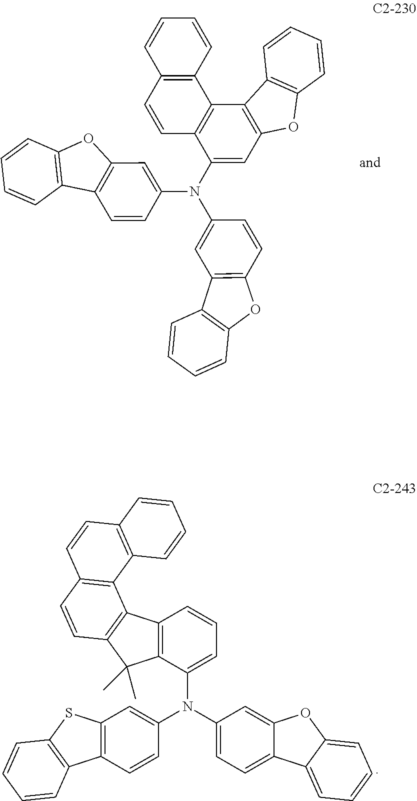 [SMILES: c1ccc2c(c1)ccc1c(N(c3ccc4c(c3)oc3ccccc34)c3ccc4oc5ccccc5c4c3)cc3oc4ccccc4c3c12 ; CC1(C)c2ccc3ccc4ccccc4c3c2-c2cccc(N(c3ccc4c(c3)oc3ccccc34)c3ccc4c(c3)sc3ccccc34)c21]

11. An organic electroluminescent compound represented by the following formula 2″: 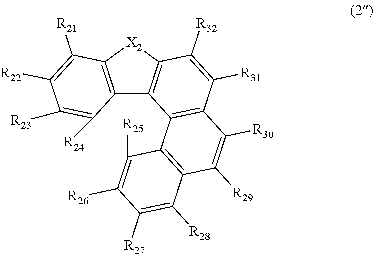
wherein,
X₂ represents O or S;
R₂₁ to R₃₂ each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a fused ring of a substituted or unsubstituted (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₂-HAr, provided that at least one of R₂₁ to R₂₄ and R₂₉ to R₃₂ is *-L₂-HAr;
L₂ represents a single bond, a substituted or unsubstituted (C6-C30)arylene, or a substituted or unsubstituted (3- to 30-membered)heteroarylene;
HAr represents a substituted or unsubstituted triazinyl or a substituted or unsubstituted quinoxalinyl; and
with the proviso that the following compounds are excluded from the organic electroluminescent compound represented by formula 2″.

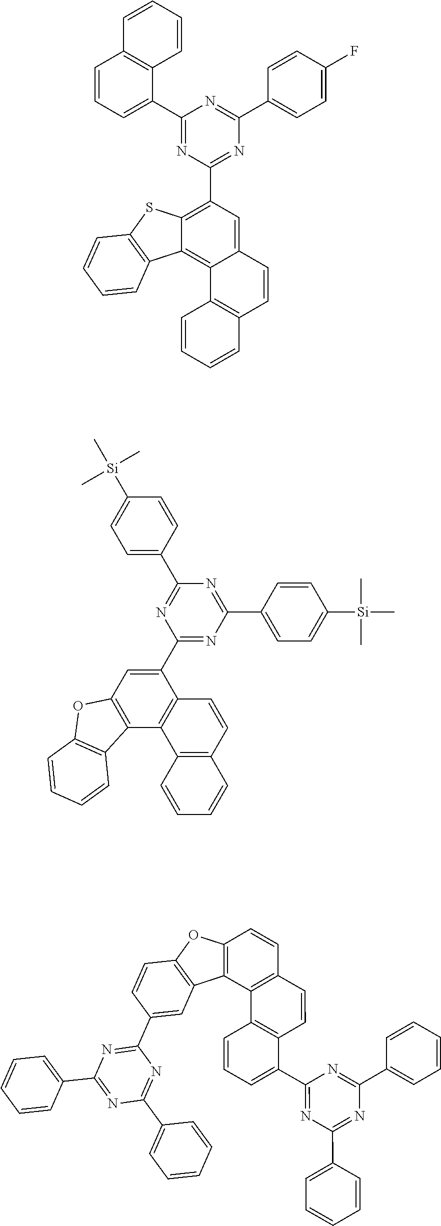 [SMILES: Fc1ccc(-c2nc(-c3cccc4ccccc34)nc(-c3cc4ccc5ccccc5c4c4c3sc3ccccc34)n2)cc1 ; C[Si](C)(C)c1ccc(-c2nc(-c3ccc([Si](C)(C)C)cc3)nc(-c3cc4oc5ccccc5c4c4c3ccc3ccccc34)n2)cc1 ; c1ccc(-c2nc(-c3ccccc3)nc(-c3ccc4oc5ccc6ccc7c(-c8nc(-c9ccccc9)nc(-c9ccccc9)n8)cccc7c6c5c4c3)n2)cc1]

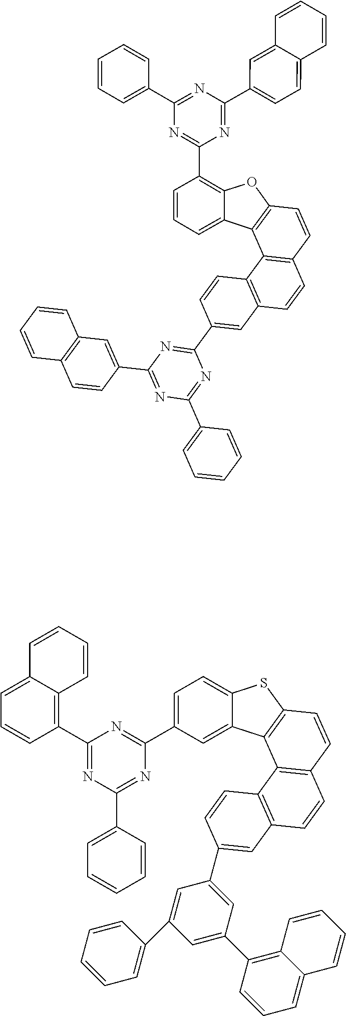 [SMILES: c1ccc(-c2nc(-c3ccc4ccccc4c3)nc(-c3ccc4c(ccc5ccc6oc7c(-c8nc(-c9ccccc9)nc(-c9ccc%10ccccc%10c9)n8)cccc7c6c54)c3)n2)cc1 ; c1ccc(-c2cc(-c3ccc4c(ccc5ccc6sc7ccc(-c8nc(-c9ccccc9)nc(-c9cccc%10ccccc9%10)n8)cc7c6c54)c3)cc(-c3cccc4ccccc34)c2)cc1]

12. The organic electroluminescent compound according to claim 11, wherein the organic electroluminescent compound represented by the formula 2″ is selected from the following compounds:

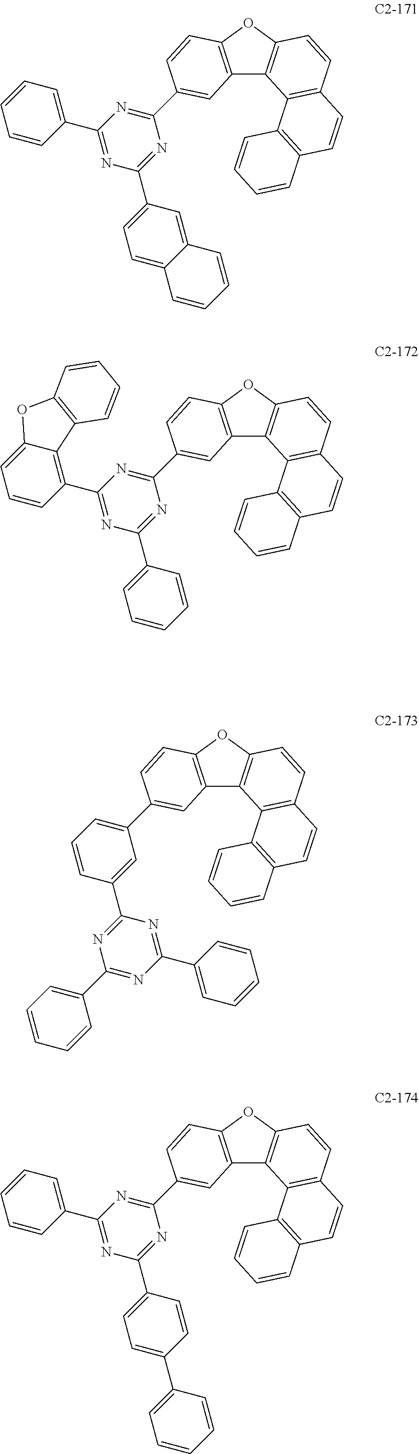 [SMILES: c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1 ; c1ccc(-c2nc(-c3ccccc3)nc(-c3cccc(-c4ccc5oc6ccc7ccc8ccccc8c7c6c5c4)c3)n2)cc1 ; c1ccc(-c2ccc(-c3nc(-c4ccccc4)nc(-c4ccc5oc6ccc7ccc8ccccc8c7c6c5c4)n3)cc2)cc1]

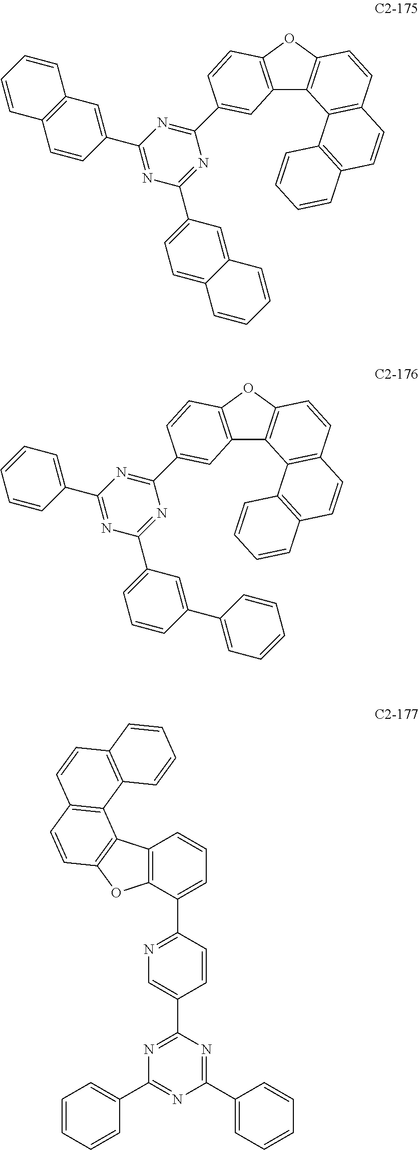 [SMILES: c1ccc2cc(-c3nc(-c4ccc5ccccc5c4)nc(-c4ccc5oc6ccc7ccc8ccccc8c7c6c5c4)n3)ccc2c1 ; c1ccc(-c2cccc(-c3nc(-c4ccccc4)nc(-c4ccc5oc6ccc7ccc8ccccc8c7c6c5c4)n3)c2)cc1 ; c1ccc(-c2nc(-c3ccccc3)nc(-c3ccc(-c4cccc5c4oc4ccc6ccc7ccccc7c6c45)nc3)n2)cc1]

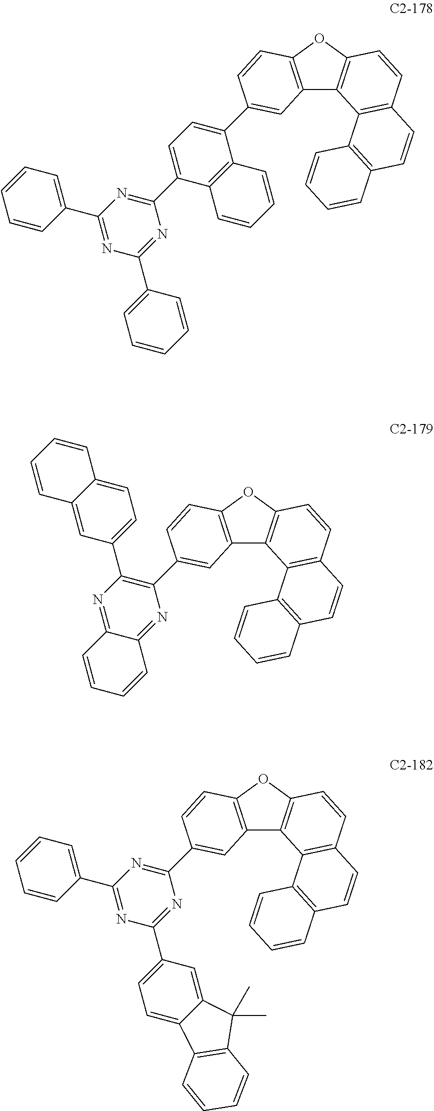 [SMILES: c1ccc(-c2nc(-c3ccccc3)nc(-c3ccc(-c4ccc5oc6ccc7ccc8ccccc8c7c6c5c4)c4ccccc34)n2)cc1 ; c1ccc2cc(-c3nc4ccccc4nc3-c3ccc4oc5ccc6ccc7ccccc7c6c5c4c3)ccc2c1 ; CC1(C)c2ccccc2-c2ccc(-c3nc(-c4ccccc4)nc(-c4ccc5oc6ccc7ccc8ccccc8c7c6c5c4)n3)cc21]

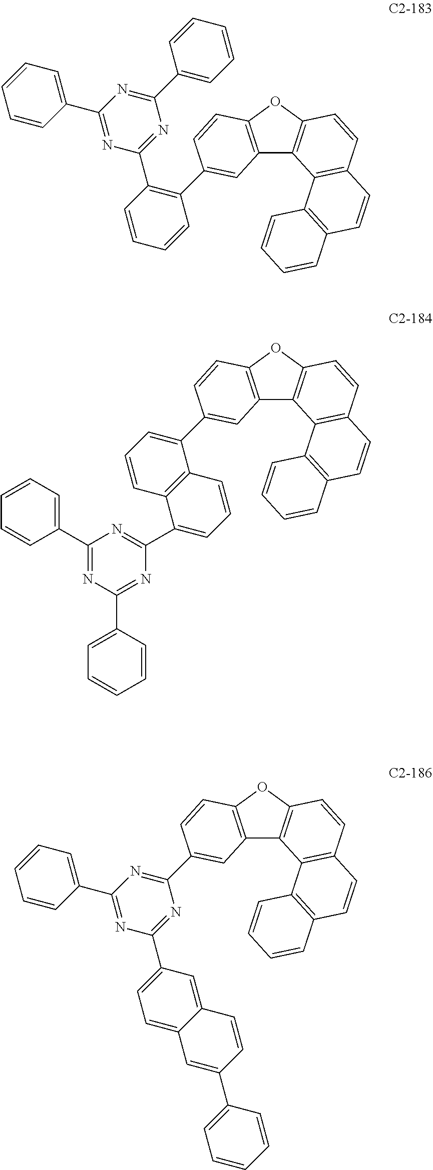 [SMILES: c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1 ; c1ccc(-c2ccc3cc(-c4nc(-c5ccccc5)nc(-c5ccc6oc7ccc8ccc9ccccc9c8c7c6c5)n4)ccc3c2)cc1]

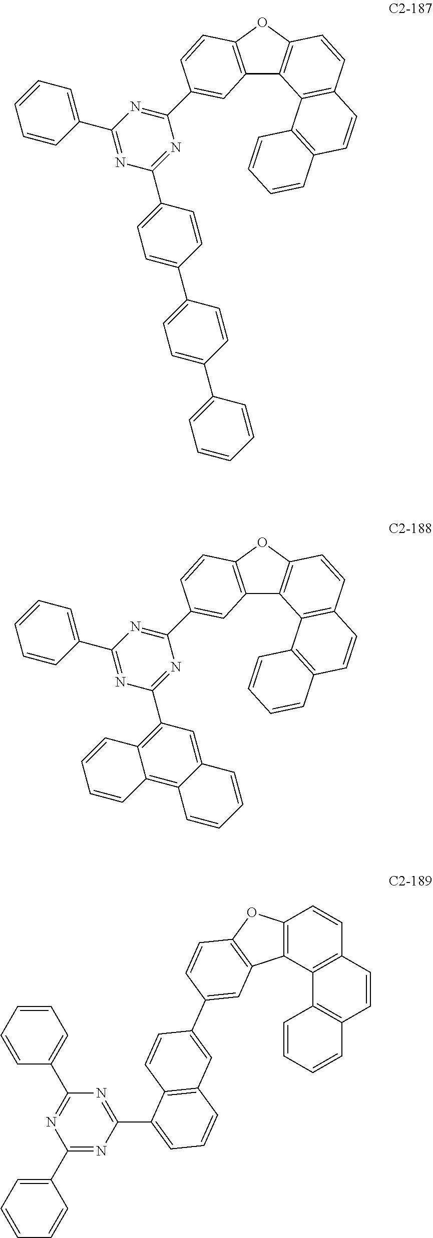 [SMILES: c1ccc(-c2ccc(-c3ccc(-c4nc(-c5ccccc5)nc(-c5ccc6oc7ccc8ccc9ccccc9c8c7c6c5)n4)cc3)cc2)cc1 ; c1ccc(-c2nc(-c3ccc4oc5ccc6ccc7ccccc7c6c5c4c3)nc(-c3cc4ccccc4c4ccccc34)n2)cc1 ; c1ccc(-c2nc(-c3ccccc3)nc(-c3cccc4cc(-c5ccc6oc7ccc8ccc9ccccc9c8c7c6c5)ccc34)n2)cc1]

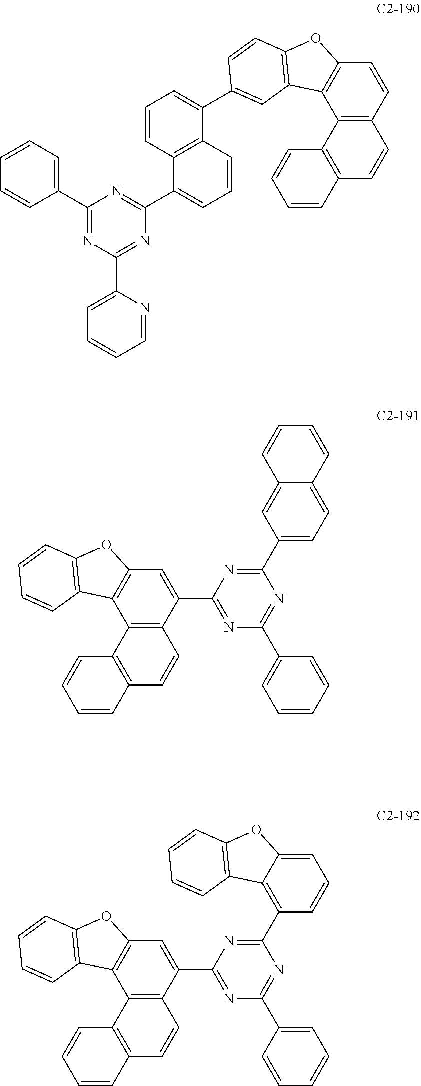 [SMILES: c1ccc(-c2nc(-c3ccccn3)nc(-c3cccc4c(-c5ccc6oc7ccc8ccc9ccccc9c8c7c6c5)cccc34)n2)cc1 ; c1ccc(-c2nc(-c3ccc4ccccc4c3)nc(-c3cc4oc5ccccc5c4c4c3ccc3ccccc34)n2)cc1 ; c1ccc(-c2nc(-c3cc4oc5ccccc5c4c4c3ccc3ccccc34)nc(-c3cccc4oc5ccccc5c34)n2)cc1]

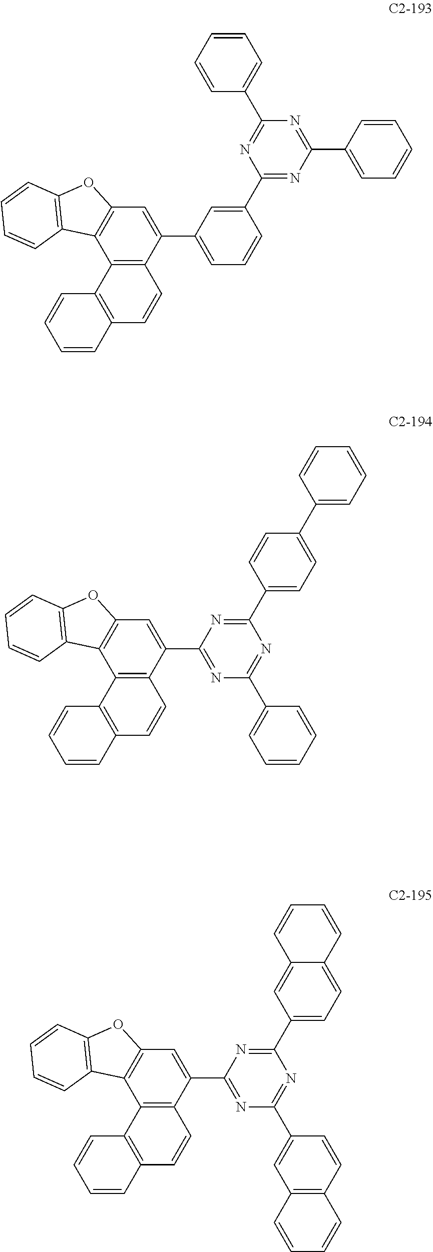 [SMILES: c1ccc(-c2nc(-c3ccccc3)nc(-c3cccc(-c4cc5oc6ccccc6c5c5c4ccc4ccccc45)c3)n2)cc1 ; c1ccc(-c2ccc(-c3nc(-c4ccccc4)nc(-c4cc5oc6ccccc6c5c5c4ccc4ccccc45)n3)cc2)cc1 ; c1ccc2cc(-c3nc(-c4ccc5ccccc5c4)nc(-c4cc5oc6ccccc6c5c5c4ccc4ccccc45)n3)ccc2c1]

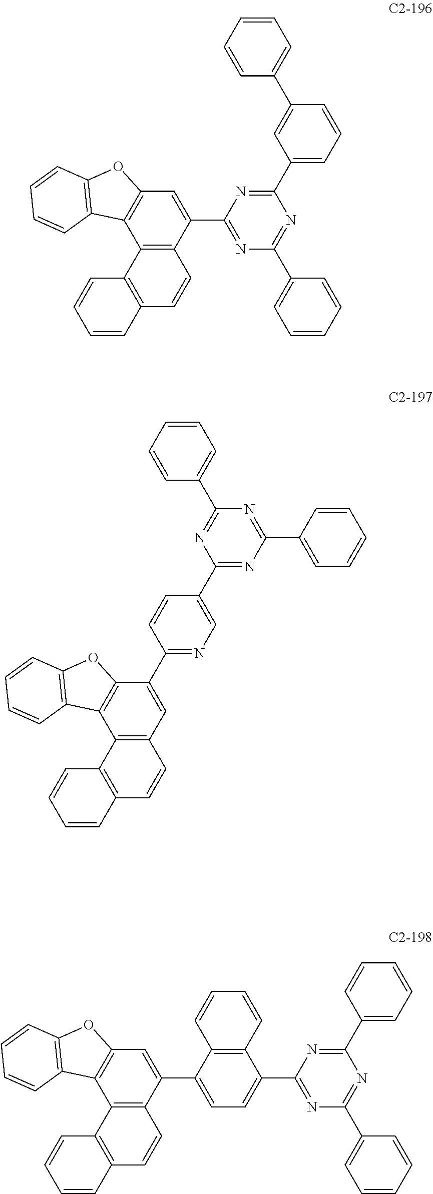 [SMILES: c1ccc(-c2cccc(-c3nc(-c4ccccc4)nc(-c4cc5oc6ccccc6c5c5c4ccc4ccccc45)n3)c2)cc1 ; c1ccc(-c2nc(-c3ccccc3)nc(-c3ccc(-c4cc5ccc6ccccc6c5c5c4oc4ccccc45)nc3)n2)cc1 ; c1ccc(-c2nc(-c3ccccc3)nc(-c3ccc(-c4cc5oc6ccccc6c5c5c4ccc4ccccc45)c4ccccc34)n2)cc1]

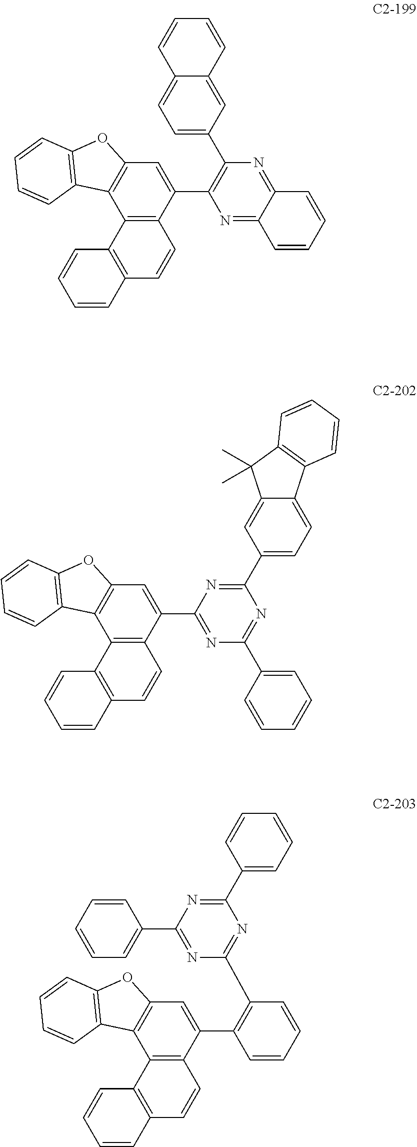 [SMILES: c1ccc2cc(-c3nc4ccccc4nc3-c3cc4oc5ccccc5c4c4c3ccc3ccccc34)ccc2c1 ; CC1(C)c2ccccc2-c2ccc(-c3nc(-c4ccccc4)nc(-c4cc5oc6ccccc6c5c5c4ccc4ccccc45)n3)cc21 ; c1ccc(-c2nc(-c3ccccc3)nc(-c3ccccc3-c3cc4oc5ccccc5c4c4c3ccc3ccccc34)n2)cc1]

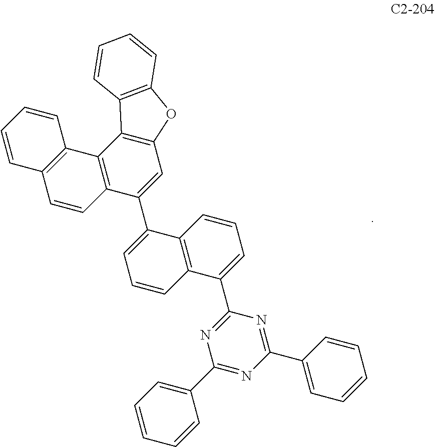 [SMILES: c1ccc(-c2nc(-c3ccccc3)nc(-c3cccc4c(-c5cc6oc7ccccc7c6c6c5ccc5ccccc56)cccc34)n2)cc1]

13. An organic electroluminescent device comprising an organic electroluminescent compound according to claim 9.

14. An organic electroluminescent device comprising an organic electroluminescent compound according to claim 11.
